(19) United States
(12) Patent Application Publication
(10) Pub. No.: US 2022/0289719 A1
(43) Pub. Date: Sep. 15, 2022

(54) HETEROCYCLIC COMPOUNDS AS MNK INHIBITORS

(71) Applicant: NANJING INNOCARE PHARMA TECH CO., LTD., Nanjing, Jiangsu (CN)

(72) Inventors: Norman Xianglong Kong, Nanjing (CN); Chao Zhou, Nanjing (CN); Zhixiang Zheng, Nanjing (CN)

(21) Appl. No.: 16/621,538

(22) PCT Filed: Jun. 8, 2018
(86) PCT No.: PCT/CN2018/090353

§ 371 Date: Dec. 11, 2019

(30) Foreign Application Priority Data
Jun. 12, 2017 (CN) 201710439265.5

(51) Int. Cl. C07D 403/12; C07D 401/14; C07D 403/14; C07D 471/04; C07D 487/04; A61P 35/00

(52) U.S. Cl. CPC C07D 403/12; C07D 401/14; C07D 403/14; C07D 471/04; C07D 487/04; A61P 35/00

(57) ABSTRACT

The present invention relates to a heterocyclic compound, a pharmaceutical composition containing the same, a preparation method thereof, and a use thereof as a mitogen-activated protein kinase interacting kinase 1 and 2(MNK1/MNK2) inhibitor. The present invention further relates to a method of treating or prevent MNK-mediated diseases, such as cancer. The compound is a heterocyclic compound as shown in Formula (I), or a pharmaceutically acceptable salt, a prodrug, a solvate, a polymorph, an isomer, or a stable isotopic derivative thereof.

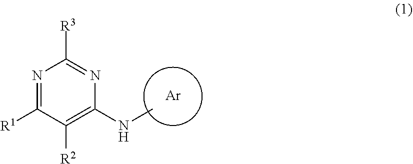

### TECHNICAL FIELD

The present invention relates to a class of heterocyclic compounds, the preparation method thereof, a pharmaceutical composition containing the same, and a use thereof as a MAP kinase-interacting kinase (MNK) inhibitor. The compounds according to the present invention can be used to treat or prevent related diseases mediated by MNK, such as cancer.

### BACKGROUND ART

The mitogen-activated protein kinase-interacting kinase (MNK) belongs to the serine/threonine protein kinase and was first discovered in 1997 as an extracellular regulatory protein kinase (ERK) substrate or binding factor (Waskiewicz A. et al. EMBO J., 1997, 16(8), 1909-1920 & 1921-1933).

Human MNK protein is encoded by two groups of genes, MKNK1 and MKNK2. Each group of genes are translated into two subtypes by selective splicing, namely MNK1a, MNK1b and MNK2a, MNK2b. From the protein sequence analysis, each of these four subtypes contains a nuclear localization signal (NLS) at the N-terminus, and a sequence that binds to eIF4G, which allows MNK kinase to enter the nucleus and function. A kinase domain with high sequence homology therein is responsible for the catalytic function of the kinase. This kinase domain, which belongs to the Ca+/calmodulin-regulated protein kinase (CaMK) family, is not affected by selective splicing and has a high degree of conservation between the four subtypes. The main structural difference is at the C-terminus, MNK1a and MNK2a contain a MAPK domain at the C-terminus, which is responsible for the activation of upstream ERK and p38. Whereas this domain is deleted in the other two subtypes which cannot be phosphorylated and activated by the upstream kinase, therefore, they have different basal activities. MNK1a has a nuclear export signal (NES) at the C-terminus, which allows the MNK1a subtype to be more widely distributed in the cytoplasm, while the other three subtypes are mostly present in nucleus (Diab S. et. al. Chem. Biol. 2014, 21(4), 441-452). eIF4E is the protein which has been studied earliest and most comprehensively in the substrates discovered so far. Through the eIF4E binding domain of N-terminal, MNK1/2 can bind to eIF4E, and then phosphorylates its serine at position 209, and regulates the translation process of related proteins. These proteins play an important role in the mechanisms of tumor cell pro-survival, anti-apoptosis, metastasis and drug resistance. MNK overactivation marked by up-regulation of p-eIF4E level can be detected in prostate cancer, breast cancer, pancreatic cancer, lung cancer, glioma, leukemia, etc. (Lim S. et. al. Proc. Natl. Acad. Sci. USA, 2013, 110(20), 2298-2307; Grzmil. M. et al. J. Clin. Invest., 2014, 124(2), 742-754; Yoshizawa A. et. al. Clin. Cancer Res. 2010, 16 (1), 204-248; Adesso L. et. al. Oncogene, 2012, 32(23), 2848-2857 et al.).

MNK is a kinase which acts downstream of the MAPK pathway, and its pro-survival effect is mainly dependent on the promotion of translation process of tumor-associated proteins. Studies have confirmed that MNK can promote the translation of related mRNA, promote tumor angiogenesis and cell proliferation, and inhibit apoptosis. Recent studies have shown that MNK maintains tumor cell survival in diffuse large B-cell lymphoma (DLBCL), and inhibition of MNK can not only block eIF4E1 phosphorylation, but also enhance eIF4E3 expression (Landon A. et. al. Nat. Commun. 2014, 5, 5413.).

Studies of molecular mechanisms have indicated that MNK-mediated up-regulation of eIF4E phosphorylation promotes translation of Snail and MMP-3 proteins, induces epithelial-mesenchymal transition (EMT), and thereby promotes tumor metastasis. The inhibition of MNK and eIF4E phosphorylation mediated by MNK is expected to be an effective solution for tumor metastasis (Robichaud N. et. al. Oncogene, 2014, 34(16), 2032-2042).

Studies have confirmed that MNK kinase plays a role in multiple drug-induced compensatory pathways, which ultimately leads to drug resistance. The resistance to mTOR inhibitor rapamycin and its analogues is related to MNK. The combination of MNK inhibitor and rapamycin can overcome this drug resistance pathway and produce a synergistic effect, effectively block the translation level of related proteins and inhibit the proliferation of tumor cells to give a better anti-tumor effect. Studies have indicated that the resistances to other chemotherapeutic drugs, such as imatinib, cytarabine, gemcitabine, etc. are all associated with MNK and eIF4E phosphorylation levels. The combination of these drugs with MNK inhibitors may effectively reverse the drug resistance.

Therefore, the combination of MNK inhibitors with some clinically standard therapeutics is an effective treatment strategy (Adesso L. et. al. Oncogene, 2012, 32(23), 2848-2857; Lim S. et al. Proc. Natl. Acad. Sci. USA, 2013, 110(20), 2298-2307; Altman J. K. et. al. Mol. Pharmacol. 2010, 78(4), 778-784).

Therefore, this invention provides novel MNK inhibitor compounds.

### SUMMARY OF THE INVENTION

The purpose of the present invention is to provide a compound as shown in Formula (I), or an isomer, a prodrug, a solvate, a stable isotopic derivative and a pharmaceutically acceptable salt thereof:

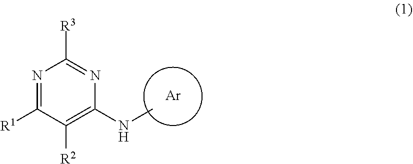

### Where

R¹, R², R³ are each independently selected from the group consisting of hydrogen, halogen, cyano, C1-C8 alkyl, C3-C8 cyclyl, 3-8 membered heterocyclyl, aryl, heteroaryl, aldehyde group, —C(O)R⁴, carboxyl, alkenyl, alkynyl, —OR⁴, —NR⁵R⁶, —OC(O)NR⁵R⁶, —C(O)OR⁴, —C(O)NR⁵R⁶, —NR⁵C(O)R⁴, —NR⁴C(O)NR⁵R⁶, —S(O)ₘR⁴, —NR⁵S(O)ₘR⁴, —SR⁴, —S(O)ₘNR⁵R⁶, —NR⁴S(O)ₘNR⁵R⁶, where said alkyl, cyclyl, hetercyclyl, aryl or heteroaryl are optionally substituted by one or more substituents selected from halogen, cyano, C1-C8 alkyl, C3-C8 cyclyl, 3-8-membered heterocyclyl, —OR⁷, —OC(O)NR⁸R⁹, —C(O)OR⁷, —C(O)NR⁸R⁹, —C(O)R⁷, —NR⁸R⁹, —NR⁸C(O)R⁷, —NR⁷C(O)NR⁸R⁹, —S(O)ₘR⁷, —NR⁸S(O)ₘR⁷, —SR⁷, —S(O)ₘNR⁸R⁹, —NR⁷S(O)ₘNR⁸R⁹;

Wherein ring Ar are each independently selected from substituted or unsubstituted aryl or heteroaryl, when Ar is substituted, it could be substituted by one or more substituents at any position, said substituents are each independently selected from the group consisting of hydrogen, halogen, cyano, C1-C8 alkyl, C3-C8 cyclyl, 3-8 membered heterocyclyl, aryl, heteroaryl, aldehyde group, —C(O)R⁴, carboxyl, alkenyl, alkynyl, OR⁴, —NR⁵R⁶′—NR⁵C(O)R⁴, —NR⁴C(O)NR⁵R⁶, —S(O)ₘR⁴, —NR⁵S(O)ₘR⁴, —SR⁴, —S(O)ₘNR⁵R⁶, —NR⁴S(O)ₘNR⁵R⁶, wherein said alkyl, cyclyl, hetercyclyl, aryl, heteroaryl are optionally substituted by one or more substituents selected from the group consisting of halogen, cyano, C1-C8 alkyl, C3-C8 cyclyl, 3-8 membered heterocyclyl, —OR⁷, —OC(O)NR⁸R⁹, —C(O)OR⁷, —C(O)NR⁸R⁹, —C(O)R⁷, —NR⁸R⁹, —NR₈C(O)R⁸, —NR⁴C(O)NR⁸R⁹, —S(O)ₘR⁷, —NR⁸S(O)ₘR⁷, —SR⁷, —S(O)ₘNR⁸R⁹, —NR⁷S(O)ₘNR⁸R⁹.

R¹ and R² may form a 5-8 membered heterocyclyl together with the carbon atom to which they are attached;

R⁴, R⁵, R⁶, R⁷, R⁸, R⁹ are each independently selected from the group consisting of hydrogen, C1-C8 alkyl, heteroalkyl, C3-C8 cyclyl, 3-8 membered monocyclic heterocyclyl, monocyclic heteroaryl or monocyclic aryl, alkenyl, alkynyl, wherein said R⁵ and R⁶, R⁸ and R⁹ may form a 3-7 membered heterocyclyl; and m is 1 or 2.

In one embodiment of the present invention, a compound as shown in general formula (I), an isomer, a prodrug, a solvate, a stable isotopic derivative thereof or a pharmaceutically acceptable salt thereof is provided, characterized in that the compound s have structures of the following Formula (II);

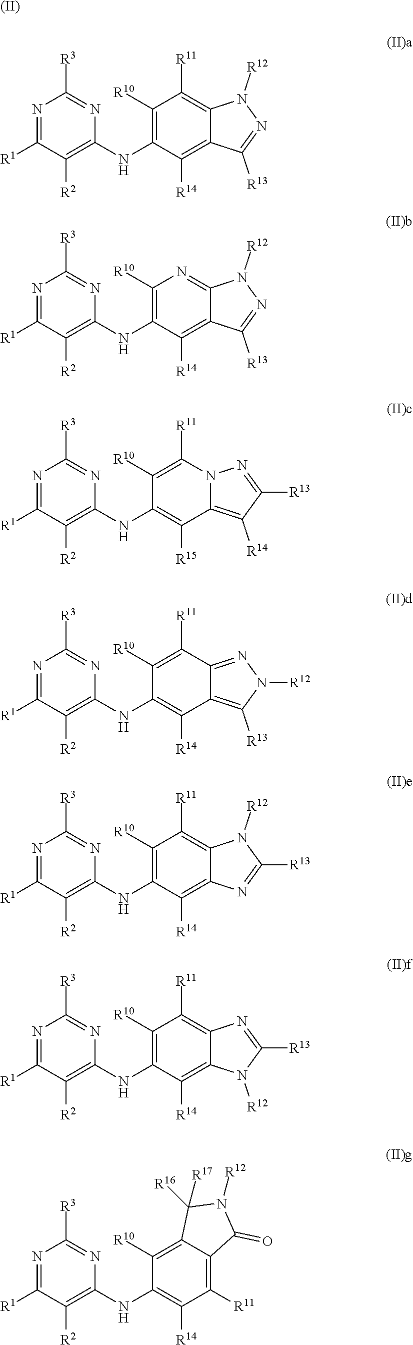

wherein:

R¹, R², R³, R¹⁰, R¹¹, R¹³, R¹⁴, R¹⁵ are each independently selected from the group consisting of hydrogen, halogen, cyano, C1-C8 alkyl, C3-C8 cyclyl, 3-8 membered heterocyclyl, aryl, heteroaryl, aldehyde group, —C(O)R⁴, carboxyl, alkenyl, alkynyl, —OR 4, —NR⁵R⁶, —NR⁵C(O)R⁴, —NR⁴C(O)NR⁵R⁶, —S(O)ₘR⁴, —NR⁵S(O)ₘ, R⁴, —SR⁴, —S(O)ₘ, NR⁵R⁶, —NR⁴S(O)ₘ, NR⁵R⁶, wherein said alkyl, cyclyl, heterocyclyl, aryl, or heteroaryl are optionally substituted by one or more substituents selected from the group consisting of halogen, cyano, C1-C8 alkyl, C3-C8 cyclyl, 3-8 membered heterocyclyl, —OR¹, —OC(O)NR⁸R⁹, —C(O)OR⁷, —C(O)NR⁸R⁹, —C(O)R⁷, —NR⁸R⁹, —NR⁸C(O)R⁷, —NR⁷C(O)NR⁸R⁹, —S(O)ₘR⁷, —NR⁸S(O)ₘR⁷, —SR⁷, —S(O)ₘNR⁸R⁹, —NR⁷S(O)ₘNR⁸R⁹;

R² was selected from the group consisting of hydrogen, C1-C8 alkyl, C3-C8 cyclyl, 3-8 membered monocyclic heterocyclyl, monocyclic heteroaryl or monocyclic aryl, wherein said alkyl, cyclyl, heterocyclyl, aryl or heteroaryl are optionally substituted by one or more substituents selected from the group consisting of halogen, cyano, C1-C8 alkyl, C3-C8 cyclyl, 3-8 membered heterocyclyl, —OR⁷, —OC(O)NR⁸R⁹, —C(O)OR⁷, —C(O)NR⁸R⁹, —C(O)R⁷, —NR⁸R⁹, —NR⁸C(O)R⁷, —NR⁷C(O)NR⁸R⁹, —S(O)ₘR⁷, —NR⁸S(O)ₘR⁷, —SR⁷, —S(O)ₘNR⁸R⁹, —NR⁷S(O)ₘNR⁸R⁹;

R¹⁶, R¹⁷ are each independently selected from the group consisting of hydrogen, C1-C8 alkyl, C3-C8 cyclyl, 3-8 membered monocyclic heterocyclyl, monocyclic heteroaryl or monocyclic aryl, wherein said alkyl, cyclyl, heterocyclyl, aryl or heteroaryl are optionally substituted by one or more substituents selected from the group consisting of halogen, cyano, C1-C8 alkyl, C3-C8 cyclyl, 3-8 membered heterocyclyl, —OR⁷, —OC(O)NR⁸R⁹, —C(O)OR⁷, —C(O)NR⁸R⁹, —C(O)R⁷, —NR⁸R⁹, —NR⁸C(O)R⁷, —NR⁷C(O)NR⁸R⁹, —S(O)ₘR⁷, —NR⁸S(O)ₘR⁷, —SR⁷, —S(O)ₘNR⁸R⁹, —NR⁷S(O)ₘNR⁸R⁹.

R¹ and R² may form a 5-8 membered heterocyclyl with the atom they are attached;

R¹⁰ and R¹¹ may form a 5-8 membered heterocyclyl with the atom they are attached;

R¹⁶ and R¹⁷ may form a 5-8 membered heterocyclyl with the atom they are attached;

The definition of R⁴⁻⁹ are as described above;

And m is 1 or 2.

In another embodiment of the present invention, a compound as shown in general formula (I) or (II), an isomer, a prodrug, a solvate, a stable isotopic derivative thereof or a pharmaceutically acceptable salt thereof is provided, characterized in that the compound has structure as shown in the following Formula (III) a-e

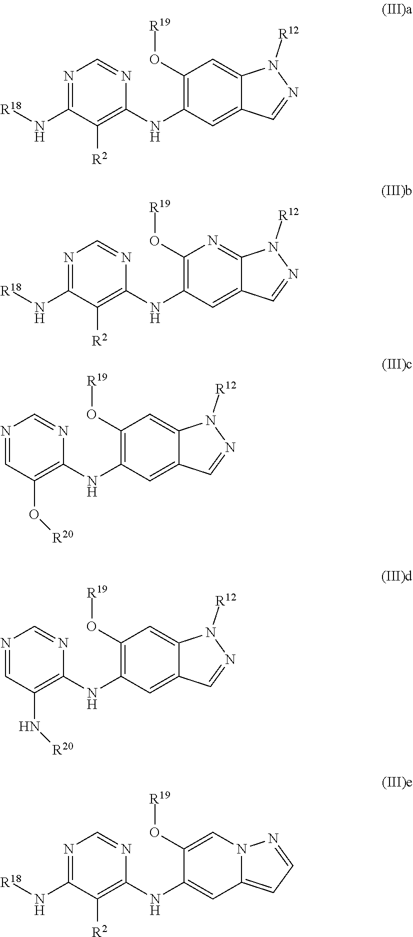

In Formula (III) a-e:

R² is selected from the group consisting of hydrogen, fluoro, cyano, C1-C3 alkyl, C5-C6 cyclyl, 5-6 membered heterocyclyl, aryl, heteroaryl, —C(O)OR⁴, —C(O)NR⁵R⁶, carboxyl, —OR⁴, —NR⁵R⁶, wherein said cyclyl, heterocyclyl are optionally substituted by one or more substituents selected from the group consisting of —C(O)OR⁷, —C(O)NR⁸R⁹; R¹² is selected from hydrogen,

alkoxycarbonyl, alkylcarbonyl, cycloalkylcarbonyl; R¹⁸ is selected from

hydrogen, C1-C5 alkyl, C3-C6 cycloalkyl, aryl, 5-6 membered heteroaryl, alkylcarbonyl, cycloalkylcarbonyl, arylcarbonyl, heteroarylcarbonyl, alkylsulfonyl, cycloalkylsulfonyl; R¹⁹ is selected from C1-C4 alkyl; R²⁰ is selected from C1-C5

alkyl, C1-C5 oxaalkyl, —CH₂CH₂NR⁵R⁶, phenyl;

R² and R¹⁸ may form a nitrogen atom-containing 5-8 membered ring with the carbon and nitrogen atoms they are attached;

R⁴, R⁵, R⁶, R⁷, R⁸, R⁹ are each independently selected from the group consisting of hydrogen, C1-C5 alkyl, C3-C8 cyclyl, 3-8 membered monocyclic heterocyclyl, monocyclic heteroaryl or monocyclic aryl, alkenyl, alkynyl, where said R⁵ and R⁶, R⁸ and R⁹ may form a 3-7 membered heterocyclyl with the nitrogen they are attached.

In another embodiment of the present invention, a compound as shown in general formula (I) or (II), an isomer, a prodrug, a solvate, a stable isotopic derivative thereof or a pharmaceutically acceptable salt thereof is provided, characterized in that the compound has structure as shown in the following Formula (III) f-g

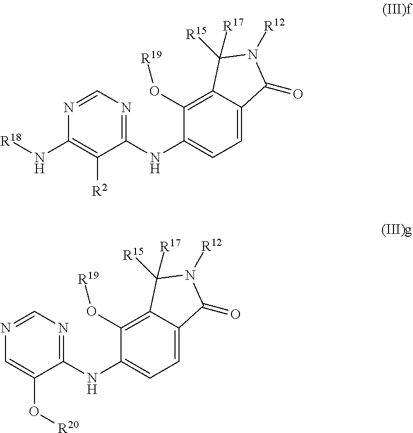

In formula IIIf-g

R² is selected from the group consisting of hydrogen, fluoro, cyano, C1-C3 alkyl, C5-C6 cyclyl, 5-6 membered heterocyclyl, aryl, heteroaryl, —C(O)OR⁴, —C(O)NR⁵R⁶, carboxyl, —OR⁴, —NR⁵R⁶, where said cyclyl, heterocyclyl are optionally substituted by one substituent selected from —C(O)OR⁷, —C(O)NR⁸R⁹;

R¹² is selected from C1-C6 alkyl, C5-C6 cyclyl.

R¹⁶, R¹⁷ are each independently selected from C1-C5 alkyl;

R¹⁸ is selected from hydrogen, C1-C5 alkyl, C3-C6 cycloalkyl, aryl, 5-6 membered heteroaryl, alkylcarbonyl, cycloalkylcarbonyl, arylcarbonyl, heteroarylcarbonyl;

R¹⁹ is selected from C1-C4 alkyl;

R²⁰ is selected from C1-C5 alkyl, C1-C5 oxaalkyl, —CH₂CH₂NR⁵R⁶;

R² and R¹⁸ may form a 5-8 membered heterocyclyl with the atom they are attached;

R¹⁶ and R¹⁷ may form a 4-6 membered ring with the atom they are attached;

R⁴, R⁵, R⁶, R⁷, R⁸, R⁹ are each independently selected from the group consisting of hydrogen, C1-C5 alkyl, C3-C8 cyclyl, 3-8 membered monocyclic heterocyclyl, monocyclic heteroaryl or monocyclic aryl, alkenyl, alkynyl, where said R⁵ and R⁶, R⁸ and R⁹ may form a 3-7 membered heterocyclyl with the nitrogen atom they are attached.

The preferred compounds according to the present invention include, but not limited to

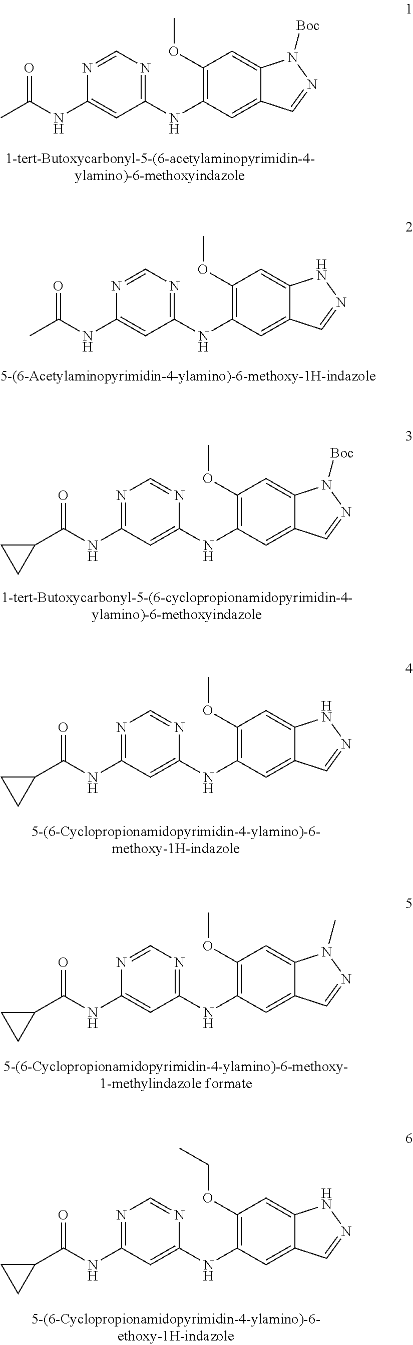 [SMILES: COc1cc2c(cnn2C(=O)OC(C)(C)C)cc1Nc1cc(NC(C)=O)ncn1 ; COc1cc2[nH]ncc2cc1Nc1cc(NC(C)=O)ncn1 ; COc1cc2c(cnn2C(=O)OC(C)(C)C)cc1Nc1cc(NC(=O)C2CC2)ncn1 ; COc1cc2[nH]ncc2cc1Nc1cc(NC(=O)C2CC2)ncn1 ; COc1cc2c(cnn2C)cc1Nc1cc(NC(=O)C2CC2)ncn1 ; CCOc1cc2[nH]ncc2cc1Nc1cc(NC(=O)C2CC2)ncn1]

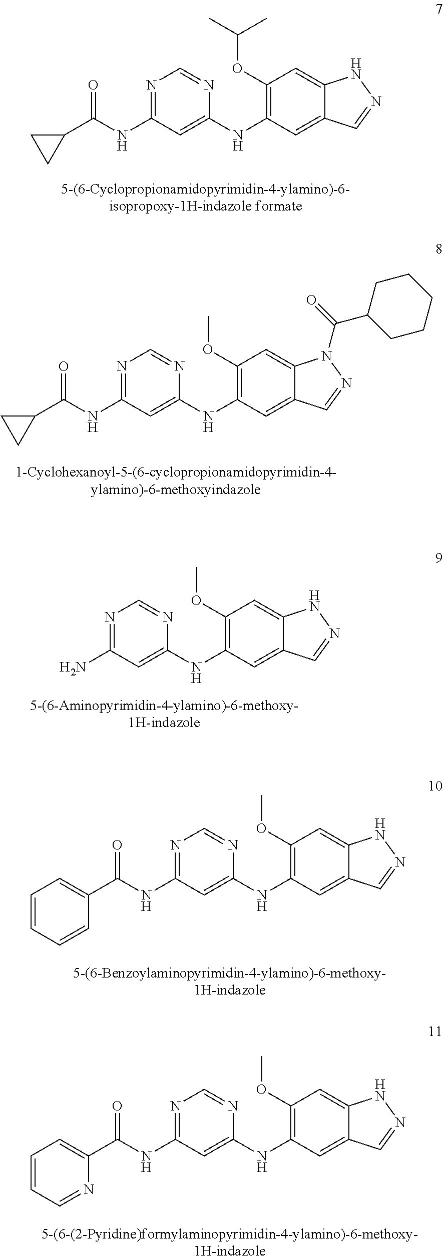 [SMILES: CC(C)Oc1cc2[nH]ncc2cc1Nc1cc(NC(=O)C2CC2)ncn1 ; COc1cc2c(cnn2C(=O)C2CCCCC2)cc1Nc1cc(NC(=O)C2CC2)ncn1 ; COc1cc2[nH]ncc2cc1Nc1cc(N)ncn1 ; COc1cc2[nH]ncc2cc1Nc1cc(NC(=O)c2ccccc2)ncn1 ; COc1cc2[nH]ncc2cc1Nc1cc(NC(=O)c2ccccn2)ncn1]

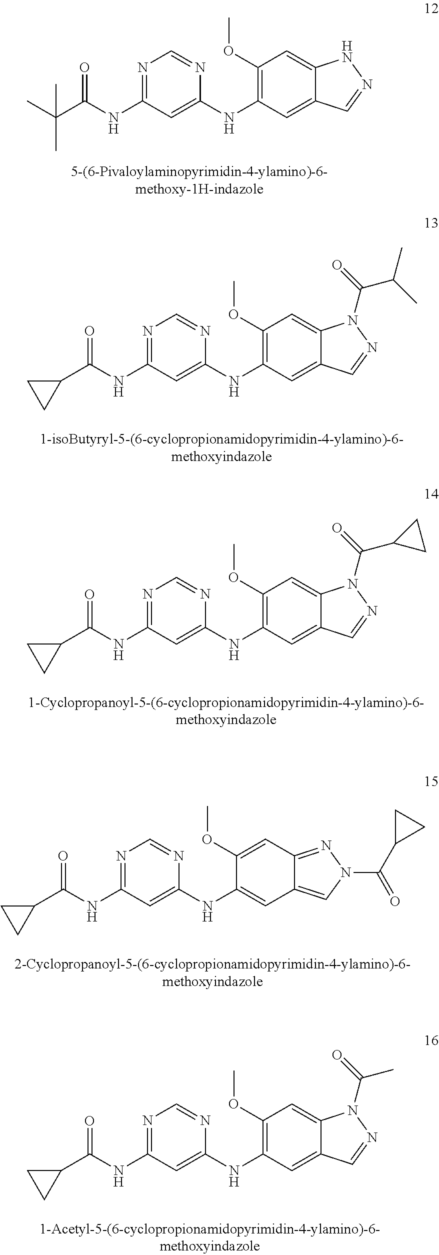 [SMILES: COc1cc2[nH]ncc2cc1Nc1cc(NC(=O)C(C)(C)C)ncn1 ; COc1cc2c(cnn2C(=O)C(C)C)cc1Nc1cc(NC(=O)C2CC2)ncn1 ; COc1cc2c(cnn2C(=O)C2CC2)cc1Nc1cc(NC(=O)C2CC2)ncn1 ; COc1cc2nn(C(=O)C3CC3)cc2cc1Nc1cc(NC(=O)C2CC2)ncn1 ; COc1cc2c(cnn2C(C)=O)cc1Nc1cc(NC(=O)C2CC2)ncn1]

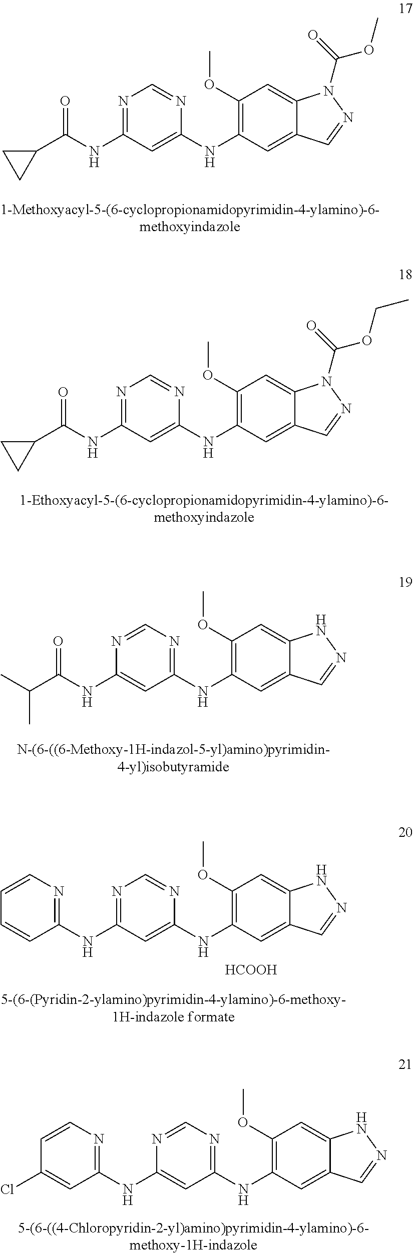

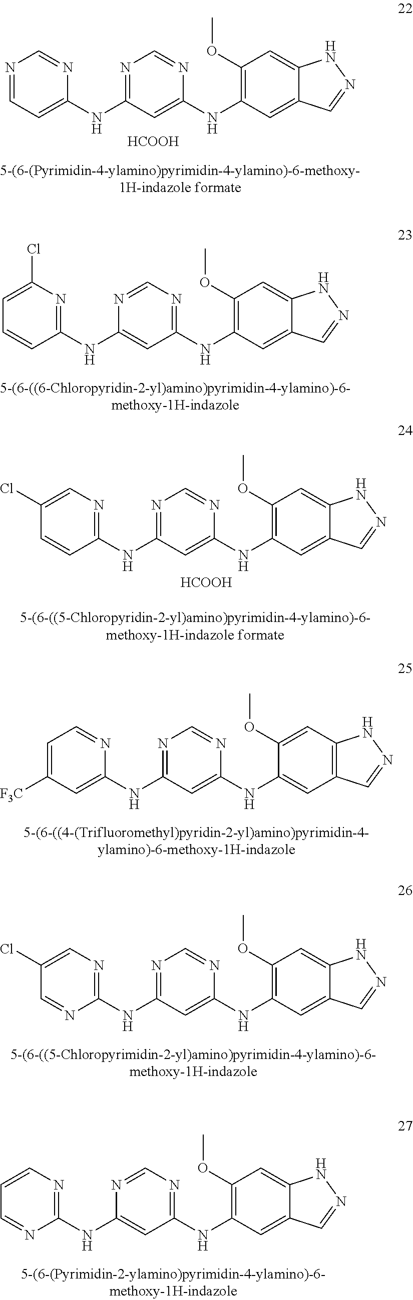

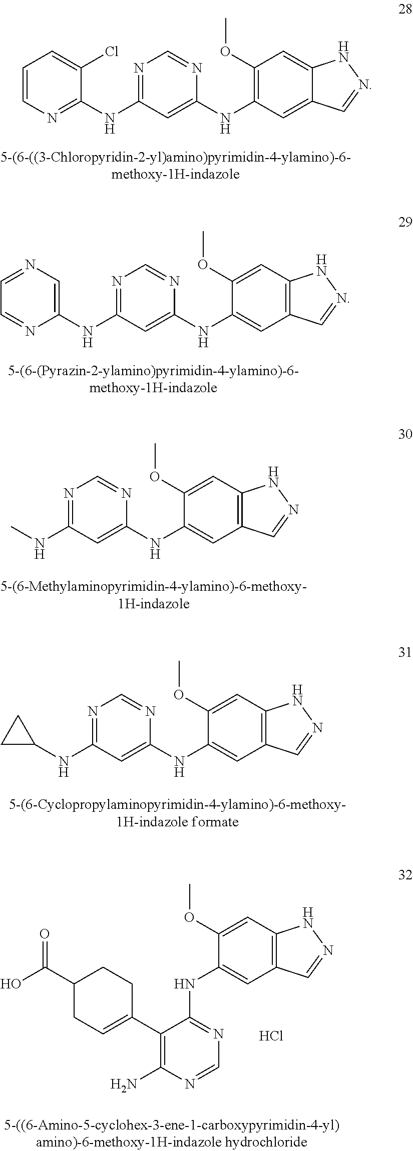 [SMILES: COc1cc2[nH]ncc2cc1Nc1cc(Nc2ncccc2Cl)ncn1 ; COc1cc2[nH]ncc2cc1Nc1cc(Nc2cnccn2)ncn1 ; CNc1cc(Nc2cc3cn[nH]c3cc2OC)ncn1 ; COc1cc2[nH]ncc2cc1Nc1cc(NC2CC2)ncn1 ; COc1cc2[nH]ncc2cc1Nc1ncnc(N)c1C1=CCC(C(=O)O)CC1 ; Cl]

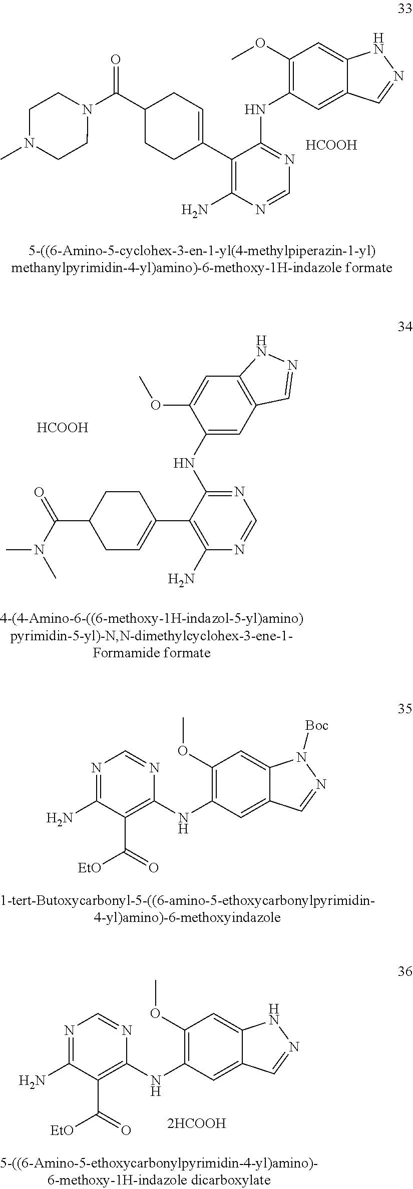 [SMILES: COc1cc2[nH]ncc2cc1Nc1ncnc(N)c1C1=CCC(C(=O)N2CCN(C)CC2)CC1 ; "HCOOH" ; COc1cc2[nH]ncc2cc1Nc1ncnc(N)c1C1=CCC(C(=O)N(C)C)CC1 ; "HCOOH" ; CCOC(=O)c1c(N)ncnc1Nc1cc2cnn(C(=O)OC(C)(C)C)c2cc1OC ; CCOC(=O)c1c(N)ncnc1Nc1cc2cn[nH]c2cc1OC]

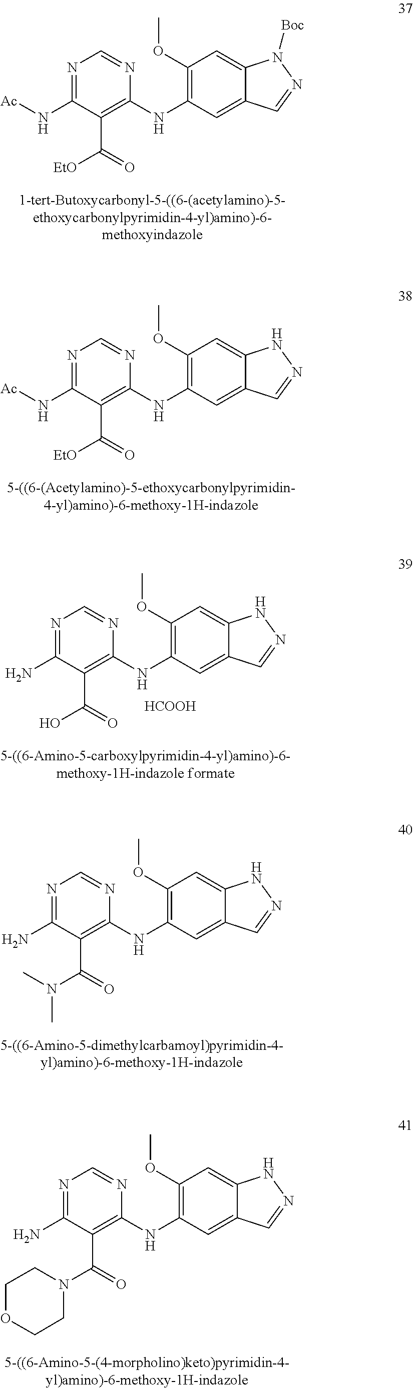 [SMILES: CCOC(=O)c1c(NC(C)=O)ncnc1Nc1cc2cnn(C(=O)OC(C)(C)C)c2cc1OC ; CCOC(=O)c1c(NC(C)=O)ncnc1Nc1cc2cn[nH]c2cc1OC ; COc1cc2[nH]ncc2cc1Nc1ncnc(N)c1C(=O)O ; "HCOOH" ; COc1cc2[nH]ncc2cc1Nc1ncnc(N)c1C(=O)N(C)C ; COc1cc2[nH]ncc2cc1Nc1ncnc(N)c1C(=O)N1CCOCC1]

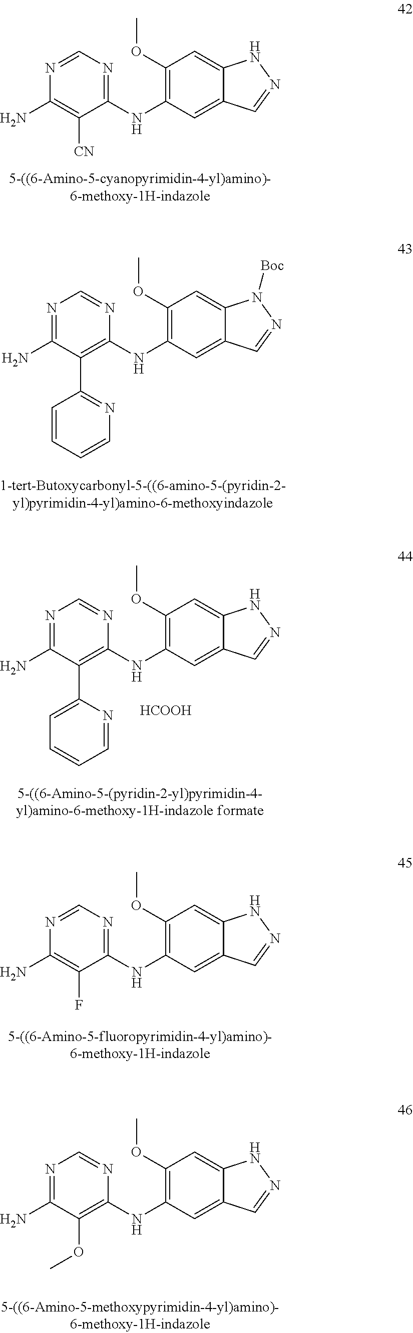 [SMILES: COc1cc2[nH]ncc2cc1Nc1ncnc(N)c1C#N ; COc1cc2c(cnn2C(=O)OC(C)(C)C)cc1Nc1ncnc(N)c1-c1ccccn1 ; COc1cc2[nH]ncc2cc1Nc1ncnc(N)c1-c1ccccn1 ; "HCOOH" ; COc1cc2[nH]ncc2cc1Nc1ncnc(N)c1F ; COc1cc2[nH]ncc2cc1Nc1ncnc(N)c1OC]

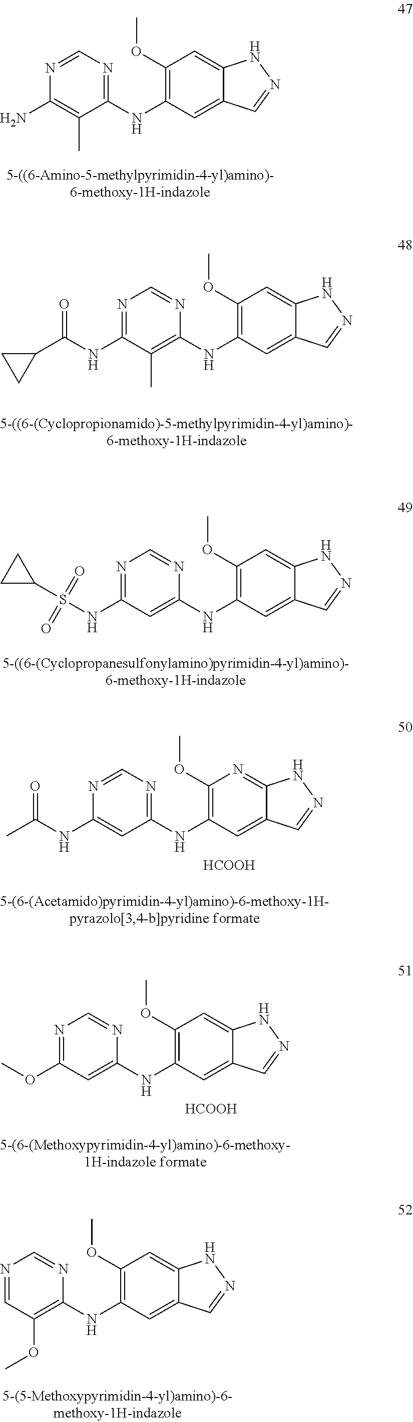

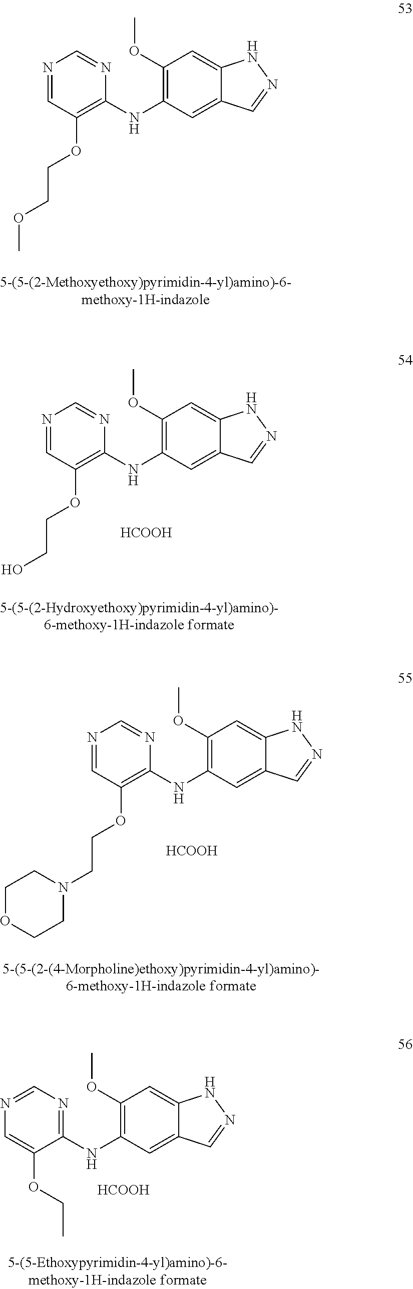

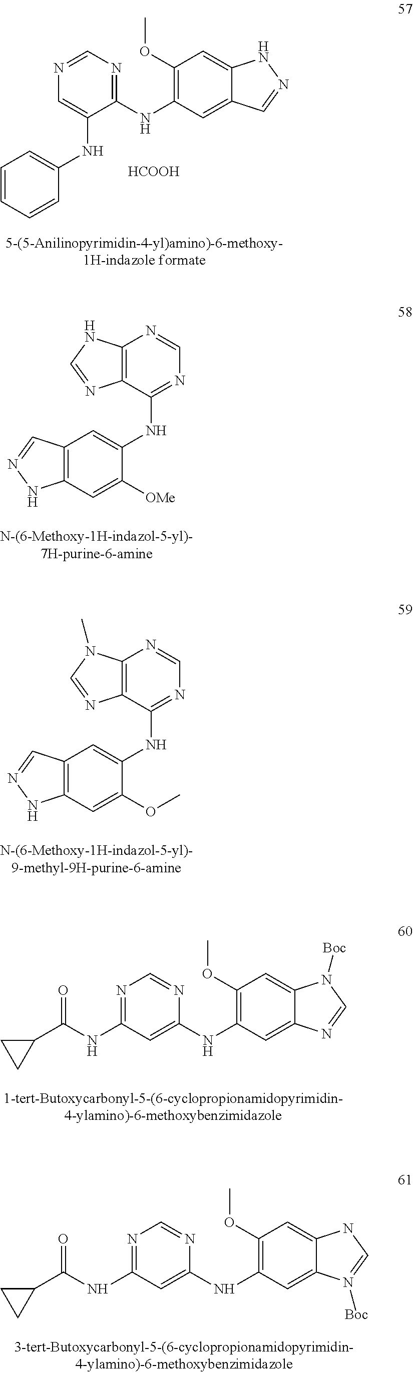

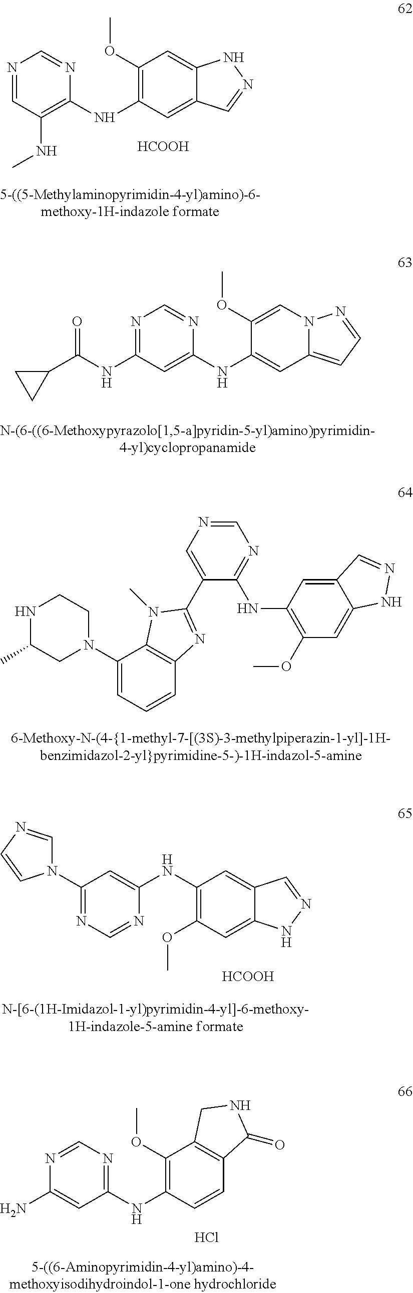

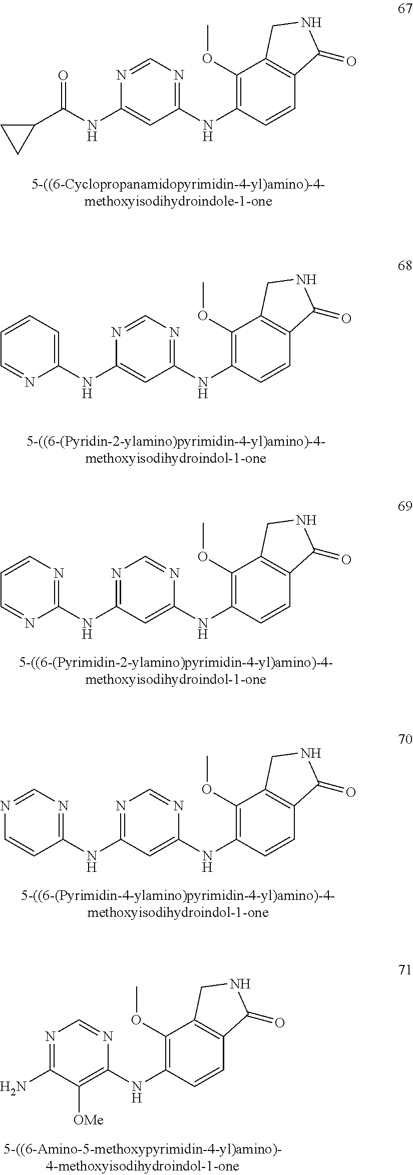 [SMILES: COc1c(Nc2cc(NC(=O)C3CC3)ncn2)ccc2c1CNC2=O ; COc1c(Nc2cc(Nc3ccccn3)ncn2)ccc2c1CNC2=O ; COc1c(Nc2cc(Nc3ncccn3)ncn2)ccc2c1CNC2=O ; COc1c(Nc2cc(Nc3ccncn3)ncn2)ccc2c1CNC2=O ; COc1c(N)ncnc1Nc1ccc2c(c1OC)CNC2=O]

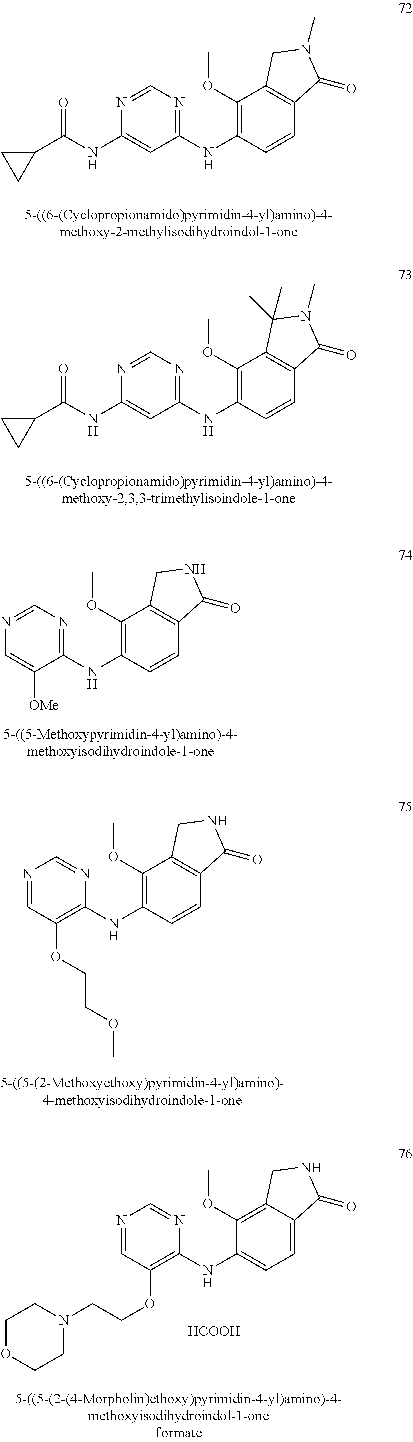 [SMILES: COc1c(Nc2cc(NC(=O)C3CC3)ncn2)ccc2c1CN(C)C2=O ; COc1c(Nc2cc(NC(=O)C3CC3)ncn2)ccc2c1C(C)(C)N(C)C2=O ; COc1cncnc1Nc1ccc2c(c1OC)CNC2=O ; COCCOc1cncnc1Nc1ccc2c(c1OC)CNC2=O ; COc1c(Nc2ncncc2OCCN2CCOCC2)ccc2c1CNC2=O ; "HCOOH"]

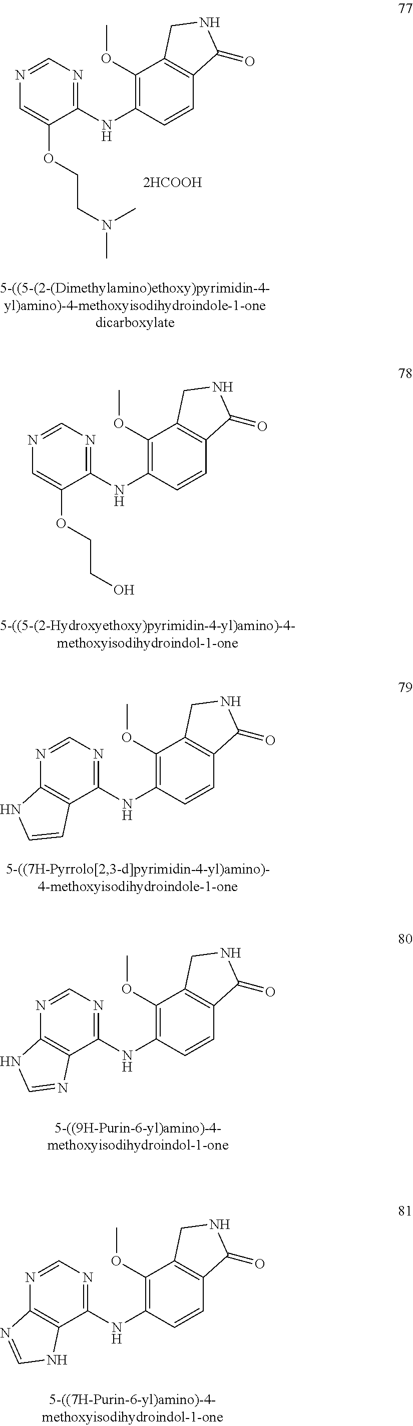 [SMILES: COc1c(Nc2ncncc2OCCN(C)C)ccc2c1CNC2=O ; COc1c(Nc2ncncc2OCCO)ccc2c1CNC2=O ; COc1c(Nc2ncnc3[nH]ccc23)ccc2c1CNC2=O ; COc1c(Nc2ncnc3[nH]cnc23)ccc2c1CNC2=O ; COc1c(Nc2ncnc3nc[nH]c23)ccc2c1CNC2=O]

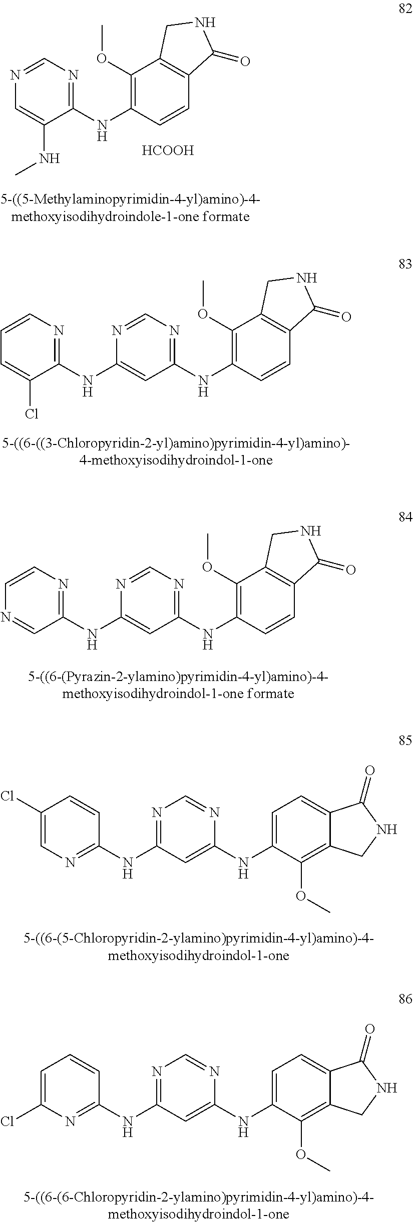 [SMILES: CNc1cncnc1Nc1ccc2c(c1OC)CNC2=O ; "HCOOH" ; COc1c(Nc2cc(Nc3ncccc3Cl)ncn2)ccc2c1CNC2=O ; COc1c(Nc2cc(Nc3cnccn3)ncn2)ccc2c1CNC2=O ; COc1c(Nc2cc(Nc3ccc(Cl)cn3)ncn2)ccc2c1CNC2=O ; COc1c(Nc2cc(Nc3cccc(Cl)n3)ncn2)ccc2c1CNC2=O]

and an isomer, a prodrug, a solvate, a stable isotopic derivative and a pharmaceutically acceptable salt thereof.

The present invention further relates to a pharmaceutical composition comprising a compound of the present invention or an isomer, a prodrug, a stable isotopic derivative or a pharmaceutically acceptable salt thereof, and a pharmaceutically acceptable carrier, diluent and excipient.

In another aspect, the present invention relates to use of the compound of formula (I) or an isomer, a prodrug, a stable isotopic derivative or a pharmaceutically acceptable salt thereof, or the pharmaceutical composition in the manufacture of a medicament, wherein the medicament is used in the treatment or prevention of MNK-mediated diseases, such as tumors, especially malignant hematological diseases, lung cancer, breast cancer, ovarian cancer, prostate cancer, pancreatic cancer, glioma.

In another aspect, the present invention relates to use of the compound of formula (I) or an isomer, a prodrug, a stable isotopic derivative or a pharmaceutically acceptable salt thereof, or the pharmaceutical composition in the manufacture of a medicament for the treatment or prevention of diseases such as cancers and inflammation.

According to the present invention, the medicament can be in any dosage form, including but not limited to tablets, capsules, solution, lyophilized preparation and injection.

The pharmaceutical preparation of the present invention may be administered in the form of a dosage unit containing a predetermined amount of an active ingredient per dosage unit. Such a unit may contain, for example, 0.5 mg to 1 g, preferably 1 mg to 700 mg, particularly preferably 5 mg to 300 mg of a compound of the present invention, or a drug depending on the condition to be treated, the method of administration and the age, weight and condition of the patient, or the pharmaceutical preparation may be administered in the form of a dosage unit containing a predetermined amount of the active ingredient per dosage unit. Preferred dosage unit formulations are those containing the daily or divided dose or active fraction thereof as indicated above. In addition, this type of pharmaceutical preparation can be prepared using methods known in the pharmaceutical art.

The pharmaceutical formulations of the present invention may be suitable for administration by any desired suitable method, such as oral (including oral or sublingual), rectal, nasal, topical (including oral, sublingual or transdermal), vaginal or parenteral (Including subcutaneous, intramuscular, intravenous or intradermal) administration. Such formulations can be prepared using all methods known in the pharmaceutical art, for example, by combining the active ingredient with one or more excipients or one or more adjuvants.

The present invention also relates to a method for treating or preventing MNK-mediated diseases (such as tumors, especially malignant hematological diseases, lung cancer, breast cancer, ovarian cancer, prostate cancer, pancreatic cancer, glioma), comprising administering to a patient in need thereof therapeutically effective amount of a compound of the present invention or an isomer, a prodrug, a solvate, a stable isotope derivative or a pharmaceutically acceptable salt thereof, or a pharmaceutical composition of the present invention.

Another aspect of the present invention relates to a compound of the general formula (I) or an isomer, a prodrug, a solvate, a stable isotope derivative or a pharmaceutically acceptable salt thereof, or a pharmaceutical composition, for use in treating or preventing MNK-mediated diseases, such as tumors, especially malignant hematological diseases, lung cancer, breast cancer, ovarian cancer, prostate cancer, pancreatic cancer, glioma.

Another aspect of the present invention relates to a compound of the general formula (I) or tautomers, mesomers, racemates, enantiomers, diastereomers, mixtures thereof, and pharmaceutically acceptable salts thereof for use in treating and/or preventing diseases such as cancer and so on.

### Preparation Schemes

The present invention further provides methods for preparing the Compounds.

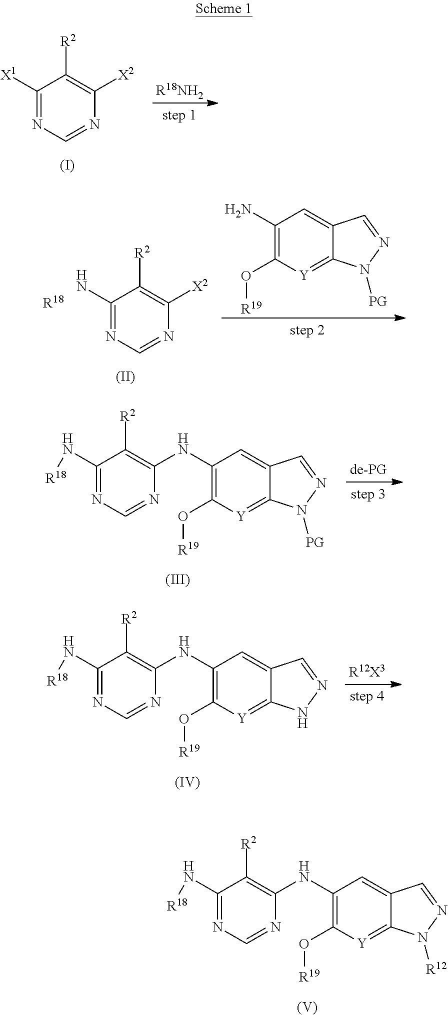

The definitions of R¹, R², R¹², R¹⁸, R¹⁹ are the same as described before.

Step 1:

X¹, X² are leaving groups such as halogen (Cl, Br, I), OTf, OTs, OMs etc.

The Buchwald reaction is carried out in 1,4-dioxane, N,N-dimethylacetamide or the like. Cesium carbonate or sodium t-butoxide is added as a base. The catalyst used is tris(dibenzylideneacetone) di-palladium or palladium acetate. The ligand used is 4,5-bisdiphenylphosphino-9,9-dimethyloxaxene or 2-(dicyclohexylphosphine)-3,6-dimethoxy-2′,4′,6′-triisopropyl-1,1′-biphenyl, etc. The reaction is carried out in microwave or oil bath at 110-150° C. The reaction gives compound (II);

Step 2:

X² is a leaving group such as halogen (Cl, Br, I), OTf, OTs, OMs etc. PG is an amino protecting group such as t-butoxycarbonyl, trimethylsilylethoxymethyl, Y is CH or N. Y and PG are unchanged during the reaction. Buchwald reaction is carried out in 1,4-dioxane, N, N-dimethylacetamide, etc. Cesium carbonate or sodium t-butoxide is added as the base. The catalyst used is tris(dibenzylideneacetone)dipalladium or palladium acetate. The ligand used is 4,5-bis-diphenylphosphino-9,9-dimethyloxaxene or 2-(dicyclohexylphosphine)-3,6-dimethoxy-2′,4′,6′-triisopropyl-1,1′-biphenyl, etc. The reaction is carried out in microwave oven or oil bath at 110 to 150° C. The reaction gives compound (Ill);

Step 3:

Y is unchanged during the reaction. Strong acid such as trifluoroacetic acid/dichloromethane or hydrochloric acid/1, 4-dioxane is employed as the de-protection reagent. The reaction is carried out under room temperature or by heating, PG protection group is removed to give compound (IV).

Step 4:

Acetyl chloride or halide is used in the reaction. Y is unchanged during the reaction. The solvent used is selected from tetrahydrofuran, N,N-dimethylformamide. Base such as cesium carbonate or sodium hydride is added. The reaction is carried out at room temperature or by heating to give compound Scheme 2

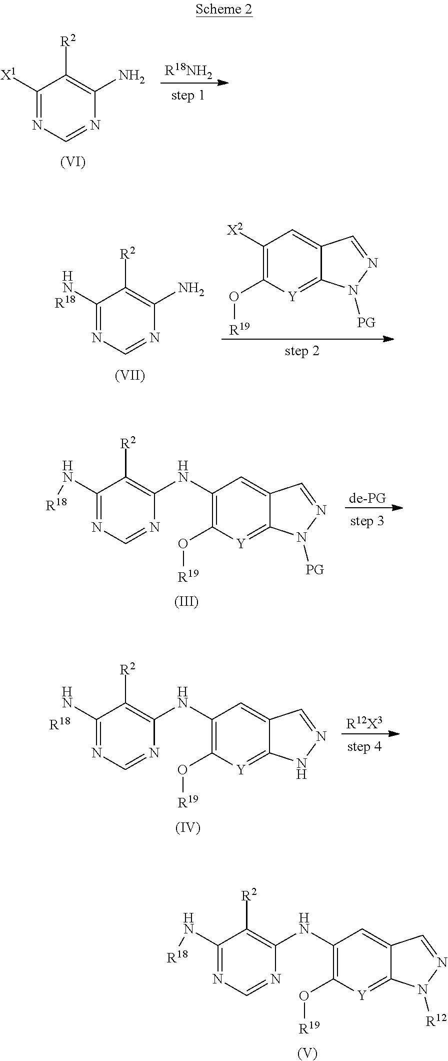

The definitions of R¹, R², R¹², R¹⁸, R¹⁹ are the same as described before.

Step 1:

X¹ is a leaving group such as halogen (Cl, Br or I), OTf, OTs, OMs etc. Buchwald reaction is carried out in 1,4-dioxane or N, N-dimethylacetamide. Base such as cesium carbonate or t-BuONa is added. The catalyst used is tris(dibenzylideneacetone)dipalladium or palladium acetate. The ligand used is 4,5-bisdiphenylphosphino-9,9-dimethyloxaxene or 2-(dicyclohexylphosphine)-3,6-dimethoxy-2′,4′, 6′-triisopropyl-1,1′-biphenyl etc.. The reaction takes place at 110˜150° C. in an oil bath or under microwave conditions to give Compound(VII).

Step 2:

X² is a leaving group such as halogen (Cl, Br, I), OTf, OTs, OMs etc. PG is t-butoxycarbonyl, trimethylsilylethoxymethyl, etc. Y is selected from N or CH. Y and PG are unchanged during the reaction. Buchwald reaction is carried out in 1,4-dioxane or N,N-dimethylacetamide. Cesium carbonate or sodium t-butoxide is added at the same time as a base. The catalyst used was tris(dibenzylideneacetone)dipalladium or palladium acetate. The ligand used is 4,5-bisdiphenylphosphino-9,9-dimethyloxaxene or 2-(dicyclohexyl) phosphine)-3,6-dimethoxy-2′,4′,6′-triisopropyl-1,1′-biphenyl, etc. The reaction takes place at 110˜150° C. in an oil bath or under microwave conditions to give Compound(III).

Step 3:

Y is unchanged during the reaction. Strong acid such as trifluoroacetic acid/dichloromethane or hydrochloric acid/1, 4-dioxane is employed as the de-protection reagent. The reaction is carried out at room temperature or by heating.

The protection group is removed to give compound (IV).

Step 4:

Acetyl chloride or halide is used in the reaction. Y is unchanged during the reaction. The solvent of the reaction is selected from tetrahydrofuran or N,N-dimethylformamide. Base such as cesium carbonate or sodium hydride is added. The reaction is carried out at room temperature or by heating to give compound (V).

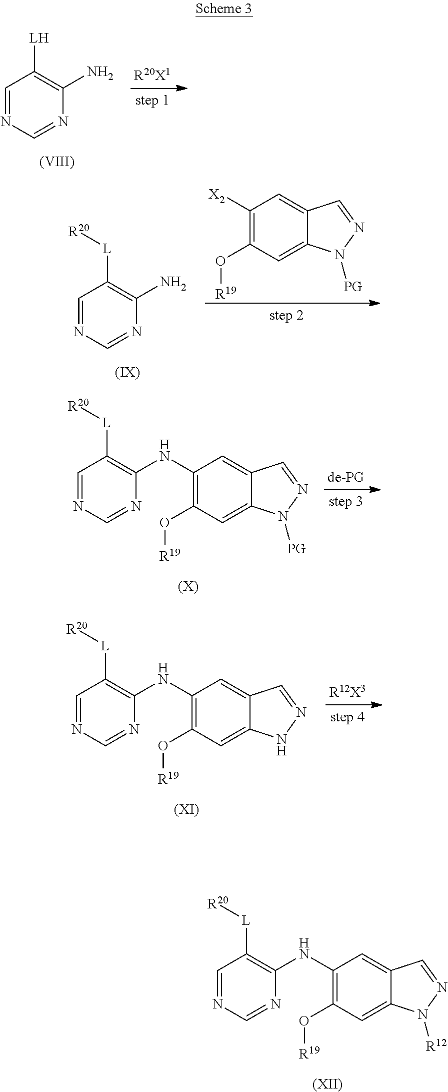

The definitions of R¹², R¹⁹, R²⁰ are the same as described before.

Step 1:

X¹ is a leaving group such as halogen (Cl, Br or I), OTf, OTs or OMs, etc. L is selected from O or NH. The solvent of the reaction is N, N-dimethylformamide.

Base such as lithium hydroxide is added. The reaction takes place at 60° C. to give compound (IX).

Step 2:

X² is a leaving group such as halogen (Cl, Br, I), OTf, OTs or OMs etc. L is selected from O or NH. PG is t-butoxycarbonyl, trimethylsilylethoxymethyl, etc. L and PG are unchanged during the reaction. Buchwald reaction is carried out in such as 1,4-dioxane or N,N-dimethylacetamide. At the same time, cesium carbonate or sodium t-butoxide is added as a base, and the catalyst used is tris(dibenzylideneacetone)dipalladium or palladium acetate. The ligand used is 4,5-bisdiphenylphosphine-9,9-dimethyl xanthene or 2-(dicyclohexylphosphine)-3,6-dimethoxy-2′,4′,6′-triisopropyl-1,1′-biphenyl, etc. The reaction takes place at 110˜150° C. in an oil bath or under microwave conditions to give compound(X).

Step 3:

L is selected from O or NH. Strong acid such as trifluoroacetic acid/dichloromethane or hydrochloric acid/1, 4-dioxane is employed as the de-protection reagent. L is unchanged during the reaction. The reaction is carried out at room temperature or by heating. Protection group is removed to give compound (XI).

Step 4:

L is selected from O or NH. Corresponding acyl chloride or acyl halide is used in the reaction. L is unchanged during the reaction. The solvent of the reaction is selected from tetrahydrofuran, N,N-dimethylformamide. Base such as cesium carbonate or sodium hydride is added, the reaction is carried out at room temperature or by heating to give compound (XII).

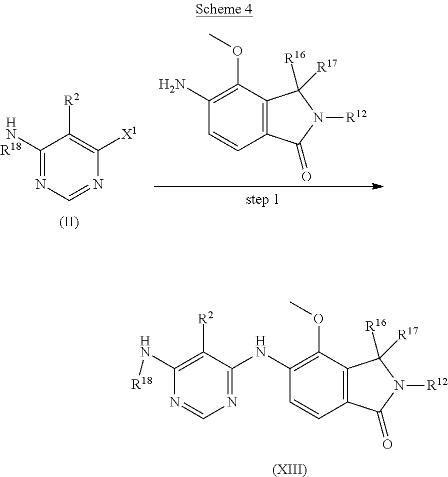

The definitions of R², R¹², R¹⁶, R¹⁷, R¹⁸ are the same as described before.

Step 1:

X¹ is a leaving group such as halogen (Cl, Br, I), OTf, OTs or OMs etc. Buchwald reaction is carried out in 1,4-dioxane or N, N-dimethylacetamide. Base such as cesium carbonate or t-BuONa is added. The catalyst used is tris(dibenzylideneacetone)dipalladium or palladium acetate. The ligand used is 4,5-bisdiphenylphosphino-9,9-dimethyloxaxene or 2-(dicyclohexylphosphine)-3,6-dimethoxy-2′,4′, 6′-triisopropyl-1,1′-biphenyl. The reaction takes place at 110˜150° C. in an oil bath or under microwave conditions to give compound (XIII). When X¹ is Cl, the substitution takes place in hydrochloric acid/1, 4-dioxane solution in a sealed tube at 100° C. to give Compound (XIII).

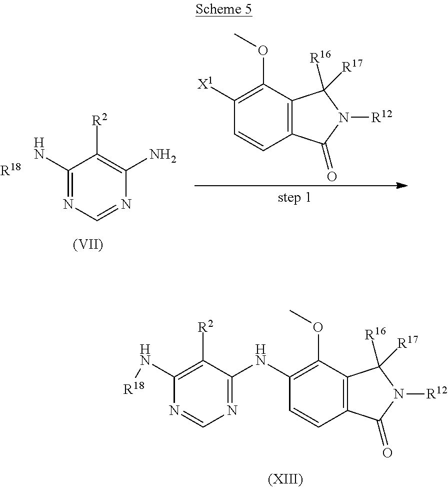

The definitions of R², R¹², R¹⁶-R¹⁸ are the same as described before.

Step 1:

X¹ is a leaving group such as halogen (Cl, Br, I), OTf, OTs, OMs etc. Buchwald reaction is carried out in 1,4-dioxane or N, N-dimethylacetamide. At the same time, cesium carbonate or sodium t-butoxide is added as a base, and the catalyst used is tris(dibenzylideneacetone)dipalladium or palladium acetate. The ligand used is 4,5-bisdiphenylphosphine-9,9-dimethyl xanthene or 2-(dicyclohexylphosphine)-3,6-dimethoxy-2′,4′,6′-triisopropyl-1,1′-biphenyl, etc. The reaction takes place at 80˜110° C. in an oil bath or under microwave conditions to give compound (XIII).

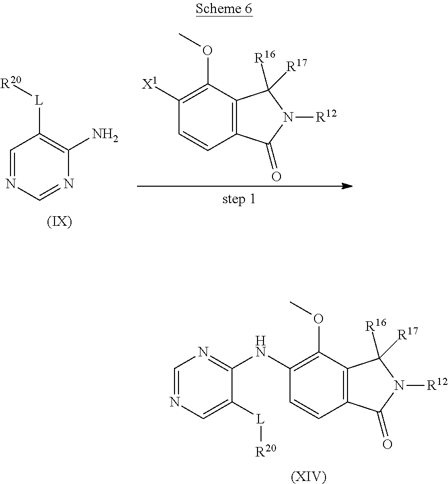

The definitions of R¹², R¹⁶, R¹⁷, R²⁰ are the same as described before.

Step 1:

X¹ is a leaving group such as halogen (Cl, Br, I), OTf, OTs, OMs etc. L is selected from O or NH. Buchwald reaction is carried out in solvents such as 1,4-dioxane or N, N-dimethylacetamide. At the same time, cesium carbonate or sodium t-butoxide is added as a base, and the catalyst used is tris(dibenzylideneacetone)dipalladium or palladium acetate. The ligand used is 4,5-bisdiphenylphosphine-9,9-dimethyl xanthene or 2-(dicyclohexylphosphine)-3,6-dimethoxy-2′,4′,6′-triisopropyl-1,1′-biphenyl, etc. The reaction takes place at 110˜150° C. in an oil bath or under microwave conditions to give Compound (XIV).

### DETAILED DESCRIPTION OF THE INVENTION

### Definition

Unless stated to the contrary, the following terms used in the description and the claims have the following meanings.

The expression “Cx-Cy” as used herein represents the range of the number of carbon atoms, where both x and y are integers. For example, C3-C8 cyclyl represents a cyclyl group having 3 to 8 carbon atoms, and —C0-C2 alkyl represents an alkyl group having 0 to 2 carbon atoms, where —CO alkyl refers to a single chemical bond.

The term “alkyl” refers to a saturated aliphatic hydrocarbon group, including linear and branched groups having 1 to 20 carbon atoms, for example, linear and branched groups having 1 to 18 carbon atoms, 1 to 12 carbon atoms, 1 to 8 carbon atoms, 1 to 6 carbon atoms or 1 to 4 carbon atoms. Non-limiting examples include methyl, ethyl, n-propyl, i-propyl, n-butyl, i-butyl, t-butyl, s-butyl, n-pentyl, 1,1-dimethylpropyl, 1,2-dimethylpropyl, 2,2-dimethyl propyl, 1-ethylpropyl, 2-methylbutyl, 3-methylbutyl, n-hexyl, 1-ethyl-2-methyl propyl, 1,1,2-trimethylpropyl, 1,1-dimethylbutyl, 1,2-dimethylbutyl, 2,2-dimethyl butyl, 1,3-dimethylbutyl, 2-ethylbutyl, and various branched isomers thereof, etc. Alkyl may be substituted or unsubstituted.

The term “cyclyl” or “cyclic group” refers to saturated or partially unsaturated monocyclic or polycyclic hydrocarbon groups, comprising 3 to 12 cyclic carbon atoms, such as 3 to 12, 3 to 10, 3 to 8 or 3 to 6 cyclic carbon atoms, or 3, 4, 5, 6-membered rings. Non-limiting examples of monocyclic cyclyl include cyclopropyl, cyclobutyl, cyclopentyl, cyclopentenyl, cyclohexyl, cyclohexenyl, cyclohexadienyl, cycloheptyl, cycloheptatrienyl, cyclooctyl and the like. Cyclyl may be substituted or unsubstituted.

The term “heterocyclyl” refers to a saturated or partially unsaturated monocyclic or polycyclic hydrocarbon group, comprising 3 to 20 ring atoms, such as 3 to 16, 3 to 12, 3 to 10, 3 to 8 or 3 to 6 ring atoms, where one or more ring atoms are heteroatoms selected from the group consisting of nitrogen, oxygen or S(O)m (where m is an integer of 0 to 2), but excluding ring parts of —O—O—, —O—S— or —S—S—, and the remaining ring atoms are carbon. Preferably 3 to 12 ring atoms, of which 1 to 4 are heteroatoms, are comprised. More preferably the heterocyclyl ring comprises 3 to 10 ring atoms, more preferably 3 to 8 ring atoms. Most preferred are 5-membered rings or 6-membered rings, where 1 to 4 members are heteroatoms, more preferably 1 to 3 are heteroatoms, and most preferably 1 to 2 are heteroatoms. Non-limiting examples of monocyclic heterocyclyl include pyrrolidinyl, piperidyl, piperazinyl, morpholinyl, thiomorpholinyl, homopiperazinyl and the like. Polycyclic heterocyclic groups include spirocyclic, fused and bridged cyclic heterocyclyl groups.

The term “spiroheterocyclic group” refers to a 5 to 20 membered polycyclic heterocyclic group with one atom (referred to as a spiro atom) shared between monocyclic rings, where one or more of the ring atoms are heteroatoms selected from the group consisting of nitrogen, oxygen or S(O)m (where m is an integer of 0 to 2), and the rest of the ring atoms are carbon. They may contain one or more double bonds, but none of the rings has a completely conjugated π electron system. They are preferably 6 to 14 membered, and more preferably 7 to 10 membered. According to the number of spiro atoms shared between rings, spirocyclyl groups are divided into mono-spiroheterocyclyl, bi-spiroheterocyclyl or poly-spiroheterocyclyl, preferably mono-spirocyclyl and bi-spirocyclyl, and more preferably 4 membered/4 membered, 4 membered/5 membered, 4 membered/6 membered, 5 membered/5 membered, or 5 membered/6 membered mono-spirocyclyl. Non-limiting examples of spirocyclyl include

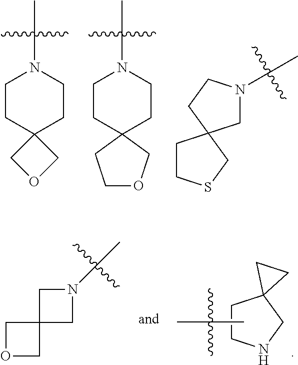

The term “fused heterocyclyl” refers to a 5 to 20 membered polycyclic heterocyclyl group where each ring in the system shares a pair of adjacent atoms with other rings in the system, one or more rings may contain one or more double bonds, but none of the rings has a completely conjugated pi electron system, where one or more ring atoms are heteroatoms selected from the group consisting of nitrogen, oxygen or S(O)m (where m is an integer of 0 to 2), and the remaining ring atoms are carbon. They are preferably 6 to 14 membered, and more preferably 7 to 10 membered. According to the number of rings, they can be divided into bicyclic, tricyclic, tetracyclic or polycyclic fused heterocyclyl, and the fused heterocyclyl groups are preferably bicyclic or tricyclic, and more preferably 5 membered/5 membered, or 5 membered/6 membered bicyclic fused heterocyclyl. Non-limiting examples of fused heterocyclyl include

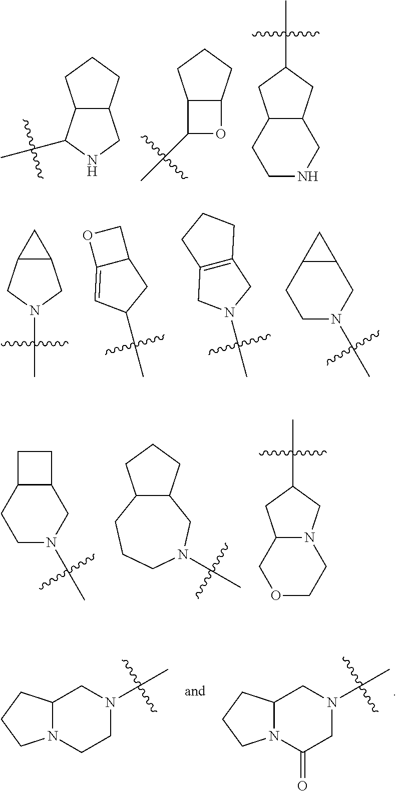

The heterocyclyl ring may be fused to an aryl, a heteroaryl or a cyclyl ring, in which the ring connected with the parent structure is a heterocyclyl group, and the non-limiting examples include:

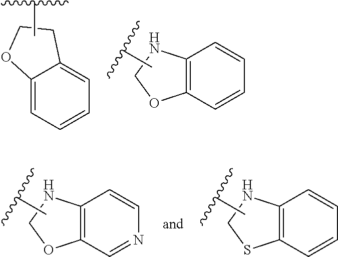

and the like.

The heterocyclyl group may be substituted or unsubstituted.

The term “aryl” refers to a 6 to 14 membered all-carbon monocyclic or condensed polycyclic (i.e., rings sharing adjacent pairs of carbon atoms) group, and a polycyclic (i.e., rings bearing adjacent pairs of carbon atoms) group having a conjugated pi-electron system, preferably 6 to 10 membered, for example, phenyl and naphthyl, and most preferably phenyl. The aryl ring may be fused to a heteroaryl, a heterocyclyl or a cyclyl ring, in which the ring connected with the parent structure is an aryl ring, and the non-limiting examples include:

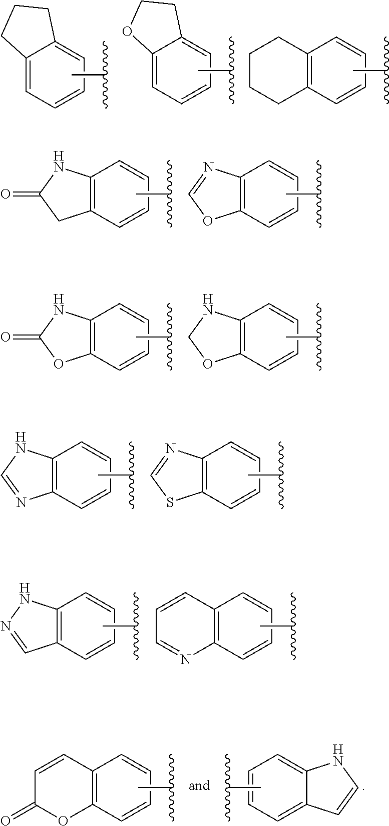

Aryl may be substituted or unsubstituted.

The term “heteroaryl” herein refers to a heteroaromatic system comprising 1 to 4 heteroatoms and 5 to 14 ring atoms, where the heteroatoms include oxygen, sulfur and nitrogen. Heteroaryl is preferably 5 to 10 membered, and more preferably 5 membered or 6 membered, e.g., furyl, thienyl, pyridyl, pyrrolyl, N-alkylpyrrolyl, pyrimidinyl, pyrazinyl, imidazolyl, tetrazyl, oxazolyl, and isoxazolyl etc. The heteroaryl ring can be fused to an aryl, a heterocyclyl or a cyclyl ring, where the ring connected with the parent structure is a heteroaryl ring, and the non-limiting examples include:

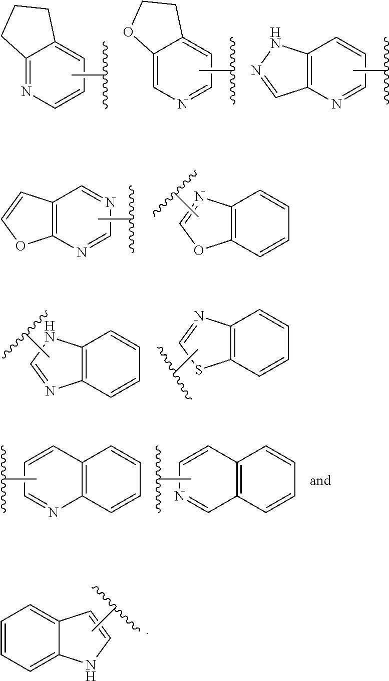

Heteroaryl may be substituted or unsubstituted.

The term “halogen” herein refers to fluorine, chlorine, bromine or iodine.

The term “cyano” herein refers to —CN.

The term “alkenyl” refers to a linear, branched hydrocarbon group containing at least one carbon-carbon double bond, including linear and branched groups having 2 to 20 carbon atoms, for example, linear and branched groups having 2 to 18 carbon atoms, 2 to 12 carbon atoms, 2 to 8 carbon atoms, 2 to 6 carbon atoms or 2 to 4 carbon atoms. Where 1 to 3 carbon-carbon double bonds may be present and preferably 1 carbon-carbon double bond may be present. The term “C2-4 alkenyl” refers to alkenyl having 2 to 4 carbon atoms, including vinyl, propenyl, butenyl, 2-methylbutenyl. The alkenyl group may optionally be substituted.

The term “alkynyl” refers to a linear, or branched hydrocarbon group containing at least one carbon-carbon triple bond, including linear and branched groups having 2 to 20 carbon atoms, for example, linear and branched groups having 2 to 18 carbon atoms, 2 to 12 carbon atoms, 2 to 8 carbon atoms, 2 to 6 carbon atoms or 2 to 4 carbon atoms. Among them, 1 to 3 carbon-carbon triple bonds may be present and preferably 1 carbon-carbon triple bond may be present. The term “C2-4 alkynyl” refers to alkynyl having 2 to 4 carbon atoms, Non-limiting examples including acetenyl, propynyl, butynyl and 3-methyl-1-butynyl.

The term “heteroalkyl” refers to a stable straight-chain or branched-chain hydrocarbon group consisting of a specified number of carbon atoms and at least one heteroatom selected from oxygen, nitrogen and sulfur. Among them, nitrogen and sulfur atoms may be oxidized optionally, nitrogen atoms may be quaternized optionally, and hetero atoms such as oxygen, nitrogen and sulfur may be located at any internal position of the heteroalkyl group, or at the position where the alkyl group is connected with the rest of the molecule. More than two heteroatoms may be independent or continuous.

The term “alkyloxy” refers to the alkyl group connected by an oxygen bridge, comprising alkyloxy group, cyclyloxy and heterocyclyloxy group. Thus, the alkyl in the therm “alkoxy” includes alkyl, heterocyclyl and cyclyl or cyclic group as defined above.

The “optional” and “optionally” means that an event or environment described subsequently may but does not necessarily occur, including cases where the event or environment occurs or does not occur. For example, “heterocyclyl optionally substituted by alkyl” means that alkyl may but does not necessarily exist, including cases where heterocyclyl is substituted by alkyl and not substituted by alkyl.

The term “substituted” means that one or more hydrogen atoms, preferably at most 5 and more preferably 1 to 3 hydrogen atoms, in a group are substituted independently with corresponding number of substituents. It goes without saying that, substituents are only located in their possible chemical positions, and a person skilled in the art can determine (experimentally or theoretically) possible or impossible substitutions without a lot of efforts. For example, amino or hydroxy groups having free hydrogen may be unstable when combined with carbon atoms having unsaturated (e.g. olefinic) bonds.

The substituent(s) include, but are not limited to, the alkyl, alkenyl, alkynyl, alkoxy, halogen, hydroxyl, amino, cyano and thiol groups.

The term “pharmaceutical composition” represents a mixture of one or more of the compounds described herein or physiologically/pharmaceutically acceptable salts or prodrugs with other chemical components, as well as other components such as physiologically/pharmaceutically acceptable carriers and excipients. An object of the pharmaceutical compositions is to promote the administration of drugs to organisms, facilitate the absorption of active ingredients and thus exert biological activity.

The term “room temperature” refers to 15 to 30° C.

The term “a stable isotopic derivative” includes: derivatives substituted with isotopes, such as derivatives obtained by substituting any hydrogen atom in Formula I with 1 to 5 deuterium atoms, derivatives substituted with isotopes obtained by substituting any carbon atom in Formula I with 1 to 3 carbon-14 atoms, or derivatives substituted with isotopes obtained by substituting any oxygen atom in Formula I with 1 to 3 ¹⁸O atoms.

The “pharmaceutically acceptable salts” as described in the present invention are discussed in Berge, et al., “Pharmaceutically acceptable salts,” J. Pharm. Sci., 66, 1-19 (1977), and it is obvious to pharmaceutical chemists that said salts are essentially non-toxic and can provide desired pharmacokinetic properties, palatability, absorption, distribution, metabolism or excretion, and the like.

The pharmaceutically acceptable salts according to the present invention can be synthesized through a general chemical method.

In general, the preparation of the salts can be achieved by reacting the compounds in the form of free alkalis or acids with equivalent chemical equivalents or excess amounts of acids (inorganic or organic acids) or alkalis in suitable solvents or solvent compositions.

The “prodrug” as described in the present invention refers to a compound that can be converted into an original active compound after being metabolized in vivo. Representatively speaking, prodrugs are inactive substances, or have activity lower than the active parent compounds but can provide convenient operation and dosage or improve metabolic characteristics.

The “isomer” of the present invention means that the compound of Formula (I) according to the present invention may have one or more asymmetric center and may be a racemate, a racemic mixture and a single diastereoisomer. The isomers such as stereoisomers and geometric isomers are all included in the present invention. The geometric isomers include cis- and trans-isomers.

The invention shall include any polymorph of the compound or its salts, as well as any kind of hydrate or other solvate.

### EXAMPLES

The present invention will be further illustrated by means of examples below, but is not indented to limited to the scope of the examples described. In the following examples, experimental methods without specific conditions noted are selected according to conventional methods and conditions or according to product instructions.

The structures of all the compounds according to the present invention can be identified by nuclear magnetic resonance (¹H NMR) and/or mass spectrometric detection (MS).

¹H NMR chemical shift (5) is recorded in PPM (unit: 10⁻⁶ PPM). NMR is carried out by a Bruker AVANCE-400 spectrometer. Appropriate solvents include deuterated chloroform (CDCl₃), deuterated methanol (CD₃OD) and deuterated dimethylsulfoxide (DMSO-d⁶), with tetramethylsilane (TMS) as an internal standard.

The low resolution mass spectrogram (MS) is determined by an Agilent 1260HPLC/6120 mass spectrometer, using Agilent ZORBAX XDB-C18, 4.6×50 mm, 3.5 μm, at a gradient elution condition I: 0:95% solvent A1 and 5% solvent B1, 1-2:5% solvent A1 and 95% solvent B1; 2.01-2.50: 95% solvent A1 and 5% solvent B1. The percentage is the volume percentage of a certain solvent based on the total solvent volume. Solvent A1: 0.01% formic acid aqueous solution; solvent B1: 0.01% formic acid solution in acetonitrile; and the percentage is the volume percentage of a solute based on the solution.

The thin-layer silica gel plate is a Yantai Yellow Sea HSGF254 or Qingdao GF254 silica gel plate. The Yantai Yellow Sea 100-200 or 200-300 mesh silica gel is generally used as the support in the column chromatography.

The prep-HPLC uses the Waters SQD2 Ms guided HPLC, XBridge-C18, 30×150 mm preparation column, 5 um. Method 1: acetonitrile-water (0.2% formic acid), flow rate: 25 ml/min, Method II: acetonitrile-water (0.8% ammonium bicarbonate), flow rate: 25 ml/min.

The known starting raw materials of the present invention can be synthesized by or in accordance with methods known in the art, or can be purchased from companies such as Acros Organics, Aldrich Chemical Company, Accela ChemBio Inc., Shanghai Bide Pharmatech, Shanghai Aladdin Chemistry, Shanghai Meryer Chemistry, Accelerating Chemistry, Energy Chemistry etc.

In the examples, unless otherwise specified, the solvents used in the reaction are all anhydrous solvents, where anhydrous tetrahydrofuran is commercially available tetrahydrofuran, sodium blocks are used as a dehydrant, benzophenone is used as an indicator, the solution is refluxed under the protection of argon until the it maintains a blue violet color, it is distilled and collected, and stored at room temperature under the protection of argon, and the other anhydrous solvents are purchased from Energy Chemistry and Accelerating Chemistry, and the transfer and use of all anhydrous solvents shall be carried out under the protection of argon Unless otherwise specified.

In the examples, the reactions are all carried out under an argon atmosphere or nitrogen atmosphere unless otherwise specified.

The argon atmosphere or nitrogen atmosphere means that the reaction flask is connected to an argon or nitrogen balloon with a volume of about 1 L.

The hydrogen atmosphere means that the reaction flask is connected to a hydrogen balloon with a volume of about 1 L.

In hydrogenation, the reaction is usually vacuumed and filled with hydrogen gas, and this procedure is repeated for 3 times.

The reaction temperature is the room temperature, and the temperature range is from 15° C. to 30° C., unless otherwise specified.

The thin-layer chromatography method (TLC) is employed to monitor the reaction process in the examples. The developer system used in the reaction includes: A, a dichloromethane and methanol system, and B: a petroleum ether and ethyl acetate system, and the ratio by volume of the solvents is adjusted according to the polarity of the compounds.

The eluent system for column chromatography and the developer system for thin-layer chromatography employed in the purification of compounds include: A, a dichloromethane and methanol system, and B: a petroleum ether and ethyl acetate system, and the ratio by volume of the solvents is adjusted according to the polarity of the compounds, and a small amount of triethyl amine and acid or alkaline reagents and the like can also be added for the adjustment.

### Example 1

### 1-tert-Butoxycarbonyl-5-(6-acetylaminopyrimidin-4-ylamino)-6-methoxyindazole

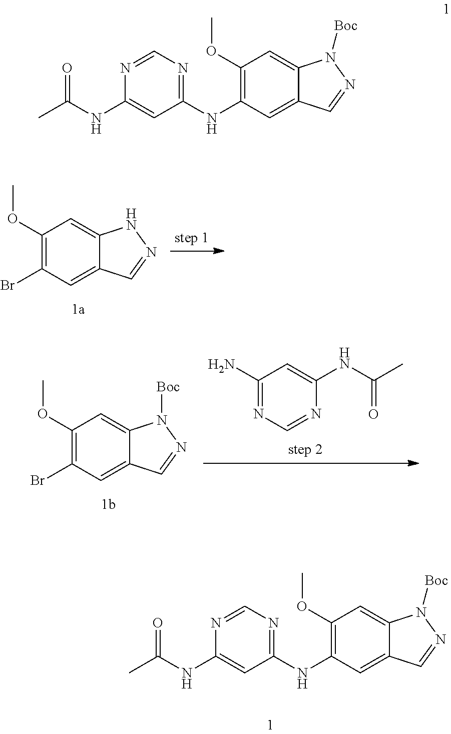 [SMILES: COc1cc2c(cnn2C(=O)OC(C)(C)C)cc1Nc1cc(NC(C)=O)ncn1 ; COc1cc2[nH]ncc2cc1Br ; COc1cc2c(cnn2C(=O)OC(C)(C)C)cc1Br ; CC(=O)Nc1cc(N)ncn1 ; COc1cc2c(cnn2C(=O)OC(C)(C)C)cc1Nc1cc(NC(C)=O)ncn1]

Step 1

### 1-tert-Butoxycarbonyl-5-bromo-6-methoxyindazole

5-bromo-6-methoxy-1H-indazole 1a (4 g, 17.6 mmol), triethylamine (5.3 g, 52.8 mmol), di-t-butyl dicarbonate (7.7 g, 35.2 mmol), 4-dimethylaminopyridine (7.7 g, 35.2 mmol) and tetrahydrofuran (40 mL) were mixed and stirred at room temperature for 1 hour under argon. The mixture was concentrated under reduced pressure. Purification of the residue using column chromatography (petroleum ether/ethyl acetate=4/1) gave 1-tert-butoxycarbonyl-5-bromo-6-methoxyindazole 1b (2.8 g, 8.59 mmol, yellow solid). Yield: 49%.

MS m/z (ESI): 327 & 329 [M+1],

Step 2

### 1-tert-Butoxycarbonyl-5-(6-acetamidopyrimidin-4-ylamino)-6-methoxyindazole

To the mixture of 1-tert-butoxycarbonyl-5-bromo-6-methoxyindazole 1b (24.0 mg, 0.08 mmol), N-(6-aminopyrimidin-4-yl)acetamide (12.0 mg, 0.08 mmol) and 1,4-dioxane (2.0 mL) were added tris(dibenzylideneacetone)dipalladium (8.0 mg, 0.008 mmol), 4,5-bis(diphenylphosphino)-9,9-dimethylxanthene (10.0 mg, 0.016 mmol) and cesium carbonate (52.0 mg, 0.16 mmol) at room temperature under argon. The mixture was stirred at 110° C. in microwave oven for 1 hour under argon, diluted with dichloromethane (10 mL) and filtered. The filtrate was concentrated under reduced pressure. Purification of the residue using Prep TLC (dichloromethane/methanol=20/1) gavel-tert-butoxycarbonyl-5-(6-acetamidopyrimidin-4-ylamino)-6-methoxyindazole 1 (10 mg, 0.025 mmol, yellow solid). Yield: 31%.

MS m/z (ESI): 399 [M+1],

¹H NMR (400 MHz, CDCl₃) δ 8.61 (s, 1H), 8.42 (s, 1H), 8.39 (s, 1H), 8.03 (s, 1H), 7.69 (s, 1H), 7.55 (s, 1H), 7.38 (s, 1H), 3.98 (s, 3H), 2.15 (s, 3H), 1.66 (s, 9H).

### Example 2

### 5-(6-Acetylaminopyrimidin-4-ylamino)-6-methoxy-1H-indazole

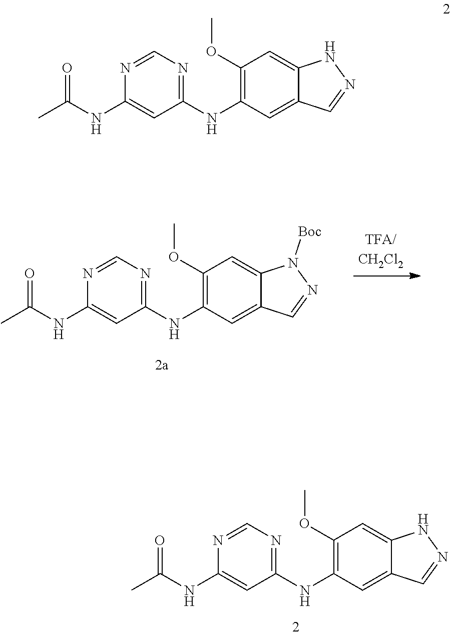 [SMILES: COc1cc2[nH]ncc2cc1Nc1cc(NC(C)=O)ncn1 ; COc1cc2c(cnn2C(=O)OC(C)(C)C)cc1Nc1cc(NC(C)=O)ncn1 ; COc1cc2[nH]ncc2cc1Nc1cc(NC(C)=O)ncn1]

The mixture of 1-tert-butoxycarbonyl-5-(6-acetylaminopyrimidin-4-ylamino)-6-methoxyindazole 2a (10 mg, 0.025 mmol) in dichloromethane (2.0 mL) and trifluoroacetic acid (1.0 mL) was stirred at room temperature for 3 hours. The mixture was quenched with saturated sodium bicarbonate aqueous solution (5 mL) and diluted with dichloromethane (5 mL). The organic layer was separated and the aqueous layer was extracted with dichloromethane (10 mL×2). The combined organic layer was washed with brine (20 mL×2), dried over anhydrous sodium sulfate and then filtered. The filtrate was concentrated under reduced pressure. Purification of the residue by Prep-TLC (dichloromethane/methanol=10/1) gave 5-(6-acetylaminopyrimidin-4-ylamino)-6-methoxy-1H-indazole 2 (5 mg, 0.017 mmol, yellow solid). Yield: 68%.

MS m/z (ESI): 299 [M+1],

¹H NMR (400 MHz, DMSO-d6) δ 12.83 (s, 1H), 10.34 (s, 1H), 8.73 (s, 1H), 8.26 (s, 1H), 7.94 (s, 1H), 7.93 (s, 1H), 7.35 (s, 1H), 7.03 (s, 1H), 3.85 (s, 3H), 3.17 (s, 3H).

### Example 3

### 1-tert-Butoxycarbonyl-5-(6-cyclopropionamidopyrimidin-4-ylamino)-6-methoxyindazole

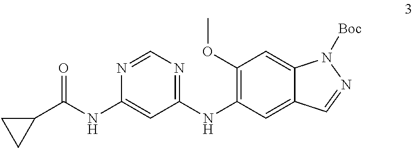 [SMILES: COc1cc2c(cnn2C(=O)OC(C)(C)C)cc1Nc1cc(NC(=O)C2CC2)ncn1]

See example 1 for the synthetic procedures. N-(6-aminopyrimidin-4-yl)cyclopropanecarboxamide is used instead of N-(6-aminopyrimidine-4-yl)ethanamide to give 1-tert-butoxycarbonyl-5-(6-cyclopropionamidopyrimidin-4-ylamino)-6-methoxyindazole 3 (1.0 g, 2.4 mmol, yellow solid). Yield: 80%.

MS m/z (ESI): 425 [M+1],

¹H NMR (400 MHz, CDCl₃) δ 8.78 (s, 1H), 8.70 (s, 1H), 8.51 (s, 1H), 8.11 (s, 1H), 7.75 (s, 1H), 7.62 (s, 1H), 7.46 (s, 1H), 4.03 (s, 3H) 1.73 (s, 9H), 1.60-1.52 (m, 1H), 0.95-0.93 (m, 4H).

### Example 4

### 5-(6-Cyclopropionamidopyrimidin-4-ylamino)-6-methoxy-1H-indazole

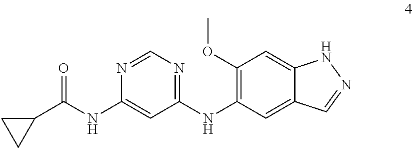 [SMILES: COc1cc2[nH]ncc2cc1Nc1cc(NC(=O)C2CC2)ncn1]

See example 2 for the synthetic procedures. 1-tert-butoxycarbonyl-5-(6-cyclopropionamidopyrimidin-4-ylamino)-6-methoxyindazole is used instead of 1-tert-butoxycarbonyl-5-(6-acylamidopyrimidin-4-ylamino)-6-methoxyindazole to give 5-(6-cyclopropionamidopyrimidin-4-ylamino)-6-methoxy-1H-indazole 4 (5.0 mg, 0.017 mmol, yellow solid). Yield: 68%.

MS m/z (ESI): 325 [M+1],

¹H NMR (400 MHz, DMSO-d6) δ 12.83 (s, 1H), 10.34 (s, 1H), 8.73 (s, 1H), 8.26 (s, 1H), 7.94 (s, 1H), 7.93 (s, 1H), 7.35 (s, 1H), 7.03 (s, 1H), 3.85 (s, 3H), 1.98-1.96 (m, 1H), 0.78-0.76 (m, 4H).

### Example 5

### 5-(6-Cyclopropionamidopyrimidin-4-ylamino)-6-methoxyl-methylindazole formate

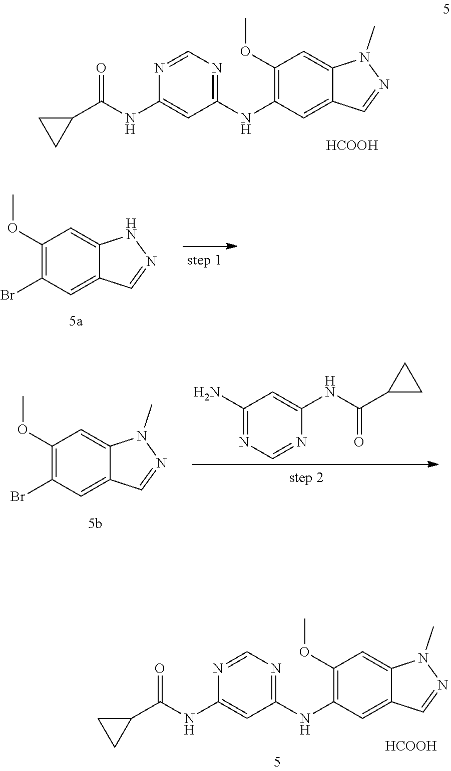

Step 1

### 5-Bromo-6-methoxy-1-methylindazole

To the mixture of 5-bromo-6-methoxy-1H-indazole 5a (113.0 mg, 0.5 mmol), cesium carbonate (326.0 mg, 1.0 mmol) and N,N-dimethylacetamide (10 mL) was added iodomethane (84.0 mg, 0.6 mmol) at room temperature. The reaction mixture was stirred at room temperature for 15 hours. It was quenched with saturated aqueous sodium bicarbonate (10 mL) and extracted with dichloromethane (30 mL×3). The combined organic layer was dried over anhydrous sodium sulfate and filtered. The filtrate was concentrated. Purification of the residue using Prep-TLC (petroleum ether/ethyl acetate=2/1) gave 5-bromo-6-methoxy-1-methylindazole 5b (15 mg, 0.062 mmol, yellow solid). Yield: 12%. MS m/z (ESI): 241 & 243 [M+1],

Step 2

5-(6-Cyclopropionamidopyrimidin-4-ylamino)-6-methoxyl-methylindazole To the mixture of 5-bromo-6-methoxy-1-methylindazole 5b (15.0 mg, 0.062 mmol), N-(6-aminopyrimidin-4-yl)cyclopropanecarboxamide (12.0 mg, 0.08 mmol) and dioxane (2.0 mL) were added tris(dibenzylideneacetone)dipalladium (5.0 mg, 0.005 mmol), 4,5-bis(diphenylphosphino)-9,9-dimethylxanthene (6.0 mg, 0.01 mmol) and cesium carbonate (98.0 mg, 0.3 mmol) under argon. The mixture was reacted at 110° C. in microwave oven for 1 hour under argon. It was cooled to room temperature, diluted with dichloromethane (10 mL) and filtered. The filtrate was concentrated under reduced pressure. Purification of the residue using Prep-HPLC (water (0.2% formic acid), 10%˜40% acetonitrile, 15 minutes) gave 5-(6-cyclopropionamidopyrimidin-4-ylamino)-6-methoxyl-methylindazole formate 5 (6.0 mg, 0.018 mmol, yellow solid). Yield: 29%.

MS m/z (ESI): 339 [M+1],

¹H NMR (400 MHz, DMSO-d6) δ 10.66 (s, 1H), 8.71 (s, 1H), 8.29 (s, 1H), 8.27 (s, 1H), 7.91 (s, 1H), 7.90 (s, 1H), 7.30 (s, 1H), 7.21 (s, 1H), 4.02 (s, 3H), 3.88 (s, 3H), 2.01-1.92 (m, 1H), 0.80-0.77 (m, 4H).

### Example 6

### 5-(6-Cyclopropionamidopyrimidin-4-ylamino)-6-ethoxy-1H-indazole

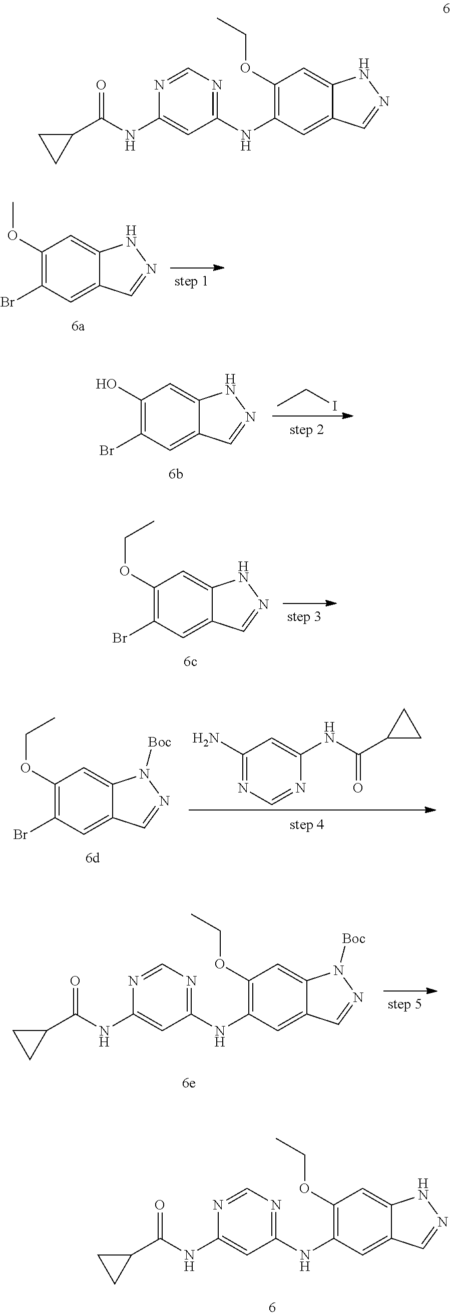

Step 1

### 5-Bromo-1H-indazol-6-ol

5-Bromo-6-methoxy-1H-indazole 6a (450.0 mg, 2.0 mmol) was mixed with aqueous solution of hydrobromic acid (10 mL) and the mixture was stirred at 100° C. for 15 hours. It was cooled to room temperature, quenched with saturated aqueous sodium bicarbonate (20 mL) and extracted with dichloromethane (30 mL×3). The combined organic layer was dried over anhydrous sodium sulfate and then filtered. The filtrate was concentrated under reduced pressure. Purification of the residue using Prep-TLC (petroleum ether/ethyl acetate=1/1) gave 5-bromo-1H-indazol-6-ol 6b (200.0 mg, 1.2 mmol, yellow solid). Yield: 61%.

MS m/z (ESI): 213 & 215 [M+1],

¹H NMR (400 MHz, DMSO-d6) δ 12.70 (s, 1H), 10.35 (s, 1H), 7.91 (s, 1H), 7.87 (s, 1H), 6.99 (s, 1H).

Step 2

### 5-Bromo-6-ethoxy-1H-indazole

To the mixture of 5-bromo-1H-indazol-6-ol 6b (42.0 mg, 0.2 mmol), potassium carbonate (138.0 mg, 1.0 mmol) and acetone (5 mL) was added iodoethane (50.0 mg, 0.3 mmol) at room temperature. The mixture was stirred at room temperature for 3 hours. It was quenched with saturated aqueous sodium bicarbonate (10 mL) and extracted with ethyl acetate (10 mL×3). The combined organic layer was dried over anhydrous sodium sulfate and then filtered. The filtrate was concentrated under reduced pressure. Purification of the residue using Prep-TLC (petroleum ether/ethyl acetate=1/1) gave 5-bromo-6-ethoxy-1H-indazole 6c (10.0 mg, 0.041 mmol, white solid). Yield: 21%.

MS m/z (ESI): 241 & 243 [M+1],

¹H NMR (400 MHz, CDCl₃) δ 10.14 (s, 1H), 7.86 (s, 1H), 7.85 (s, 1H), 6.81 (s, 1H), 4.08-4.02 (m, 2H), 1.46 (t, J=7.6 Hz, 3H).

Step 3

### 1-t-Butoxycarbonyl-5-bromo-6-ethoxyindazole

To the mixture of 5-bromo-6-ethoxy-1H-indazole 6c (10.0 mg, 0.04 mmol), triethylamine (10.0 mg, 0.1 mmol) and tetrahydrofuran (2 mL) was added di-t-butyldicarbonate (8.0 mg, 0.04 mmol) at room temperature. The reaction mixture was stirred at room temperature for 3 hours. It was quenched with saturated aqueous sodium bicarbonate (10 mL) and extracted with ethyl acetate (10 mL×3). The combined organic layer was dried over anhydrous sodium sulfate and then filtered. The filtrate was concentrated under reduced pressure. Purification of the residue using Prep-TLC (petroleum ether/ethyl acetate=5/1) gave 1-t-Butoxycarbonyl-5-bromo-6-ethoxyindazole 6d (10.0 mg, 0.03 mmol, white solid).

Yield: 75%. MS m/z (ESI): 341 & 343 [M+1],

Step 4

### 1-t-Butoxycarbonyl-5-(6-cyclopropanecarboxamido)pyrimidin-4-ylamino)-6-ethoxyindazole

To the mixture of 1-t-Butoxycarbonyl-5-bromo-6-ethoxyindazole 6d (10.0 mg, 0.03 mmol), N-(6-aminopyrimidin-4-yl)cyclopropanecarboxamide (5.0 mg, 0.03 mmol) and 1,4-dioxane (1.0 mL) were added tris(dibenzylideneacetone)dipalladium(0) (3.0 mg, 0.003 mmol), 4,5-bis(diphenylphosphino)-9,9-dimethylxanthene (6.0 mg, 0.01 mmol) and cesium carbonate (32.0 mg, 0.1 mmol) under argon. The mixture was stirred at 110° C. in microwave oven for 1 hour under argon and cooled to room temperature. The mixture was diluted with dichloromethane (10 mL) and filtered. The filtrate was concentrated under reduced pressure. Purification of the residue using Prep-TLC (dichloromethane/methanol=20/1) gave 1-t-Butoxycarbonyl-5-(6-cyclopropanecarboxamidopyrimidin-4-ylamino)-6-ethoxyindazole 6e (4.5 mg, 0.01 mmol, white solid). Yield: 33%.

MS m/z (ESI): 439 [M+1].

Step 5

### 5-(6-Cyclopropionamidopyrimidin-4-ylamino)-6-ethoxy-1H-indazole

1-t-Butoxycarbonyl-5-(6-cyclopropanecarboxamidopyrimidin-4-ylamino)-6-ethoxyindazole 6e (4.5 mg, 0.01 mmol), dichloromethane (1.0 mL) and trifluoroacetic acid (1.0 mL) were mixed and stirred at room temperature for 3 hours. The mixture was quenched with saturated aqueous sodium bicarbonate (5 mL) and diluted with dichloromethane (5 ml). The organic layer is separated, and the aqueous layer is extracted with dichloromethane (5 mL). The combined organic layer was washed with brine (20 mL×2), dried over anhydrous sodium sulfate and filtered. The filtrate was concentrated under reduced pressure. Purification of the residue using Prep-TLC (dichloromethane/methanol=10:1) gave 5-(6-cyclopropionamidopyrimidin-4-ylamino)-6-ethoxy-1H-indazole 6 (2.0 mg, 0.06 mmol, white solid). Yield: 60%.

MS m/z (ESI): 339 [M+1],

¹H NMR (400 MHz, DMSO-d6) δ 10.66 (s, 1H), 8.62 (s, 1H), 8.27 (s, 1H), 8.23 (s, 1H), 7.93 (s, 1H), 7.88 (s, 1H), 7.30 (s, 1H), 7.01 (s, 1H), 4.12-4.07 (m, 2H), 2.01-1.92 (m, 1H), 1.32-1.28 (d, J=4.8 Hz, 3H), 0.80-0.78 (m, 4H).

### Example 7

### 5-(6-cyclopropionamidopyrimidin-4-ylamino)-6-isopropoxy-1H-indazole formate

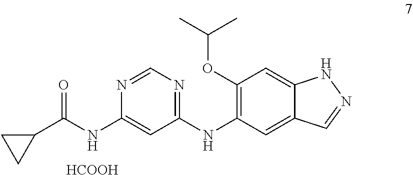 [SMILES: CC(C)Oc1cc2[nH]ncc2cc1Nc1cc(NC(=O)C2CC2)ncn1 ; "HCOOH"]

With similar procedures to those in Example 6, 2-bromopropane is used instead of iodoethane, the desired compound 7: 5-(6-cyclopropionamidopyrimidin-4-ylamino)-6-isopropoxy-1H-indazole formate (1.3 mg, 0.004 mmol, white solid) was obtained. Yield: 40%.

Prep HPLC condition: (water (0.2% formic acid), 15%˜35% acetonitrile, 15 minutes)

MS m/z (ESI): 352 [M+1],

¹H NMR (400 MHz, DMSO-d6) δ 12.77 (s, 1H), 10.65 (s, 1H), 8.54 (s, 1H), 8.28 (s, 1H), 8.24 (s, 1H), 7.93 (s, 1H), 7.89 (s, 1H), 7.30 (s, 1H), 7.02 (s, 1H), 4.68-4.62 (m, 1H), 1.99-1.96 (m, 1H), 1.25 (d, J=5.6 Hz, 6H), 0.77-0.75 (m, 4H).

### Example 8

### 1-Cyclohexanoyl-5-(6-cyclopropionamidopyrimidin-4-ylamino)-6-methoxyindazole

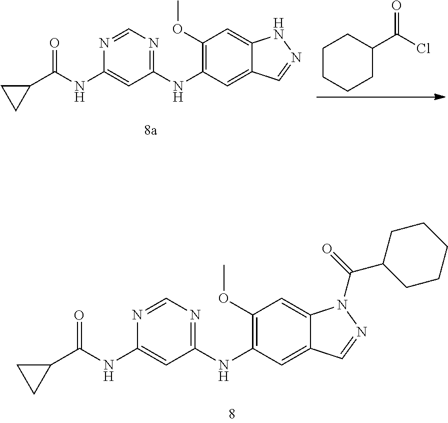

To the mixture of 5-(6-cyclopropionamidopyrimidin-4-ylamino)-6-methoxy-1H-indazole 8a (10.0 mg, 0.03 mmol), sodium hydride (60% dispersion in mineral oil, 3.0 mg, 0.1 mmol) and tetrahydrofuran (2.0 mL) was added cyclohexanecarbonyl chloride (6.0 mg, 0.03 mmol). The mixture was reacted at room temperature for 10 minutes. The mixture was quenched with saturated aqueous sodium bicarbonate (10 mL) and extracted with dichloromethane (10 mL×3). The combined organic layer was dried over anhydrous sodium sulfate and filtered. The filtrate was concentrated under reduced pressure. Purification of the residue using Prep-TLC (dichloromethane/methanol=10/1) gave 1-cyclohexanoyl-5-(6-cyclopropionamidopyrimidin-4-ylamino)-6-methoxyindazole 8 (2.0 mg, 0.005 mmol, yellow solid). Yield: 15%.

MS m/z (ESI): 435 [M+1],

¹H NMR (400 MHz, DMSO-d6) δ 12.88 (s, 1H), 9.16 (s, 1H), 8.54 (s, 1H), 8.12 (s, 1H), 7.97 (s, 1H), 7.06 (s, 1H), 6.65 (s, 1H), 3.88 (s, 3H), 2.84-2.81 (m, 1H), 2.03-2.00 (m, 2H), 1.89-1.86 (m, 2H), 1.80-1.77 (m, 3H), 1.73-1.71 (m, 1H), 1.46-1.42 (m, 2H), 0.97-0.94 (m, 4H), 0.88-0.84 (m, 1H).

### Example 9

### 5-(6-aminopyrimidin-4-ylamino)-6-methoxy-1H-indazole

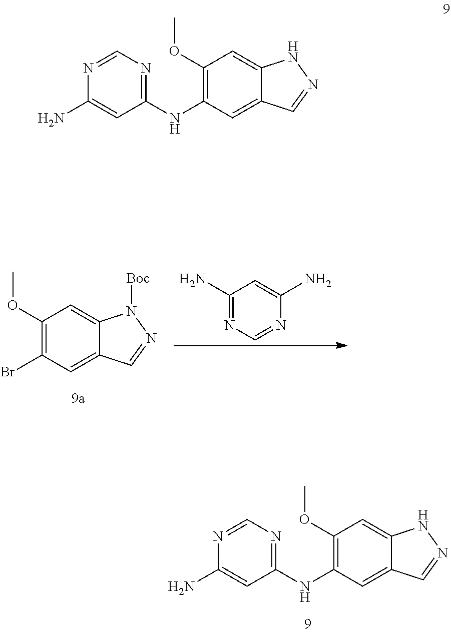 [SMILES: COc1cc2[nH]ncc2cc1Nc1cc(N)ncn1 ; COc1cc2c(cnn2C(=O)OC(C)(C)C)cc1Br ; Nc1cc(N)ncn1 ; COc1cc2[nH]ncc2cc1Nc1cc(N)ncn1]

To the mixture of 1-tert-butoxycarbonyl-5-bromo-6-methoxyindazole 9a (50.0 mg, 0.15 mmo), 4,6-diaminopyrimidine (22.0 mg, 0.2 mmol) and 1,4-dioxane (1.0 mL) was added tris(dibenzylideneacetone)dipalladium(0) (14.0 mg, 0.015 mmol), 4,5-bis(diphenylphosphino)-9,9-dimethylxanthene (17.0 mg, 0.03 mmol) and cesium carbonate (98.0 mg, 0.3 mmol) under argon. The mixture was reacted at 125° C. in microwave oven for 1 hour and cooled to room temperature. The mixture was diluted with dichloromethane (10 mL) and filtered, the filtrate was concentrated under reduced pressure. Purification of the residue using Prep-TLC (dichloromethane/methanol=10/1) gave 5-(6-aminopyrimidin-4-ylamino)-6-methoxy-1H-indazole 9 (2.0 mg, 0.01 mmol, white solid). Yield: 7%.

MS m/z (ESI): 257 [M+1],

¹H NMR (400 MHz, DMSO-d6) δ 8.28 (s, 1H), 7.96 (s, 1H), 7.92 (s, 1H), 7.87 (s, 1H), 7.84 (s, 1H), 7.02 (s, 1H), 6.17 (s, 2H), 5.58 (s, 1H), 3.86 (s, 3H).

### Example 10

### 5-(6-benzoylaminopyrimidin-4-ylamino)-6-methoxy-1H-indazole

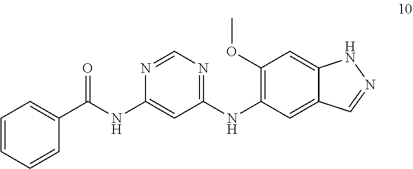 [SMILES: COc1cc2[nH]ncc2cc1Nc1cc(NC(=O)c2ccccc2)ncn1]

The synthetic procedure is similar to that in Example 1. using N-(6-aminopyrimidine-4-yl)benzamide instead of N-(6-aminopyrimidine-4-yl)ethanamide, The desired compound 10 5-(6-benzoylaminopyrimidin-4-ylamino)-6-methoxy-1H-indazole was obtained. Yield: 42%.

MS m/z (ESI): 361 [M+1],

¹H NMR (400 MHz, DMSO-d6) δ 12.88 (s, 1H), 10.69 (s, 1H), 8.86 (s, 1H), 8.35 (s, 1H), 8.11-7.89 (m, 4H), 7.70-7.35 (m, 4H), 7.05 (s, 1H), 3.87 (s, 3H).

### Example 11

### 5-(6-(2-Pyridine) formylaminopyrimidin-4-ylamino)-6-methoxy-1H-indazole

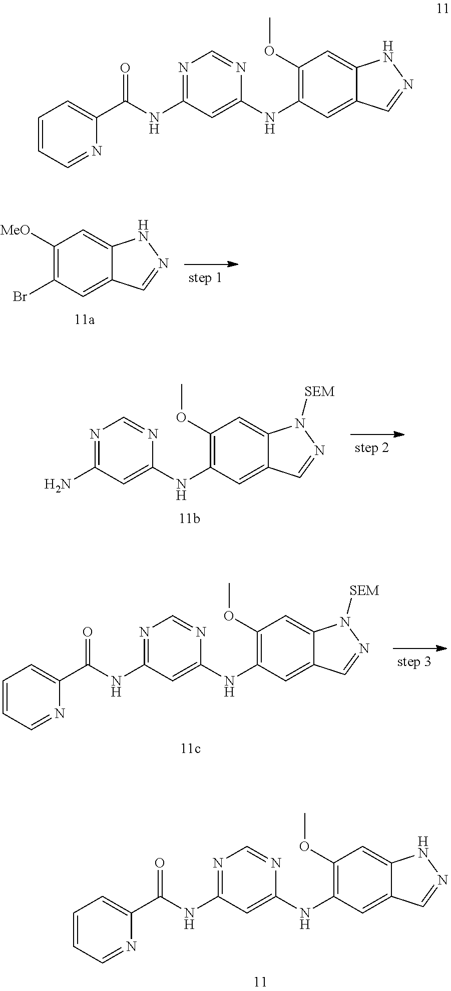 [SMILES: COc1cc2[nH]ncc2cc1Nc1cc(NC(=O)c2ccccn2)ncn1 ; COc1cc2[nH]ncc2cc1Br ; COc1cc2c(cnn2COCC[Si](C)(C)C)cc1Nc1cc(N)ncn1 ; COc1cc2c(cnn2COCC[Si](C)(C)C)cc1Nc1cc(NC(=O)c2ccccn2)ncn1 ; COc1cc2[nH]ncc2cc1Nc1cc(NC(=O)c2ccccn2)ncn1]

Step 1

### 5-Bromo-6-methoxy-1-((2-(trimethylsilyl) ethoxy) methyl)-1H-indazole

5-Bromo-6-methoxy-1H-indazole 11a (320 mg, 1.4 mmol), N,N-diisopropylethylamine (903 mg, 7 mmol), 2-(trimethylsilyl) ethoxymethyl chloride (500 mg, 3.0 mmol) and dichloromethane (10 mL) were mixed and reacted at room temperature for 3 hours under argon. The mixture was concentrated under reduced pressure. Purification of the residue using column chromatography (petroleum ether/ethyl acetate=4/1) gave 5-bromo-6-methoxy-1-((2-(trimethylsilyl) ethoxy) methyl)-1H-indazole 11 b (320 mg, 0.9 mmol, yellow solid).

Yield: 64%.

MS m/z (ESI): 357 & 359 [M+1],

Step 2

### 1-((2-(Trimethylsilyl) ethoxy) methyl)-5-(6-(2-pyridine) formylamino) pyrimidin-4-ylamino)-6-methoxyindazole

5-Bromo-6-methoxy-1-((2-(trimethylsilyl) ethoxy) methyl)-1H-indazole 11b (18.0 mg, 0.05 mmol), 2-(7-azabenzotriazol-1-yl)-N,N,N′,N′tetramethyluronium hexafluorophosphate (38.0 mg, 0.10 mmol), N,N-diisopropylethylamine (13.0 mg, 0.10 mmol) were dissolved into tetrahydrofuran (1 mL) and stirred at room temperature for 15 minutes, then picolinic acid (12.0 mg, 0.10 mmol) was added. The reaction mixture was stirred at room temperature for 12 hours. The mixture was concentrated under reduced pressure. Purification of the residue using Prep-HPLC (water (0.2% formic acid), 30%-70% acetonitrile, 15 minutes) gavel-((2-(trimethylsilyl) ethoxy) methyl)-5-(6-(2-pyridine) formylamino) pyrimidin-4-ylamino)-6-methoxyindazole 11c (6.0 mg, 0.012 mmol, white solid). Yield: 24%.

MS m/z (ESI): 492 [M+1],

Step 3

### 5-(6-(2-pyridine) formylaminopyrimidin-4-ylamino)-6-methoxy-1H-indazole

1-((2-(trimethylsilyl) ethoxy) methyl)-5-(6-(2-pyridine) formylamino) pyrimidin-4-ylamino)-6-methoxyindazole 11c (6.0 mg, 0.012 mmol) was dissolved in dichloromethane (1.0 mL) and trifluoroacetic acid (1.0 mL) was added, the mixture was stirred at room temperature for 1.5 hours. The mixture was concentrated under reduced pressure. Purification of the residue using Prep-HPLC (water (0.2% formic acid), 30%˜70% acetonitrile, 15 minutes) gave 5-(6-(2-pyridine) formylaminopyrimidin-4-ylamino)-6-methoxy-1H-indazole 11 (3.0 mg, 0.008 mmol, white solid). Yield: 67%.

MS m/z (ESI): 362 [M+1],

¹H NMR (400 MHz, DMSO-d6) δ 12.93 (brs, 1H), 10.19 (s, 1H), 9.03 (s, 1H), 8.76 (s, 1H), 8.35 (s, 1H), 8.31 (s, 1H), 8.18-8.09 (m, 2H), 7.97 (s, 1H), 7.76-7.72 (m, 1H), 7.52 (s, 1H), 7.07 (s, 1H), 3.87 (s, 3H).

### Example 12

### 5-(6-Pivaloylaminopyrimidin-4-ylamino)-6-methoxy-1H-indazole

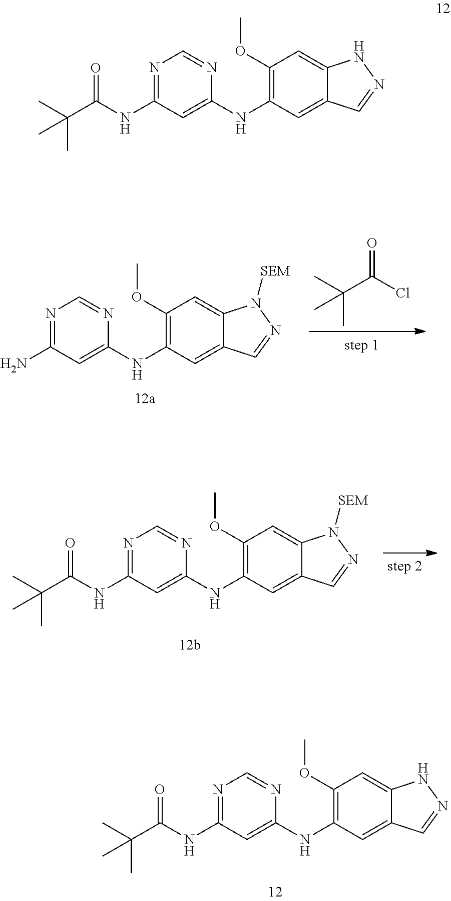

Step 1

### 1-((2-(Trimethylsilyl) ethoxy) methyl)-5-(6-pivaloylamino) pyrimidin-4-ylamino)-6-methoxyindazole

1-((2-(Trimethylsilyl) ethoxy) methyl)-5-(6-aminopyrimidin-4-ylamino)-6-methoxyindazole 12a (100.0 mg, 0.26 mmol) and triethylamine (55.0 mg, 0.52 mmol) are dissolved in anhydrous dichloromethane (4.0 mL), pivaloyl chloride (50.0 mg, 0.39 mmol) was added. The mixture was stirred at room temperature for 30 minutes. It was quenched with saturated aqueous sodium bicarbonate (0.5 mL). The mixture was concentrated under reduced pressure. Purification of the residue using Prep-TLC (dichloromethane/methanol=20:1) gavel-((2-(trimethylsilyl) ethoxy) methyl)-5-(6-pivaloylamino) pyrimidin-4-ylamino)-6-methoxyindazole 12b (100.0 mg, 0.21 mmol, yellow solid). Yield: 81%.

MS m/z (ESI): 471 [M+1],

Step 2

### 5-(6-Pivaloylaminopyrimidin-4-ylamino)-6-methoxy-1H-indazole

1-((2-(Trimethylsilyl) ethoxy) methyl)-5-(6-pivaloylamino) pyrimidin-4-ylamino)-6-methoxyindazole 12b (50.0 mg, 0.10 mmol) was dissolved in dichloromethane (1.0 mL), and trifluoroacetic acid (1.0 mL) was added and stirred at room temperature for 30 minutes. The mixture was concentrated under reduced pressure. Purification of the residue using Prep-HPLC (water (0.2% formic acid), 30%˜70% acetonitrile, 15 minutes) gave 5-(6-pivaloylaminopyrimidin-4-ylamino)-6-methoxy-1H-indazole 12 (20.0 mg, 0.06 mmol, white solid). Yield: 60%.

MS m/z (ESI): 341 [M+1],

¹H NMR (400 MHz, CDCl₃) δ 8.52-8.43 (m, 3H), 8.21 (brs, 1H), 8.01 (s, 1H), 7.68 (s, 1H), 7.44 (s, 1H), 6.92 (s, 1H), 3.95 (s, 3H), 1.29 (s, 9H).

### Example 13

### 1-Isobutyryl-5-(6-cyclopropionamidopyrimidin-4-ylamino)-6-methoxyindazole

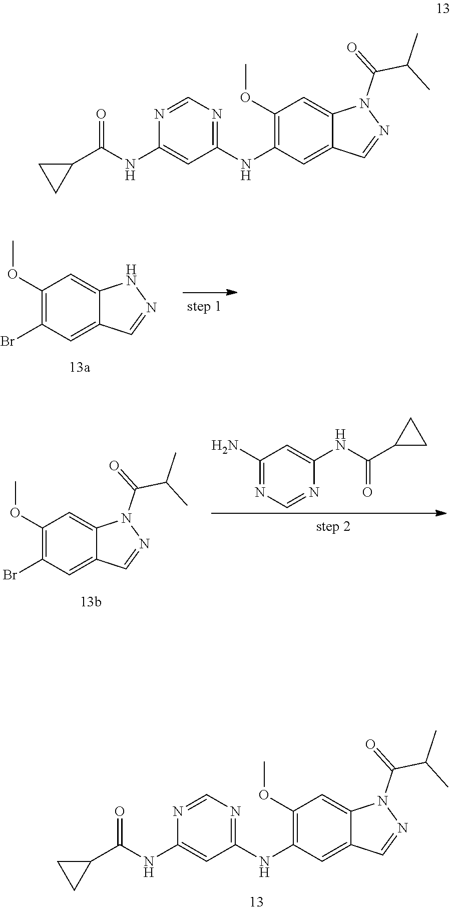 [SMILES: COc1cc2c(cnn2C(=O)C(C)C)cc1Nc1cc(NC(=O)C2CC2)ncn1 ; COc1cc2[nH]ncc2cc1Br ; COc1cc2c(cnn2C(=O)C(C)C)cc1Br ; Nc1cc(NC(=O)C2CC2)ncn1 ; COc1cc2c(cnn2C(=O)C(C)C)cc1Nc1cc(NC(=O)C2CC2)ncn1]

Step 1

### 5-Bromo-6-methoxy-1-isobutyrylindazole

To the mixture of 5-bromo-6-methoxy-1H-indazole 13a (113.0 mg, 0.5 mmol), triethylamine (101.0 mg, 1.0 mmol) and dichlormethane (10 mL) was added isobutyryl chloride (84.0 mg, 0.6 mmol) at room temperature, and was reacted at room temperature for 1 hour. The mixture was quenched with saturated aqueous sodium bicarbonate (10 mL) and extracted with dichlormethane (30 mL×3). The combined organic layer was washed with brinebrine (30 mL), dried over anhydrous sodium sulfate and then filtered. The mixture was concentrated under reduced pressure. Purification of the residue using Prep-TLC (petroleum ether/ethyl acetate=5/1) gave 5-bromo-6-methoxy-1-isobutyrylindazole 13b (41 mg, 0.14 mmol, yellow solid). Yield: 28%.

MS m/z (ESI): 297 & 299 [M+1],

Step 2

### 1-Isobutyryl-5-(6-cyclopropionamidopyrimidin-4-ylamino)-6-methoxyindazole

To the mixture of 5-bromo-6-methoxy-1-isobutyrylindazole 13b (15.0 mg, 0.050 mmol), N-(6-aminopyrimidin-4-yl)cyclopropanecarboxamide (11.0 mg, 0.060 mmol) and 1,4-dioxane (2 mL) were added tris(dibenzylideneacetone)-dipalladium(0) (5.0 mg, 0.005 mmol), 4,5-bis(diphenylphosphino)-9,9-dimethylxanthene (6.0 mg, 0.010 mmol) and cesium carbonate (49.0 mg, 0.150 mmol) under argon. The mixture was stirred at 110° C. in microwave oven for 1 hour under argon and cooled to room temperature. the mixture was diluted with dichloromethane (10 mL) and filtered. The filtrate was concentrated under reduced pressure. Purification of the residue using Prep-TLC (dichlormethane/methanol=20/1) gave 1-isobutyryl-5-(6-cyclopropionamidopyrimidin-4-ylamino)-6-methoxyindazole 13 (7.0 mg, 0.018 mmol, yellow solid). Yield: 36%.

MS m/z (ESI): 395 [M+1],

¹H NMR (400 MHz, DMSO-d6) δ 8.87 (s, 1H), 8.69 (s, 1H), 8.50 (s, 1H), 8.08 (s, 1H), 8.01 (s, 1H), 7.63 (s, 1H), 7.46 (s, 1H), 4.01 (s, 3H), 4.01-3.94 (m, 1H), 2.06-1.98 (m, 1H), 1.36 (d, J=7.2 Hz, 6H), 1.16-1.08 (m, 2H), 0.99-0.89 (m, 2H).

### Example 14

### 1-Cyclopropanoyl-5-(6-cyclopropionamidopyrimidin-4-ylamino)-6-methoxyindazole

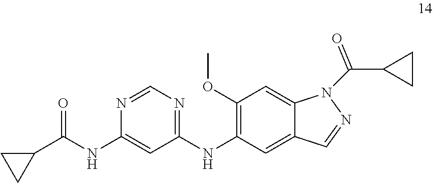 [SMILES: COc1cc2c(cnn2C(=O)C2CC2)cc1Nc1cc(NC(=O)C2CC2)ncn1]

The synthetic procedure is similar to that in Example 13. using cyclopropionyl chloride instead of isobutyryl chloride, the desired compound 14:1-cyclopropanoyl-5-(6-cyclopropionamidopyrimidin-4-ylamino)-6-methoxyindazole (3.0 mg, 0.008 mmol) was obtained. Yield: 27%.

MS m/z (ESI): 393 [M+1],

¹H NMR (400 MHz, CDCl₃) δ 8.74 (s, 1H), 8.51 (s, 1H), 8.17 (s, 1H), 8.13 (s, 1H), 7.97 (s, 1H), 7.61 (s, 1H), 7.43 (s, 1H), 4.01 (s, 3H), 1.33-1.30 (m, 2H), 1.16-1.10 (m, 4H), 0.97-0.85 (m, 4H).

### Example 15

### 2-Cyclopropanoyl-5-(6-cyclopropionamidopyrimidin-4-ylamino)-6-methoxyindazole

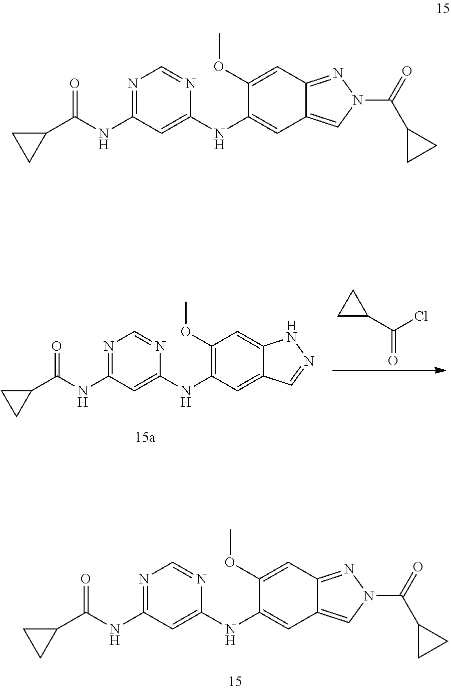 [SMILES: COc1cc2nn(C(=O)C3CC3)cc2cc1Nc1cc(NC(=O)C2CC2)ncn1 ; COc1cc2[nH]ncc2cc1Nc1cc(NC(=O)C2CC2)ncn1 ; O=C(Cl)C1CC1 ; COc1cc2nn(C(=O)C3CC3)cc2cc1Nc1cc(NC(=O)C2CC2)ncn1]

To the mixture of 5-(6-cyclopropionamidopyrimidin-4-ylamino)-6-methoxy-1H-indazole 15a (50.0 mg, 0.15 mmol), N,N-diisopropylethylamine (60.0 mg, 0.45 mmol) and N,N-dimethylformamide (4 mL) was added cyclopropanecarbonyl chloride (48.0 mg, 0.45 mmol). The mixture was stirred at room temperature for 12 hours and was quenched with saturated aqueous sodium bicarbonate (10 mL), then extracted with dichloromethane (10 mL×3). The combined organic layer was dried over anhydrous sodium sulfate and then filtered. The filtrate was concentrated under reduced pressure. Purification of the residue using Prep-HPLC (water (0.2% formic acid), 30%˜70% acetonitrile, 15 minutes) gave 2-cyclopropanoyl-5-(6-cyclopropionamidopyrimidin-4-ylamino)-6-methoxyindazole 15 (15 mg, 0.04 mmol, white solid). Yield: 25%.

MS m/z (ESI): 393 [M+1],

¹H NMR (400 MHz, CDCl₃) δ 10.82 (s, 1H), 8.90 (s, 1H), 8.80 (s, 1H), 8.43 (s, 1H), 8.33 (s, 1H), 7.70 (s, 1H), 7.06 (s, 1H), 3.93 (s, 3H), 3.30-3.28 (m, 1H), 2.03-2.02 (m, 1H), 1.24-1.21 (m, 4H), 0.86-0.84 (m, 4H). ¹³C NMR (400 MHz, CDCl₃) δ 173.89, 162.40, 157.91, 157.08, 154.83, 149.33, 129.57, 121.81, 118.45, 110.82, 95.20, 94.32, 56.45, 14.76, 12.73, 11.94, 8.53.

### Example 16

### 1-Acetyl-5-(6-cyclopropionamidopyrimidin-4-ylamino)-6-methoxyindazole

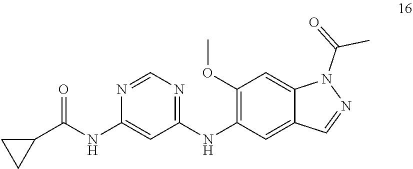 [SMILES: COc1cc2c(cnn2C(C)=O)cc1Nc1cc(NC(=O)C2CC2)ncn1]

The synthetic procedure is similar to that in Example 13. acetic anhydride instead of isobutyryl chloride, the desired compound 16: 1-acetyl-5-(6-cyclopropionamidopyrimidin-4-ylamino)-6-methoxyindazole (6.0 mg, 0.016 mmol, yellow solid) was obtained. Yield: 32%.

MS m/z (ESI): 367 [M+1],

¹H NMR (400 MHz, DMSO-d6) δ 8.83 (s, 1H), 8.50 (s, 1H), 8.17 (s, 1H), 8.08 (s, 1H), 7.96 (s, 1H), 7.61 (s, 1H), 7.42 (s, 1H), 4.03 (s, 3H), 2.79 (s, 3H), 2.06-1.97 (m, 1H), 1.15-1.09 (m, 2H), 0.99-0.92 (m, 2H).

### Example 17

### 1-Methoxyacyl-5-(6-cyclopropionamidopyrimidin-4-ylamino)-6-methoxyindazole

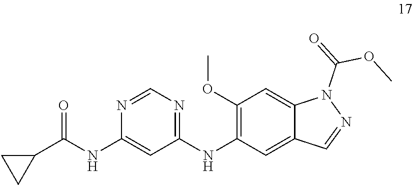 [SMILES: COC(=O)n1ncc2cc(Nc3cc(NC(=O)C4CC4)ncn3)c(OC)cc21]

The synthetic procedure is similar to that in Example 13. using methyl chloroformate instead of isobutyryl chloride acetic anhydride, The desired compound17:1-methoxyacyl-5-(6-cyclopropionamidopyrimidin-4-ylamino)-6-methoxyindazole (2.0 mg, 0.005 mmol, yellow solid) was obtained. Yield: 10%.

MS m/z (ESI): 383 [M+1],

¹H NMR (400 MHz, DMSO-d6) δ 8.74 (s, 1H), 8.50 (s, 1H), 8.14 (s, 1H), 8.12 (s, 1H), 7.77 (s, 1H), 7.61 (s, 1H), 7.40 (s, 1H), 4.13 (s, 3H), 4.03 (s, 3H), 2.06-1.99 (m, 1H), 1.16-1.08 (m, 2H), 0.99-0.91 (m, 2H).

### Example 18

### 1-Ethoxyacyl-5-(6-cyclopropionamidopyrimidin-4-ylamino)-6-methoxyindazole

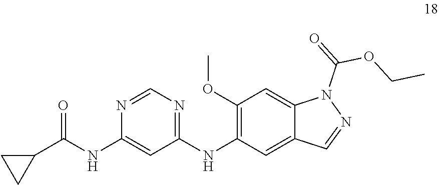 [SMILES: CCOC(=O)n1ncc2cc(Nc3cc(NC(=O)C4CC4)ncn3)c(OC)cc21]

The synthetic procedure is similar to that in Example 13. using ethyl chloroformate instead of isobutyryl chloride, the desired compound 18: 1-ethoxyacyl-5-(6-cyclopropionamidopyrimidin-4-ylamino)-6-methoxyindazole 18 (5.0 mg, 0.013 mmol, yellow solid) was obtained. Yield: 26%.

MS m/z (ESI): 397 [M+1],

¹H NMR (400 MHz, DMSO-d6) δ 8.74 (s, 1H), 8.50 (s, 1H), 8.16 (s, 1H), 8.13 (s, 1H), 7.78 (s, 1H), 7.61 (s, 1H), 7.40 (s, 1H), 4.83 (q, J=7.2 HZ, 2H), 4.03 (s, 3H), 2.06-1.99 (m, 1H), 1.54 (t, J=7.2 HZ, 3H), 1.16-1.08 (m, 2H), 0.99-0.91 (m, 2H).

### Example 19

### N-(6-((6-Methoxy-1H-indazol-5-yl)amino)pyrimidin-4-yl)isobutyramide

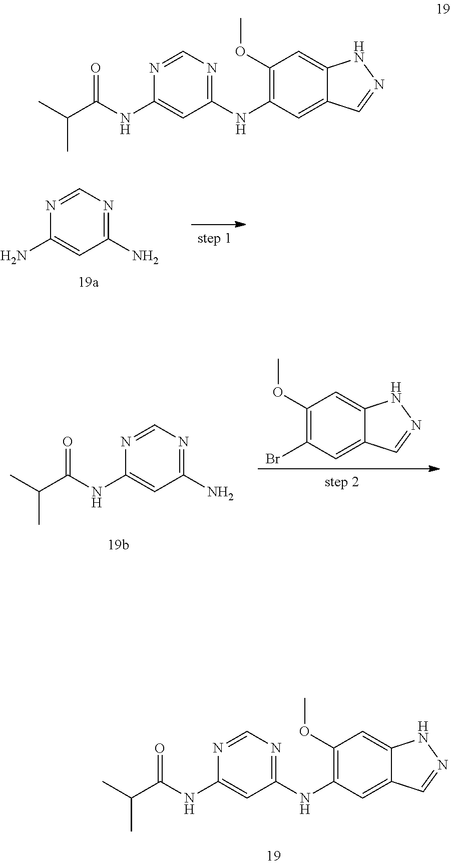

Step 1

### N-(6-Aminopyrimidin-4-yl)isobutyramide

To the solution of 4,6-diamino pyrimidine 19a (550 mg, 5.0 mmol) in tetrahydrofuran (5 mL) were added isobutyryl chloride (1.07 g, 10.0 mmol) and potassium carbonate (2.07 g, 15.0 mmol) at room temperature. The reaction mixture was kept at room temperature overnight and concentrated under reduced pressure. Purification of the residue using flash column chromatography (dichloromethane/methanol=1:0˜10:1) gave N-(6-Aminopyrimidin-4-yl)isobutyramide 19b (80.6 mg, 0.4 mmol, white solid). Yield: 9%.

MS m/z (ESI): 181 [M+1],

Step 2

### N-(6-((6-Methoxy-1H-indazol-5-yl)amino)pyrimidin-4-yl)isobutyramide

To the mixture of N-(6-aminopyrimidin-4-yl)isobutyramide 19b (3.6 mg, 0.02 mmol), 5-bromo-6-methoxy-1H-indazole (6 mg, 0.02 mmol), cesium carbonate (20 mg, 0.06 mmol) and 1,4-dioxane (1 mL) were added tris(dibenzylideneacetone)dipalladium(0) (2.2 mg, 0.002 mmol) and 2-(dicyclohexylphosphine)-3,6-dimethoxy-2′-4′-6′-tri-isopropyl-1,1′-biphenyl(2.2 mg, 0.004 mmol) under argon. The mixture was stirred at 110° C. in oil bath for 1 hour under argon. Then it was cooled to room temperature, diluted with methanol (5 mL) and filtered. The filtrate was concentrated under reduced pressure. Purification of the residue using Prep-HPLC gave N-(6-((6-methoxy-1H-indazol-5-yl)amino)pyrimidin-4-yl)isobutyramide 19 (2.0 mg, 0.006 mmol, white solid). Yield: 30%.

MS m/z (ESI): 327 [M+1],

¹H NMR (400 MHz, DMSO-d6) δ 12.87 (s, 1H), 10.32 (s, 1H), 8.73 (s, 1H), 8.26 (s, 1H), 7.95-7.91 (m, 2H), 7.34 (s, 1H), 7.03 (s, 1H), 3.83 (s, 3H), 2.05-1.90 (m, 1H), 1.03 (d, J=6.7 Hz, 6H).

### Example 20

### 5-(6-(Pyridin-2-ylamino) pyrimidin-4-ylamino)-6-methoxy-1H-indazole formate

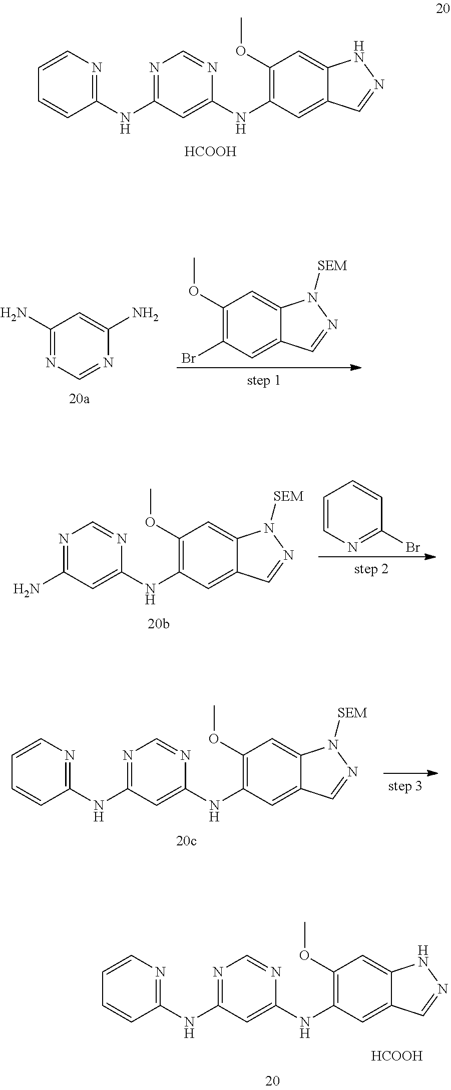

Step 1

### 1-((2-(Trimethylsilyl) ethoxy) methyl)-5-(6-aminopyrimidin-4-ylamino)-6-methoxyindazole

To a mixture of 4,6-diaminopyrimidine 20a (11.0 mg, 0.1 mmol), 5-bromo-6-methoxy-1-((2-(trimethylsilyl)ethoxy)methyl)-1H-indazole (36.0 mg, 0.05 mmol) and 1,4-dioxane (2.0 mL) were added tris(dibenzylideneacetone)dipalladium(0) (9.0 mg, 0.01 mmol), 4,5-bis(diphenylphosphino)-9,9-dimethylxanthene (12.0 mg, 0.02 mmol) and cesium carbonate (65.0 mg, 0.2 mmol) at room temperature under agon. The mixture was stirred at 120° C. for 16 hours. It was cooled to room temperature and quenched with water (10 mL). The organic layer was separated and the aqueous phase was extracted with dichloromethane (15 mL×3). The combined organic layer was washed with brine (30 mL), dried over anhydrous sodium sulfate and then filtered, the filtrate was concentrated under reduced pressure. Purification of the residue using Prep TLC (dichloromethane/methanol=20/1) gave 1-((2-(trimethylsilyl) ethoxy) methyl)-5-(6-aminopyrimidin-4-ylamino)-6-methoxyindazole 20b (22.0 mg, 0.057 mmol, yellow solid). Yield: 57%.

MS m/z (ESI): 387 [M+1],

Step 2

### 1-((2-(Trimethylsilyl) ethoxy) methyl)-5-(6-(pyridin-2-ylamino) pyrimidin-4-ylamino)-6-methoxyindazole

To the mixture of 1-((2-(trimethylsilyl) ethoxy) methyl)-5-(6-aminopyrimidin-4-ylamino)-6-methoxyindazole 20b (11.0 mg, 0.03 mmol), 2-bromopyridine (5.0 mg, 0.05 mmol) and 1,4-dioxane (1.0 mL) were added tris(dibenzylideneacetone)dipalladium(0) (9.0 mg, 0.01 mmol), 4,5-bis(diphenylphosphino)-9,9-dimethylxanthene (12.0 mg, 0.02 mmol) and cesium carbonate (65.0 mg, 0.2 mmol) at room temperature under argon. The mixture was stirred at 120° C. in microwave oven under argon for 1 hour, and then was cooled to room temperature, quenched with water (10 mL). The organic layer was separated and the aqueous layer was extracted with dichloromethane (15 mL×2). The combined organic layer was washed with saturated brine (50 mL×2), dried over anhydrous sodium sulfate and then filtered. The filtrate was concentrated under reduced pressure. Purification of the residue using Prep-TLC (dichloromethane/methanol=20/1) gavel-((2-(trimethylsilyl) ethoxy) methyl)-5-(6-(pyridin-2-ylamino) pyrimidin-4-ylamino)-6-methoxyindazole 20c (6.0 mg, 0.013 mmol, yellow oil). Yield: 46%.

MS m/z (ESI): 464 [M+1],

Step 3

### 5-(6-(Pyridin-2-ylamino) pyrimidin-4-ylamino)-6-methoxy-1H-indazole

1-((2-(trimethylsilyl) ethoxy) methyl)-5-(6-(pyridin-2-ylamino) pyrimidin-4-ylamino)-6-methoxyindazole 20c (6.0 mg, 0.013 mmol), dichloromethane (1 mL) and trifluoroacetic acid (1 mL) were mixed and stirred at room temperature for 3 hours. Then it was quenched with saturated aqueous sodium bicarbonate (10 mL). The organic layer was separated and the aqueous layer was extracted with dichloromethane (15 mL×2). The combined organic layer was washed with brine (50 mL×2), dried over anhydrous sodium sulfate and then filtered. The filtrate was concentrated under reduced pressure. Purification of the residue using Prep-HPLC (water (0.2% formic acid), 10%˜40% acetonitrile, 15 minutes) gave 5-(6-(pyridin-2-ylamino) pyrimidin-4-ylamino)-6-methoxy-1H-indazole formate 20 (2.0 mg, 0.006 mmol, white solid). Yield: 46%.

MS m/z (ESI): 334 [M+1],

¹H NMR (400 MHz, DMSO-d6) δ 12.83 (s, 1H), 9.65 (s, 1H), 8.42 (s, 1H), 8.20 (s, 1H,), 8.17 (d, J=7.2 Hz, 1H), 8.14 (s, 1H), 7.96 (s, 1H), 7.94 (s, 1H), 7.68-7.64 (m, 1H), 7.52 (d, J=8.4 Hz, 1H), 7.17 (s, 1H), 7.04 (s, 1H), 6.90-6.85 (m, 1H), 3.87 (s, 3H).

### Example 21

### 5-(6-((4-Chloropyridin-2-yl) amino) pyrimidin-4-ylamino)-6-methoxy-1H-indazole

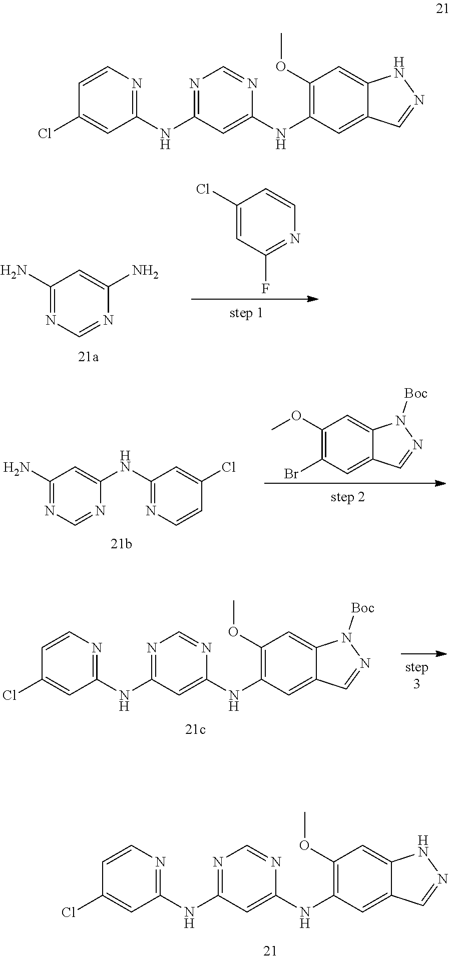

Step 1

### 4-(4-Chloropyridin-2-yl) amino-6-aminopyrimidine

To the mixture of 4,6-diaminopyrimidine 21a (70.0 mg, 0.6 mmol), 4-chloro-2-fluoropyridine (65.0 mg, 0.5 mmol) and N,N-dimethylacetamide (5 mL) was added cesium carbonate (327.0 mg, 1 mmol) at room temperature. The reaction was stirred at 110° C. for 3 hours and quenched with water (20 mL). The organic layer was separated and the aqueous layer was extracted with dichloromethane (15 mL×2). The combined organic layer was washed with saturated brine (50 mL×2), dried over anhydrous sodium sulfate and then filtered. The filtrate was concentrated under reduced pressure. Purification of the residue using Prep-TLC (dichloromethane/methanol=10/1) gave 4-(4-chloropyridin-2-yl) amino-6-aminopyrimidine 21b (18.0 mg, 0.08 mmol, yellow solid). Yield: 16%.

MS m/z (ESI): 222 & 224 [M+1],

Step 2

### 1-tert-Butoxycarbonyl-5-(6-((4-chloropyridin-2-yl) amino) pyrimidin-4-ylamino)-6-methoxyindazole

To the mixture of 4-(4-chloropyridin-2-yl) amino-6-aminopyrimidine 21b (18.0 mg, 0.08 mmol), 1-tert-butoxycarbonyl-5-bromo-6-methoxyindazole (33.0 mg, 0.1 mmol) and 1,4-dioxane (2.0 mL) were added tris(dibenzylideneacetone)-dipalladium(0) (9.0 mg, 0.01 mmol), 4,5-bis(diphenylphosphino)-9,9-dimethylxanthene (11.0 mg, 0.02 mmol) and cesium carbonate (65.0 mg, 0.2 mmol) at room temperature under argon. The mixture was stirred at 130° C. in microwave oven under argon for 1 hour and cooled to room temperature, then quenched with water (10 mL). The organic layer was separated and the aqueous layer was extracted with dichloromethane (15 mL×2). The combined organic layer was washed with saturated brine (50 mL×2), dried over anhydrous sodium sulfate and then filtered. The filtrate was concentrated under reduced pressure. Purification of the residue using Prep TLC (dichloromethane/methanol=20/1) gave 1-tert-butoxycarbonyl-5-(6-((4-chloropyridin-2-yl) amino) pyrimidin-4-ylamino)-6-methoxyindazole 21c (20 mg, 0.043 mmol, yellow solid). Yield: 54%.

MS m/z (ESI): 468 & 470 [M+1],

Step 3

### 5-(6-((4-Chloropyridin-2-yl) amino) pyrimidin-4-ylamino)-6-methoxy-1H-indazole

The mixture of 1-tert-butoxycarbonyl-5-(6-((4-chloropyridin-2-yl) amino) pyrimidin-4-ylamino)-6-methoxyindazole 21c (20.0 mg, 0.043 mmol), dichloromethane (1.0 mL) and trifluoroacetic acid (1.0 mL) was stirred for 3 hours at room temperature. Then it was quenched with saturated aqueous sodium bicarbonate (10 mL). The organic layer was separated and the aqueous layer was extracted with dichloromethane (15 mL×2). The combined organic layer was washed with saturated brine (50 mL×2), dried over anhydrous sodium sulfate and filtered. The filtrate was concentrated under reduced pressure. Purification of the residue using Prep TLC (dichloromethane/methanol=10/1) gave 5-(6-((4-chloropyridin-2-yl) amino) pyrimidin-4-ylamino)-6-methoxy-1H-indazole 21 (3.0 mg, 0.008 mmol, white solid). Yield: 19%.

MS m/z (ESI): 368 & 370 [M+1],

¹H NMR (400 MHz, DMSO-d6) δ 12.86 (s, 1H), 9.91 (s, 1H), 8.56 (s, 1H), 8.27 (s, 1H), 8.18 (d, J=5.2 Hz, 1H), 7.95 (d, J=5.2 Hz, 1H), 7.93 (s, 1H), 7.83 (s, 1H), 7.04 (s, 1H), 7.02 (s, 1H), 6.98 (s, 1H), 3.87 (s, 3H).

### Example 22

### 5-(6-(pyrimidin-4-ylamino) pyrimidin-4-ylamino)-6-methoxy-1H-indazole formate

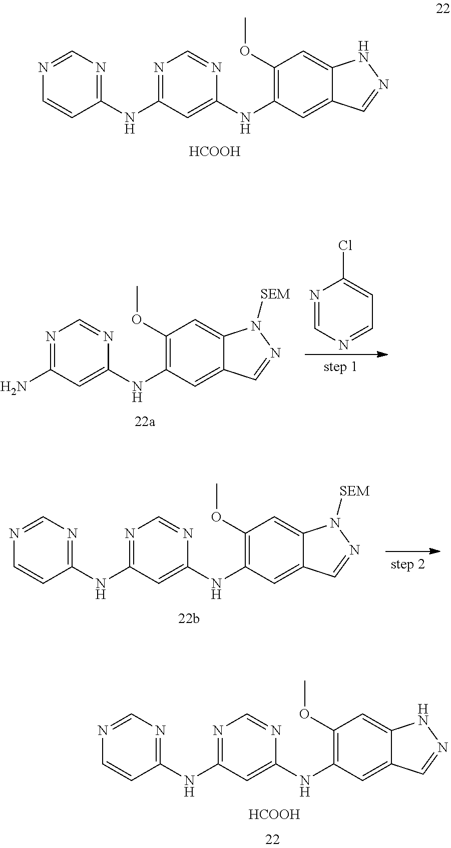

Step 1

### 1-((2-(trimethylsilyl) ethoxy) methyl)-5-(6-(pyrimidin-4-ylamino) pyrimidin-4-ylamino)-6-methoxyindazole

To the mixture of 1-((2-(trimethylsilyl) ethoxy) methyl)-5-(6-aminopyrimidin-4-ylamino)-6-methoxyindazole 22a (15.0 mg, 0.04 mmol), 4-chloropyrimidine (6.0 mg, 0.04 mmol) and 1,4-dioxane (1.0 mL) were added tris(dibenzylideneacetone)-dipalladium(0) (3.0 mg, 0.004 mmol), 4,5-bis(diphenylphosphino)-9,9-dimethylxanthene (4.0 mg, 0.008 mmol) and sodium t-butoxide (20.0 mg, 0.2 mmol) at room temperature under argon. The mixture was reacted at 90° C. in microwave oven under argon for 1 hour. It was cooled to room temperature and quenched with water (10 mL). The organic layer was separated and the aqueous layer was extracted with dichloromethane (15 mL×2). The combined organic layer was washed with saturated brine (50 mL×2), dried over anhydrous sodium sulfate and then filtered, the filtrate was concentrated under reduced pressure. Purification of the residue using Prep TLC (dichloromethane/methanol=20/1) gavel-((2-(trimethylsilyl) ethoxy) methyl)-5-(6-(pyrimidin-4-ylamino) pyrimidin-4-ylamino)-6-methoxyindazole 22b (10 mg, 0.021 mmol, yellow solid). Yield: 54%.

MS m/z (ESI): 465 [M+1],

Step 2

### 5-(6-(pyrimidin-4-ylamino) pyrimidin-4-ylamino)-6-methoxy-1H-indazole formate

1-((2-(trimethylsilyl) ethoxy) methyl)-5-(6-(pyrimidin-4-ylamino) pyrimidin-4-ylamino)-6-methoxyindazole 22b (10.0 mg, 0.021 mmol), dichloromethane (1 mL) and trifluoroacetic acid (1 mL) were mixed and stirred at room temperature for 3 hours. The mixture was quenched with saturated aqueous sodium bicarbonate (10 mL). The organic layer was separated and the aqueous layer was extracted with dichloromethane (15 mL×2). The combined organic layer was washed with saturated brine (30 mL), dried over anhydrous sodium sulfate and filtered. The filtrate was concentrated under reduced pressure. Purification of the residue using Prep-HPLC (water (0.2% formic acid), 10%˜40% acetonitrile, 15 minutes) gave 5-(6-(pyrimidin-4-ylamino) pyrimidin-4-ylamino)-6-methoxy-1H-indazole formate 22 (3.0 mg, 0.009 mmol, white solid). Yield: 46%.

MS m/z (ESI): 335 [M+1],

¹H NMR (400 MHz, DMSO-d6) δ 12.99 (s, 1H), 10.15 (s, 1H), 8.67 (s, 2H), 8.44 (d, J=6.0 Hz, 1H), 8.29 (s, 1H), 8.17 (s, 1H), 7.96 (s, 1H), 7.94 (s, 1H), 7.66 (d, J=6.0 Hz, 1H), 7.08 (s, 1H), 7.05 (s, 1H), 3.87 (s, 3H).

### Example 23

### 5-(6-((6-Chloropyridin-2-yl) amino) pyrimidin-4-ylamino)-6-methoxy-1H-indazole

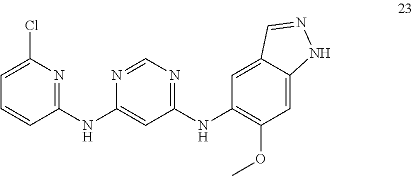 [SMILES: COc1cc2[nH]ncc2cc1Nc1cc(Nc2cccc(Cl)n2)ncn1]

The synthetic procedure is similar to that in Example 22. using 2,4-dichloropyridine instead of 4-chloropyrimidine, the desired compound 23: 5-(6-((6-chloropyridin-2-yl) amino) pyrimidin-4-ylamino)-6-methoxy-1H-indazole (4.0 mg, 0.011 mmol, white solid) was obtained. Yield: 79%.

MS m/z (ESI): 368 [M+1],

¹H NMR (400 MHz, DMSO-d6) δ 12.79 (s, 1H), 9.91 (s, 1H), 8.53 (s, 1H), 8.24 (s, 1H), 7.94 (s, 1H), 7.82 (s, 1H), 7.70-7.68 (m, 1H), 7.58 (d, J=8.2 Hz, 1H), 7.06 (s, 1H), 6.94 (d, J=7.5 Hz, 1H), 6.91 (s, 1H), 3.86 (s, 3H).

### Example 24

### 5-(6-((5-Chloropyridin-2-yl) amino) pyrimidin-4-ylamino)-6-methoxy-1H-indazole formate

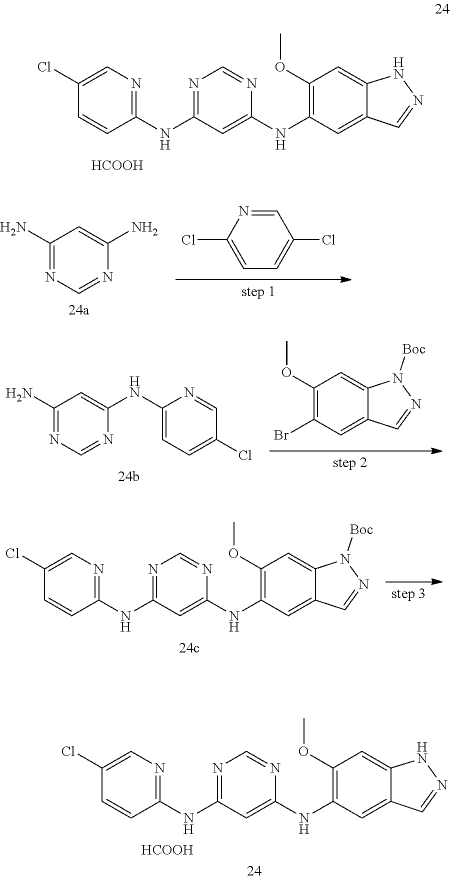

Step 1

### N-(5-Chloropyridin-2-yl) pyrimidine-4,6-diamine

To the mixture of 4,6-diaminopyrimidine 24a (110.0 mg, 1.0 mmol), sodium hydride (60% dispersion in mineral oil, 120.0 mg, 5.0 mmol) and N,N-dimethylacetamide (10.0 mL) was added 2,5-dichloropyridine (6.0 mg, 0.03 mmol) at room temperature. The mixture was reacted at 70° C. for 2 hours. Then it was cooled to room temperature and quenched with saturated aqueous sodium bicarbonate (10 mL). The mixture was extracted with dichloromethane (30 mL×3). The combined organic layer was washed with saturated brine (30 mL), dried over anhydrous sodium sulfate and filtered. The filtrate was concentrated under reduced pressure. Purification of the residue using flash column chromatography (dichloromethane/methanol=10/1) gave N-(5-chloropyridin-2-yl) pyrimidine-4,6-diamine 24b (45.0 mg, 0.20 mmol, white solid). Yield: 20%.

MS m/z (ESI): 222 & 224 [M+1],

Step 2

### 1-tert-Butoxycarbonyl-5-(6-((5-chloropyridin-2-yl) amino) pyrimidin-4-ylamino)-6-methoxyindazole

To the mixture of N-(5-chloropyridin-2-yl) pyrimidine-4,6-diamine 24b (22.0 mg, 0.10 mmol), 1-tert-butoxycarbonyl-5-bromo-6-methoxyindazole (32.0 mg, 0.10 mmol) and 1,4-dioxane (2.0 mL) were added tris(dibenzylideneacetone)-dipalladium(0) (9.0 mg, 0.01 mmol), 4,5-bis(diphenylphosphino)-9,9-dimethylxanthene (12.0 mg, 0.02 mmol) and cesium carbonate (98.0 mg, 0.3 mmol) under argon. The reaction mixture was reacted at 110° C. in microwave oven under argon for 1 hour. Then it was diluted with dichloromethane (10 mL) and filtered. The filtrate was concentrated under reduced pressure. Purification of the residue using Prep TLC (dichloromethane/methanol=20/1) gavel-tert-butoxycarbonyl-5-(6-((5-chloropyridin-2-yl) amino) pyrimidin-4-ylamino)-6-methoxyindazole 24c (10 mg, 0.02 mmol, white solid). Yield: 20%.

MS m/z (ESI): 468 & 470 [M+1],

Step 3

### 5-(6-((5-Chloropyridin-2-yl) amino) pyrimidin-4-ylamino)-6-methoxy-1H-indazole formate

1-tert-Butoxycarbonyl-5-(6-((5-chloropyridin-2-yl) amino) pyrimidin-4-ylamino)-6-methoxyindazole 24c (10.0 mg, 0.02 mmol), dichloromethane (2 mL) and trifluoroacetic acid (1 mL) were mixed and stirred at room temperature for 3 hours. Then the mixture was quenched with saturated aqueous sodium bicarbonate (5 mL). The organic layer was separated and the aqueous layer was extracted with dichloromethane (10 mL×2). The combined organic layer was washed with saturated brine (10 mL), dried over anhydrous sodium sulfate and filtered. The filtrate was concentrated under reduced pressure. Purification of the residue using Prep-HPLC (water (0.2% formic acid), 10%˜40% acetonitrile, 15 minutes) gave 5-(6-((5-chloropyridin-2-yl) amino) pyrimidin-4-ylamino)-6-methoxy-1H-indazole formate 24 (5.0 mg, 0.014 mmol, white solid). Yield: 70%.

MS m/z (ESI): 368 & 370 [M+1],

¹H NMR (400 MHz, DMSO-d6) δ 12.85 (s, 1H), 9.85 (s, 1H), 8.50 (s, 1H), 8.24 (s, 2H), 8.19 (s, 1H), 7.95 (s, 1H), 7.94 (s, 1H), 7.77 (s, 1H), 7.68 (s, 1H), 7.04 (d, J=9.2 Hz, 1H), 6.98 (d, J=9.2 Hz, 1H), 3.87 (s, 3H).

### Example 25

### 5-(6-((4-(Trifluoromethyl) pyridin-2-yl) amino) pyrimidin-4-ylamino)-6-methoxy-1H-indazole

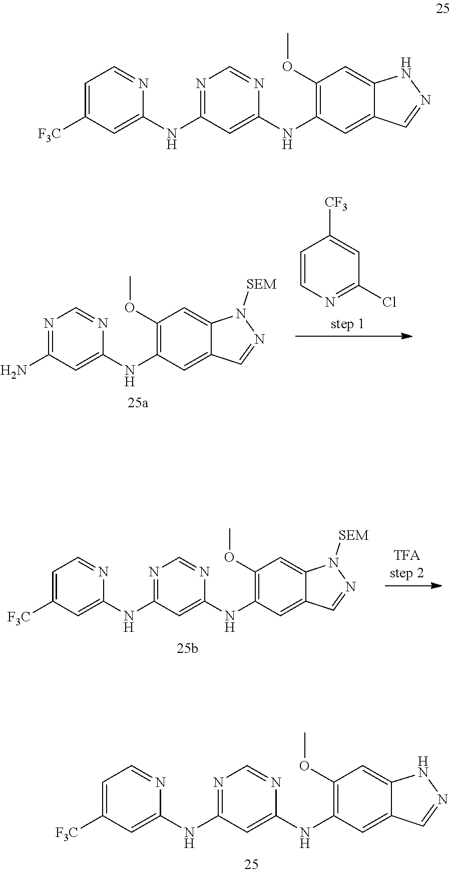

Step 1

### 1-((2-(Trimethylsilyl) ethoxy) methyl)-5-(6-((4-(trifluoromethyl) pyridin-2-yl) amino) pyrimidin-4-yl Amino)-6-methoxyindazole

To the mixture of 1-((2-(trimethylsilyl) ethoxy) methyl)-5-(6-aminopyrimidin-4-ylamino)-6-methoxyindazole 25a (15.0 mg, 0.039 mmol), 2-chloro-4-(trifluoromethyl)pyridine (10.7 mg, 0.059 mmol) and 1,4-dioxane (1 mL) were added tris(dibenzylideneacetone)dipalladium(0) (3.6 mg, 0.004 mmol), 4,5-bis(diphenylphosphino)-9,9-dimethylxanthene (2.3 mg, 0.004 mmol) and sodium t-butoxide (7.5 mg, 0.078 mmol) under argon. The reaction mixture was stirred at 80° C. in microwave oven under argon for 1 hour. Then it was cooled to room temperature, diluted with dichloromethane (10 mL) and filtered. The filtrate was concentrated under reduced pressure. Purification of the residue using Prep-HPLC (water (0.2% formic acid), 40%-60% acetonitrile, 15 minutes) gave 1-((2-(trimethylsilyl) ethoxy) methyl)-5-(6-((4-(trifluoromethyl) pyridin-2-yl) amino) pyrimidin-4-yl Amino)-6-methoxyindazole 25b (5.5 mg, 0.01 mmol, white solid).

Yield: 26%.

MS m/z (ESI): 532 [M+1],

Step 2

### 5-(6-((4-(Trifluoromethyl) pyridin-2-yl) amino) pyrimidin-4-ylamino)-6-methoxy-1H-indazole

1-((2-(Trimethylsilyl) ethoxy) methyl)-5-(6-((4-(trifluoromethyl) pyridin-2-yl) amino) pyrimidin-4-yl Amino)-6-methoxyindazole 25b (5.5 mg, 0.010 mmol) was dissolved in dichloromethane (1 mL) and trifluoroacetic acid (1 mL) was added. The mixture was stirred at room temperature for 2 hours. Then it was concentrated under reduced pressure. The residue was dissolved in dimethyl sulfoxide (1 mL) and adjusted to pH=8˜9 with one drop of saturated sodium hydroxide solution. Purification of the mixture using Prep-HPLC (water (0.2% formic acid), 30%˜70% acetonitrile, 15 minutes) gave 5-(6-((4-(trifluoromethyl) pyridin-2-yl) amino) pyrimidin-4-ylamino)-6-methoxy-1H-indazole 25 (1.6 mg, 0.004 mmol, white solid). Yield: 40%.

MS m/z (ESI): 402 [M+1],

¹H NMR (400 MHz, DMSO-d6) δ 12.85 (s, 1H), 10.09 (s, 1H), 8.58 (s, 1H), 8.43 (d, J=5.2 Hz, 1H), 8.28 (s, 1H), 8.06 (s, 1H), 8.02-7.88 (m, 2H), 7.20 (d, J=5.2 Hz, 1H), 7.04 (s, 1H), 7.00 (s, 1H), 3.87 (s, 3H).

### Example 26

### 5-(6-((5-chloropyrimidin-2-yl) amino) pyrimidin-4-ylamino)-6-methoxy-1H-indazole

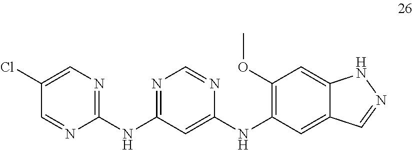 [SMILES: COc1cc2[nH]ncc2cc1Nc1cc(Nc2ncc(Cl)cn2)ncn1]

The synthetic procedure is similar to that of example 22. Using 2,5-dichloropyrimidine instead of 2, 5-dichloropyridine and the desired compound 26: 5-(6-((5-chloropyrimidin-2-yl) amino) pyrimidin-4-ylamino)-6-methoxy-1H-indazole (4.0 mg, 0.01 mmol, white solid) was obtained. Yield: 50%.

MS m/z (ESI): 369 & 371 [M+1],

¹H NMR (400 MHz, DMSO-d6) δ 12.85 (s, 1H), 10.29 (s, 1H), 8.80 (s, 1H), 8.64 (s, 2H), 8.28 (s, 1H), 8.00 (s, 1H), 7.96 (s, 1H), 7.47 (s, 1H), 7.05 (s, 1H), 3.87 (s, 3H).

### Example 27

### 5-(6-(Pyrimidin-2-ylamino) pyrimidin-4-ylamino)-6-methoxy-1H-indazole

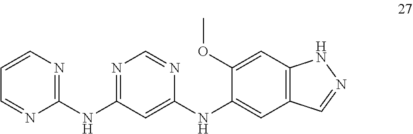 [SMILES: COc1cc2[nH]ncc2cc1Nc1cc(Nc2ncccn2)ncn1]

The synthetic procedure is similar to that of example 20. Using 2-chloropyrimidine instead of 2-bromopyridine, and the desired compound 5-(6-(pyrimidin-2-ylamino) pyrimidin-4-ylamino)-6-methoxy-1H-indazole 27 was obtained. Yield: 63%.

MS m/z (ESI): 335 [M+1],

¹H NMR (400 MHz, DMSO-d6) δ 13.03 (s, 1H), 11.25 (s, 1H), 10.07 (s, 1H), 8.70 (s, 1H), 8.69 (s, 1H), 8.51 (s, 1H), 8.02 (s, 1H), 7.82 (s, 1H), 7.23 (t, J=5.2 Hz, 1H), 7.13 (s, 1H), 7.04 (s, 1H), 3.88 (s, 3H). Example 28

### 5-(6-((3-Chloropyridin-2-yl) amino) pyrimidin-4-ylamino)-6-methoxy-1H-indazole

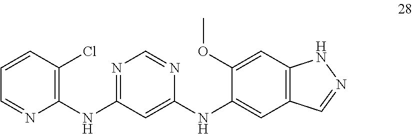 [SMILES: COc1cc2[nH]ncc2cc1Nc1cc(Nc2ncccc2Cl)ncn1]

The synthetic procedure is similar to that of example 24. Using 2,3-dichloropyridine instead of 2,5-dichloropyridine, and the desired compound: 5-(6-((3-chloropyridin-2-yl) amino) pyrimidin-4-ylamino)-6-methoxy-1H-indazole 28 was obtained. Yield: 76%.

MS m/z (ESI): 368 & 370 [M+1],

¹H NMR (400 MHz, DMSO-d6) δ 12.85 (s, 1H), 8.65 (s, 1H), 8.40 (s, 1H), 8.30 (s, 1H), 8.22 (d, J=6.4 Hz, 1H), 8.01 (s, 1H), 7.93 (d, J=10 Hz, 1H), 7.38 (s, 1H), 7.09-7.05 (m, 1H), 7.03 (s, 1H), 6.68 (s, 1H), 3.87 (s, 3H).

### Example 29

### 5-(6-(Pyrazin-2-ylamino) pyrimidin-4-ylamino)-6-methoxy-1H-indazole

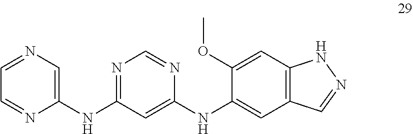 [SMILES: COc1cc2[nH]ncc2cc1Nc1cc(Nc2cnccn2)ncn1]

The synthetic procedure is similar to that of example 20. Using 2-chloropyrazine instead of 2-chloropyridine, and the desired compound: 5-(6-(pyrazin-2-ylamino) pyrimidin-4-ylamino)-6-methoxy-1H-indazole 29 was obtained. Yield: 31%.

MS m/z (ESI): 335 [M+1],

¹H NMR (400 MHz, DMSO-d6) δ 12.86 (s, 1H), 10.02 (s, 1H), 8.90 (s, 1H), 8.61 (s, 1H), 8.31 (s, 1H), 8.27 (s, 1H), 8.20 (d, J=2.4 Hz, 1H), 8.10 (d, J=2.4 Hz, 1H), 7.95 (s, 1H), 7.07 (s, 1H), 7.04 (s, 1H), 3.87 (s, 3H).

### Example 30

### 5-(6-Methylaminopyrimidin-4-ylamino)-6-methoxy-1H-indazole

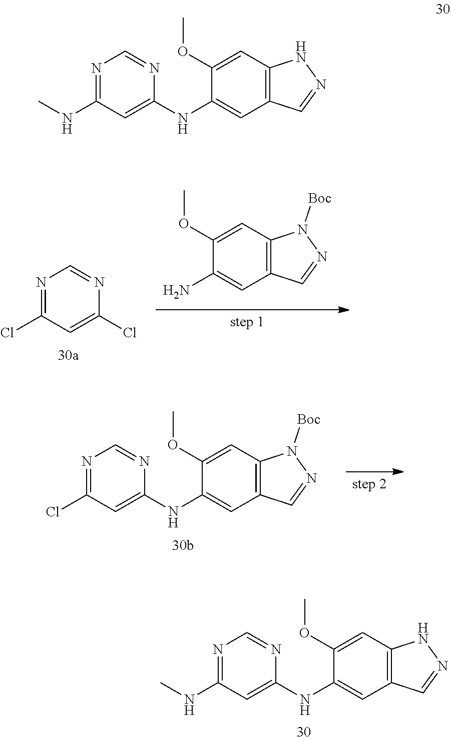 [SMILES: CNc1cc(Nc2cc3cn[nH]c3cc2OC)ncn1 ; Clc1cc(Cl)ncn1 ; COc1cc2c(cnn2C(=O)OC(C)(C)C)cc1N ; COc1cc2c(cnn2C(=O)OC(C)(C)C)cc1Nc1cc(Cl)ncn1 ; CNc1cc(Nc2cc3cn[nH]c3cc2OC)ncn1]

Step 1

### 1-tert-Butoxycarbonyl-5-(6-chloropyrimidin-4-ylamino)-6-methoxyindazole

4,6-Dichloropyrimidine 30a (26.3 mg, 0.1 mmol), 1-tert-butoxycarbonyl-5-amino-6-methoxyindazole (29.8 mg, 0.2 mmol), cesium carbonate (97.8 mg, 0.3 mmol) and dimethyl acetamide (1 mL) were mixed and stirred at 100° C. in microwave oven for 1 hour. Then it was diluted water (10 mL) and extracted with dichloromethane (10 mL×3). The combined organic layer was washed with saturated brine (10 mL×2), dried over anhydrous sodium sulfate and filtered. The filtrate was concentrated under reduced pressure. Purification of the residue using Prep-TLC (dichloromethane/methanol=20/1) gavel-tert-butoxycarbonyl-5-(6-chloropyrimidin-4-ylamino)-6-methoxyindazole 30b (10 mg, 0.027 mmol, yellow solid). Yield: 27%.

MS m/z (ESI): 376 & 378 [M+1],

Step 2

### 5-(6-Methylaminopyrimidin-4-ylamino)-6-methoxy-1H-indazole

1-tert-Butoxycarbonyl-5-(6-chloropyrimidin-4-ylamino)-6-methoxyindazole 30b (10 mg, 0.027 mmol) and methanamine (2 mL, 8 mmol, 4 M solution in ethanol) were mixed and stirred at 70° C. for 5 hours. Then it was concentrated under reduced pressure. Purification of the residue using Prep-HPLC (water (0.2% formic acid), 20%˜70% acetonitrile, 15 minutes) gave 5-(6-methylamino-pyrimidin-4-ylamino)-6-methoxy-1H-indazole 30 (4 mg, 0.015 mmol, white solid).

Yield: 56%.

MS m/z (ESI): 271 [M+1],

¹H NMR (400 MHz, DMSO-d6) δ 12.82 (s, 1H), 8.02-7.91 (m, 4H), 7.00 (s, 1H), 6.67-6.61 (m, 1H), 5.61 (s, 1H), 3.87 (s, 3H), 2.68-2.67 (d, J=4.4 Hz, 3H).

### Example 31

### 5-(6-Cyclopropylaminopyrimidin-4-ylamino)-6-methoxy-1H-indazole formate

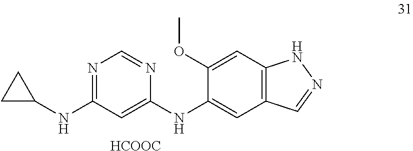

The synthetic procedure is similar to that of example 30. Using cyclopropylamine instead of methylamine, and the desired compound 5-(6-cyclopropylamino-pyrimidin-4-ylamino)-6-methoxy-1H-indazole formate 31 was obtained. Yield: 37%, Prep HPLC conditions (water (0.2% formic acid), 20% to 70% acetonitrile, 15 minutes).

MS m/z (ESI): 297 [M+1],

¹H NMR (400 MHz, DMSO-d6) δ 12.80 (s, 1H), 8.09 (s, 1H), 8.05 (s, 1H), 8.00 (s, 1H), 7.91 (s, 1H), 7.01 (s, 1H), 6.97 (s, 1H), 6.07 (s, 1H), 5.89 (s, 1H), 3.88 (s, 3H), 2.40-2.31 (m, 1H), 0.72-0.56 (m, 2H), 0.44-0.38 (m, 2H).

### Example 32

### 5-((6-Amino-5-cyclohex-3-ene-1-carboxypyrimidin-4-yl) amino)-6-methoxy-1H-indazole hydrochloride

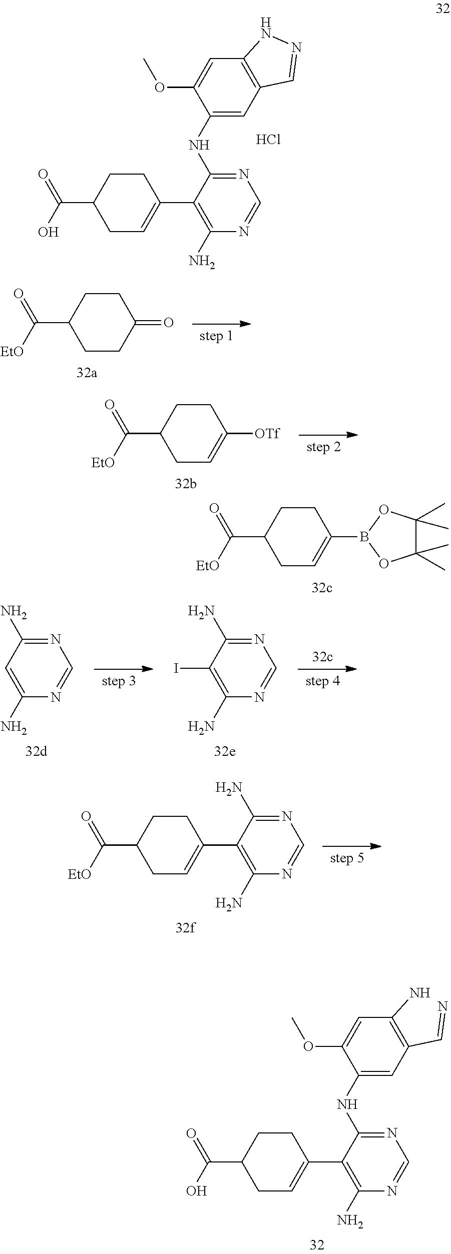

Step 1

### Ethyl 4-(((trifluoromethyl)sulfonyl)oxy)cyclohex-3-ene-1-carboxylate

To the mixture of pyridine (1.7 g, 21.18 mmol) in anhydrous toluene (15.0 mL) was added dropwise trifluoromethanesulfonic anhydride (5.9 g, 21.28 mmol) in an ice bath. The mixture was stirred at room temperature for 30 minutes, then the solution of ethyl 4-oxocyclohexane-1-carboxylate 32a (3.0 g, 17.65 mmol) in anhydrous toluene (15 mL) was added to the mixture. It was stirred at 50° C. for 12 hours. Then it was quenched with water (30 mL). The organic layer was separated. Silica gel (15 g) was added to the organic layer and filtered. The filter cake was washed with toluene (10 mL×3). The combined organic layer was dried over anhydrous sodium sulfate and filtered. The filtrate was concentrated under reduced pressure to give ethyl 4-(((trifluoromethyl)sulfonyl)oxy)cyclohex-3-ene-1-carboxylate 32b (3 g, 9.93 mmol, yellow oil). Yield: 56%.

¹H NMR (400 MHz, CDCl₃) δ 5.79-5.77 (m, 1H), 4.16 (q, J=7.1 Hz, 2H), 2.64-2.53 (m, 1H), 2.48-2.38 (m, 4H), 2.18-2.09 (m, 1H), 1.99-1.86 (m, 1H), 1.27 (t, J=7.1 Hz, 3H).

Step 2

### 1-Ethoxycarbonylcyclohex-3-ene-4-boronic acid pinacol ester

To the mixture of ethyl 4-(((trifluoromethyl)sulfonyl)oxy)cyclohex-3-ene-1-carboxylate 32b (1.0 g, 3.31 mmol), bis(pinacolato)diboron (1.2 g, 5.30 mmol) and potassium acetate (0.7 g, 6.62 mmol) in 1,4-dioxane (10.0 mL) was added [1,1′-bis(diphenylphosphino)ferrocene]dichloropalladium(II) (0.24 g, 0.16 mmol) under agon. The reaction mixture was stirred at 90° C. for 12 hours. Then it was cooled to room temperature and filtered. The filtrate was concentrated under reduced pressure. Purification of the residue using Prep-TLC (n-hexane/ethyl acetate=10/1) gave 1-ethoxycarbonylcyclohex-3-ene-4-boronic acid pinacol ester 32c (0.20 g, 0.71 mmol, colorless oil). Yield: 21%.

MS m/z (ESI): 281 [M+1],

Step 3

### 4,6-Diamino-5-iodopyrimidine

To the mixture of 4,6-diaminopyrimidine 32d (1.0 g, 9.10 mmol) and potassium carbonate (1.9 g, 13.70 mmol) in water (20.0 mL) and N,N-dimethylformamide (10.0 mL) was added iodine (2.6 g, 10.00 mmol) at room temperature. The mixture was stirred at 45° C. for 18 hours. Then it was quenched with saturated aqueous sodium bisulfite (10 mL) and filtered. The filter cake was washed with water (40 mL) to give 4,6-diamino-5-iodopyrimidine 32e (1.2 g, 5.08 mmol, white solid). Yield: 56%.

MS m/z (ESI): 237 [M+1],

¹H NMR (400 MHz, CDCl₃) δ 7.72 (s, 1H), 6.35-6.25 (brs, 4H).

Step 4

### Ethyl 4-(4,6-diaminopyrimidin-5-yl)cyclohex-3-ene-1-carboxylate

To the solution of 4,6-diamino-5-iodopyrimidine 32e (80.0 mg, 0.34 mmol), 1-ethoxycarbonylcyclohex-3-ene-4-boronic acid pinacol ester 32c (120.0 mg, 0.44 mmol) and cesium carbonate (320.0 mg, 0.10 mmol) in 1,4-dioxane (5.0 mL) and water (1.0 mL) were added palladium(II) acetate (8.0 mg, 0.03 mmol) and 2-dicyclohexylphosphino-2′,6′-dimethoxybiphenyl (28.0 mg, 0.06 mol) under argon. The mixture was stirred at 50° C. under argon for 12 hours. Then it was cooled to room temperature and filtered. The filtrate was concentrated under reduced pressure. Purification of the residue using Prep-TLC (dichloromethane/methanol=15/1) gave ethyl 4-(4,6-diaminopyrimidin-5-yl)cyclohex-3-ene-1-carboxylate 32f (15 mg, 0.19 mmol, yellow solid). Yield: 56%.

MS m/z (ESI): 263 [M+1],

Step 5

### 5-((6-Amino-5-cyclohex-3-ene-1-carboxypyrimidin-4-yl) amino)-6-methoxy-1H-indazole hydrochloride

To the solution of ethyl 4-(4,6-diaminopyrimidin-5-yl)cyclohex-3-ene-1-carboxylate 32f (20 mg, 0.08 mmol), 1-tert-butoxycarbonyl-5-((6-acetylaminopyrimidin-4-yl) amino)-6-methoxy-1H-indazole (25 mg, 0.08 mmol) and sodium t-butoxide (30 mg, 0.30 mmol) in 1,4-dioxane (1.0 mL) were added tris(dibenzylideneacetone)dipalladium(0) (7 mg, 0.008 mmol) and 2-(dicyclohexylphosphine) 3,6-dimethoxy-2′, 4′, 6′-triisopropyl-1,1′-biphenyl(9 mg, 0.015 mmol) under argon. The mixture was stirred at 100° C. under argon for 12 hours. Then it was cooled to room temperature and adjusted to pH=5˜6 with diluted hydrochloric acid (1 M). The mixture was concentrated under reduced pressure. Purification of the residue using Prep-HPLC (water (0.2% formic acid), 20%-40% acetonitrile, 15 minutes) gave 5-((6-amino-5-cyclohex-3-ene-1-carboxypyrimidin-4-yl) amino)-6-methoxy-1H-indazole hydrochloride 32 (2 mg, 0.005 mmol, white solid). Yield: 6%.

MS m/z (ESI): 381 [M+1],

### Example 33

### 5-((6-Amino-5-cyclohex-3-en-1-yl (4-methylpiperazin-1-yl) methanylpyrimidin-4-yl) amino)-6-methoxy-1H-indazole formate

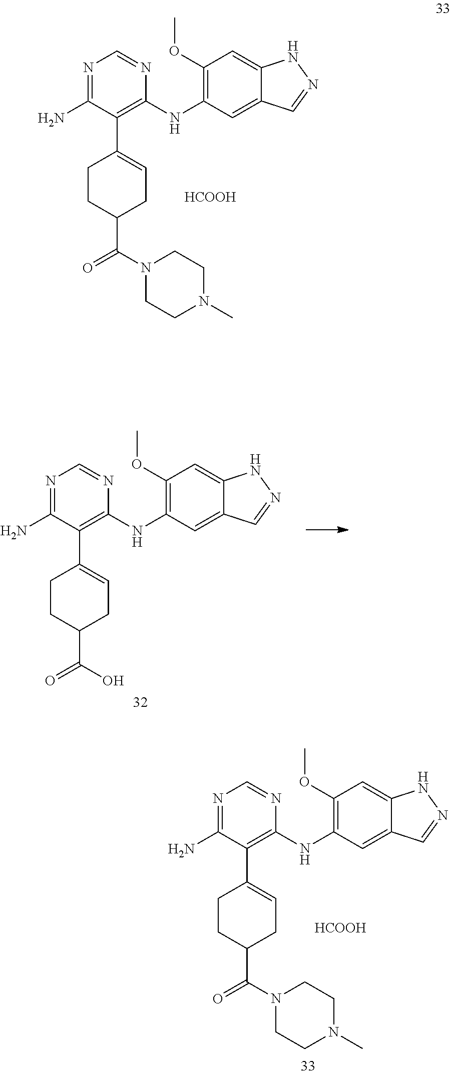 [SMILES: COc1cc2[nH]ncc2cc1Nc1ncnc(N)c1C1=CCC(C(=O)N2CCN(C)CC2)CC1 ; "HCOOH" ; COc1cc2[nH]ncc2cc1Nc1ncnc(N)c1C1=CCC(C(=O)O)CC1 ; COc1cc2[nH]ncc2cc1Nc1ncnc(N)c1C1=CCC(C(=O)N2CCN(C)CC2)CC1 ; "HCOOH"]

5-((6-Amino-5-cyclohex-3-ene-1-carboxypyrimidin-4-yl) amino)-6-methoxy-1H-indazole hydrochloride 32 (7.0 mg, 0.02 mmol), 2-(7-azabenzotriazol-1-yl)-N,N,N′,N′-tetramethyluronium hexafluorophosphate (16.0 mg, 0.04 mmol), N,N-diisopropylethylamine (13.0 mg, 0.1 mmol) and N,N-dimethylformamide (1.0 mL) were mixed and stirred at room temperature for 10 minutes. Then N-methylpiperazine (20.0 mg, 0.2 mmol) was added to the above mixture and stirred at room temperature for 24 hours. The mixture was diluted with water (10 mL) and extracted with dichloromethane (10 mL×3). The combined organic layer was washed with saturated brine (10 mL), dried over anhydrous sodium sulfate and filtered. The filtrate was concentrated under reduced pressure. Purification of the residue using Prep-HPLC (water (0.2% formic acid), 10%˜40% acetonitrile, 15 minutes) gave 5-((6-amino-5-cyclohex-3-en-1-yl (4-methylpiperazin-1-yl) methanylpyrimidin-4-yl) amino)-6-methoxy-1H-indazole formate 33 (2.0 mg, 0.005 mmol, white solid). Yield: 25%.

MS m/z (ESI): 463 [M+1],

¹H NMR (400 MHz, CD₃OD) b 8.39 (s, 1H), 8.25 (s, 1H), 8.05 (s, 1H), 8.01 (s, 1H), 7.95 (s, 1H), 7.07 (s, 1H), 6.02 (s, 1H), 3.96 (s, 3H), 3.87-3.80 (m, 4H), 3.65-3.60 (m, 1H), 3.24-3.16 (m, 2H), 2.95-2.88 (m, 4H), 2.65 (s, 3H), 2.52-3.40 (m, 3H), 2.24-2.19 (m, 1H).

### Example 34

### 4-(4-Amino-6-((6-methoxy-1H-indazol-5-yl)amino)pyrimidin-5-yl)-N,N-dimethylcyclohex-3-ene-1-carboxamide formate

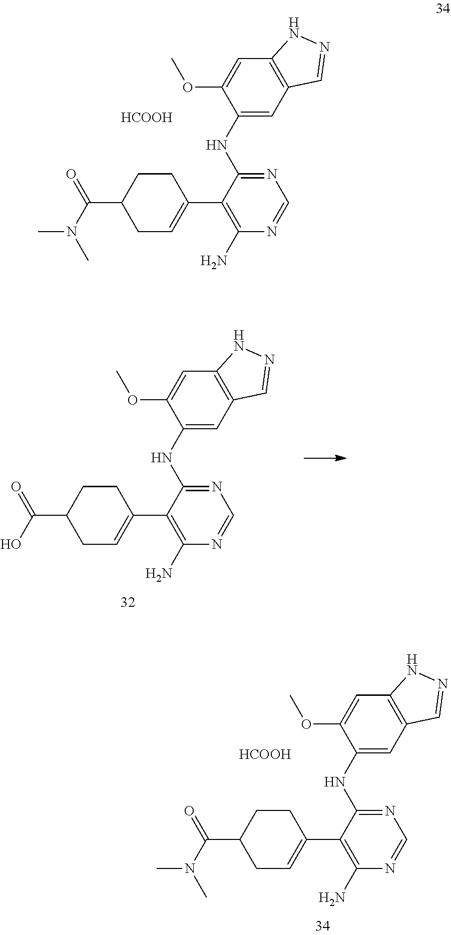 [SMILES: COc1cc2[nH]ncc2cc1Nc1ncnc(N)c1C1=CCC(C(=O)N(C)C)CC1 ; "HCOOH" ; COc1cc2[nH]ncc2cc1Nc1ncnc(N)c1C1=CCC(C(=O)O)CC1 ; COc1cc2[nH]ncc2cc1Nc1ncnc(N)c1C1=CCC(C(=O)N(C)C)CC1 ; "HCOOH"]

5-((6-Amino-5-cyclohex-3-ene-1-carboxypyrimidin-4-yl) amino)-6-methoxy-1H-indazole hydrochloride 32 (7.0 mg, 0.02 mmol), 2-(7-azabenzotriazol-1-yl)-N,N,N′,N′-tetramethyluronium hexafluorophosphate (11.0 mg, 0.03 mmol), dimethylamine hydrochloride (5.0 mg, 0.06 mmol), diisopropylethylamine (10.0 mg, 0.07 mmol) were dissolved into N,N-dimethylformamide (1.0 mL) and stirred at room temperature for 12 hours. Then it was concentrated under reduced pressure. Purification of the residue using Prep-HPLC (water (0.2% formic acid), 20%-50% acetonitrile, 15 minutes) gave 4-(4-amino-6-((6-methoxy-1H-indazol-5-yl)amino)pyrimidin-5-yl)-N,N-dimethylcyclohex-3-ene-1-carboxamide formate 34 (2.5 mg, 0.006 mmol white solid). Yield: 31%.

MS m/z (ESI): 408 [M+1],

¹H NMR (400 MHz, DMSO-d6) δ 12.65 (brs, 2H), 8.59 (s, 1H), 8.07 (s, 1H), 8.01 (s, 1H), 7.85 (s, 1H), 7.42-7.32 (m, 1H), 6.93 (s, 1H), 6.03-5.95 (m, 2H), 3.86 (s, 3H), 3.05 (s, 3H), 2.80 (s, 3H), 2.32-2.04 (m, 4H), 1.94-1.62 (m, 3H).

### Example 35

### 1-tert-Butoxycarbonyl-5-((6-amino-5-ethoxycarbonylpyrimidin-4-yl) amino)-6-methoxyindazole

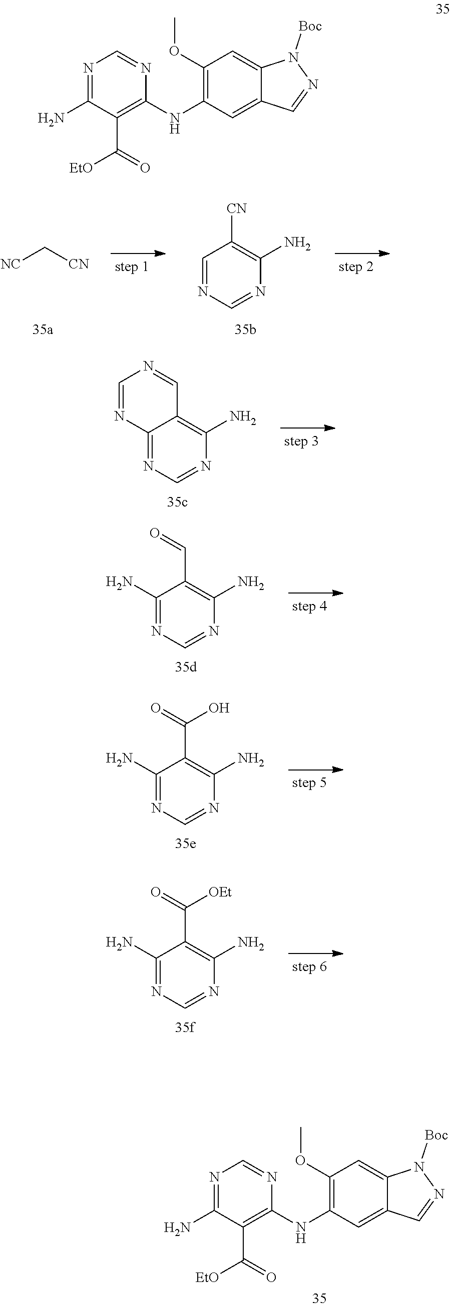

Step 1

### 4-Aminopyrimidine-5-carbonitrile

To the mixture of malononitrile 35a (20.8 g, 200 mmol), formamidine acetate (6.6 g, 100 mmol) and methanol (400 mL) was added in portions sodium methanolate (13.5 g, 250 mmol) at room temperature. The mixture was stirred at room temperature for 48 hours. Then it was filtered. The filter cake was washed with ice water (200 mL) and dried to give 4-aminopyrimidine-5-carbonitrile 35b (5.4 g, 45 mmol, yellow solid). Yield: 45%.

¹H NMR (400 MHz, DMSO-d6) δ 8.59 (s, 1H), 8.53 (s, 1H), 7.91 (s, 2H).

Step 2

### Pyrimido[4,5-d]pyrimidin-4-amine

4-aminopyrimidine-5-carbonitrile 35b (2.4 g, 20 mmol), formamidine acetate (3.1 g, 30 mmol) and ethylene glycol (20 mL) were mixed and stirred at 140° C. under argon for 0.5 hour. Then it was filtered. The filter cake was washed with methanol (50 mL) and dried to give pyrimido[4,5-d]pyrimidin-4-amine 35c (1.5 g, 10 mmol, yellow solid). Yield: 50%.

¹H NMR (400 MHz, DMSO-d6) δ 9.71 (s, 1H), 9.34 (s, 1H), 8.71-8.49 (m, 3H).

Step 3

### 4,6-Diaminopyrimidine-5-carbaldehyde

Pyrimido[4,5-d]pyrimidin-4-amine 35c (1.0 g, 7 mmol) and hydrochloric acid aqueous solution (20 mL, 0.5 M) were mixed at room temperature and stirred at 100° C. for 2 hours. Then it was quenched with 1 M aqueous sodium hydroxide. The solid formed was filtered. The filter cake was washed with water (50 mL) and dried to give 4,6-diaminopyrimidine-5-carbaldehyde 35d (450.0 mg, 3.4 mmol, yellow solid). Yield: 48%.

¹H NMR (400 MHz, DMSO-d6) δ 10.06 (s, 1H), 7.90 (s, 1H), 7.74 (s, 4H).

Step 4

### 4,6-Diaminopyrimidine-5-carboxylic acid

To the mixture of 4,6-diaminopyrimidine-5-carbaldehyde 35d (0.4 g, 3.0 mmol) and ammonium chlorite (0.14 g, 15 mmol) in dichloromethane (20 mL) and water (20 mL) was added dropwise phosphoric acid (1.5 mL) and dimethyl sulfoxide (3 mL) at room temperature. The mixture was stirred at room temperature for 24 hours. Then it was quenched with saturated aqueous sodium bicarbonate (50 mL) and filtered. The filter cake was washed with water (50 mL) and dried to give 4,6-diaminopyrimidine-5-carboxylic acid 35e (0.3 g, 1.8 mmol, yellow solid). Yield: 60%.

¹H NMR (400 MHz, DMSO-d6) δ 13.14 (s, 1H), 8.70 (s, 4H), 8.26 (s, 1H).

Step 5

### Ethyl 4,6-diaminopyrimidine-5-carboxylate

To the mixture of 4,6-diaminopyrimidine-5-carboxylic acid 35e (462.0 mg, 3 mmol) in dichloromethane (20 mL) was added slowly N,N-dimethylformamide (5 d) and oxalyl chloride (1.5 mL) at room temperature. The mixture was stirred at room temperature for 0.5 hour. Then anhydrous ethanol was added and stirred at room temperature for 0.5 hour. Then it was quenched with saturated aqueous sodium dicarbonate (50 mL). The organic layer was separated, and the aqueous layer was extracted with dichloromethane (50 mL×3). The combined organic layer was washed with saturated brine (50 mL), dried with anhydrous sodium sulfate and filtered. The filtrate was concentrated under reduced pressure. Purification of the residue using flash column chromatography (dichloromethane/methanol=20:1) gave ethyl 4,6-diaminopyrimidine-5-carboxylate 35f (280.0 mg, 1.5 mmol, white solid). Yield: 51%.

¹H NMR (400 MHz, CDCl₃) δ 8.02 (s, 1H), 7.66 (s, 2H), 5.68 (s, 2H), 4.39 (q, J=7.2 Hz, 2H), 1.42 (t, J=7.2 Hz, 3H).

Step 6

### 1-tert-Butoxycarbonyl-5-((6-amino-5-ethoxycarbonylpyrimidin-4-yl) amino)-6-methoxyindazole

To the mixture of ethyl 4,6-diaminopyrimidine-5-carboxylate 35f (18.0 mg, 0.1 mmol), 1-tert-butoxycarbonyl-5-bromo-6-methoxyindazole (232.0 mg, 0.10 mmol) and 1,4-dioxane (2.0 mL) were added tris(dibenzylideneacetone)dipalladium(0) (9 mg, 0.01 mmol), 4,5-bis(diphenylphosphino)-9,9-dimethylxanthene (12 mg, 0.02 mmol) and cesium carbonate (98.0 mg, 0.30 mmol) under argon. The reaction mixture was stirred at 110° C. in microwave oven under argon for 1 hour. Then it was diluted with dichloromethane (10 mL) and filtered. The filtrate was concentrated under reduced pressure. Purification of the residue using Prep-TLC (hexane/ethyl acetate=1/1) gave 1-tert-butoxycarbonyl-5-((6-amino-5-ethoxycarbonylpyrimidin-4-yl) amino)-6-methoxyindazole 35 (5 mg, 0.01 mmol, white solid). Yield: 10%.

MS m/z (ESI): 429 [M+1],

¹H NMR (400 MHz, CDCl₃) δ 10.76 (s, 1H), 8.94 (s, 1H), 8.24 (s, 1H), 8.10 (s, 1H), 7.75 (s, 1H), 4.50 (q, J=7.2 Hz, 2H), 4.07 (s, 3H), 1.73 (s, 9H), 1.49 (t, J=7.2 Hz, 3H).

### Example 36

### 5-((6-Amino-5-ethoxycarbonylpyrimidin-4-yl) amino)-6-methoxy-1H-indazole dicarboxylate

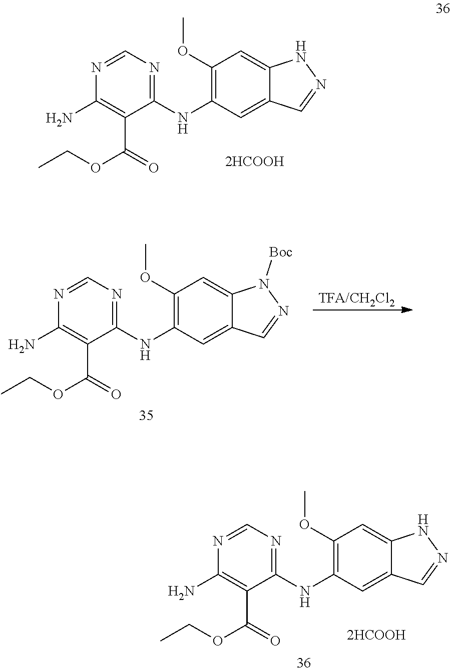 [SMILES: CCOC(=O)c1c(N)ncnc1Nc1cc2cn[nH]c2cc1OC ; CCOC(=O)c1c(N)ncnc1Nc1cc2cnn(C(=O)OC(C)(C)C)c2cc1OC ; CCOC(=O)c1c(N)ncnc1Nc1cc2cn[nH]c2cc1OC]

1-tert-Butoxycarbonyl-5-((6-amino-5-ethoxycarbonylpyrimidin-4-yl) amino)-6-methoxyindazole 35 (4.0 mg, 0.01 mmol), dichloromethane (2 mL) and trifluoroacetic acid (1 mL) were mixed and stirred at room temperature for 3 hours. Then it was quenched with saturated aqueous sodium bicarbonate (5 mL) and diluted with dichloromethane (5 mL). The organic layer was separated and the aqueous layer was extracted with dichloromethane (10 mL×3). The combined organic layer was washed with saturated brine (10 mL), dried over anhydrous sodium sulfate and filtered. The filtrate was concentrated. Purification of the residue using Prep-HPLC (water (0.2% formic acid), 30%˜70% acetonitrile, 15 minutes) gave 5-((6-amino-5-ethoxycarbonylpyrimidin-4-yl) amino)-6-methoxy-1H-indazole dicarboxylate 36 (1.3 mg, 0.004 mmol, white solid).

Yield: 40%.

MS m/z (ESI): 329 [M+1],

¹H NMR (400 MHz, DMSO-d6) δ 12.74 (s, 1H), 10.69 (s, 1H), 8.72 (s, 1H), 8.09 (s, 1H), 7.88 (s, 1H), 7.53 (s, 2H), 7.13 (s, 1H), 6.98 (s, 1H), 6.59 (s, 1H), 4.35 (q, J=7.2 Hz, 2H), 3.90 (s, 3H), 1.31 (t, J=7.2 Hz, 3H).

### Example 37

### 1-tert-Butoxycarbonyl-5-((6-(acetylamino)-5-ethoxycarbonylpyrimidin-4-yl) amino)-6-methoxyindazole

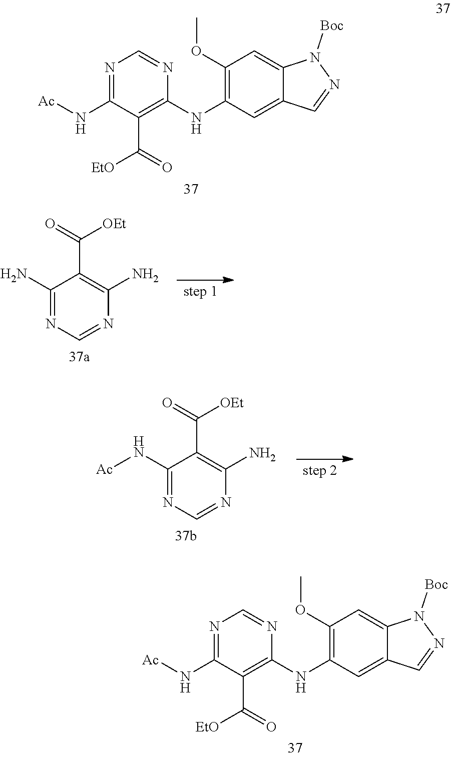 [SMILES: CCOC(=O)c1c(NC(C)=O)ncnc1Nc1cc2cnn(C(=O)OC(C)(C)C)c2cc1OC ; CCOC(=O)c1c(N)ncnc1N ; CCOC(=O)c1c(N)ncnc1NC(C)=O ; CCOC(=O)c1c(NC(C)=O)ncnc1Nc1cc2cnn(C(=O)OC(C)(C)C)c2cc1OC]

Step 1

### Ethyl 4-acetamido-6-aminopyrimidine-5-carboxylate

Ethyl 4,6-diaminopyrimidine-5-carboxylate 37a (128.0 mg, 0.7 mmol), acetic anhydride (72.0 mg, 0.7 mmol) and dioxane (10 mL) were mixed and stirred at 80° C. for 6 hours. Then it was cooled to room temperature and concentrated under reduced pressure. The mixture was extracted with dichloromethane (20 mL×3). The combined organic layer was washed with saturated brine (20 mL), dried with anhydrous sodium sulfate and filtered. The filtrate was concentrated under reduced pressure. Purification of the residue using Prep-TLC (dichloromethane/methanol=20:1) gave ethyl 4-acetamido-6-aminopyrimidine-5-carboxylate 37b (60.0 mg, 0.27 mmol, white solid). Yield: 38%.

MS m/z (ESI): 225 [M+1],

Step 2

### 1-tert-Butoxycarbonyl-5-((6-(acetylamino)-5-ethoxycarbonylpyrimidin-4-yl) amino)-6-methoxyindazole

To the mixture of ethyl 4-acetamido-6-aminopyrimidine-5-carboxylate 37b (23.0 mg, 0.1 mmol), 1-tert-butoxycarbonyl-5-bromo-6-methoxyindazole (32.0 mg, 0.10 mmol) and 1,4-dioxane (2.0 mL) were added tris(dibenzylideneacetone)-dipalladium(0) (9 mg, 0.01 mmol), 4,5-bis(diphenylphosphino)-9,9-dimethylxanthene (12 mg, 0.02 mmol) and cesium carbonate (98.0 mg, 0.30 mmol) under argon. The mixture was reacted at 110° C. in microwave oven under argon for 1 hour. Then it was diluted with dichloromethane (10 mL) and filtered. The filtrate was concentrated under reduced pressure. Purification of the residue using Prep-TLC (hexane/ethyl acetate=1:1) gave 1-tert-butoxycarbonyl-5-((6-(acetylamino)-5-ethoxycarbonylpyrimidin-4-yl) amino)-6-methoxyindazole 37 (6 mg, 0.015 mmol, white solid). Yield: 15%.

MS m/z (ESI): 471 [M+1],

¹H NMR (400 MHz, CDCl₃) δ 10.60 (s, 1H), 10.56 (s, 1H), 8.90 (s, 1H), 8.56 (s, 1H), 8.12 (s, 1H), 7.78 (s, 1H), 4.55 (q, J=7.2 Hz, 2H), 4.07 (s, 3H), 2.51 (s, 3H), 1.74 (s, 9H), 1.52 (t, J=7.2 Hz, 3H).

### Example 38

### 5-((6-(Acetylamino)-5-ethoxycarbonylpyrimidin-4-yl) amino)-6-methoxy-1H-indazole

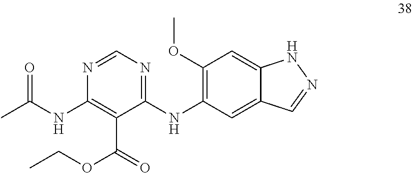 [SMILES: CCOC(=O)c1c(NC(C)=O)ncnc1Nc1cc2cn[nH]c2cc1OC]

The synthetic procedure is similar to that of example 2. Using 1-t-butoxyacyl-5-((6-(acetylamino)-5-ethoxycarbonylpyrimidine-4-yl)amino)-6-methoxyindazole instead of 1-t-butoxyacyl-5-(6-acetamidopyrimidine-4-ylamino)-6-methoxyindazole, the desired compound: 5-((6-(acetylamino)-5-ethoxycarbonylpyrimidin-4-yl) amino)-6-methoxy-1H-indazole 38 (1.5 mg, 0.004 mmol, white solid) was obtained. Yield: 40%.

MS m/z (ESI): 371 [M+1],

¹H NMR (400 MHz, CDCl₃) δ 10.57 (s, 1H), 10.45 (s, 1H), 8.76 (s, 1H), 8.54 (s, 1H), 8.03 (s, 1H), 6.96 (s, 1H), 4.54 (q, J=7.2 Hz, 2H), 4.00 (s, 3H), 2.51 (s, 3H), 1.52 (t, J=7.2 Hz, 3H).

### Example 39

### 5-((6-Amino-5-carbonylpyrimidin-4-yl) amino)-6-methoxy-1H-indazole formate

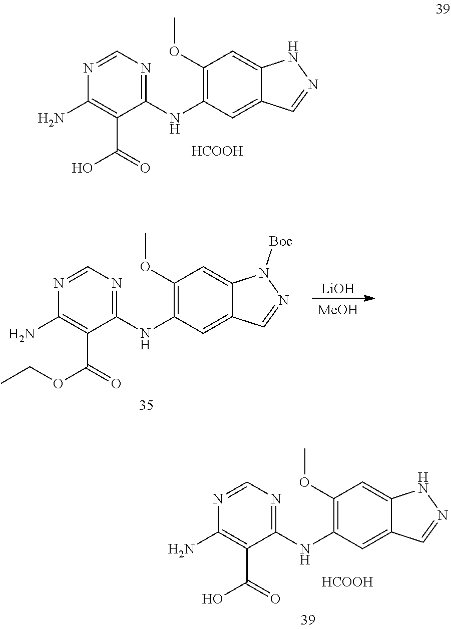

1-tert-Butoxycarbonyl-5-((6-amino-5-ethoxycarbonylpyrimidin-4-yl) amino)-6-methoxyindazole 35 (20.0 mg, 0.05 mmol), lithium hydroxide (1 mL, 1 M in water) and ethanol (5 mL) were mixed and stirred at room temperature for 1 hour. Then it was concentrated under reduced pressure. The residue was diluted with dichloromethane. The mixture was adjusted to pH=5˜6 with 0.5 M hydrochloric acid and extracted with dichloromethane (20 mL×3). The combined organic layer was washed with saturated brine (20 mL), dried with anhydrous sodium sulfate and filtered. The filtrate was concentrated under reduced pressure. Purification of the residue using Prep-HPLC (water (0.2% formic acid), 10%˜40% acetonitrile, 15 minutes) gave 5-((6-amino-5-carbonylpyrimidin-4-yl) amino)-6-methoxy-1H-indazole formate 39 (6.0 mg, 0.02 mmol, white solid). Yield: 40%.

MS m/z (ESI): 301 [M+1],

¹H NMR (400 MHz, CD₃OD) δ 8.71 (s, 1H), 8.63 (s, 1H), 8.37 (s, 1H), 7.29 (s, 1H), 4.10 (s, 3H).

### Example 40

### 5-((6-Amino-5-dimethylcarbamoyl) pyrimidin-4-yl) amino)-6-methoxy-1H-indazole

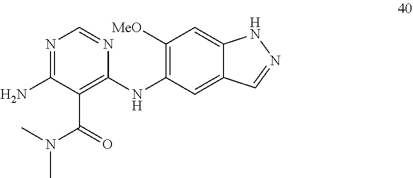 [SMILES: COc1cc2[nH]ncc2cc1Nc1ncnc(N)c1C(=O)N(C)C]

The synthetic procedure is similar to that of example 34. Using 4-amino-6-[(6-methoxy-1H-indazol-5-yl)amino]pyrimidine-5-methanoic acid instead of 4-(4-amino-6-((6-methoxy-1H-indazol-5-yl)amino)pyrimidine-5-yl)cyclohex-3-ene-1-methanoic acid. The desired compound: 5-((6-amino-5-dimethylcarbamoyl) pyrimidin-4-yl) amino)-6-methoxy-1H-indazole 40 (1.7 mg, 0.005 mmol, white solid) was obtained. Yield: 25%.

MS m/z (ESI): 328 [M+1],

¹H NMR (400 MHz, CD₃OD) b 8.09 (s, 1H), 8.00 (s, 1H), 7.98 (s, 1H), 7.19 (s, 1H), 3.95 (s, 3H), 3.10 (s, 6H).

### Example 41

### 5-((6-Amino-5-(4-morpholino) keto) pyrimidin-4-yl) amino)-6-methoxy-1H-indazole

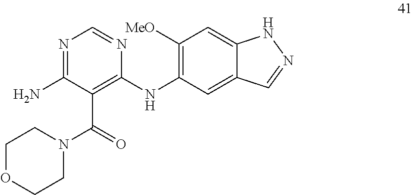 [SMILES: COc1cc2[nH]ncc2cc1Nc1ncnc(N)c1C(=O)N1CCOCC1]

The synthetic procedure is similar to that of example 34. Using 4-amino-6-[(6-methoxy-1H-indazol-5-yl)amino]pyrimidine-5-methanoic acid instead of 4-(4-amino-6-((6-methoxy-1H-indazol-5-yl)amino)pyrimidine-5-yl)cyclohex-3-ene-1-methanoic acid. The desired compound: 5-((6-amino-5-(4-morpholino) keto) pyrimidin-4-yl) amino)-6-methoxy-1H-indazole 41 (1.5 mg, 0.004 mmol, white solid) was obtained. Yield: 20%.

MS m/z (ESI): 370 [M+1];

¹H NMR (400 MHz, CD₃OD) δ 8.10 (s, 1H), 8.03 (s, 1H), 7.95 (s, 1H), 6.98 (s, 1H), 3.85 (s, 3H), 3.67-3.53 (m, 8H).

### Example 42

### 5-((6-Amino-5-cyanopyrimidin-4-yl) amino)-6-methoxy-1H-indazole

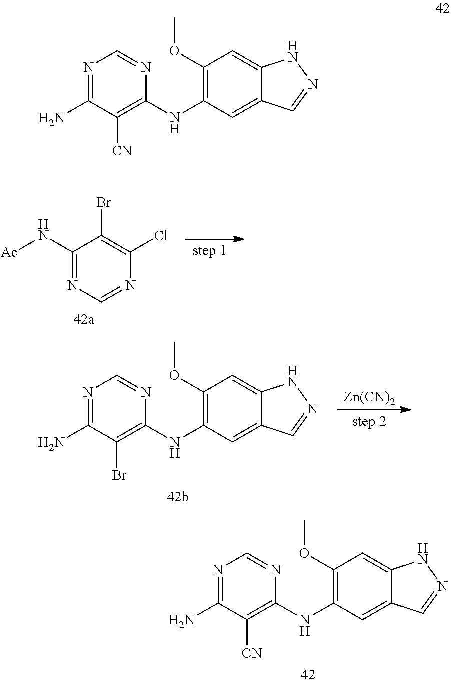

Step 1

### 5-((6-Amino-5-bromopyrimidin-4-yl) amino)-6-methoxy-1H-indazole

N-(5-bromo-6-chloropyrimidin-4-yl)acetamide 42a (84.0 mg, 0.4 mmol), 1-t-butoxycarbonyl-5-amino-6-methoxyindazole (105.0 mg, 0.4 mmol), cesium carbonate (391.0 mg, 1.2 mmol) and N,N-dimethylacetamide (5 mL) were mixed and stirred at 120° C. for 24 hours. Then it was cooled to room temperature and extracted with dichloromethane (30 mL×3). The combined organic layer was washed with saturated brine (30 mL), dried over anhydrous sodium sulfate and filtered. The filtrate was concentrated under reduced pressure. Purification of the residue using Prep HPLC (water (with 0.2% formic acid), 10%˜50% acetonitrile, 15 minutes) gave 5-((6-amino-5-bromopyrimidin-4-yl) amino)-6-methoxy-1H-indazole 42b (18.0 mg, 0.05 mmol, white solid), yield: 25%.

MS m/z (ESI): 335 & 337 [M+1].

Step 2

### 5-((6-Amino-5-cyanopyrimidin-4-yl) amino)-6-methoxy-1H-indazole

To the mixture of 5-((6-amino-5-bromopyrimidin-4-yl) amino)-6-methoxy-1H-indazole 42b (10.0 mg, 0.4 mmol), zinc cyanide (7.0 mg, 0.06 mmol) and N,N-dimethylacetamide (1 mL) were added copper(I) iodide (5.0 mg, 0.03 mmol) and tetrakis(triphenylphosphine)palladium(0) under argon. The mixture was reacted at 150° C. for 1 hour under argon in microwave oven. Then it was cooled to room temperature and filtered. The filtrate was concentrated. Purification of the residue using prep HPLC (water (0.2% formic acid), 20%˜60% acetonitrile, 15 minutes) gave 5-((6-amino-5-cyanopyrimidin-4-yl) amino)-6-methoxy-1H-indazole 42 (3.0 mg, 0.01 mmol, dark yellow solid). Yield: 36%.

MS m/z (ESI): 282 [M+1].

¹H NMR (400 MHz, DMSO-d6) δ 8.52 (s, 1H), 8.25 (s, 1H), 8.07 (s, 1H), 6.94 (s, 1H), 4.98 (s, 2H), 3.90 (s, 3H).

### Example 43&44

### 1-tert-Butoxycarbonyl-5-((6-amino-5-(pyridin-2-yl) pyrimidin-4-yl) amino-6-methoxyindazole 5-((6-amino-5-(pyridin-2-yl) pyrimidin-4-yl) amino-6-methoxy-1H-indazole formate

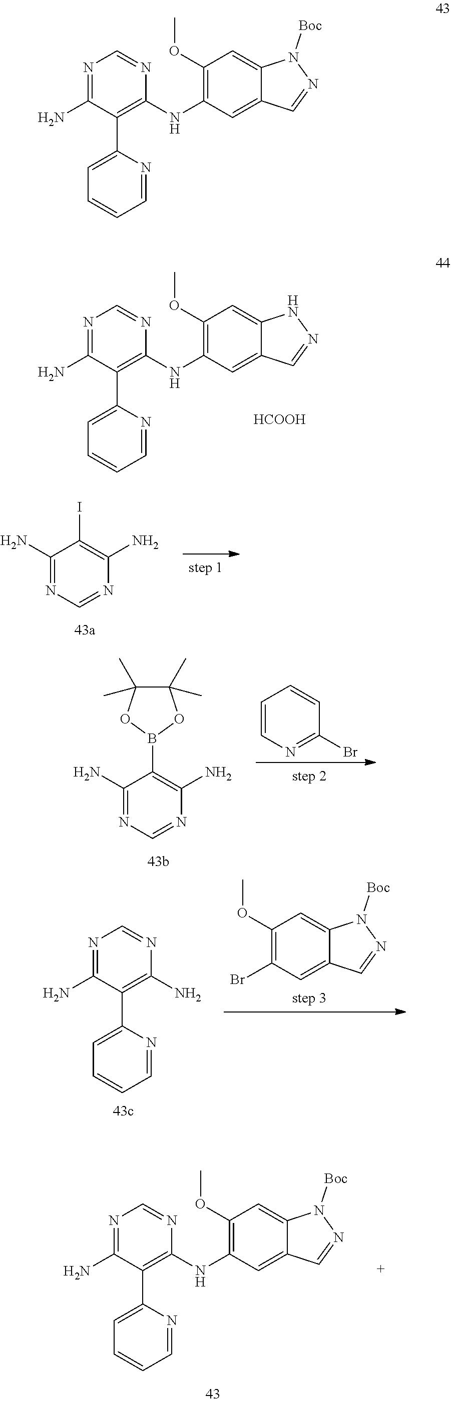

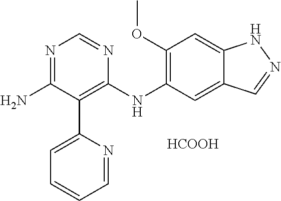 [SMILES: COc1cc2[nH]ncc2cc1Nc1ncnc(N)c1-c1ccccn1 ; "HCOOH"]

Step 1

### 4,6-Diamino-5-(4,4,5,5-tetramethyl-1,3,2-dioxoborolan-2-yl) pyrimidine

To the mixture of 4,6-diamino-5-iodopyrimidine 43a (125.0 mg, 0.5 mmol), bis(pinacolato)diboron (127.0 mg, 0.5 mmol) and 1,4-dioxane (5 mL) was added cesium carbonate (500.0 mg, 1.5 mmol), palladium(II) acetate (11.0 mg, 0.05 mmol) and 2-dicyclohexylphosphino-2′,6′-dimethoxybiphenyl (41.0 mg, 0.1 mmol) at room temperature under argon. The mixture was stirred at 60° C. for 16 hours under argon. Then it was quenched with water (10 mL). The organic layer was separated and the aqueous layer was extracted with dichloromethane (15 mL×2). The combined organic layer was washed with saturated brine (50 mL×2), dried over anhydrous sodium sulfate and filtered. The filtrate was concentrated under reduced pressure to give 4,6-diamino-5-(4,4,5,5-tetramethyl-1,3,2-dioxoborolan-2-yl) pyrimidine 43b as a crude product. It was used directly in next step without further purification.

MS m/z (ESI): 237 [M+1].

Step 2

### 4,6-Diamino-5-(pyridin-2-yl)pyrimidine

To the mixture of 4,6-diamino-5-(4,4,5,5-tetramethyl-1,3,2-dioxoborolan-2-yl) pyrimidine 43b (118.0 mg, 0.5 mmol), 2-bromopyridine (30.0 mg, 0.2 mmol) and 1,4-dioxane (5 mL) were added potassium acetate (98.0 mg, 1 mmol), palladium(II) acetate (9.0 mg, 0.01 mmol) and 2-dicyclohexylphosphino-2′,6′-dimethoxybiphenyl (8.0 mg, 0.02 mmol) at room temperature under argon. The mixture was stirred at 100° C. for 16 hours under argon. Then it was quenched with water (10 mL). The organic layer was separated and the aqueous layer was extracted with dichloromethane (15 mL×2). The combined organic layer was washed with saturated brine (50 mL×2), dried over anhydrous sodium sulfate and filtered. The filtrate was concentrated under reduced pressure. Purification of the residue using Prep HPLC (water (0.2% formic acid), 0%˜10% acetonitrile, 15 minutes) gave 4,6-diamino-5-(pyridin-2-yl)pyrimidine 43c (10.0 mg, 0.05 mmol, yellow solid). Yield: 10%.

MS m/z (ESI): 188 [M+1].

Step 3

### 1-tert-Butoxycarbonyl-5-((6-amino-5-(pyridin-2-yl) pyrimidin-4-yl) amino-6-methoxyindazole & 5-((6-Amino-5-(pyridin-2-yl) pyrimidin-4-yl) amino-6-methoxy-1H-indazole

To the mixture of 4,6-diamino-5-(pyridin-2-yl)pyrimidine 43c (10.0 mg, 0.05 mmol), 1-tert-butoxyacyl-5-bromo-6-methoxyindazole (16.0 mg, 0.05 mmol) and 1,4-dioxane (5 mL) were added cesium carbonate (32 mg, 0.1 mmol), tris(dibenzylideneacetone)dipalladium(0) (5 mg, 0.005 mmol) and 4,5-bis(diphenylphosphino)-9,9-dimethylxanthene (6 mg, 0.01 mmol) at room temperature under argon. The mixture was stirred at 120° C. for 16 hours under argon. Then it was quenched with water (10 mL). The organic layer was separated and the aqueous layer was extracted with dichloromethane (15 mL×2). The combined organic layer was washed with saturated brine (50 mL×2), dried over anhydrous sodium sulfate and filtered. The filtrate was concentrated under reduced pressure. Purification of the residue using Prep HPLC (water (with 0.8% ammonia bicarbonate), 20%-70% acetonitrile, 15 minutes) gavel-tert-butoxycarbonyl-5-((6-amino-5-(pyridin-2-yl) pyrimidin-4-yl) amino-6-methoxyindazole 43 (1.0 mg, 0.02 mmol, white solid). Yield: 4%. Further purification of the residue using Prep HPLC (water (with 0.2% formic acid), 20%˜70% acetonitrile, 15 minutes) gave 5-((6-amino-5-(pyridin-2-yl) pyrimidin-4-yl) amino-6-methoxy-1H-indazole formate 44 (1.0 mg, 0.003 mmol, white solid). Yield: 6%.

MS m/z (ESI): 434 [M+1],

¹H NMR (400 MHz, CDCl₃) δ 10.58 (s, 1H), 8.98 (s, 1H), 8.56 (d, J=4.4 Hz, 1H), 8.31 (s, 1H), 8.22 (s, 2H), 7.95-7.93 (m, 1H), 7.82 (d, J=8.4 Hz, 1H), 7.58 (s, 1H), 7.43-7.39 (m, 1H), 6.59 (s, 1H), 4.00 (s, 3H), 2.09 (s, 9H).

MS m/z (ESI): 334 [M+1],

¹H NMR (400 MHz, DMSO-d6) δ 12.78 (s, 1H), 10.29 (s, 1H), 8.81 (s, 1H), 8.28 (s, 1H), 8.17 (s, 2H), 7.94-7.91 (m, 2H), 7.80-7.78 (d, J=7.2 Hz, 1H), 7.40-7.38 (d, J=5.6 Hz, 1H), 6.96 (s, 1H), 6.49 (s, 2H), 3.91 (s, 3H).

### Example 45

### 5-((6-Amino-5-fluoropyrimidin-4-yl)amino)-6-methoxy-1H-indazole

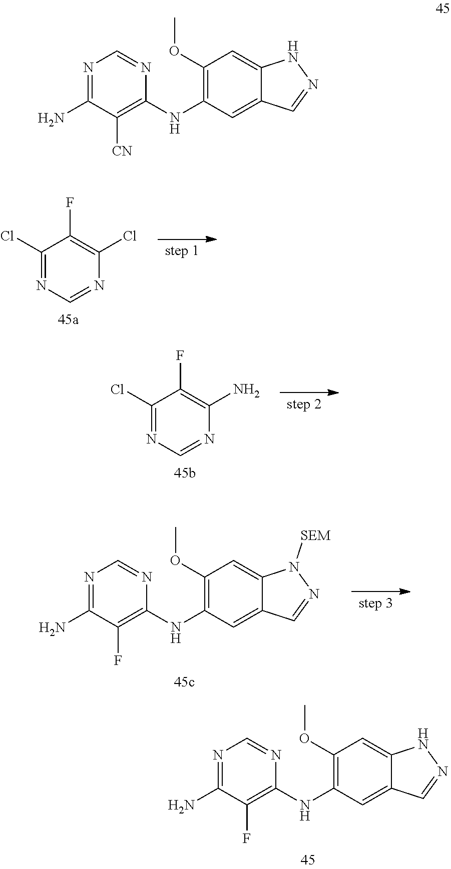 [SMILES: COc1cc2[nH]ncc2cc1Nc1ncnc(N)c1C#N ; Fc1c(Cl)ncnc1Cl ; Nc1ncnc(Cl)c1F ; COc1cc2c(cnn2COCC[Si](C)(C)C)cc1Nc1ncnc(N)c1F ; COc1cc2[nH]ncc2cc1Nc1ncnc(N)c1F]

Step 1

### 6-Chloro-5-fluoropyrimidin-4-amine

4,6-Dichloro-5-fluoropyrimidine 45a (0.5 g, 3.0 mmol), aqueous ammonia (3 mL) and butan-1-ol (2 mL) were mixed and stirred at 90° C. for 3 hours. White solid was formed and filtered. The filter cake was washed with acetonitrile (50 mL) and dried to give 6-chloro-5-fluoropyrimidin-4-amine 45b (0.2 g, 0.14 mmol, white solid). Yield: 48%.

MS m/z (ESI): 148 & 150 [M+1].

Step 2

### 1-((2-(Trimethylsilyl) ethoxy) methyl)-5-((6-amino-5-fluoropyrimidin-4-yl) amino)-6-methoxyindazole

To the mixture of 6-chloro-5-fluoropyrimidin-4-amine 45b (30.0 mg, 0.2 mmol), 6-methoxy-1-{[2-(trimethylsilyl)ethoxy]methyl}-1H-indazol-5-amine (32.0 mg, 0.1 mmol) and N,N-dimethylacetamide (2 mL) were added tris(dibenzylidene-acetone)dipalladium(0) (9.0 mg, 0.01 mmol), 4,5-bis(diphenylphosphino)-9,9-dimethylxanthene (12 mg, 0.02 mmol) and cesium carbonate (98 mg, 0.3 mmol) at room temperature under argon. The mixture was stirred at 125° C. for 1 hour under argon in microwave oven. Then it was cooled to room temperature and extracted with dichloromethane (20 mL×3). The combined organic layer was washed with brine (20 mL), dried over anhydrous sodium sulfate and filtered. The filtrate was concentrated under reduced pressure. Purification of the residue using Prep HPLC (water (with 0.2% formic acid), 10%˜50% acetonitrile, 15 minutes) gave 1-((2-(trimethylsilyl) ethoxy) methyl)-5-((6-amino-5-fluoropyrimidin-4-yl) amino)-6-methoxyindazole 45c (6.0 mg, 0.015 mmol, white solid). Yield: 15%.

MS m/z (ESI): 405 [M+1].

Step 3

### 5-((6-Amino-5-fluoropyrimidin-4-yl) amino)-6-methoxy-1H-indazole

1-((2-(Trimethylsilyl) ethoxy) methyl)-5-((6-amino-5-fluoropyrimidin-4-yl) amino)-6-methoxyindazole 45c (6.0 mg, 0.015 mmol), dichloromethane (2 mL) and trifluoroacetic acid (1 mL) were mixed and stirred at room temperature for 1 hour. Then it was quenched by saturated aqueous sodium bicarbonate (5 mL) and diluted with dichloromethane (5 mL). The organic layer was separated and the aqueous layer was extracted with dichloromethane (10 mL×3). The combined organic layer was washed with brine (10 mL), dried over anhydrous sodium sulfate and filtered. The filtrate was concentrated under reduced pressure. Purification of the residue using Prep HPLC (water (with 0.2% formic acid), 30%˜70% acetonitrile, 15 minutes) gave 5-((6-amino-5-fluoropyrimidin-4-yl) amino)-6-methoxy-1H-indazole 45 (2.0 mg, 0.007 mmol, white solid). Yield: 50%.

MS m/z (ESI): 275 [M+1],

¹H NMR (400 MHz, DMSO-d6) δ 12.83 (s, 1H), 8.15 (s, 1H), 7.94 (s, 1H), 7.80 (s, 1H), 7.77 (s, 1H), 7.02 (s, 1H), 6.63 (s, 2H), 3.89 (s, 3H).

### Example 46

### 5-((6-Amino-5-methoxypyrimidin-4-yl) amino)-6-methoxy-1H-indazole

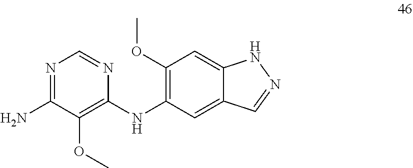 [SMILES: COc1cc2[nH]ncc2cc1Nc1ncnc(N)c1OC]

The synthetic procedure is similar to that of example 45. Using 6-chloro-5-methoxypyrimidine-4-amine instead of 5-fluorinepyrimidin-4-amine. The desired compound: 5-((6-amino-5-methoxypyrimidin-4-yl) amino)-6-methoxy-1H-indazole 46 (2.0 mg, 0.007 mmol, white solid) was obtained. Yield: 27%.

MS m/z (ESI): 287 [M+1],

¹H NMR (400 MHz, DMSO-d6) δ 12.87 (s, 1H), 8.56 (s, 1H), 7.94 (s, 1H), 7.89 (s, 1H), 7.60 (s, 1H), 7.04 (s, 1H), 6.39 (s, 2H), 3.96 (s, 3H), 3.69 (s, 3H).

### Example 47

### 5-((6-Amino-5-methylpyrimidin-4-yl) amino)-6-methoxy-1H-indazole

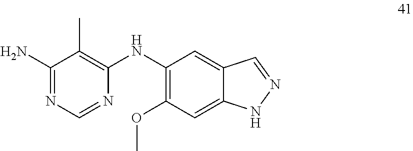 [SMILES: COc1cc2[nH]ncc2cc1Nc1ncnc(N)c1C]

The synthetic procedure is similar to that of example 45. Using 6-chloro-5-methylpyrimidine-4-amine instead of 5-fluorinepyrimidine-4-amine. The desired compound: 5-((6-amino-5-methylpyrimidin-4-yl) amino)-6-methoxy-1H-indazole 47 (1.0 mg, 0.004 mmol, white solid) was obtained. Yield: 36%.

MS m/z (ESI): 271 [M+1],

¹H NMR (400 MHz, DMSO-d6) δ 12.75 (s, 1H), 8.32 (s, 1H), 8.00-7.81 (m, 2H), 7.27 (s, 1H), 7.00 (s, 1H), 6.15 (s, 2H), 3.91 (s, 3H), 1.96 (s, 3H).

### Example 48

### 5-((6-(Cyclopropionamido)-5-methylpyrimidin-4-yl) amino)-6-methoxy-1H-indazole

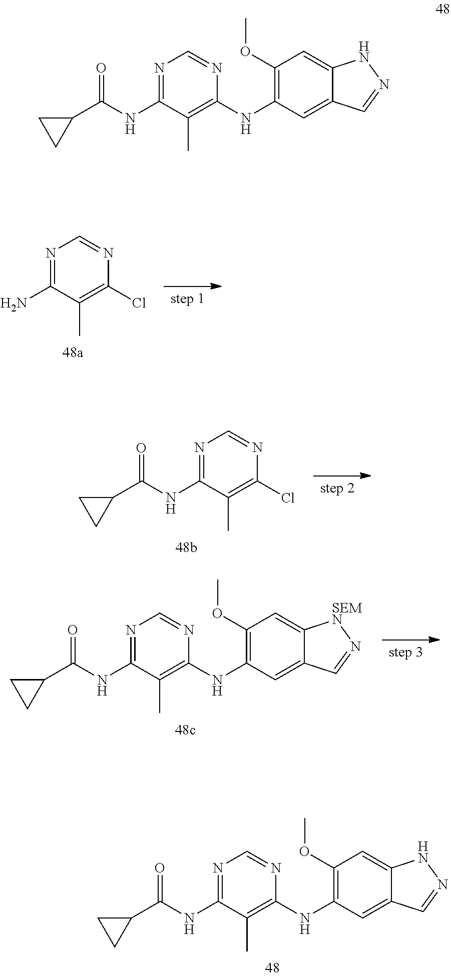

Step 1

N-(6-Chloro-5-methylpyrimidin-4-yl)cyclopropanecarboxamide 6-Chloro-5-methylpyrimidin-4-amine 48a (100.0 mg, 0.7 mmol), cyclopropylformyl chloride (87 mg, 0.839 mmol), pyridine (3 mL) and tetrahydrofuran (3 mL) were mixed at room temperature and stirred at 60° C. for 16 hours. Then it was diluted with water (20 mL) and extracted with ethyl acetate (20 mL×2). The organic layer was washed with brine (20 mL×2), dried over anhydrous sodium sulfate and filtered. The filtrate was concentrated under reduced pressure. Purification of the residue using Prep TLC (petroleum ether/ethyl acetate=4:1) gave N-(6-chloro-5-methylpyrimidin-4-yl)cyclopropanecarboxamide 48b (40.0 mg, 0.19 mmol, white solid), yield: 27%.

MS m/z (ESI): 212 & 214 [M+1];

Step 2

### 1-((2-(Trimethylsilyl) ethoxy) methyl)-5-((6-(cyclopropionylamino)-5-methylpyrimidin-4-yl) amino)-6-Methoxyindazole

To the solution of N-(6-chloro-5-methylpyrimidin-4-yl)cyclopropanecarboxamide 48b (10.0 mg, 0.05 mmol), 6-methoxy-1-{[2-(trimethylsilyl)ethoxy]methyl}-1H-indazol-5-amine (20.0 mg, 0.071 mmol) and cesium carbonate (46 mg, 0.142 mmol) in 1,4-dioxane (1 mL) were added tris(dibenzylideneacetone)dipalladium(0) (4 mg, 0.005 mmol) and 4,5-bis(diphenylphosphino)-9,9-dimethylxanthene (5 mg, 0.009 mmol) at room temperature under argon. The mixture was stirred at 110° C. for 1 hour under argon in microwave oven. Then it was diluted with water (10 mL) and extracted with ethyl acetate (10 mL×3). The combined organic layer was washed with brine (10 mL×2), dried over anhydrous sodium sulfate and filtered. The filtrate was concentrated under reduced pressure. Purification of the residue using Prep TLC (petroleum ether/ethyl acetate=3:2) gave 1-((2-(trimethylsilyl) ethoxy) methyl)-5-((6-(cyclopropionylamino)-5-methylpyrimidin-4-yl) amino)-6-Methoxyindazole 48c (10.0 mg, 0.021 mmol, pale yellow solid).

Yield: 46%.

MS m/z (ESI): 469 [M+1].

Step 3

### 5-((6-(Cyclopropionamido)-5-methylpyrimidin-4-yl) amino)-6-methoxy-1H-indazole

1-((2-(Trimethylsilyl) ethoxy) methyl)-5-((6-(cyclopropionylamino)-5-methylpyrimidin-4-yl) amino)-6-Methoxyindazole 48c (10.0 mg, 0.021 mmol), dichloromethane (0.5 mL) and trifluoroacetic acid (1 mL) were mixed and stirred at room temperature for 1 hour. Then it was diluted with water (10 mL) and extracted with dichloromethane (10 mL×3). The combined organic layer was dried over anhydrous sodium sulfate and filtered. The filtrate was concentrated under reduced pressure. Purification of the residue using Prep HPLC (water (with 0.8% ammonia bicarbonate), 40%˜60% acetonitrile, 15 minutes) gave 5-((6-(cyclopropionamido)-5-methylpyrimidin-4-yl) amino)-6-methoxy-1H-indazole 48 (3.0 mg, 0.009 mmol, white solid). Yield: 42%.

MS m/z (ESI): 339 [M+1],

¹H NMR (400 MHz, DMSO-d6) δ 12.85 (s, 1H), 10.29 (s, 1H), 8.21 (s, 1H), 8.04 (s, 1H), 7.99 (s, 1H), 7.96 (s, 1H), 7.04 (s, 1H), 3.86 (s, 3H), 1.96 (s, 3H), 1.95-1.92 (m, 1H), 0.83-0.81 (m, 4H).

### Example 49

### 5-((6-(Cyclopropanesulfonylamino) pyrimidin-4-yl) amino)-6-methoxy-1H-indazole

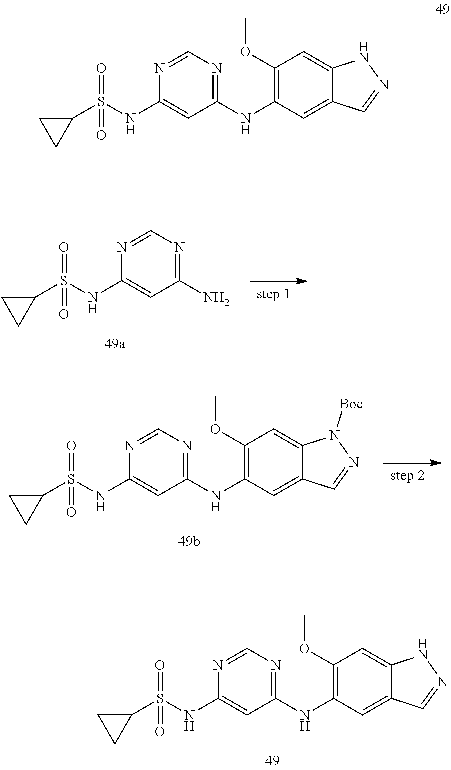 [SMILES: COc1cc2[nH]ncc2cc1Nc1cc(NS(=O)(=O)C2CC2)ncn1 ; Nc1cc(NS(=O)(=O)C2CC2)ncn1 ; COc1cc2c(cnn2C(=O)OC(C)(C)C)cc1Nc1cc(NS(=O)(=O)C2CC2)ncn1 ; COc1cc2[nH]ncc2cc1Nc1cc(NS(=O)(=O)C2CC2)ncn1]

Step 1

### 1-tert-Butoxyacyl-5-((6-(cyclopropanesulfonylamino) pyrimidin-4-yl) amino)-6-methoxyindazole

To the mixture of N-(6-aminopyrimidin-4-yl)cyclopropanesulfonamide 49a (21.0 mg, 0.1 mmol), 1-tert-butoxyacyl-5-bromo-6-methoxyindazole (33.0 mg, 0.1 mmol) and 1,4-dioxane (2 mL) were added tris(dibenzylideneacetone)-dipalladium(0) (9.0 mg, 0.01 mmol), cesium carbonate (65.0 mg, 0.2 mmol) and 4,5-bis(diphenylphosphino)-9,9-dimethylxanthene (12.0 mg, 0.02 mmol) at room temperature under argon. The mixture was stirred at 110° C. for 15 hours under argon. Then it was quenched with water (10 mL). The organic layer was separated and the aqueous layer was extracted with dichloromethane (15 mL×2). The combined organic layer was washed with brine (50 mL×2), dried over anhydrous sodium sulfate and filtered. The filtrate was concentrated under reduced pressure. Purification of the residue using Prep TLC (dichloromethane/methanol=20:1) gave 1-tert-butoxyacyl-5-((6-(cyclopropanesulfonylamino) pyrimidin-4-yl) amino)-6-methoxyindazole 49b (20.0 mg, 0.043 mmol, yellow solid). Yield: 43%.

MS m/z (ESI): 461 [M+1].

Step 2

### 5-((6-(Cyclopropanesulfonylamino) pyrimidin-4-yl) amino)-6-methoxy-1H-indazole

1-tert-Butoxyacyl-5-((6-(cyclopropanesulfonylamino) pyrimidin-4-yl) amino)-6-methoxyindazole 49b (20.0 mg, 0.043 mmol), dichloromethane (1.0 mL) and trifluoroacetic acid (1.0 mL) were mixed and stirred at room temperature for 3 hours. Then it was quenched by saturated aqueous sodium bicarbonate solution (10 mL). The organic layer was separated and the aqueous layer was extracted with dichloromethane (15 mL×2). The combined organic layer was washed with brine (50 mL×2), dried over anhydrous sodium sulfate and filtered. The filtrate was concentrated under reduced pressure. Purification of the residue using Prep TLC (dichloromethane/methaol=10:1) gave 5-((6-(cyclopropanesulfonylamino) pyrimidin-4-yl) amino)-6-methoxy-1H-indazole 49 (2.0 mg, 0.006 mmol, white solid). Yield: 14%.

MS m/z (ESI): 361 [M+1];

¹H NMR (400 MHz, DMSO-d6) δ 12.89 (s, 1H), 8.98 (s, 1H), 8.32 (s, 1H), 8.16 (s, 1H), 7.95 (s, 1H), 7.80 (s, 1H), 7.04 (s, 1H), 6.15 (s, 1H), 3.78 (s, 3H), 2.67-2.56 (m, 1H), 0.90-0.88 (m, 4H).

### Example 50

### 5-(6-(Acetamido) pyrimidin-4-yl) amino)-6-methoxy-1H-pyrazolo [3,4-b] pyridine formate

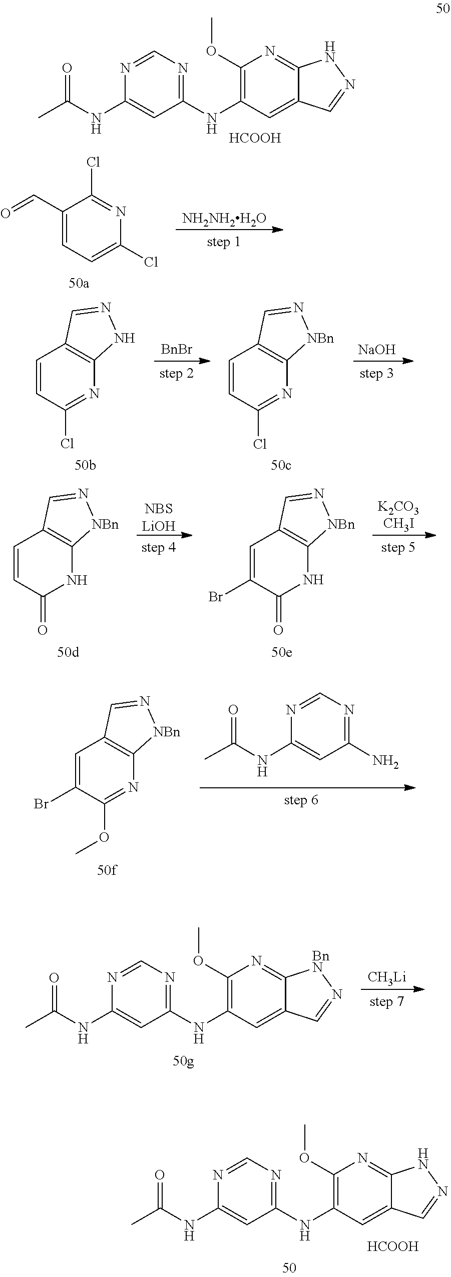

Step 1

### 6-Chloro-1H-pyrazolo[3,4-b]pyridine

To the solution of 2,6-dichloronicotinaldehyde 50a (4.0 g, 22.86 mmol) in butan-1-ol (1 mL) was added hydrazine hydrate (3.3 mL, 68.57 mmol) at room temperature. The mixture was stirred at 120° C. for 8 hours. Then it was concentrated in vacuum. The residue was diluted with water (100 mL) and extracted with ethyl acetate (100 mL×3). The combined organic layer was washed with brine (100 mL×2), dried over anhydrous sodium sulfate and filtered.

The filtrate was concentrated under reduced pressure. Purification of the residue using flash column chromatography (petroleum ether˜petroleum ether/ethyl acetate=7:1) gave 6-chloro-1H-pyrazolo[3,4-b]pyridine 50b (0.75 g, 4.90 mmol, yellow solid). Yield: 21%.

MS m/z (ESI): 154 & 156 [M+1];

Step 2

### 1-Benzyl-6-chloro-1H-pyrazolo[3,4-b]pyridine

6-Chloro-1H-pyrazolo[3,4-b]pyridine 50b (0.60 g, 3.92 mmol), benzyl bromide (738 mg, 4.31 mmol), cesium carbonate (1.4 g, 4.31 mmol) and N,N-dimethylformamide (3 mL) were mixed and stirred at room temperature for 2 hours. Then it was quenched with saturated aqueous sodium thiosulfate solution (50 mL) and extracted with ethyl acetate (50 mL×2). The combined organic layer was washed with brine (50 mL×2), dried over anhydrous sodium sulfate and filtered. The filtrate was concentrated under reduced pressure. Purification of the residue using flash column chromatography (petroleum ether-petroleum ether/ethyl acetate=100:0-100:2) gave 1-benzyl-6-chloro-1H-pyrazolo[3,4-b]pyridine 50c (0.75 g, 3.09 mmol, yellow solid), yield: 27%.

Step 3

### 1-Benzyl-1,7-dihydro-6H-pyrazolo[3,4-b]pyridin-6-one

To the solution of 1-benzyl-6-chloro-1H-pyrazolo[3,4-b]pyridine 50c (0.75 g, 3.09 mmol) in water (8 mL) and dimethyl sulfoxide (8 mL) was added sodium hydroxide (1.2 g, 30.86 mmol). The resulting mixture was stirred at 100° C. for 16 hours. Then it was cooled to room temperature and adjusted to pH a 8 with concentrated hydrochloric acid. The mixture was diluted with water (50 mL) and extracted with ethyl acetate (50 mL×3). The combined organic layer was washed with water (50 mL×3) and saturated brine (50 mL×2), dried over anhydrous sodium sulfate and filtered. The filtrate was concentrated under reduced pressure to give 1-benzyl-1,7-dihydro-6H-pyrazolo[3,4-b]pyridin-6-one 50d (0.6 g, 2.67 mmol, yellow solid). Yield: 86%.

MS m/z (ESI): 226 [M+1],

Step 4

### 1-Benzyl-5-bromo-1,7-dihydro-6H-pyrazolo[3,4-b]pyridin-6-one

To the solution of 1-benzyl-1,7-dihydro-6H-pyrazolo[3,4-b]pyridin-6-one 50d (0.38 g, 1.69 mmol) in water (6 mL) and acetonitrile (8 mL) was added lithium hydroxide (81 mg, 3.38 mol) and N-bromosuccinimide (0.60 g, 3.38 mol). The mixture was stirred at room temperature for 2 hours. Then it was quenched with saturated aqueous sodium thiosulfate solution (20 mL) and extracted with ethyl acetate (20 mL×3). The combined organic layer was washed with brine (20 mL×2), dried over anhydrous sodium sulfate and filtered. The filtrate was concentrated under reduced pressure. Purification of the residue using flash column chromatography (petroleum ether/ethyl acetate=3:1˜2:3) gave 1-benzyl-5-bromo-1,7-dihydro-6H-pyrazolo[3,4-b]pyridin-6-one 50e (0.23 g, 0.76 mmol, pale yellow solid). Yield: 45%.

MS m/z (ESI): 304 & 306 [M+1],

¹H NMR (400 MHz, CDCl₃) δ 8.16 (s, 1H), 7.76 (s, 1H), 7.52-7.29 (m, 5H), 5.61 (s, 2H).

Step 5

### 1-Benzyl-5-bromo-6-methoxy-1H-pyrazolo[3,4-b]pyridine

1-Benzyl-5-bromo-1,7-dihydro-6H-pyrazolo[3,4-b]pyridin-6-one 50e (0.23 g, 0.76 mmol), potassium carbonate (0.21 g, 1.52 mmol), iodomethane (0.22 g, 1.518 mmol) and N,N-dimethylformamide (4 mL) were mixed and stirred at room temperature for 2 hours. Then it was diluted with water (20 mL) and extracted with ethyl acetate (20 mL×3). The combined organic layer was washed with water (20 mL×3) and saturated brine (20 mL×2), dried over anhydrous sodium sulfate and filtered. The filtrate was concentrated under reduced pressure. Purification of the residue using flash column chromatography (petroleum ether/ethyl acetate=1:0-25:1) gave 1-benzyl-5-bromo-6-methoxy-1H-pyrazolo[3,4-b]pyridine 50f (95 mg, 0.30 mmol, pale yellow solid). Yield: 39%.

MS m/z (ESI): 318 & 320 [M+1],

Step 6

### N-(6-((1-Benzyl-6-methoxy-1H-pyrazolo[3,4-b]pyridin-5-yl)amino)pyrimidin-4-yl)acetamide

To the solution of 1-benzyl-5-bromo-6-methoxy-1H-pyrazolo[3,4-b]pyridine 50f (20.0 mg, 0.063 mmol), N-(6-aminopyrimidin-4-yl)acetamide (19.0 mg, 0.126 mmol) and cesium carbonate (62 mg, 0.189 mmol) in 1,4-dioxane (1 mL) was added tris(dibenzylideneacetone)dipalladium(0) (6 mg, 0.006 mmol) and 4,5-bis(diphenylphosphino)-9,9-dimethylxanthene (7 mg, 0.013 mmol) at room temperature under argon. The mixture was stirred at 100° C. for 1 hour under argon in microwave oven. Then it was diluted with water (10 mL) and extracted with ethyl acetate (10 mL×3). The combined organic layer was washed with brine (10 mL×2), dried over anhydrous sodium sulfate and filtered. The filtrate was concentrated under reduced pressure. Purification of the residue using Prep TLC (petroleum ether/ethyl acetate=3:1) gave N-(6-((1-benzyl-6-methoxy-1H-pyrazolo[3,4-b]pyridin-5-yl)amino)pyrimidin-4-yl)acetamide 50g (10.0 mg, 0.026 mmol, pale yellow solid). Yield: 41%.

MS m/z (ESI): 390 [M+1].

Step 7

### 5-(6-(Acetamido) pyrimidin-4-yl) amino)-6-methoxy-1H-pyrazolo [3,4-b] pyridine formate

To the solution of N-(6-((1-benzyl-6-methoxy-1H-pyrazolo[3,4-b]pyridin-5-yl)amino)pyrimidin-4-yl)acetamide 50g (10.0 mg, 0.026 mmol) in tetrahydrofuran (2 mL) was added dropwise methyllithium (0.16 mL, 0.257 mmol, 2 M) under argon. The mixture was stirred at room temperature for 2 hours. Then it was quenched with water (10 mL) and extracted with dichloromethane (10 mL×3). The combined organic layer was dried over anhydrous sodium sulfate and filtered. The filtrate was concentrated under reduced pressure. Purification of the residue using Prep HPLC (water (with 0.2% formic acid), 10%˜60% acetonitrile, 15 minutes) gave 5-(6-(acetamido) pyrimidin-4-yl) amino)-6-methoxy-1H-pyrazolo [3,4-b] pyridine formate 50 (1.1 mg, 0.003 mmol, white solid). Yield: 13%.

MS m/z (ESI): 300 [M+1],

¹H NMR (400 MHz, DMSO-d6) δ 13.27 (s, 1H), 10.38 (s, 1H), 8.91 (s, 1H), 8.43 (s, 1H), 8.40 (s, 1H), 8.27 (s, 1H), 7.98 (s, 1H), 7.46 (s, 1H), 3.87 (s, 3H), 2.07 (s, 3H).

### Example 51

### 5-(6-Methoxypyrimidin-4-yl) amino)-6-methoxy-1H-indazole formate

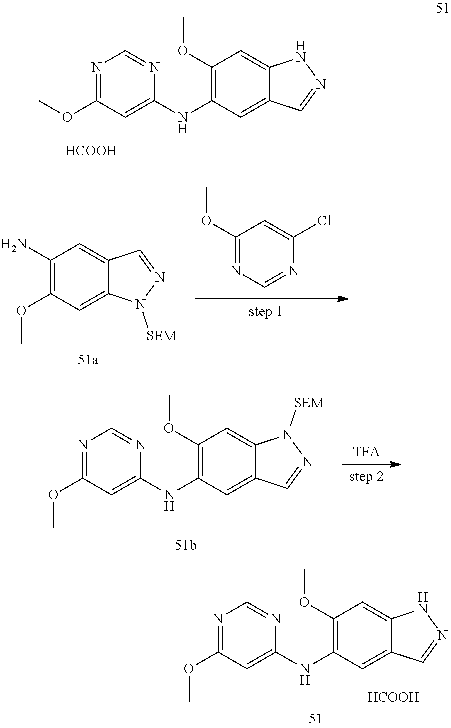

Step 1

### 1-((2-(Trimethylsilyl) ethoxy) methyl)-5-(6-methoxypyrimidin-4-yl) amino)-6-methoxyindazole

To the mixture of 1-((2-(trimethylsilyl) ethoxy) methyl)-5-amino-6-methoxyindazole 51a (30.0 mg, 0.10 mmol), 4-chloro-6-methoxypyrimidine (14.4 mg, 0.10 mmol) and 1, 4-dixane (1 mL) were added tris(dibenzylideneacetone)dipalladium(0) (9.2 mg, 0.01 mmol), 4,5-bis(diphenylphosphino)-9,9-dimethylxanthene (5.8 mg, 0.01 mmol) and cesium carbonate (65.2 mg, 0.2 mmol) at room temperature under argon. The mixture was stirred at 120° C. for 1 hour under argon in microwave oven. Then it was cooled to room temperature, diluted with dichloromethane (10 mL) and filtered. The filtrate was concentrated under reduced pressure. Purification of the residue using Prep HPLC (water (with 0.2% formic acid), 60%˜80% acetonitrile, 15 minutes) gave 1-((2-(trimethylsilyl) ethoxy) methyl)-5-(6-methoxypyrimidin-4-yl) amino)-6-methoxyindazole 51b (4.6 mg, 0.011 mmol, white solid). Yield: 12%.

MS m/z (ESI): 402 [M+1].

Step 2

### 5-(6-Methoxypyrimidin-4-yl) amino)-6-methoxy-1H-indazole formate

1-((2-(Trimethylsilyl) ethoxy) methyl)-5-(6-methoxypyrimidin-4-yl) amino)-6-methoxyindazole 51b (4.6 mg, 0.011 mmol) was dissolved into dichloromethane (1 mL) and trifluoroacetic acid (1 mL) was added, and the resultant mixture is stirred at room temperature for 2 hours. Then it was concentrated under reduced pressure. The residue was dissolved in dimethyl sulfoxide (1 mL). The mixture was adjusted to pH=8˜9 with saturated aqueous sodium hydroxide solution. Purification of the mixture using Prep HPLC (water (with 0.2% formic acid), 10%˜40% acetonitrile, 15 minutes) gave 5-(6-methoxypyrimidin-4-yl) amino)-6-methoxy-1H-indazole formate 51 (1.5 mg, 0.006 mmol, white solid). Yield: 55%.

MS m/z (ESI): 272 [M+1],

¹H NMR (400 MHz, DMSO-d6) δ 12.89 (s, 1H), 8.61 (s, 1H), 8.28 (s, 1H), 8.24 (s, 1H), 8.02 (s, 1H), 7.94 (s, 1H), 7.03 (s, 1H), 5.97 (s, 1H), 3.87 (s, 3H), 3.80 (s, 3H).

### Example 52

### 5-(5-Methoxypyrimidin-4-yl) amino)-6-methoxy-1H-indazole

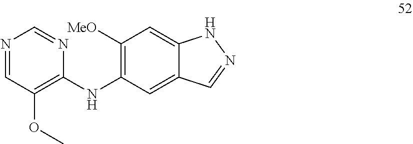

The synthetic procedures were similar to those in Example 49. Using 5-methoxypyrimidine-4-amine instead of N-(6-aminopyrimidine-4-yl)cyclopropylsulfonamide, the desired compound 5-(5-methoxypyrimidin-4-yl) amino)-6-methoxy-1H-indazole 52 (9.0 mg, 0.003 mmol, white solid) was obtained. Yield: 47%.

MS m/z (ESI): 272 [M+1],

¹H NMR (400 MHz, DMSO-d6) δ 12.84 (s, 1H), 8.71 (s, 1H), 8.33 (s, 1H), 8.08 (s, 1H), 8.05 (s, 1H), 7.99 (s, 1H), 7.08 (s, 1H), 3.98 (s, 3H), 3.96 (s, 3H).

### Example 53

### 5-(5-(2-Methoxyethoxy) pyrimidin-4-yl) amino)-6-methoxy-1H-indazole

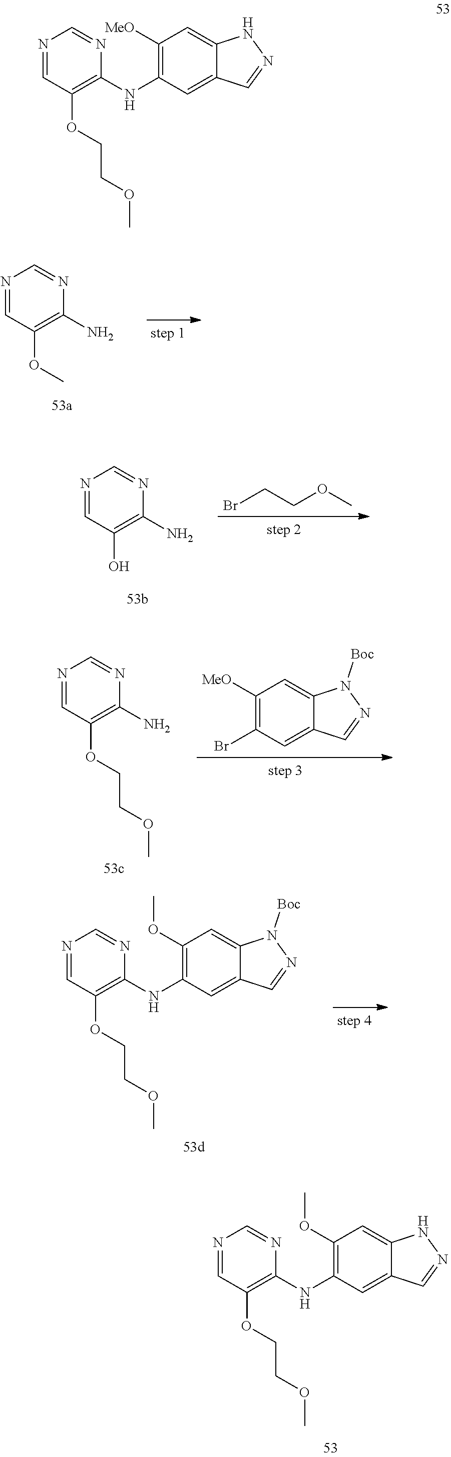

Step 1

### 4-Amino-5-hydroxypyrimidine

To the solution of 4-amino-5-methoxypyrimidine 53a (500.0 mg, 4.0 mmol) and sodium methanolate (432.0 mg, 8.0 mmol) in anhydrous N,N-dimethylformamide (6 mL) was added 1-dodecanethiol (1.61 g, 8.00 mmol). The mixture was stirred at 120° C. for 12 hours. Then it was cooled to room temperature and concentrated under reduced pressure. The residue was adjusted pH to 5˜6 with 5.0 mL of water and 0.5 mL of acetic acid. The aqueous layer was washed with ethyl acetate (10 mL×5) and concentrated in vacuum to give 4-amino-5-hydroxypyrimidine 53b (0.4 g, 3.6 mmol, off-white solid), yield: 90%.

MS m/z (ESI): 112 [M+1],

¹H NMR (400 MHz, DMSO-d6) δ 9.64 (brs, 1H), 7.91 (s, 1H), 7.63 (s, 1H), 6.42 (brs, 2H).

Step 2

### 5-(2-Methoxyethoxy)pyrimidin-4-amine

The solution of 4-amino-5-hydroxypyrimidine 53b (100.0 mg, 0.9 mmol) and anhydrous lithium hydroxide (65.0 mg, 2.7 mmol) in N,N-dimethylformamide (6 mL) was stirred at room temperature for 30 minutes. 1-Bromo-2-methoxyethane (125.0 mg, 0.9 mmol) was added. The reaction mixture was stirred at 60° C. for 5 hours. Then it was cooled to room temperature and filtered. The filtrate was concentrated under reduced pressure. Purification of the residue using Prep TLC (dichloromethane/methanol=10:1) gave 5-(2-methoxyethoxy)pyrimidin-4-amine 53c (60.0 mg, 0.36 mmol, clear oil), yield: 40%.

MS m/z (ESI): 170 [M+1],

¹H NMR (400 MHz, CDCl₃) δ 8.16 (s, 1H), 7.79 (s, 1H), 5.52 (brs, 2H), 4.10-4.12 (m, 2H), 3.67-3.69 (m, 2H), 3.37 (s, 3H).

Step 3

### 1-tert-Butoxycarbonyl-5-(5-(2-methoxyethoxy) pyrimidin-4-yl) amino)-6-methoxyindazole

To the mixture of 5-(2-methoxyethoxy)pyrimidin-4-amine 53c (30.0 mg, 0.17 mmol), 1-tert-butoxycarbonyl-5-amino-6-methoxyindazole (110.0 mg, 0.34 mmol) and cesium carbonate (180 mg, 0.51 mmol) in 1,4-dioxane (2 mL) were added tris(dibenzylideneacetone)dipalladium(0) (15.0 mg, 0.016 mmol) and 4,5-bis(diphenylphosphino)-9,9-dimethylxanthene (18 mg, 0.032 mmol) at room temperature under argon. The mixture was stirred at 120° C. for 1 hour under argon in microwave oven. Then it was cooled to room temperature and filtered. The filtrate was concentrated under reduced pressure. Purification of the residue using Prep TLC (dichloromethane/methanol=10:1) gave 1-tert-butoxycarbonyl-5-(5-(2-methoxyethoxy) pyrimidin-4-yl) amino)-6-methoxyindazole 53d (13.0 mg, 0.031 mmol, white solid). Yield: 18%.

MS m/z (ESI): 416 [M+1],

¹H NMR (400 MHz, CDCl₃) δ 9.08 (s, 1H), 8.49 (s, 1H), 8.18 (s, 1H), 8.13 (s, 1H), 7.98 (s, 1H), 7.76 (s, 1H), 4.08-4.05 (m, 2H), 3.96 (s, 3H), 3.86-3.83 (m, 2H), 3.49 (s, 3H), 1.73 (s, 9H).

Step 4

### 5-(5-(2-Methoxyethoxy) pyrimidin-4-yl) amino)-6-methoxy-1H-indazole

To the solution of 1-tert-butoxycarbonyl-5-(5-(2-methoxyethoxy) pyrimidin-4-yl) amino)-6-methoxyindazole 53d (13.0 mg, 0.031 mmol) in dichloromethane (1 mL) was added the solution of hydrogen chloride in 1,4-dioxane (4 M, 1.0 mL). The mixture was stirred at room temperature for 12 hours. Then it was concentrated under reduced pressure. Purification of the residue using Prep HPLC (water (with 0.2% formic acid), 20%˜40% acetonitrile, 15 minutes) gave 5-(5-(2-methoxyethoxy) pyrimidin-4-yl) amino)-6-methoxy-1H-indazole 53 (6.0 mg, 0.019 mmol, white solid). Yield: 61%.

MS m/z (ESI): 316 [M+1],

¹H NMR (400 MHz, DMSO-d6) δ 12.87 (brs, 1H), 8.77 (s, 1H), 8.36 (s, 1H), 8.15 (s, 1H), 8.11 (s, 1H), 7.99 (s, 1H), 7.09 (s, 1H), 4.33-4.30 (m, 2H), 3.96 (s, 3H), 3.78-3.75 (m, 2H), 3.40 (s, 3H).

### Example 54

### 5-(5-(2-Hydroxyethoxy) pyrimidin-4-yl) amino)-6-methoxy-1H-indazole formate

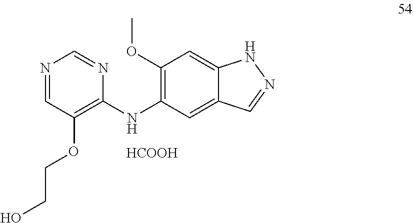 [SMILES: COc1cc2[nH]ncc2cc1Nc1ncncc1OCCO ; "HCOOH"]

The synthetic procedures were similar to that in Example 53. Using (2-bromoethoxy)-t-butyldimethylsilane instead of 1-bromine-2-methoxyethane. The desired compound: 5-(5-(2-hydroxyethoxy) pyrimidin-4-yl) amino)-6-methoxy-1H-indazole formate 54 (1.0 mg, 0.003 mmol, white solid) was obtained. Yield: 28%. Purification condition: prep HPLC (water (with 0.2% formic acid), 10%˜60% acetonitrile, 15 minutes).

MS m/z (ESI): 302 [M+1],

¹H NMR (400 MHz, DMSO-d6) δ 12.88 (s, 1H), 8.55 (s, 1H), 8.28-8.23 (m, 3H), 8.07 (s, 1H), 7.99 (s, 1H), 7.08 (s, 1H), 5.32 (br, 1H), 4.21-4.19 (m, 2H), 3.94 (s, 3H), 3.82-3.79 (m, 2H).

### Example 55

### 5-(5-(2-(4-Morpholine) ethoxy) pyrimidin-4-yl) amino)-6-methoxy-1H-indazole formate

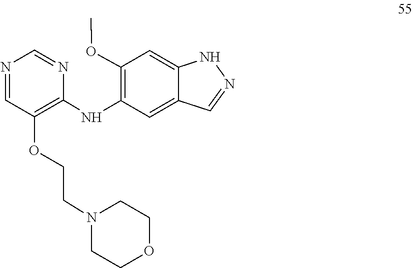 [SMILES: COc1cc2[nH]ncc2cc1Nc1ncncc1OCCN1CCOCC1]

The synthetic procedures were similar to that in Example 53. Using 4-(2-chloroethyl)morpholine instead of 1-bromine-2-methoxyethane. The desired compound: 5-(5-(2-(4-morpholine) ethoxy) pyrimidin-4-yl) amino)-6-methoxy-1H-indazole formate 55 was obtained. Yield: 25%. Purification condition: prep HPLC (water (with 0.2% formic acid), 10%˜40% acetonitrile, 15 minutes).

MS m/z (ESI): 371 [M+1],

¹H NMR (400 MHz, DMSO-d6) δ 12.88 (s, 1H), 8.79 (s, 1H), 8.36 (s, 1H), 8.17 (s, 1H), 8.11 (s, 1H), 7.99 (s, 1H), 7.09 (s, 1H), 4.33-4.24 (m, 2H), 3.97 (s, 3H), 3.59-3.52 (m, 4H), 3.18-3.10 (m, 4H), 2.92-2.61 (m, 2H).

### Example 56

### 5-(5-Ethoxypyrimidin-4-yl) amino)-6-methoxy-1H-indazole formate

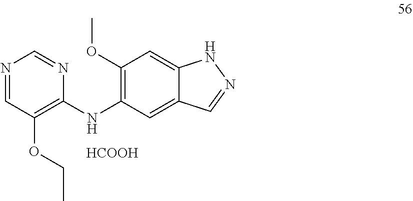 [SMILES: CCOc1cncnc1Nc1cc2cn[nH]c2cc1OC ; "HCOOH"]

The synthetic procedures were similar to that in Example 53. Using iodoethane instead of 1-bromine-2-methoxyethane. The desired compound: 5-(5-ethoxypyrimidin-4-yl) amino)-6-methoxy-1H-indazole formate 56 was obtained.

Yield: 25%. Purification condition: prep HPLC (water (with 0.2% formic acid), 10%˜50% acetonitrile, 15 minutes).

MS m/z (ESI): 286 [M+1],

¹H NMR (400 MHz, DMSO-d6) δ 12.91 (brs, 1H), 8.73 (s, 1H), 8.33 (s, 1H), 8.21 (s, 1H), 8.11 (s, 1H), 8.07 (s, 1H), 7.99 (s, 1H), 7.09 (s, 1H), 4.28-4.21 (m, 2H), 3.97 (s, 3H), 1.44 (t, J=6.4, 3H).

### Example 57

### 5-(5-Anilinopyrimidin-4-yl) amino)-6-methoxy-1H-indazole formate

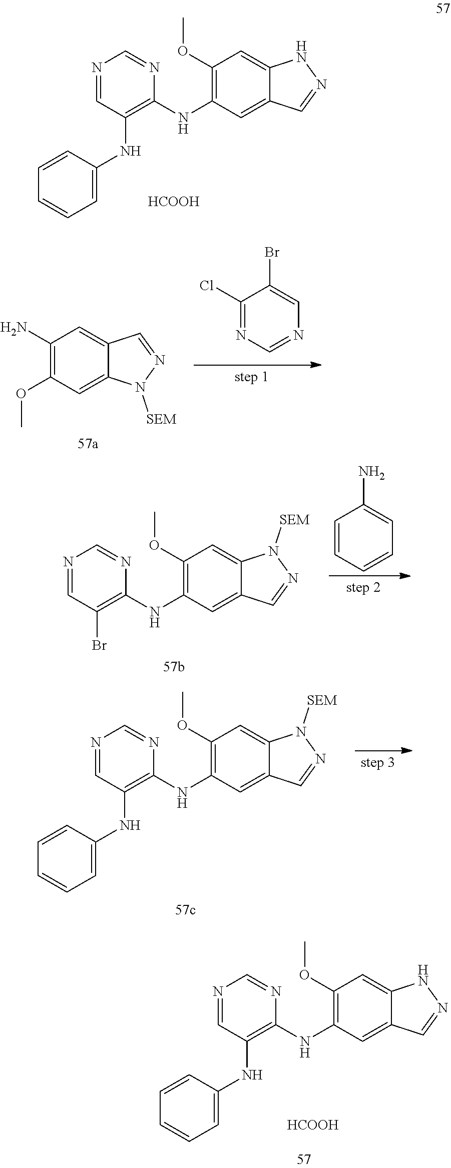

Step 1

### 1-((2-(Trimethylsilyl) ethoxy) methyl)-5-(5-bromopyrimidin-4-yl) amino)-6-methoxyindazole

1-((2-(Trimethylsilyl) ethoxy) methyl)-5-amino-6-methoxyindazole 57a (200 mg, 0.68 mmol), 5-bromo-4-chloropyrimidine (164 mg, 0.9 mmol), cesium carbonate (445 mg, 1.4 mmol) and N,N-dimethylformamide (5 mL) were mixed and stirred at 150° C. for 1 hour in microwave oven. Then it was diluted with dichloromethane (20 mL) and washed with brine (50 mL×2). The organic layer was dried over anhydrous sodium sulfate and filtered. The filtrate was concentrated under reduced pressure. Purification of the residue using flash column chromatography (dichloromethane/methanol=20:1) gave 1-((2-(trimethylsilyl) ethoxy) methyl)-5-(5-bromopyrimidin-4-yl) amino)-6-methoxyindazole 57b (102.0 mg, 0.23 mmol, yellow oil). Yield: 34%.

MS m/z (ESI): 450 & 452 [M+1];

Step 2

### 1-((2-(Trimethylsilyl) ethoxy) methyl)-5-(5-anilinopyrimidin-4-yl) amino)-6-methoxyindazole

To the mixture of 1-((2-(trimethylsilyl) ethoxy) methyl)-5-(5-bromopyrimidin-4-yl) amino)-6-methoxyindazole 57b (30.0 mg, 0.067 mmol), aniline (9.3 mg, 0.10 mmol) and 1,4-dioxane (1 mL) were added tris(dibenzylideneacetone)-dipalladium(0) (6.1 mg, 0.007 mmol), 2-(dicyclohexylphosphino)3,6-dimethoxy-2′,4′,6′-triisopropyl-1,1′-biphenyl (7.2 mg, 0.013 mmol) and cesium carbonate (87.4 mg, 0.27 mmol) under argon. The mixture was stirred at 90° C. for 1 hour under argon in microwave oven. Then it was cooled to room temperature, diluted with dichloromethane (10 mL) and filtered. The filtrate was concentrated under reduced pressure. Purification of the residue using Prep HPLC (water (with 0.2% formic acid), 30%˜80% acetonitrile, 15 minutes) gave 1-((2-(trimethylsilyl) ethoxy) methyl)-5-(5-anilinopyrimidin-4-yl) amino)-6-methoxyindazole 57c (1.7 mg, 0.0033 mmol, white solid). Yield: 5%.

MS m/z (ESI): 463 [M+1];

Step 3

### 5-(5-Anilinopyrimidin-4-yl) amino)-6-methoxy-1H-indazole formate

1-((2-(Trimethylsilyl) ethoxy) methyl)-5-(5-anilinopyrimidin-4-yl) amino)-6-methoxyindazole 57c (1.7 mg, 0.0033 mmol) was dissolved into hydrogen chloride in methanol (2 M, 3.0 mL) and was stirred at 80° C. for 0.5 hour. Then it was concentrated under reduced pressure. Purification of the residue using Prep HPLC (water (with 0.2% formic acid), 20%˜40% acetonitrile, 15 minutes) gave 5-(5-Anilinopyrimidin-4-yl) amino)-6-methoxy-1H-indazole formate 57 (1.0 mg, 0.0026 mmol, white solid). Yield: 80%.

MS m/z (ESI): 333 [M+1],

¹H NMR (400 MHz, DMSO-d6) δ 12.92 (s, 1H), 8.65 (s, 1H), 8.50 (s, 1H), 8.21 (s, 1H), 8.02 (s, 1H), 7.98 (s, 1H), 7.86 (s, 1H), 7.27-7.19 (m, 2H), 7.02 (s, 1H), 6.86-6.78 (m, 3H), 6.68 (s, 1H), 3.76 (s, 3H).

### Example 58

### N-(6-Methoxy-1H-indazol-5-yl)-purin-6-amine

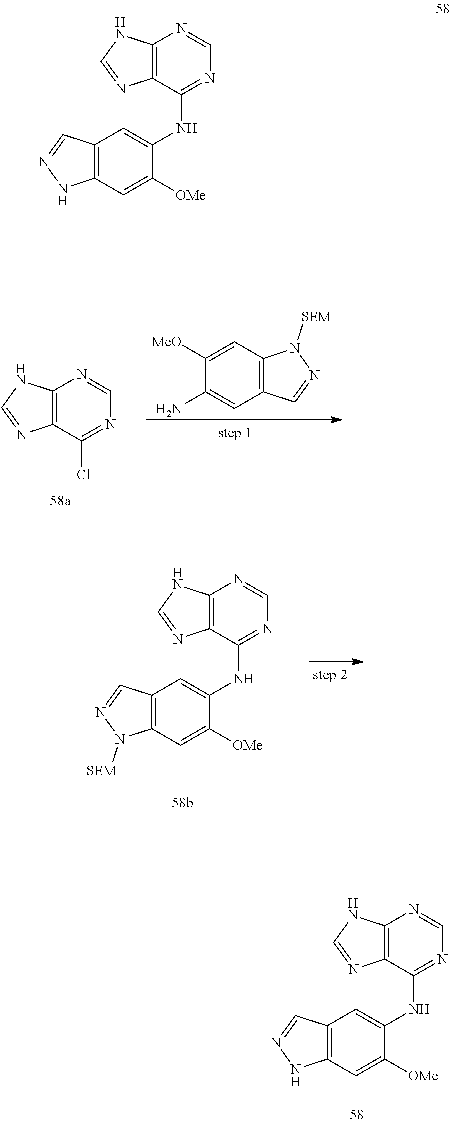 [SMILES: COc1cc2[nH]ncc2cc1Nc1ncnc2[nH]cnc12 ; Clc1ncnc2[nH]cnc12 ; COc1cc2c(cnn2COCC[Si](C)(C)C)cc1N ; COc1cc2c(cnn2COCC[Si](C)(C)C)cc1Nc1ncnc2[nH]cnc12 ; COc1cc2[nH]ncc2cc1Nc1ncnc2[nH]cnc12]

Step 1

### N-(6-Methoxy-1-((2-(trimethylsilyl)ethoxy)methyl)-1H-indazol-5-yl)-9H-purin-6-amine

6-Chloro-9H-purine 58a (154.0 mg, 1.0 mmol), 6-methoxy-1-((2-(trimethylsilyl)ethoxy)methyl)-1H-indazol-5-amine (293.0 mg, 1.0 mmol) and acetic acid (2 mL) were mixed and stirred at 70° C. for 1 hour. Evaporation of the solvents and the residue was diluted with dichloromethane (20 mL). It was adjusted to pH=8˜10 by saturated aqueous sodium bicarbonate solution. The mixture was extracted with dichloromethane (50 mL×3). The combined organic layer was washed with brine (50 mL), dried over anhydrous sodium sulfate and filtered. The filtrate was concentrated under reduced pressure. Purification of the residue using flash column chromatography (dichloromethane/methanol=40:1), N-(6-methoxy-1-((2-(trimethylsilyl)ethoxy)methyl)-1H-indazol-5-yl)-9H-purin-6-amine 58b (50.0 mg, 0.12 mmol, white solid) was obtained. Yield: 12%.

MS m/z (ESI): 412 [M+1];

Step 2

### N-(6-Methoxy-1H-indazol-5-yl)-9H-purin-6-amine

N-(6-Methoxy-1-((2-(trimethylsilyl)ethoxy)methyl)-1H-indazol-5-yl)-9H-purin-6-amine 58b (50.0 mg, 0.12 mmol), dichloromethane (2 mL) and trifluoroacetic acid (1 mL) were mixed and stirred at room temperature for 3 hours. Then it was quenched by 5 mL of saturated aqueous sodium bicarbonate solution and diluted with dichloromethane (5 mL). The organic layer was separated and the aqueous layer was extracted with dichloromethane (20 mL×2). The combined organic layer was washed with brine (20 mL), dried over anhydrous sodium sulfate and filtered. The filtrate was concentrated under reduced pressure. Purification of the residue using Prep HPLC (water (with 0.2% formic acid), 20%˜50% acetonitrile, 15 minutes) gave N-(6-methoxy-1H-indazol-5-yl)-9H-purin-6-amine 58 (30.0 mg, 0.11 mmol, white solid). Yield: 80%.

MS m/z (ESI): 282 [M+1],

¹H NMR (400 MHz, DMSO-d6) δ 11.08 (s, 1H), 8.70 (s, 1H), 8.55 (s, 1H), 8.06 (s, 1H), 7.98 (s, 1H), 7.16 (s, 1H), 3.87 (s, 3H).

### Example 59

### N-(6-Methoxy-1H-indazol-5-yl)-9-methyl-9H-purin-6-amine

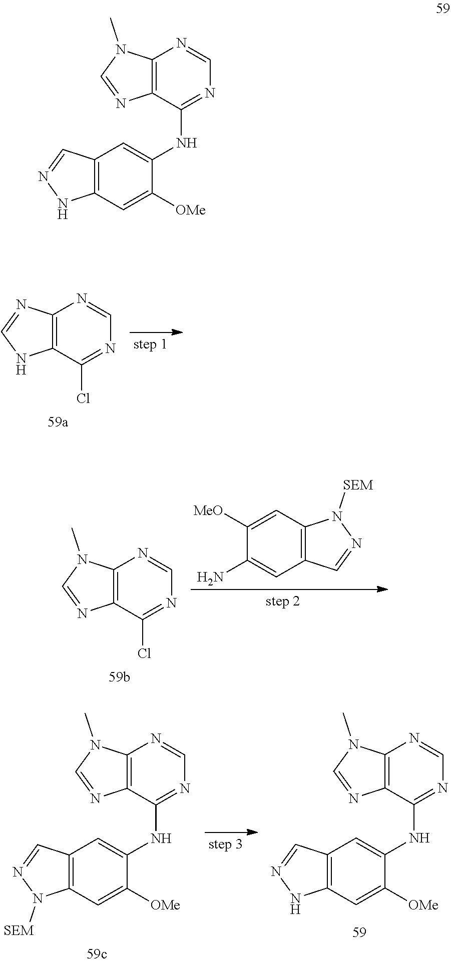 [SMILES: COc1cc2[nH]ncc2cc1Nc1ncnc2c1ncn2C ; Clc1ncnc2nc[nH]c12 ; Cn1cnc2c(Cl)ncnc21 ; COc1cc2c(cnn2COCC[Si](C)(C)C)cc1N ; COc1cc2c(cnn2COCC[Si](C)(C)C)cc1Nc1ncnc2c1ncn2C ; COc1cc2[nH]ncc2cc1Nc1ncnc2c1ncn2C]

Step 1

### 6-Chloro-9-methyl-9H-purine

To the mixture of 6-chloro-7H-purine 59a (154.0 mg, 1.0 mmol), sodium hydride (60% in mineral oil, 120.0 mg, 5.0 mmol) and N,N-dimethylacetamide (2 mL) was added iodomethane (426.0 mg, 3.0 mmol) at room temperature. The mixture was stirred at room temperature for 24 hours. Then it was quenched with 5 mL of water and extracted with dichloromethane (30 mL×3). The combined organic layer was washed with brine (30 mL), dried over anhydrous sodium sulfate and filtered. The filtrate was concentrated under reduced pressure. Purification of the residue using flash column chromatography gave 6-chloro-9-methyl-9H-purine 59b (80.0 mg, 0.48 mmol, white solid). Yield: 48%.

¹H NMR (400 MHz, DMSO-d6) δ 8.78 (s, 1H), 8.68 (s, 1H), 3.90 (s, 3H).

Step 2

### N-(6-Methoxy-1-((2-(trimethylsilyl)ethoxy)methyl)-1H-indazol-5-yl)-9-methyl-9H-purin-6-amine

6-Chloro-9-methyl-9H-purine 59b (17.0 mg, 0.1 mmol), 6-methoxy-1-((2-(trimethylsilyl)ethoxy)methyl)-1H-indazol-5-amine (29.0 mg, 10.1 mmol) and acetic acid (2 mL) were mixed and stirred at 90° C. for 1 hour. Evaporation of the solvents and the residue was diluted with dichloromethane (20 mL). The pH value was adjusted to 8˜10 by saturated aqueous sodium bicarbonate solution. The organic layer was separated and the aqueous layer was extracted with dichloromethane (10 mL×3). The combined organic layer was washed with brine (10 mL), dried over anhydrous sodium sulfate and filtered. The filtrate was concentrated under reduced pressure. Purification of the residue using prep-TLC (dichloromethane/methanol=20:1) gave N-(6-methoxy-1-((2-(trimethylsilyl)ethoxy)methyl)-1H-indazol-5-yl)-9-methyl-9H-purin-6-amine 59c (13.0 mg, 0.03 mmol). Yield: 30%.

MS m/z (ESI): 426 [M+1].

Step 3

### N-(6-Methoxy-1H-indazol-5-yl)-9-methyl-9H-purin-6-amine

N-(6-Methoxy-1-((2-(trimethylsilyl)ethoxy)methyl)-1H-indazol-5-yl)-9-methyl-9H-purin-6-amine 59c (13.0 mg, 0.03 mmol), dichloromethane (2 mL) and trifluoroacetic acid (1 mL) were mixed and stirred at room temperature for 3 hours. Then it was quenched by 5 mL of saturated aqueous sodium bicarbonate solution and diluted with dichloromethane (5 mL). The organic layer was separated and the aqueous layer was extracted with dichloromethane (10 mL×3). The combined organic layer was washed with brine (10 mL), dried over anhydrous sodium sulfate and filtered. The filtrate was concentrated under reduced pressure. Purification of the residue using Prep HPLC (water (with 0.2% formic acid), 20%˜50% acetonitrile, 15 minutes) gave N-(6-methoxy-1H-indazol-5-yl)-9-methyl-9H-purin-6-amine 59 (6.0 mg, 0.02 mmol, white solid).

Yield: 66%.

MS m/z (ESI): 296 [M+1],

¹H NMR (400 MHz, DMSO-d6) δ 12.88 (s, 1H), 8.69 (s, 1H), 8.49 (s, 1H), 8.44 (s, 1H), 8.26 (s, 1H), 7.99 (s, 1H), 7.09 (s, 1H), 4.05 (s, 3H), 3.94 (s, 3H).

### Example 60&61

### 1-tert-Butoxycarbonyl-5-(6-cyclopropionamidopyrimidin-4-ylamino)-6-methoxybenzimidazole

3-tert-Butoxycarbonyl-5-(6-cyclopropionamidopyrimidin-4-ylamino)-6-methoxybenzimidazole

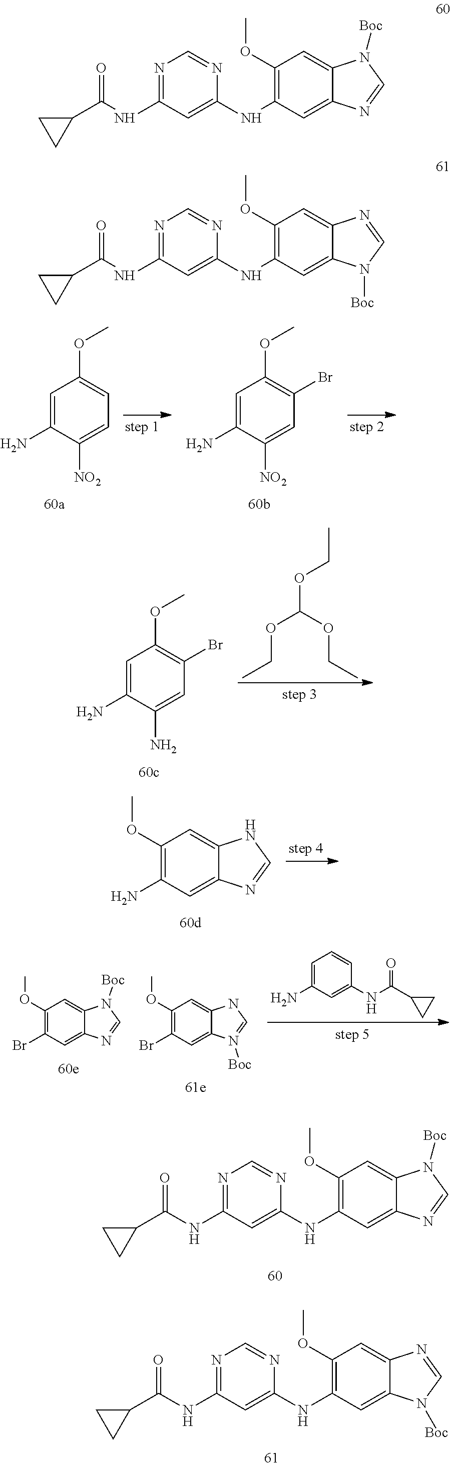

### Step 1

### 4-Bromo-5-methoxy-2-nitroaniline

To the mixture of 5-methoxy-2-nitroaniline 60a (2.0 g, 11.9 mmol) with acetonitrile (30.0 mL) was added N-bromosuccinimide (2.3 g, 13.1 mmol) at room temperature. The resulting mixture was stirred at room temperature for 3 hours. Then it was quenched with saturated aqueous sodium sulfite (60 mL) and diluted with water until large amount of precipitate formed. The precipitate was filtered. The filter cake was dissolved in ethyl acetate (100 mL), dried over anhydrous sodium sulfate and filtered. The filtrate was concentrated to give 4-bromo-5-methoxy-2-nitroaniline 60b (2.5 g, 10.1 mmol, yellow solid) as the desired product. Yield: 85%.

MS m/z (ESI): 247 & 249 [M+1].

Step 2

### 4-Bromo-5-methoxybenzene-1,2-diamine

4-Bromo-5-methoxy-2-nitroaniline 60b (0.5 g, 2.0 mmol) was mixed with dichloromethane (10.0 mL), methanol (10.0 mL) and saturated aqueous ammonium chloride (20.0 mL), zinc powder (1.3 g, 20.0 mmol) was added at room temperature. The resulting mixture was stirred at room temperature for 2 hours. Then it was filtered. The filtrate was diluted with dichloromethane (100 mL), dried over anhydrous sodium sulfate and filtered. The filtrate was concentrated to give 4-bromo-5-methoxybenzene-1,2-diamine 60c (450.0 mg, 2.0 mmol, brown solid). Yield: 100%.

MS m/z (ESI): 217 & 219 [M+1].

Step 3

### 5-Bromo-6-methoxy-1H-benzo[d]imidazole

To the mixture of 4-bromo-5-methoxybenzene-1,2-diamine 60c (450.0 mg, 2.0 mmol) and triethoxymethane (10.0 mL) was added formic acid (0.5 mL) at room temperature. The resulting mixture was stirred at 90° C. for 3 hours. Then it was evaporated to remove excess triethoxymethane and the crude product is achieved. The crude product was diluted with ethyl acetate (50 mL), washed with aqueous sodium bicarbonate and saturated brine successively. The organic layer was dried over anhydrous sodium sulfate and filtered. The filtrate was concentrated to give 5-bromo-6-methoxy-1H-benzo[d]imidazole 60d (430.0 mg, 1.9 mmol, brown solid) as the desired product. Yield: 95%.

MS m/z (ESI): 227 & 229 [M+1].

Step 4

### 1-tert-Butoxycarbonyl-5-bromo-6-methoxy-benzoimidazole

### 3-tert-Butoxycarbonyl-5-bromo-6-methoxy-benzoimidazole

To the mixture of 5-bromo-6-methoxy-1H-benzoimidazole 60d (430.0 mg, 1.9 mmol), triethylamine (383.0 mg, 3.8 mmol), 4-dimethylaminopyridine (23.0 mg, 0.2 mmol) and tetrahydrofuran (15.0 mL) was added di-t-butyl dicarbonate (585.0 mg, 2.9 mmol) at room temperature. The resulting mixture was stirred at 45° C. for 12 hours. Then it was diluted with dichloromethane (100.0 mL) and washed with brine (20.0 mL×3). The organic layer was dried over anhydrous sodium sulfate and filtered. The filtrate was concentrated to give a mixture of 1-tert-butoxycarbonyl-5-bromo-6-methoxy-benzoimidazole 60e and 3-tert-butoxycarbonyl-5-bromo-6-methoxy-benzoimidazole 61e (450.0 mg, 1.4 mmol, white solid). Yield: 73%.

MS m/z (ESI): 327 & 329 [M+1];

Step 5

### 1-tert-Butoxycarbonyl-5-(6-cyclopropionamidopyrimidin-4-ylamino)-6-methoxybenzimidazole

### 3-tert-Butoxycarbonyl-5-(6-cyclopropionamidopyrimidin-4-ylamino)-6-methoxybenzimidazole

To the mixture of 1-tert-butoxycarbonyl-5-bromo-6-methoxy-benzoimidazole 60e and 3-tert-butoxycarbonyl-5-bromo-6-methoxy-benzoimidazole 61e (200.0 mg, 0.61 mmol), N-(6-aminopyrimidin-4-yl)cyclopropanecarboxamide (145.0 mg, 0.81 mmol), cesium carbonate (480.0 mg, 1.47 mmol) and 1,4-dioxane (2 mL) were added tris(dibenzylideneacetone)dipalladium(0) (7.0 mg, 0.07 mmol) and dicyclohexyl(2′,4′,6′-triisopropyl-3,6-dimethoxy-[1,1′-biphenyl]-2-yl)phosphine (79.0 mg, 0.15 mmol) at room temperature under argon. Then the resulting mixture was stirred at 100° C. for 1.5 hours under argon. It was cooled to room temperature, diluted with dichloromethane (20 mL) and filtered. The filtrate was concentrated under reduced pressure. Purification of the residue using Prep TLC (dichloromethane/methanol=25:1) gavel-tert-butoxycarbonyl-5-(6-cyclopropionamidopyrimidin-4-ylamino)-6-methoxybenzimidazole 60 (40.0 mg, 0.094 mmol, white solid) and 3-tert-butoxycarbonyl-5-(6-cyclopropionamidopyrimidin-4-ylamino)-6-methoxybenzimidazole 61 (60.0 mg, 0.142 mmol, white solid). Yield: 38%.

### 1-tert-butoxycarbonyl-5-(6-cyclopropionamidopyrimidin-4-ylamino)-6-methoxybenzimidazole 60

MS m/z (ESI): 425 [M+1],

¹H NMR (400 MHz, CDCl₃) δ 8.72 (s, 1H), 8.68 (brs, 1H), 8.45 (s, 1H), 8.31 (s, 1H), 7.58 (s, 2H), 7.36 (s, 1H), 3.94 (s, 3H), 1.72 (s, 9H), 1.60-1.55 (m, 1H), 1.11-1.07 (m, 2H), 0.94-0.87 (m, 2H).

### 3-tert-butoxycarbonyl-5-(6-cyclopropionamidopyrimidin-4-ylamino)-6-methoxybenzimidazole 61

MS m/z (ESI): 425 [M+1],

¹H NMR (400 MHz, CDCl₃) δ 9.91 (s, 1H), 8.95 (s, 1H), 8.46 (s, 1H), 8.42 (s, 1H), 7.71 (s, 1H), 7.65 (s, 1H), 7.28 (s, 1H), 3.98 (s, 3H), 1.71 (s, 9H), 1.69-1.64 (m, 1H), 1.14-1.10 (m, 2H), 0.94-0.88 (m, 2H).

### Example 62

### 5-(5-Methylaminopyrimidin-4-yl) amino)-6-methoxy-1H-indazole formate

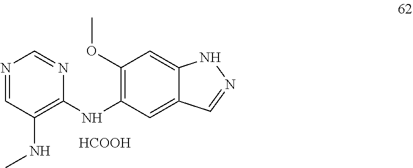 [SMILES: CNc1cncnc1Nc1cc2cn[nH]c2cc1OC ; "HCOOH"]

The synthetic procedures were similar to that in Example 55. Using 4-chloro-5-methylaminopyrimidine instead of 4-chloro-6-methoxypyrimidine. The desired product: 5-(5-methylaminopyrimidin-4-yl) amino)-6-methoxy-1H-indazole formate (1.4 mg, 0.005 mmol, white solid) was obtained. Yield: 25%.

MS m/z (ESI): 271 [M+1],

¹H NMR (400 MHz, DMSO-d6) δ 12.97 (s, 1H), 9.13 (s, 1H), 8.20 (s, 1H), 7.98 (s, 1H), 7.95 (s, 1H), 7.56 (s, 1H), 7.08 (s, 1H), 6.28 (s, 1H), 3.86 (s, 3H), 2.82 (d, J=3.6 Hz, 3H).

### Example 63

### N-(6-((6-Methoxypyrazolo[1,5-a]pyridin-5-yl)amino)pyrimidin-4-yl)cyclopropanecarboxamide

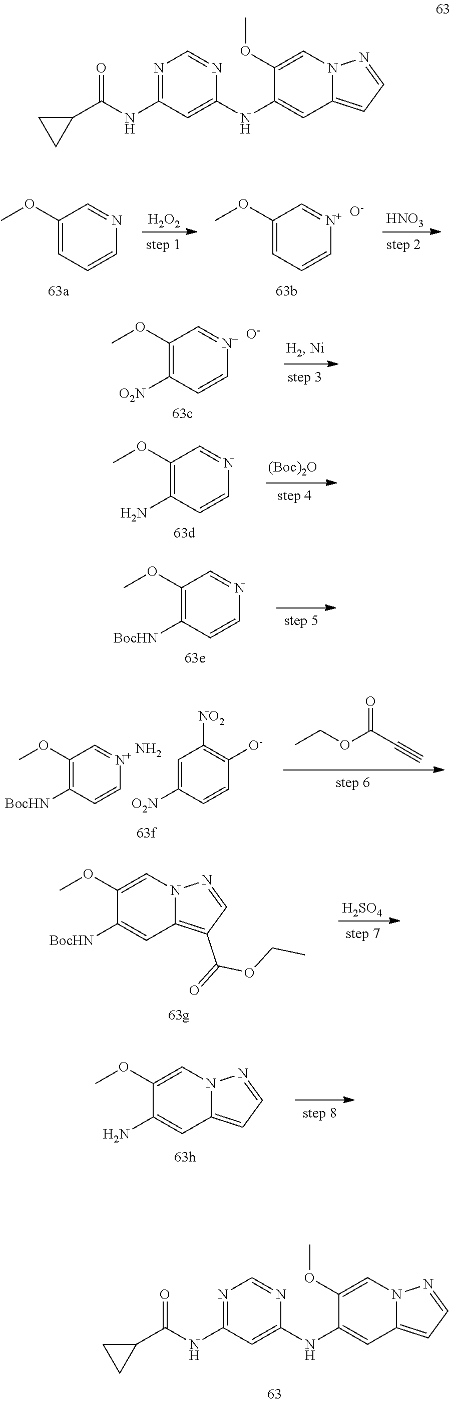

Step 1

### 3-Methoxypyridine 1-oxide

To the solution of 3-methoxypyridine 63a (1.1 g, 10.0 mmol) in acetic acid (20 mL) was added hydrogen peroxide (2.0 mL, 20.0 mmol, 30% aqueous solution) at room temperature. Then the resulting mixture was stirred at 80° C. for 5 hours. The mixture was concentrated, and 4 M aqueous sodium hydroxide was added until pH >8. It was extracted with dichloromethane (10 mL×3). The combined organic layer was washed with water (100 mL), dried over anhydrous sodium sulfate and filtered. The filtrate was concentrated to give pure 3-methoxypyridine N-oxide 63b (0.42 g, 3.4 mmol, white solid). Yield: 34%. MS m/z (ESI): 26 [M+1].

Step 2

### 3-Methoxy-4-nitropyridine N-oxide

3-Methoxypyridine N-oxide 63b (0.42 g, 3.4 mmol) was dissolved in concentrated sulfuric acid (1.25 mL, 98%), concentrated nitric acid (1.0 mL, 68%) was added slowly with stirring in an ice bath. Then the resulting mixture was stirred at 85° C. for 6 hours. The mixture was poured into ice water, 4 M aqueous sodium hydroxide was added to adjust the pH until pH >7. The mixture was extracted with ethyl acetate (3×10 mL). The combined organic layer was washed with water (100 mL), dried over anhydrous sodium sulfate and filtered. The filtrate was concentrated under reduced pressure. Purification of the residue using flash column chromatography (dichloromethane/methanol=1:0-10:1) gave desired 3-methoxy-4-nitropyridine N-oxide 63c (0.37 g, 2.2 mmol, yellow solid). Yield: 65%.

MS m/z (ESI): 172 [M+1],

Step 3

### 3-Methoxy-4-amino pyridine

To the solution of 3-methoxy-4-nitropyridine 1-oxide 63c (0.37 g, 2.2 mmol) in methanol (100 mL) was added Raney nickle (100 mg). The resulting mixture was stirred at room temperature for 1.5 hours under hydrogen atmosphere. Then it was filtered through celite to remove Raney nickle. The filtrate was concentrated to give 3-methoxy-4-amino pyridine 63d (0.14 g, 1.1 mmol, pale yellow liquid) as a crude product. Yield: 50%.

MS m/z (ESI): 125 [M+1],

Step 4

### 1-tert-Butoxycarbonyl-3-methoxy-4-aminopyridine

To the solution of 3-methoxy-4-amino pyridine 63d (0.14 g, 1.1 mmol) in tetrahydrofuran (10 mL) were added N,N-diisopropylethylamine (173 mg, 1.3 mmol) and di-t-butyl dicarbonate (366 mg, 1.7 mmol) in an ice bath. Then the resulting mixture was stirred at room temperature overnight. It was concentrated under reduced pressure. Purification of the residue using flash column chromatography (dichloromethane/methanol=1:0-10:1) gave 1-tert-butoxycarbonyl-3-methoxy-4-aminopyridine 63e (0.22 g, 1.0 mmol, white solid).

Yield: 91%.

MS m/z (ESI): 225 [M+1],

Step 5

### 1-Amino-4-((t-butoxycarbonyl)amino)-3-methoxypyridin-1-ium 2,4-dinitrophenolate

To the solution of 1-tert-butoxycarbonyl-3-methoxy-4-aminopyridine 63e (0.22 g, 1.0 mmol) in acetonitrile (30 mL) was added O-(2,4-dinitrophenyl)hydroxylamine (225 mg, 1.1 mmol). Then the resulting mixture was stirred at 40° C. for overnight. It was concentrated to give 1-amino-4-((t-butoxycarbonyl)amino)-3-methoxypyridin-1-ium 2,4-dinitrophenolate 63f (0.45 g, 1.0 mmol, light yellow liquid). Yield: 100%.

MS m/z (ESI): 240 [M+1];

Step 6

### Ethyl 5-((t-butoxycarbonyl)amino)-6-methoxypyrazolo[1,5-a]pyridine-3-carboxylate

To a solution of 1-amino-4-((t-butoxycarbonyl)amino)-3-methoxypyridin-1-ium 2,4-dinitrophenolate 63f (0.45 g, 1.0 mmol) and ethyl propiolate (108 mg, 1.1 mmol) in N,N-dimethylformamide (10 mL) in an ice bath was added potassium carbonate (193 mg, 1.4 mmol). The resulting mixture was stirred at room temperature overnight. Then it was quenched with water and extracted with ethyl acetate (10 mL×3). The organic layer was dried over anhydrous sodium sulfate and filtered. The filtrate was concentrated under reduced pressure. Purification of the residue using flash column chromatography (petroleum ether/ethyl acetate=1:0-5:1) gave ethyl 5-((t-butoxycarbonyl)amino)-6-methoxypyrazolo[1,5-a]pyridine-3-carboxylate 63g (0.15 g, 0.45 mmol, yellow solid). Yield: 40%.

MS m/z (ESI): 336 [M+1],

Step 7

### 6-Methoxypyrazolo[1,5-a]pyridin-5-amine

The solution of ethyl 5-((t-butoxycarbonyl)amino)-6-methoxypyrazolo[1,5-a]pyridine-3-carboxylate 63g (0.15 g, 0.45 mmol) in concentrated sulfuric acid (2 mL, 98%) and water (2 mL) was stirred at 100° C. for 4 hours. Then 6 M aqueous sodium hydroxide was added until pH >7. Then it was extracted with dichloromethane (10 mL×3). The combined organic layer was dried over anhydrous sodium sulfate and filtered. The filtrate was concentrated to give 6-methoxypyrazolo[1,5-a]pyridin-5-amine 63h (72 mg, 0.44 mmol, light yellow solid) as a crude product. Yield: 99%.

MS m/z (ESI): 164 [M+1],

Step 8

### N-(6-((6-Methoxypyrazolo[1,5-a]pyridin-5-yl)amino)pyrimidin-4-yl)cyclopropanecarboxamide

To the mixture of N-(6-chloropyrimidine-4-yl)cyclopropylcarboxamide 63h (5 mg, 0.03 mmol) 6-methoxypyrazolo[1,5-a]pyridin-5-amine (6 mg, 0.03 mmol), and cesium carbonate (30 mg, 0.09 mmol) in 1,4-dioxane (1 mL) were added tris(dibenzylideneacetone)dipalladium(0) (3.2 mg, 0.003 mmol) and dicyclohexyl(2′,4′,6′-triisopropyl-3,6-dimethoxy-[1,1′-biphenyl]-2-yl)phosphine (3.6 mg, 0.006 mmol) at room temperature under argon. Then the resulting mixture was stirred at 110° C. for 1 hour under argon in an oil bath. It was cooled to room temperature, diluted with methanol (5.0 mL) and filtered. The filtrate was concentrated to give crude product which was purified with Prep HPLC (water (with 0.8% ammonia bicarbonate), 10%˜40% acetonitrile, 15 minutes) to give N-(6-((6-methoxypyrazolo[1,5-a]pyridin-5-yl)amino)pyrimidin-4-yl)cyclopropanecarboxamide 63 (2.0 mg, 0.006 mmol, white solid). Yield: 20%.

MS m/z (ESI): 325 [M+1];

¹H NMR (400 MHz, DMSO-d6) δ 10.83 (s, 1H), 9.23 (s, 1H), 8.43 (d, J=7.6 Hz, 1H), 8.34 (s, 1H), 7.94 (s, 1H), 7.61 (s, 1H), 7.41 (d, J=7.6 Hz, 1H), 6.66 (s, 1H), 3.90 (s, 3H), 2.02-2.00 (m, 1H), 0.85-0.75 (m, 4H).

### Example 64

### 6-Methoxy-N-(4-{1-methyl-7-[(3S)-3-methylpiperazin-1-yl]-1H-benzimidazol-2-yl} pyrimidine-5-)-1H-indazol-5-amine

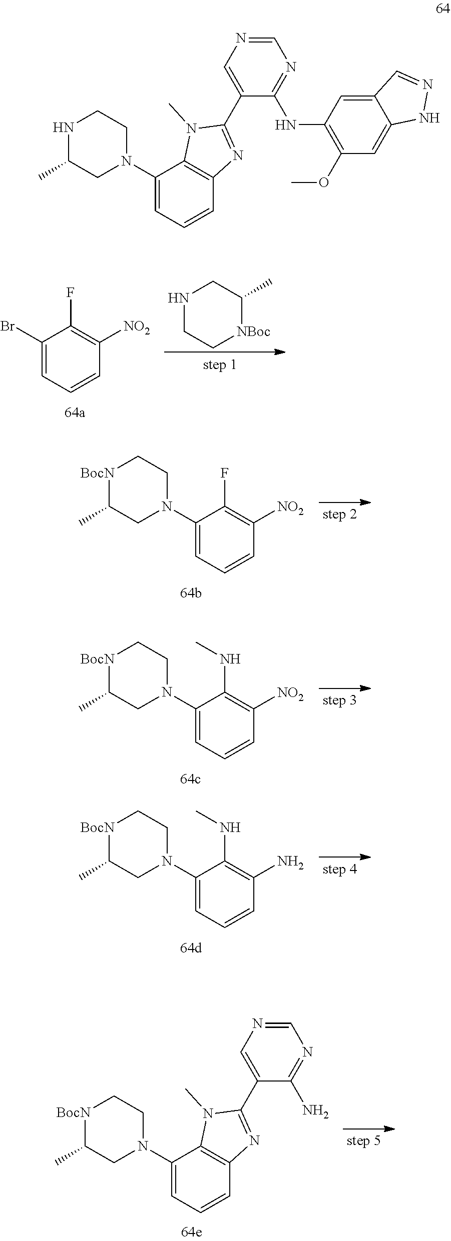

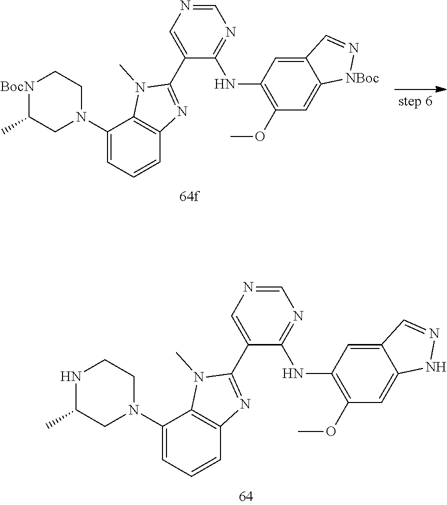 [SMILES: COc1cc2c(cnn2C(=O)OC(C)(C)C)cc1Nc1ncncc1-c1nc2cccc(N3CCN(C(=O)OC(C)(C)C)[C@@H](C)C3)c2n1C ; COc1cc2[nH]ncc2cc1Nc1ncncc1-c1nc2cccc(N3CCN[C@@H](C)C3)c2n1C]

Step 1

### 1-tert-Butoxycarbonyl (2S)-4-(2-fluoro-3-nitrophenyl)-2-methylpiperazine

To the mixture of 1-bromo-2-fluoro-3-nitrobenzene 64a (3.1 g, 14.0 mmol), 1-tert-butoxycarbonyl (2S)-2-methylpiperazine (4.2 g, 21.0 mmol) and 1,4-dioxane (50 mL) were added tris(dibenzylideneacetone)dipalladium(0) (0.6 g, 0.7 mmol), 4,5-bis(diphenylphosphino)-9,9-dimethylxanthene (0.8 g, 1.4 mmol) and cesium carbonate (9.1 g, 28 mmol) at room temperature under argon. The resulting mixture was stirred at 110° C. for 16 hours under argon. Then it was cooled to room temperature and extracted with dichloromethane (100 mL×3). The combined organic layer was washed with brine (100 mL), dried over anhydrous sodium sulfate and filtered. The filtrate was concentrated and the residue was purified by flash column chromatography (petroleum ether/ethyl acetate=5:1) to give 1-tert-butoxycarbonyl (2S)-4-(2-fluoro-3-nitrophenyl)-2-methylpiperazine 64b (2.6 g, 7.7 mmol, red oil). Yield: 55%.

MS m/z (ESI): 340 [M+1].

Step 2

### 1-tert-Butoxycarbonyl (2S)-2-methyl-4-[2-(methylamino)-3-nitrophenyl]piperazine

1-tert-Butoxycarbonyl (2S)-4-(2-fluoro-3-nitrophenyl)-2-methylpiperazine 64b (2.4 g, 7.0 mmol) was mixed with methanamine (2 M in tetrahydrofuran, 15 mL) and N,N-dimethylformamide (20 mL), the resultant mixture was stirred at 130° C. for 16 hours. Then it was cooled to room temperature and extracted with dichloromethane (100 mL×3). The combined organic layer was washed with brine (100 mL), dried over anhydrous sodium sulfate and filtered. The filtrate was concentrated and the residue was purified by flash column chromatography (n-hexane/ethyl acetate=5:1) to give 1-tert-butoxycarbonyl (2S)-2-methyl-4-[2-(methylamino)-3-nitrophenyl] piperazine 64c (1.9 g, 5.4 mmol, red oil). Yield: 78%.

MS m/z (ESI): 351 [M+1],

Step 3

### 1-tert-Butoxycarbonyl (2S)-2-methyl-4-[2-(methylamino)-3-aminophenyl]piperazine

1-tert-Butoxycarbonyl (2S)-2-methyl-4-[2-(methylamino)-3-nitrophenyl]piperazine 64c (0.7 g, 2.0 mmol), Pd/C (containing 55% of water, 0.1 g) and methanol (20 mL) were mixed and stirred at room temperature for 2 hours under hydrogen atmosphere. Then it was filtered. The filtrate was concentrated under reduced pressure and the residue was purified by flash column chromatography (petroleum ether/ethyl acetate=2:1). 1-tert-butoxycarbonyl (2S)-2-methyl-4-[2-(methylamino)-3-aminophenyl] piperazine 64d (0.4 g, 1.2 mmol, yellow oil) was obtained. Yield: 60%.

MS m/z (ESI): 321 [M+1],

Step 4

### 1-tert-Butoxycarbonyl (2S)-4-[2-(4-aminopyrimidin-5-yl)-1-methyl-1H-benzimidazol-7-yl]-2-methylpiperazine

1-tert-Butoxycarbonyl (2S)-2-methyl-4-[2-(methylamino)-3-aminophenyl]piperazine 64d (160.0 mg, 0.5 mmol), 2-(7-benzotriazole)-N, N, N′,N′-tetramethylurea hexafluorophosphate (266.0 mg, 0.7 mmol), N,N-diisopropylethylamine (194.0 mg, 1.5 mmol) and N,N-dimethylformamide (10 mL) were mixed and stirred at room temperature for 10 minutes at room temperature. Then 4-aminopyrimidine-5-methanoic acid (84.0 mg, 0.6 mmol) was added to the above mixture. The resulting mixture was stirred at room temperature for 2 hours. Then it was diluted with water and extracted with dichloromethane (50 mL×3). The combined organic layer was washed with brine (100 mL), dried over anhydrous sodium sulfate and filtered. The filtrate was concentrated and the residue was dissolved in 10 ml of acetic acid directly and reacted for 1 hour at 130° C., cooled to room temperature, concentrated under reduced pressure and diluted with dichloromethane. The pH of the resultant mixture is adjusted to pH=8˜10 by addition of saturated sodium bicarbonate aqueous solution, further extracted with dichloromethane (50 mL×3). The combined organic layer was washed with brine (100 mL), dried over anhydrous sodium sulfate and filtered. The filtrate was concentrated and the residue was purified by flash column chromatography (petroleum ether/ethyl acetate=2:1), and 1-tert-butoxycarbonyl (2S)-4-[2-(4-aminopyrimidin-5-yl)-1-methyl-1H-benzimidazol-7-yl]-2-methylpiperazine 64e (85.0 mg, 0.2 mmol, yellow solid) was obtained. Yield: 40%.

MS m/z (ESI): 424 [M+1],

Step 5

### 1-tert-Butoxycarbonyl-5-[(5-{7-[(3S)-4-(tert-butoxycarbonyl)-3-methylpiperazin-1-yl]-1-methyl-1H-Benzimidazol-2-yl} pyrimidin-4-yl) amino]-6-methoxyindazole

To the mixture of 1-tert-butoxycarbonyl (2S)-4-[2-(4-aminopyrimidin-5-yl)-1-methyl-1H-benzimidazol-7-yl]-2-methylpiperazine 64e (42.0 mg, 0.1 mmol), t-butyl 5-bromo-6-methoxy-1H-indazole-1-carboxylate (32.0 mg, 0.10 mmol) and 1,4-dioxane (2 mL) were added tris(dibenzylideneacetone)dipalladium(0) (9.0 mg, 0.01 mmol), 4,5-bis(diphenylphosphino)-9,9-dimethylxanthene (12.0 mg, 0.02 mmol) and cesium carbonate (98 mg, 0.3 mmol) at room temperature under argon. The resulting mixture was stirred at 120° C. for 1 hour with argon under microwave conditions. Then it was cooled to room temperature, diluted with dichloromethane (10 mL) and filtered. The filtrate was concentrated and the residue was purified by Prep TLC (petroleum ether/ethyl acetate=1:3). 1-tert-butoxycarbonyl-5-[(5-{7-[(3S)-4-(tert-butoxycarbonyl)-3-methylpiperazin-1-yl]-1-methyl-1H-Benzimidazol-2-yl} pyrimidin-4-yl) amino]-6-methoxyindazole 64f (14.0 mg, 0.02 mmol, yellow solid) was obtained. Yield: 20%.

MS m/z (ESI): 670 [M+1],

Step 6

### 6-Methoxy-N-(4-{1-methyl-7-[(3S)-3-methylpiperazin-1-yl]-1H-benzimidazol-2-yl} pyrimidine-5-)-1H-indazol-5-amine

1-tert-Butoxycarbonyl-5-[(5-{7-[(3S)-4-(tert-butoxycarbonyl)-3-methylpiperazin-1-yl]-1-methyl-1H-Benzimidazol-2-yl} pyrimidin-4-yl) amino]-6-methoxyindazole 64f (9.0 mg, 0.015 mmol), dichloromethane (2 mL) and trifluoroacetic acid (1 mL) were mixed and stirred at room temperature for 1 hour. Then it was concentrated and diluted with dichloromethane. The pH value was adjusted to 8˜10 with saturated aqueous sodium bicarbonate solution. The organic layer was separated and the aqueous layer was extracted with dichloromethane (10 mL×3). The combined organic layer was washed with brine (10 mL), dried over anhydrous sodium sulfate and filtered. The filtrate was concentrated under reduced pressure. Purification of the residue using Prep TLC (dichloromethane/methanol=8:1) gave 6-methoxy-N-(4-{1-methyl-7-[(3S)-3-methylpiperazin-1-yl]-1H-benzimidazol-2-yl} pyrimidine-5-)-1H-indazol-5-amine 64 (4.0 mg, 0.01 mmol, yellow solid). Yield: 60%.

MS m/z (ESI): 470 [M+1],

¹H NMR (400 MHz, CDCl₃) δ 11.18 (s, 1H), 8.99 (s, 1H), 8.77 (s, 1H), 8.56 (s, 1H), 7.96 (s, 1H), 7.53 (d, J=8.0 Hz, 1H), 7.23 (d, J=8.0 Hz, 1H), 7.01 (d, J=8.0 Hz, 1H), 6.86 (s, 1H), 4.19 (s, 3H), 3.95 (s, 3H), 3.20-3.09 (m, 4H), 2.59-2.49 (m, 2H), 2.28-2.23 (m, 1H), 1.13 (d, J=6.0 Hz, 3H).

### Example 65

### N-(6-(1H-Imidazol-1-yl)pyrimidin-4-yl)-6-methoxy-1H-indazol-5-amine formate

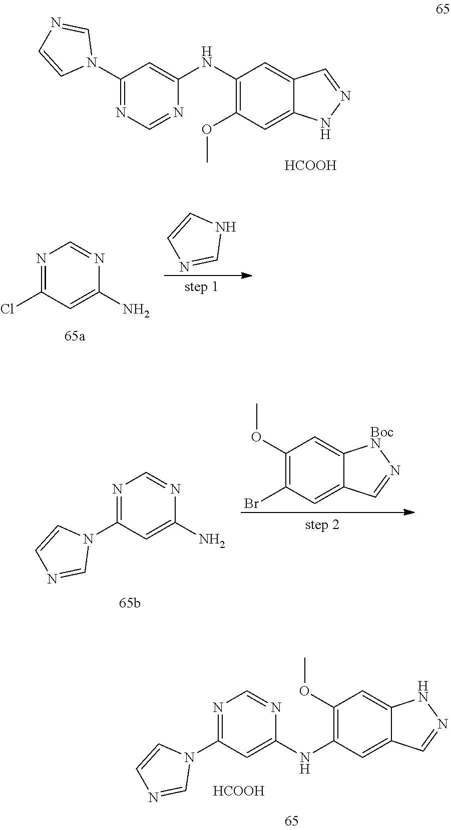

### Step 1

### 6-(1H-Imidazol-1-yl)pyrimidin-4-amine

6-Chloropyrimidin-4-amine 65a (200 mg, 1.5 mmol), imidazole (116 mg, 1.7 mmol), cesium carbonate (978 mg, 3.0 mmol) and N,N-dimethylformamide (2 mL) were mixed and stirred at 120° C. for 8 hours. Then it was cooled to room temperature, diluted with dichloromethane (10 mL) and filtered. The filtrate was concentrated to give the crude product. The crude product was washed with water (50 mL) and dried to give 6-(1H-imidazol-1-yl)pyrimidin-4-amine 65b (90 mg, 0.56 mmol, white solid). Yield: 36%.

MS m/z (ESI): 162 [M+1],

¹H NMR (400 MHz, DMSO-d6) δ 8.45 (s, 1H), 8.33 (s, 1H), 7.82 (s, 1H), 7.20 (s, 2H), 7.12 (s, 1H), 6.57 (s, 1H).

Step 2

### N-(6-(1H-Imidazol-1-yl)pyrimidin-4-yl)-6-methoxy-1H-indazol-5-amine formate

To the mixture of 6-(1H-imidazol-1-yl)pyrimidin-4-amine 65b (30.0 mg, 0.18 mmol), t-butyl 5-bromo-6-methoxy-1H-indazole-1-carboxylate (66.4 mg, 0.20 mmol) and 1,4-dioxane (1 mL) were added tris(dibenzylideneacetone)dipalladium(0) (16.5 mg, 0.018 mmol), 4,5-bis(diphenylphosphino)-9,9-dimethylxanthene (10.5 mg, 0.018 mmol) and cesium carbonate (117 mg, 0.36 mmol) at room temperature under argon. The resulting mixture was stirred at 140° C. for 1 hour with argon under microwave conditions. Then it was cooled to room temperature, diluted with dichloromethane (10 mL) and filtered. The filtrate was concentrated under reduced pressure. Purification of the residue using Prep HPLC (water (with 0.2% formic acid), 10%˜30% acetonitrile, 15 minutes) gave N-(6-(1H-imidazol-1-yl)pyrimidin-4-yl)-6-methoxy-1H-indazol-5-amine formate salt 65 (1.2 mg, 0.004 mmol, white solid). Yield: 2%.

MS m/z (ESI): 308 [M+1],

¹H NMR (400 MHz, DMSO-d6) δ 12.85 (s, 1H), 9.11 (s, 1H), 8.62-8.41 (m, 2H), 8.21-8.04 (m, 2H), 7.97 (s, 1H), 7.85 (s, 1H), 7.17 (s, 1H), 7.07 (s, 1H), 6.92 (s, 1H), 3.89 (s, 3H).

### Example 66

### 5-((6-Aminopyrimidin-4-yl)amino)-4-methoxyisoindolin-1-one hydrochloride

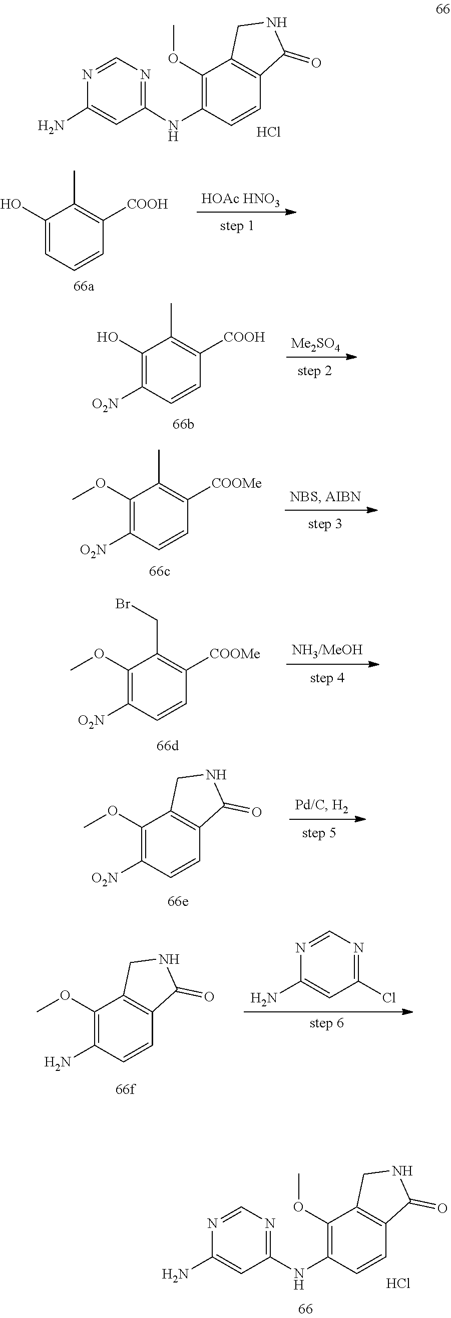

### Step 1 3-Hydroxy-2-methyl-4-nitrobenzoic acid

To the solution of 3-hydroxy-2-methylbenzoic acid 66a (20 g, 131.6 mmol) in acetic acid (160 mL) was added nitric acid (19.6 mL, 70%) slowly at 0° C. The mixture was stirred at room temperature for 1 hour. Then it was poured into ice water (500 mL). The precipitated yellow solid was filtered. 3-Hydroxy-2-methyl-4-nitrobenzoic acid 66b (10.4 g, 52.5 mmol, yellow solid) was obtained. Yield: 40%.

MS m/z (ESI): 198 [M+1],

¹H NMR (400 MHz, CDCl₃) δ 11.09 (s, 1H), 8.04 (d, J=9.0 Hz, 1H), 7.50 (d, J=9.0 Hz, 1H), 2.60 (s, 3H).

Step 2

### Methyl 3-methoxy-2-methyl-4-nitrobenzoate

To the mixture of 3-hydroxy-2-methyl-4-nitrobenzoic acid 66b (10.3 g, 52.0 mmol) with acetone (240 mL) were added dimethyl sulfate (17.4 g, 140.5 mmol) and potassium carbonate (31 g, 234.0 mmol) at room temperature. The mixture was stirred at 60° C. for 2 hours. Then it was concentrated under reduced pressure. Purification of the residue using flash column chromatography (n-Hexane/ethyl acetate=1:0-10:1) gave methyl 3-methoxy-2-methyl-4-nitrobenzoate 66c (11.2 g, 50.0 mmol, yellow solid). Yield: 94%.

MS m/z (ESI): 226 [M+1],

¹H NMR (400 MHz, CDCl₃) δ 7.68 (d, J=8.5 Hz, 1H), 7.61 (d, J=8.5 Hz, 1H), 3.94 (s, 3H), 3.91 (s, 3H), 2.55 (s, 3H).

Step 3

### Methyl 2-(bromomethyl)-3-methoxy-4-nitrobenzoate

To the solution of methyl 3-methoxy-2-methyl-4-nitrobenzoate 66c (5.6 g, 24.9 mmol) in acetonitrile (100 mL) were added N-bromosuccinimide (5.4 g, 30.5 mmol) and azodiisobutyronitrile (82 mg, 0.5 mmol) at room temperature. The mixture was stirred at 80° C. overnight. Then it was quenched with water (100 mL) and extracted with ethyl acetate (50 mL×3). The combined organic layer was washed with brine (50 mL×2), dried over anhydrous sodium sulfate and filtered. The filtrate was concentrated under reduced pressure. Purification of the residue using flash column chromatography (n-hexane/ethyl acetate=1:0˜5:1) gave methyl 2-(bromomethyl)-3-methoxy-4-nitrobenzoate 66d (7.5 g, 24.8 mmol, yellow oil). Yield: 99%.

MS m/z (ESI): 224 [M-Br]

¹H NMR (400 MHz, CDCl₃) δ 7.83-7.75 (m, 2H), 5.05 (s, 2H), 4.05 (s, 3H), 3.99 (s, 3H).

Step 4

### 4-Methoxy-5-nitroisoindolin-1-one

To the solution of methyl 2-(bromomethyl)-3-methoxy-4-nitrobenzoate 66d (7.5 g, 24.8 mmol) in methanol (80 mL) were added triethylamine (3.0 g, 30.0 mmol) and ammonia (25 mL, 175 mmol, 7 M in methanol) at room temperature. The mixture was stirred at 70° C. for 4 hours. Then it was concentrated under reduced pressure. After the residue was recrystallized in methanol. 4-Methoxy-5-nitroisoindolin-1-one 66e (3.8 g, 18.3 mmol, yellow solid) was obtained. Yield: 74%.

MS m/z (ESI): 209 [M+1],

¹H NMR (400 MHz, DMSO-d6) δ 9.07 (s, 1H), 7.92 (d, J=8.0 Hz, 1H), 7.46 (d, J=8.0 Hz, 1H), 4.75 (s, 2H), 4.08 (s, 3H).

Step 5

### 5-Amino-4-methoxyisoindolin-1-one

To the mixture of 4-methoxy-5-nitroisoindolin-1-one 66e (3.8 g, 18.3 mmol) in methanol (300 mL) was added Pd/C (1.0 g, 26 wt %, 55% moisture content) at room temperature. The mixture was stirred at 50° C. under hydrogen atmosphere for overnight. Then it was filtered with celite to remove Pd/C. The filtrate was concentrated under reduced pressure. Purification of the residue using column chromatography (dichloromethane/methanol=1:0˜ 10:1) gave 5-amino-4-methoxyisoindolin-1-one 66f (3.2 g, 18.3 mmol, light yellow solid). Yield: 98%.

MS m/z (ESI): 179 [M+1],

¹H NMR (400 MHz, DMSO-d6) δ 8.02 (s, 1H), 7.12 (t, J=8.0 Hz, 1H), 6.73 (d, J=8.0 Hz, 1H), 5.43 (s, 2H), 4.36 (s, 2H), 3.86-3.70 (m, 3H).

Step 6

### 5-((6-Aminopyrimidin-4-yl)amino)-4-methoxyisoindolin-1-one hydrochloride

To the solution of 5-amino-4-methoxyisoindolin-1-one 66f (0.13 g, 0.73 mmol) in dioxane (10 mL) was added a solution of hydrochlorie in methanol (3.5 mL, 1 M, 3.5 mmol). The reaction tube was sealed, and the mixture was reacted at 130° C. for overnight. Then it was concentrated under reduced pressure. Purification of the residue using Prep-HPLC (water (0.2% formic acid), 10%˜30% acetonitrile, 15 minutes) gave 5-((6-aminopyrimidin-4-yl)amino)-4-methoxyisoindolin-1-one hydrochloride salt 66 (5 mg, 0.018 mmol, white solid). Yield: 2.5%.

MS m/z (ESI): 272 [M+1],

¹H NMR (400 MHz, DMSO-d6) δ 8.45 (s, 1H), 8.38 (s, 1H), 8.09 (d, J=8.2 Hz, 1H), 8.05 (s, 1H), 7.31 (d, J=8.2 Hz, 1H), 6.39 (s, 2H), 5.92 (s, 1H), 4.52 (s, 2H), 3.88 (s, 3H).

### Example 67

### 5-((6-Cyclopropanamidopyrimidin-4-yl) amino)-4-methoxyisodihydroindole-1-one

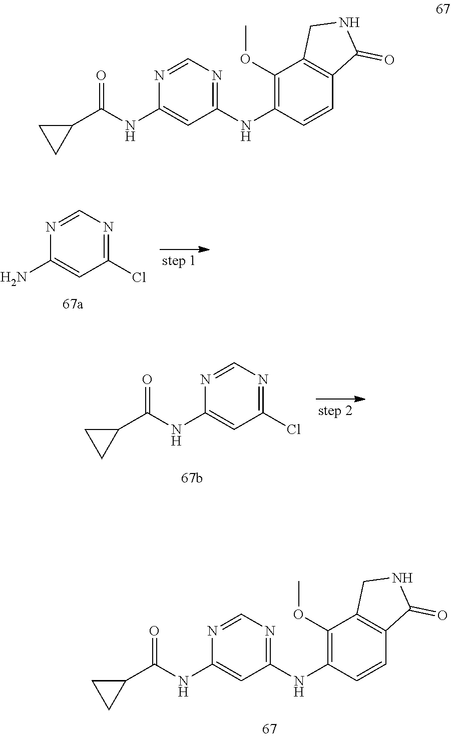 [SMILES: COc1c(Nc2cc(NC(=O)C3CC3)ncn2)ccc2c1CNC2=O ; Nc1cc(Cl)ncn1 ; O=C(Nc1cc(Cl)ncn1)C1CC1 ; COc1c(Nc2cc(NC(=O)C3CC3)ncn2)ccc2c1CNC2=O]

Step 1

### N-(6-Chloropyrimidin-4-yl)cyclopropanecarboxamide

To the solution of 6-chloropyrimidin-4-amine 67a (129 mg, 1.0 mmol) in tetrahydrofuran (5 mL) were added cyclopropanecarbonyl chloride (208 mg, 2.0 mmol) and potassium carbonate (414 mg, 3.0 mmol) at room temperature. The mixture was stirred at 70° C. for overnight. Then it was concentrated under reduced pressure. Purification of the residue using column chromatography (petroleum ether/ethyl acetate=1:0-1:1) gave N-(6-chloropyrimidin-4-yl)cyclopropanecarboxamide 67b (88.6 mg, 0.3 mmol, white solid). Yield: 45%.

MS m/z (ESI): 198 & 200 [M+1],

Step 2

### 5-((6-Cyclopropanamidopyrimidin-4-yl) amino)-4-methoxyisodihydroindole-1-one

To the solution of N-(6-chloropyrimidin-4-yl)cyclopropanecarboxamide 67b (49 mg, 0.25 mmol) in acetic acid (5 mL) was added 5-amino-4-methoxyisoindolin-1-one (45 mg, 0.25 mmol) at room temperature. The mixture was stirred at 110° C. overnight. Then it was concentrated under reduced pressure. Purification of the residue using Prep-HPLC (water (0.2% formic acid), 10%˜30% acetonitrile, 15 minutes) gave 5-((6-cyclopropanamidopyrimidin-4-yl) amino)-4-methoxyisodihydroindole-1-one 67 (2.9 mg, 0.0085 mmol, white solid). Yield: 3%.

MS m/z (ESI): 340 [M+1],

¹H NMR (400 MHz, DMSO-d6) δ 10.83 (s, 1H), 9.10 (s, 1H), 8.54 (s, 1H), 8.37 (s, 1H), 8.10 (d, J=8.1 Hz, 1H), 7.66 (s, 1H), 7.34 (d, J=8.1 Hz, 1H), 4.55 (s, 2H), 3.89 (s, 3H), 2.06-2.02 (m, 1H), 0.88-0.80 (m, 4H).

### Example 68

### 5-((6-(Pyridin-2-ylamino) pyrimidin-4-yl) amino)-4-methoxyisodihydroindol-1-one

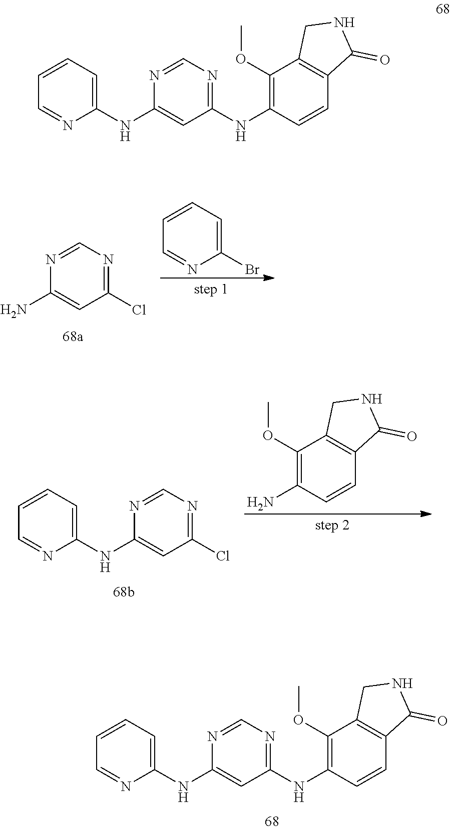 [SMILES: COc1c(Nc2cc(Nc3ccccn3)ncn2)ccc2c1CNC2=O ; Nc1cc(Cl)ncn1 ; Brc1ccccn1 ; Clc1cc(Nc2ccccn2)ncn1 ; COc1c(N)ccc2c1CNC2=O ; COc1c(Nc2cc(Nc3ccccn3)ncn2)ccc2c1CNC2=O]

Step 1

### 6-Chloro-N-(pyridin-2-yl)pyrimidin-4-amine

6-Chloropyrimidin-4-amine 68a (129 mg, 1.0 mmol), 2-bromopyridine (17.2 mg, 1.1 mmol), cesium carbonate (978 mg, 3 mmol), tris(dibenzylideneacetone)-dipalladium(0) (92 mg, 0.1 mmol), 4,5-bis(diphenylphosphino)-9,9-dimethylxanthene (116 mg, 0.2 mmol) and dioxane (5 mL) were mixed and stirred at 110° C. under argon for 1 hour. Then it was filtered. The filtrate was concentrated under reduced pressure. Purification of the residue using column chromatography (petroleum ether/ethyl acetate=0:1-1:1) gave 6-chloro-N-(pyridin-2-yl)pyrimidin-4-amine 68b (116 mg, 0.56 mmol, white solid). Yield: 56%.

MS m/z (ESI): 207 & 209 [M+1],

¹H NMR (400 MHz, CDCl₃) δ 8.57 (s, 1H), 8.37 (d, J=4.7 Hz, 1H), 7.96 (s, 1H), 7.77-7.58 (m, 2H), 7.29 (d, J=8.4 Hz, 1H), 7.06-6.99 (m, 1H).

Step 2

### 5-((6-(Pyridin-2-ylamino) pyrimidin-4-yl) amino)-4-methoxyisodihydroindol-1-one

To the mixture of 6-chloro-N-(pyridin-2-yl)pyrimidin-4-amine 68b (65 mg, 0.5 mmol) in butan-1-ol (2 mL) were added 5-amino-4-methoxyisoindolin-1-one (90 mg, 0.5 mmol) and a solution of hydrochloric acid in methanol (2 mL, 4 M). The reaction tube was sealed, and the mixture was stirred at 130° C. overnight. Then it was concentrated under reduced pressure. Purification of the residue using Prep-HPLC (water (0.2% formic acid), 10%˜30% acetonitrile, 15 minutes) gave 5-((6-(pyridin-2-ylamino) pyrimidin-4-yl) amino)-4-methoxyisodihydroindol-1-one 68 (4.4 mg, 0.0126 mmol, white solid). Yield: 3%.

MS m/z (ESI): 349 [M+1],

¹H NMR (400 MHz, DMSO-d6) δ 9.83 (s, 1H), 8.87 (s, 1H), 8.50 (s, 1H), 8.31 (s, 1H), 8.27 (d, J=5.2 Hz, 1H), 8.10 (d, J=8.1 Hz, 1H), 7.69 (t, J=7.8 Hz, 1H), 7.48 (d, J=7.8 Hz, 2H), 7.34 (d, J=8.1 Hz, 1H), 6.94 (t, J=5.2 Hz, 1H), 4.56 (s, 2H), 3.91 (s, 3H).

### Example 69

### 5-((6-(Pyrimidin-2-ylamino) pyrimidin-4-yl) amino)-4-methoxyisodihydroindol-1-one

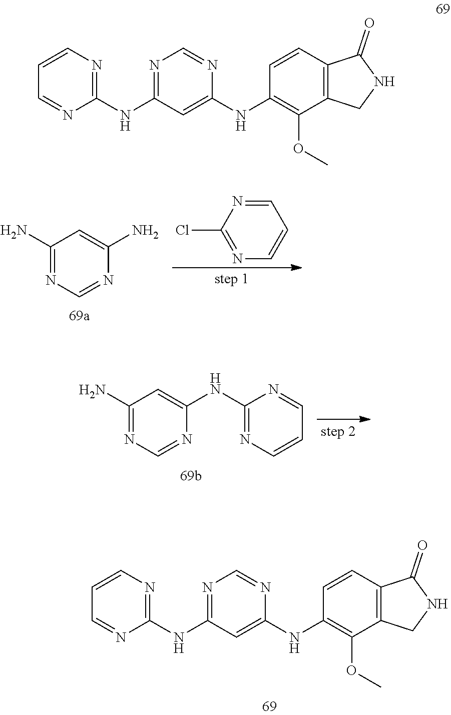 [SMILES: COc1c(Nc2cc(Nc3ncccn3)ncn2)ccc2c1CNC2=O ; Nc1cc(N)ncn1 ; Clc1ncccn1 ; Nc1cc(Nc2ncccn2)ncn1 ; COc1c(Nc2cc(Nc3ncccn3)ncn2)ccc2c1CNC2=O]

Step 1

### N-(Pyrimidin-2-yl)pyrimidine-4,6-diamine

To the mixture of pyrimidine-4,6-diamine 69a (110.0 mg, 1.0 mmol) and sodium hydride (60% dispersion in mineral oil, 120.0 mg, 5.0 mmol) in N, N-dimethylacetamide (10.0 mL) was added 2-chloropyrimidine (114.0 mg, 1.0 mmol) at room temperature. The mixture was stirred at 70° C. for 2 hours. Then it was cooled to room temperature and quenched with saturated aqueous sodium bicarbonate (10 mL) and extracted with dichloromethane (30 mL×3). The combined organic layer was washed with brine (30 mL), dried over anhydrous sodium sulfate and then filtered. The filtrate was concentrated under reduced pressure. Purification of the residue using column chromatography (dichloromethane/methanol=10:1) gave N-(Pyrimidin-2-yl)pyrimidine-4,6-diamine 69b (47.0 mg, 0.20 mmol, white solid). Yield: 25%.

MS m/z (ESI): 189 [M+1],

Step 2

### 5-((6-(Pyrimidin-2-ylamino) pyrimidin-4-yl) amino)-4-methoxyisodihydroindol-1-one

To the mixture of N-(Pyrimidin-2-yl)pyrimidine-4,6-diamine 69b (19.0 mg, 0.10 mmol), 5-bromo-4-methoxyisoindolin-1-one (25.0 mg, 0.10 mmol) and 1,4-dioxane (2.0 mL) were added tris(dibenzylideneacetone)dipalladium(0) (9.0 mg, 0.01 mmol) and 4,5-bis(diphenylphosphino)-9,9-dimethylxanthene (12.0 mg, 0.02 mmol) at room temperature under argon. The mixture was stirred at 110° C. for 1 hour in microwave oven with argen. Then it was diluted with dichloromethane (10 mL) and filtered. The filtrate was concentrated under reduced pressure. Purification of the residue using Prep-TLC (dichloromethane/methanol=20:1) gave 5-((6-(pyrimidin-2-ylamino) pyrimidin-4-yl) amino)-4-methoxyisodihydroindol-1-one 69 (7.0 mg, 0.02 mmol, white solid). Yield: 20%.

MS m/z (ESI): 350 [M+1],

¹H NMR (400 MHz, DMSO-d6) δ 10.08 (s, 1H,), 9.03 (s, 1H,), 8.63 (d, J=4.4 Hz, 2H), 8.53 (s, 1H), 8.36 (s, 1H), 8.16 (d, J=8.0 Hz, 1H), 7.91 (s, 1H), 7.35 (d, J=8.0 Hz, 1H), 7.05 (t, J=4.4 Hz, 1H), 4.58 (s, 2H), 3.93 (s, 3H).

### Example 70

### 5-((6-(Pyrimidin-4-ylamino) pyrimidin-4-yl) amino)-4-methoxyisodihydroindol-1-one

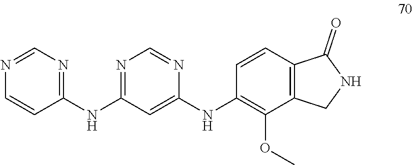 [SMILES: COc1c(Nc2cc(Nc3ccncn3)ncn2)ccc2c1CNC2=O]

The synthetic procedure is similar to that of example 69. Using 4-chloropyrimidine instead of 2-chloropyrimidine. The desired compound 5-((6-(pyrimidin-4-ylamino) pyrimidin-4-yl) amino)-4-methoxyisodihydroindol-1-one (7 mg, 0.01 mmol, white solid) was obtained. Yield: 20%.

MS m/z (ESI): 350 [M+1],

¹H NMR (400 MHz, DMSO-d6) δ 10.32 (s, 1H), 9.10 (s, 1H), 8.77 (s, 1H), 8.54 (s, 1H), 8.46 (d, J=6.0 Hz, 1H), 8.38 (s, 1H), 8.06 (d, J=8.0 Hz, 1H), 7.59 (d, J=6.0 Hz, 1H), 7.49 (s, 1H), 7.34 (d, J=8.0 Hz, 1H), 4.57 (s, 2H), 3.92 (s, 3H).

### Example 71

### 5-((6-Amino-5-methoxypyrimidin-4-yl)amino)-4-methoxyisoindolin-1-one

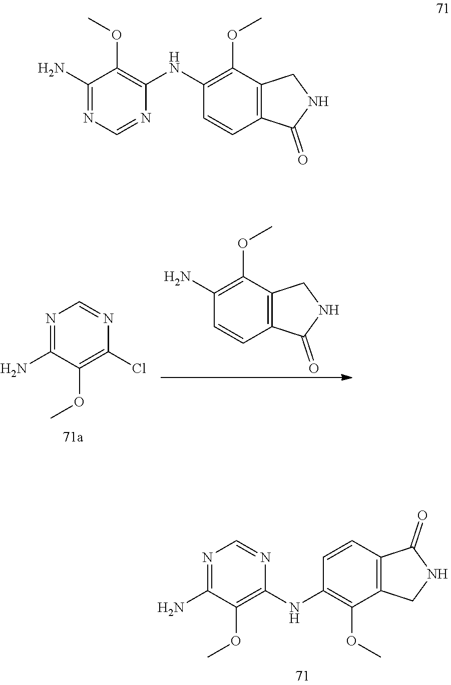

To the mixture of 6-chloro-5-methoxy-4-aminopyrimidine 71a (16.0 mg, 0.1 mmol), 5-amino-4-methoxyisoindolin-1-one (18.0 mg, 0.1 mmol) and dioxane (1 mL) was added a solution of hydrochloric acid in dioxane (0.1 mL, 4 M) at room temperature. The mixture was stirred at 100° C. under argon for 16 hours. Then it was quenched with saturated aqueous sodium bicarbonate (10 mL). The organic layer was separated and the aqueous layer was extracted with dichloromethane (15 mL×2). The combined organic layer was washed with brine (50 mL×2), dried over anhydrous sodium sulfate and filtered. The filtrate was concentrated under reduced pressure. Purification of the residue using Prep-TLC (dichloromethane/methanol=10:1) gave 5-((6-amino-5-methoxypyrimidin-4-yl)amino)-4-methoxyisoindolin-1-one 71 (3 mg, 0.01 mmol, yellow solid). Yield: 10%.

MS m/z (ESI): 302 [M+1],

¹H NMR (400 MHz, DMSO-d6) δ 8.58 (d, J=8.4 Hz, 1H), 8.46 (s, 1H), 7.93 (s, 1H), 7.83 (s, 1H), 7.35 (d, J=8.4 Hz, 1H), 6.58 (s, 2H), 4.59 (s, 2H), 4.01 (s, 3H), 3.71 (s, 3H).

### Example 72

### 5-((6-(Cyclopropionamido) pyrimidin-4-yl) amino)-4-methoxy-2-methylisodihydroindol-1-one

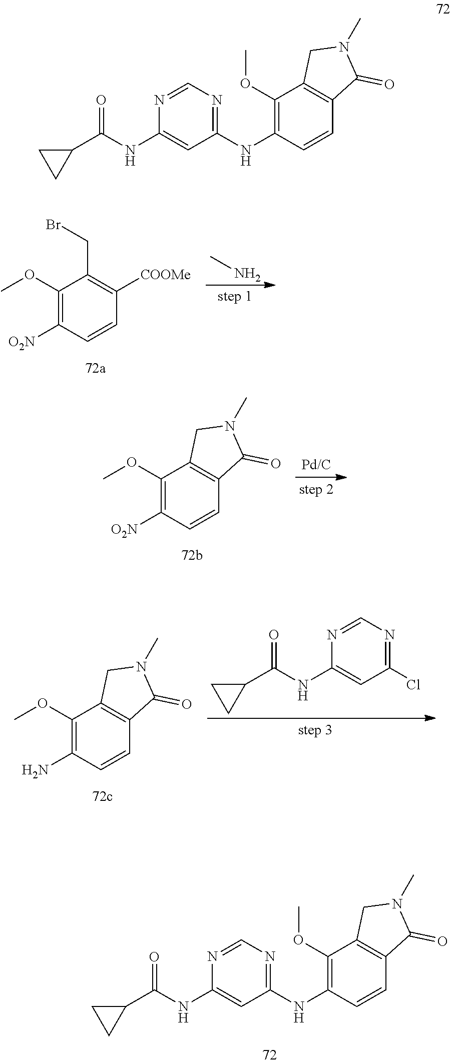

Step 1

### 4-Methoxy-2-methyl-5-nitroisoindolin-1-one

To the mixture of methyl 2-(bromomethyl)-3-methoxy-4-nitrobenzoate 72a (0.3 g, 1.0 mmol) in methanol (10 mL) was added methylamine (1 mL, 10 M in water) at room temperature. The mixture was stirred at room temperature for 1 hour. Then it was concentrated under reduced pressure. The residue was recrystallized in methanol. 4-Methoxy-2-methyl-5-nitroisoindolin-1-one 72b (0.2 g, 0.9 mmol, yellow solid) was obtained. Yield: 90%.

MS m/z (ESI): 223 [M+1].

Step 2

### 5-Amino-4-methoxy-2-methylisoindolin-1-one

To the mixture of 4-methoxy-2-methyl-5-nitroisoindolin-1-one 72b (0.2 g, 0.9 mmol) in methanol (50 mL) was added Pd/C (50 mg, 26% wt, 55% moisture content) at room temperature. The mixture was stirred at 40° C. under hydrogen for 12 hours. Then it was filtered with celite to remove Pd/C. The filtrate was concentrated under reduced pressure. Purification of the residue using column chromatography (dichloromethane/methanol=1:0-10:1) gave 5-amino-4-methoxy-2-methylisoindolin-1-one 72c (0.15 g, 0.78 mmol, light yellow solid).

Yield: 87%.

MS m/z (ESI): 193 [M+1],

¹H NMR (400 MHz, DMSO-d6) δ 7.09 (d, J=7.8 Hz, 1H), 6.72 (d, J=7.8 Hz, 1H), 5.47 (s, 2H), 4.47 (s, 2H), 3.80 (s, 3H), 3.00 (s, 3H).

### Step 3

### 5-((6-(Cyclopropionamido) pyrimidin-4-yl) amino)-4-methoxy-2-methylisodihydroindol-1-one

To the mixture of 5-amino-4-methoxy-2-methylisoindolin-1-one 72c (10 mg, 0.05 mmol), N-(6-chloropyrimidin-4-yl)cyclopropanecarboxamide (10 mg, 0.05 mmol) and cesium carbonate (50 mg, 0.15 mmol) in 1,4-dioxane (1.0 mL) were added tris(dibenzylideneacetone)dipalladium(0) (4.2 mg, 0.005 mmol) and 4,5-bis(diphenylphosphino)-9,9-dimethylxanthene (5.6 mg, 0.01 mmol) at room temperature under argon. The mixture was stirred at 110° C. for 1 hour in an oil bath. Then it was cooled to room temperature, diluted with methanol (5 mL) and filtered. The filtrate was concentrated under reduced pressure. Purification of the residue using Prep-HPLC (water (0.2% formic acid), 20%˜60% acetonitrile, 15 minutes) gave 5-((6-(cyclopropionamido) pyrimidin-4-yl) amino)-4-methoxy-2-methylisodihydroindol-1-one 72 (5.0 mg, 0.014 mmol, white solid). Yield: 28%.

MS m/z (ESI): 354 [M+1],

¹H NMR (400 MHz, DMSO-d6) δ 10.82 (s, 1H), 9.09 (s, 1H), 8.38 (s, 1H), 8.10 (d, J=8.1 Hz, 1H), 7.66 (s, 1H), 7.34 (d, J=8.1 Hz, 1H), 4.72-4.66 (m, 2H), 3.90 (s, 3H), 3.10-3.06 (m, 3H), 2.05-1.98 (m, 1H), 0.96-0.78 (m, 4H).

### Example 73

### 5-((6-(Cyclopropionamido) pyrimidin-4-yl) amino)-4-methoxy-2,3,3-trimethylisoindole-1-one

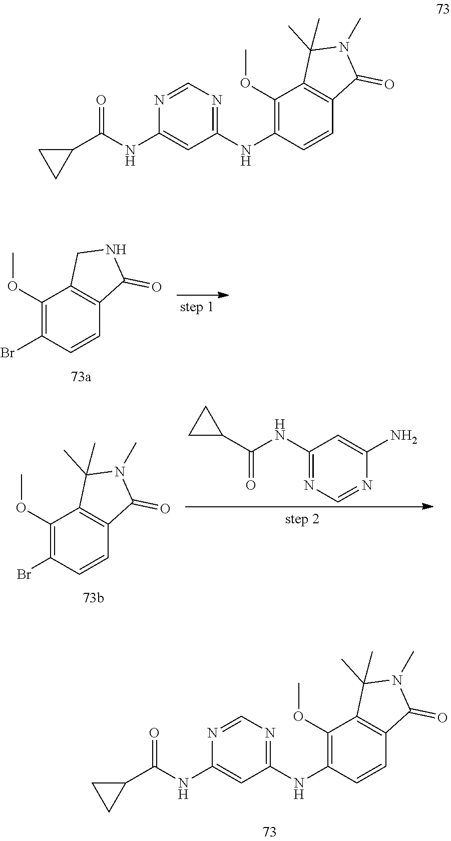 [SMILES: COc1c(Nc2cc(NC(=O)C3CC3)ncn2)ccc2c1C(C)(C)N(C)C2=O ; COc1c(Br)ccc2c1CNC2=O ; COc1c(Br)ccc2c1C(C)(C)N(C)C2=O ; Nc1cc(NC(=O)C2CC2)ncn1 ; COc1c(Nc2cc(NC(=O)C3CC3)ncn2)ccc2c1C(C)(C)N(C)C2=O]

Step 1

### 5-Bromo-4-methoxy-2,3,3-trimethylisoindolin-1-one

To the solution of 5-bromo-4-methoxyisoindolin-1-one 73a (30.0 mg, 0.12 mmol) in N,N-dimethylformamide (10.0 mL) was added sodium hydride (60% dispersion in mineral oil, 40.0 mg, 0.96 mmol) in several portions at 0° C. The mixture was stirred at room temperature for 15 minutes. Then iodomethane (106.0 mg, 0.72 mmol) was added dropwise and the resulting mixture was stirred at 60° C. for 1 hour. Then it was cooled to room temperature, quenched with water (30 mL) and extracted with dichloromethane (15 mL×3). The combined organic layer was washed with brine (30 mL), dried over anhydrous sodium sulfate and then filtered. The filtrate was concentrated under reduced pressure. Purification of the residue using Prep-TLC (dichloromethane/methanol=30:1) gave 5-bromo-4-methoxy-2,3,3-trimethylisoindolin-1-one 73b (20.0 mg, 0.071 mmol, yellow oil). Yield: 59%.

MS m/z (ESI): 284 & 286 [M+1],

Step 2

### 5-((6-(Cyclopropionamido) pyrimidin-4-yl) amino)-4-methoxy-2,3,3-trimethylisoindole-1-one

To the mixture of 5-bromo-4-methoxy-2,3,3-trimethylisoindolin-1-one 73b (20.0 mg, 0.07 mmol), N-(6-aminopyrimidin-4-yl)cyclopropanecarboxamide (19.0 mg, 0.11 mmol) and cesium carbonate (92.6 mg, 0.28 mmol) in 1,4-dioxane (1.0 mL) were added tris(dibenzylideneacetone)dipalladium(0) (6.1 mg, 0.007 mmol) and 4,5-bis(diphenylphosphino)-9,9-dimethylxanthene (7.8 mg, 0.014 mmol) at room temperature under argon. The mixture was stirred at 80° C. for 3 hours. Then it was cooled to room temperature, diluted with dichloromethane (10 mL) and filtered. The filtrate was concentrated under reduced pressure. Purification of the residue using Prep-TLC (dichloromethane/methanol=15:1) gave 5-((6-(cyclopropionamido) pyrimidin-4-yl) amino)-4-methoxy-2,3,3-trimethylisoindole-1-one 73 (10.0 mg, 0.026 mmol, white solid). Yield: 37%.

MS m/z (ESI): 382 [M+1],

¹H NMR (400 MHz, DMSO-d6) δ 10.82 (s, 1H), 9.31 (s, 1H), 8.34 (s, 1H), 7.80 (d, J=8.0 Hz, 1H), 7.59 (s, 1H), 7.39 (d, J=8.0 Hz, 1H), 3.74 (s, 3H), 2.90 (s, 3H), 2.09-1.91 (m, 1H), 1.49 (s, 6H), 7.65-7.63 (m, 4H).

### Example 74

### 5-((5-Methoxypyrimidin-4-yl) amino)-4-methoxyisodihydroindole-1-one

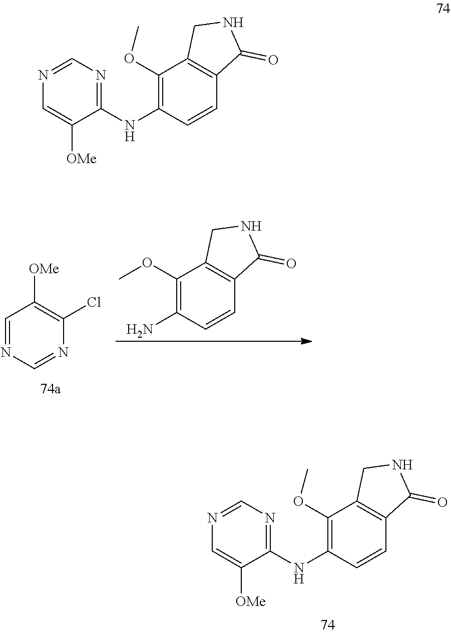

4-Chloro-5-methoxypyrimidine 74a (15.0 mg, 0.1 mmol), 5-amino-4-methoxyisoindolin-1-one (18.0 mg, 0.1 mmol) and acetic acid (2 mL) were mixed and stirred at 70° C. for 1 hour. Then it was cooled to room temperature. White solid was formed and filtered. The filter cake was washed with acetic acid (20 mL) and dried to give 5-((5-methoxypyrimidin-4-yl) amino)-4-methoxyisodihydroindole-1-one 74 (8.0 mg, 0.03 mmol, white solid). Yield: 30%.

MS m/z (ESI): 287 [M+1],

¹H NMR (400 MHz, DMSO-d6) δ 9.93 (s, 1H), 8.74 (s, 1H), 8.59 (s, 1H), 8.24 (s, 1H), 7.80 (d, J=7.2 Hz, 1H), 7.41 (d, J=7.2 Hz, 1H), 4.66 (s, 2H), 4.05 (s, 3H), 3.94 (s, 3H).

### Example 75

### 5-((5-(2-Methoxyethoxy) pyrimidin-4-yl) amino)-4-methoxyisodihydroindole-1-one

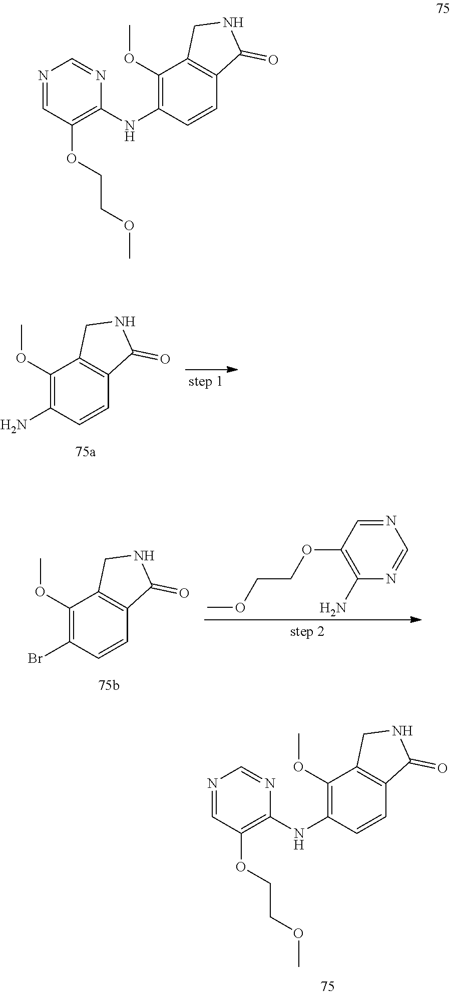

### Step 1

### 5-Bromo-4-methoxyisoindolin-1-one

To the solution of 5-amino-4-methoxyisoindolin-1-one 75a (370.0 mg, 2.1 mmol) and copper(I) bromide (450.0 mg, 3.1 mmol) in acetonitrile (10.0 mL) was added t-butyl nitrite (430.0 mg, 4.2 mmol) at room temperature. The resulting mixture was stirred at 50° C. for 2 hours. Then it was cooled to room temperature and quenched with diluted hydrochloric acid (5 mL, 1 M). The mixture was diluted with dichloromethane (50 mL). The organic layer was washed with brine (20 mL×3), dried over anhydrous sodium sulfate and then filtered. The filtrate was concentrated under reduced pressure. Purification of the residue using column chromatography (dichloromethane/methanol=20/1) gave 5-bromo-4-methoxyisoindolin-1-one 75b (200 mg, 0.83 mmol, yellow solid). Yield: 40%.

MS m/z (ESI): 242 & 244 [M+1],

¹H NMR (400 MHz, CDCl₃) δ 7.68 (d, J=8.0 Hz, 1H), 7.48 (d, J=8.0 Hz, 1H), 7.10 (brs, 1H), 4.56 (s, 2H), 3.98 (s, 3H).

Step 2

### 5-((5-(2-Methoxyethoxy) pyrimidin-4-yl) amino)-4-methoxyisodihydroindole-1-one

To the mixture of 5-bromo-4-methoxyisoindolin-1-one 75b (28.0 mg, 0.12 mmol), 5-(2-methoxyethoxy)-4-aminopyrimidine (20.0 mg, 0.12 mmol) and cesium carbonate (72.0 mg, 0.23 mmol) in 1,4-dioxane (2.0 mL) were added tris(dibenzylideneacetone)dipalladium(0) (1.0 mg, 0.012 mmol) and 4,5-bis(diphenylphosphino)-9,9-dimethylxanthene (12.0 mg, 0.023 mmol) at room temperature under argon. The mixture was stirred at 120° C. for 1 hour in microwave oven. It was cooled to room temperature and filtered. The filtrate was concentrated. Purification of the residue using Prep-HPLC (water (0.2% formic acid), 0%˜15% acetonitrile, 15 minutes) gave 5-((5-(2-methoxyethoxy) pyrimidin-4-yl) amino)-4-methoxyisodihydroindole-1-one 75 (4.0 mg, 0.012 mmol, white solid). Yield: 10%.

MS m/z (ESI): 331 [M+1],

¹H NMR (400 MHz, DMSO-d6) δ 8.71 (d, J=8.2 Hz, 1H), 8.55 (s, 1H), 8.40 (s, 1H), 8.32 (s, 1H), 8.21 (brs, 1H), 7.41 (d, J=8.2 Hz, 1H), 4.63 (s, 2H), 4.35-4.33 (m, 2H), 4.03 (s, 3H), 3.78-3.74 (m, 2H), 3.39 (s, 3H).

### Example 76

### 5-((5-(2-(4-morpholin) ethoxy) pyrimidin-4-yl) amino)-4-methoxyisodihydroindol-1-one formate

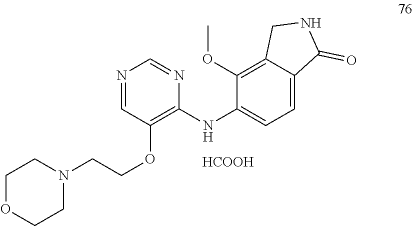 [SMILES: COc1c(Nc2ncncc2OCCN2CCOCC2)ccc2c1CNC2=O ; "HCOOH"]

The synthetic procedure is similar to that of example 75. Using 4-(2-chloroethyl)morpholine instead of 1-bromine-2-methoxyethane. The desired compound 5-((5-(2-(4-morpholin) ethoxy) pyrimidin-4-yl) amino)-4-methoxyisodihydroindol-1-one formate was obtained. Yield: 25%.

MS m/z (ESI): 386 [M+1],

¹H NMR (400 MHz, DMSO-d6) δ 8.73 (d, J=8.0 Hz, 1H), 8.54 (s, 1H), 8.40 (s, 1H), 8.30 (s, 1H), 8.21 (s, 1H), 7.41 (d, J=8.0 Hz, 1H), 4.64 (s, 2H), 4.34-4.32 (m, 2H), 4.04 (s, 3H), 3.60-3.54 (m, 4H), 3.37-3.32 (m, 4H), 2.81-2.79 (m, 2H).

### Example 77

### 5-((5-(2-(Dimethylamino) ethoxy) pyrimidin-4-yl) amino)-4-methoxyisodihydroindole-1-one dicarboxylate

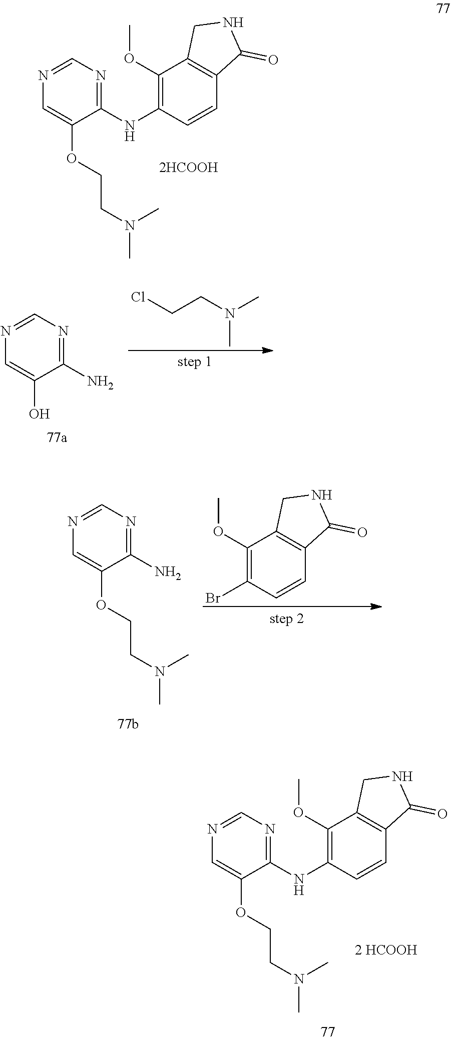

Step 1

### 5-(2-(Dimethylamino)ethoxy)-4-aminopyrimidine

4-Amino-5-hydroxypyrimidine 77a (100.0 mg, 0.90 mmol), anhydrous lithium hydroxide (65.0 mg, 2.70 mmol) and N,N-dimethylformamide (3.0 mL) were mixed and stirred at room temperature for 30 minutes. Then N,N-dimethyl-3-chloroethylamine (125.0 mg, 0.90 mmol) was added to the above mixture. The resulting mixture was stirred at 60° C. for 5 hours. Then it was cooled to room temperature and filtered. The filtrate was concentrated under reduced pressure. Purification of the residue using Prep-TLC (dichloromethane/methanol=10:1) gave 5-(2-(dimethylamino)ethoxy)-4-aminopyrimidine 77b (60.0 mg, 0.36 mmol, colorless oil). Yield: 40%.

MS m/z (ESI): 183 [M+1],

¹H NMR (400 MHz, CDCl₃) δ 8.24 (s, 1H), 7.81 (s, 1H), 5.93 (brs, 2H), 4.23-4.18 (m, 2H), 3.08-3.04 (m, 2H), 2.60 (s, 6H).

Step 2

### 5-((5-(2-(Dimethylamino)ethoxy)pyrimidin-4-yl)amino)-4-methoxyisoindolin-1-one diformate

To the mixture of 5-(2-(Dimethylamino)ethoxy)-4-aminopyrimidine 77b (12.0 mg, 0.06 mmol), 5-bromo-4-methoxyisoindolin-1-one (10.0 mg, 0.04 mmol) and cesium carbonate (40.0 mg, 0.12 mmol) in 1,4-dioxane (2.0 mL) were added tris(dibenzylideneacetone)dipalladium(0) (4.0 mg, 0.004 mmol) and 4,5-bis(diphenylphosphino)-9,9-dimethylxanthene (4.0 mg, 0.008 mmol) at room temperature under argon. The mixture was stirred at 140° C. for 1 hour in microwave oven. It was cooled to room temperature and filtered. The filtrate was concentrated under reduced pressure. Purification of the residue using Prep-HPLC (water (0.2% formic acid), 0%-15% acetonitrile, 15 minutes) gave 5-((5-(2-(Dimethylamino)ethoxy)pyrimidin-4-yl)amino)-4-methoxyisoindolin-1-one diformate 77 (2.0 mg, 0.006 mmol, white solid). Yield: 10%.

MS m/z (ESI): 344 [M+1],

¹H NMR (400 MHz, DMSO-d6) δ 8.70 (d, J=8.0 Hz, 1H), 8.55 (s, 1H), 8.42 (s, 1H), 8.40 (s, 1H), 8.26 (s, 1H), 8.21 (s, 1H), 7.96 (s, 1H), 7.42 (d, J=8.0 Hz, 1H), 4.64 (s, 2H), 4.30 (t, J=12.0 Hz, 2H), 2.90 (s, 3H), 2.78 (t, J=12.0 Hz, 2H), 2.76 (s, 6H).

### Example 78

### 5-((5-(2-Hydroxyethoxy)pyrimidin-4-yl)amino)-4-methoxyisoindolin-1-one

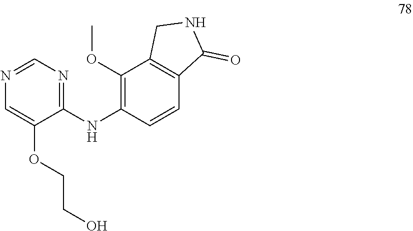 [SMILES: COc1c(Nc2ncncc2OCCO)ccc2c1CNC2=O]

The synthetic procedure is similar to that of example 54. Using 5-bromine-4-methoxyisoindolin-1-one instead of 1-t-butyloxoyl-5-amino-6-methoxy indazole. The desired compound: 5-((5-(2-Hydroxyethoxy)pyrimidin-4-yl)amino)-4-methoxyisoindolin-1-one was obtained. Yield: 25%.

MS m/z (ESI): 317 [M+1],

¹H NMR (400 MHz, DMSO-d6) δ 8.53 (s, 1H), 8.51 (d, J=8.0, 1H), 8.41 (s, 1H), 8.35 (s, 1H), 8.18 (s, 1H), 7.41 (d, J=8.0, 1H), 5.32-4.79 (brs, 1H), 4.62 (s, 2H), 4.22-4.21 (m, 2H), 3.80 (s, 3H), 3.82-3.79 (m, 2H).

### Example 79

### 5-((7H-Pyrrolo[2,3-d]pyrimidin-4-yl)amino)-4-methoxyisoindolin-1-one

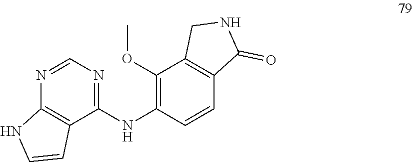 [SMILES: COc1c(Nc2ncnc3[nH]ccc23)ccc2c1CNC2=O]

The synthetic procedure is similar to that of example 74. Using 4-chloropyrrolo[2,3-d]pyrimidine instead of 4-chloro-5-methoxypyrimidine. The desired compound: 5-((7H-Pyrrolo[2,3-d]pyrimidin-4-yl)amino)-4-methoxyisoindolin-1-one (15.0 mg, 0.05 mmol, red solid) was obtained. Yield: 18%.

MS m/z (ESI): 296 [M+1],

¹H NMR (400 MHz, DMSO-d6) δ 11.80 (brs, 1H), 8.78 (brs, 1H), 8.56 (s, 1H), 8.24 (s, 1H), 8.14 (d, J=8.0 Hz, 1H), 7.38 (d, J=8.0 Hz, 1H), 7.25 (brd, 1H), 6.71 (brd, 1H), 4.64 (s, 2H), 3.93 (s, 3H).

### Example 80

### 5-((9H-Purin-6-yl)amino)-4-methoxyisoindolin-1-one

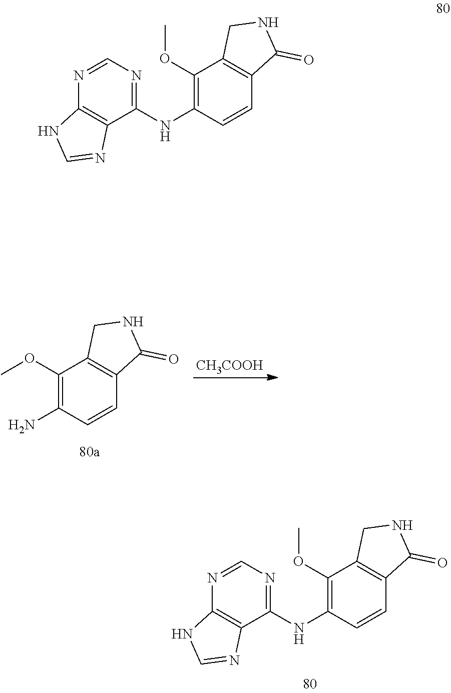

To the solution of 5-amino-4-methoxyisoindolin-1-one 80a (22 mg, 0.125 mmol) in acetic acid (2 mL) was added 6-chloro-9H-purine (40 mg, 0.25 mmol) at room temperature. The reaction tube was sealed and the resulting solution was stirred at 100° C. for 1 hour. Then it was concentrated under reduced pressure. Purification of the residue using Prep-HPLC (water (0.2% formic acid), 10%˜% acetonitrile, 15 minutes) gave 5-((9H-Purin-6-yl)amino)-4-methoxyisoindolin-1-one 80 (2.2 mg, 0.0074 mmol, white solid). Yield: 6%.

MS m/z (ESI): 297 [M+1],

¹H NMR (400 MHz, DMSO-d6) δ 13.62 (s, 1H), 9.71 (s, 1H), 8.66 (s, 1H), 8.31 (s, 1H), 8.12 (s, 1H), 7.87 (d, J=8.0 Hz, 1H), 7.40 (d, J=8.0 Hz, 1H), 4.62 (s, 2H), 3.90 (s, 3H).

### Example 81

### 5-((1H-Pyrazolo[3,4-d]pyrimidin-4-yl)amino)-4-methoxyisoindolin-1-one

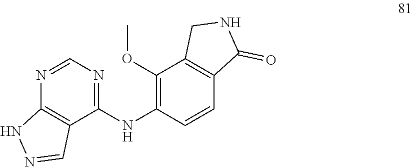

The synthetic procedure is similar to that of example 74. Using 4-chloro-1H-pyrazolo[3,4-d]pyrimidine instead of 6-chloro-9H-purine, the desired compound: 5-((1H-pyrazolo[3,4-d]pyrimidin-4-yl)amino)-4-methoxyisoindolin-1-one (6 mg, 0.02 mmol, white solid) was obtained. Yield: 16%.

MS m/z (ESI): 297 [M+1],

¹H NMR (400 MHz, DMSO-d6) δ 13.32 (s, 1H), 8.68-8.84 (m, 2H), 8.57 (s, 1H), 8.46 (s, 1H), 8.34 (s, 1H), 7.42 (d, J=8.4 Hz, 1H), 4.65 (s, 2H), 4.05 (s, 3H).

### Example 82

### 5-((5-Methylaminopyrimidin-4-yl) amino)-4-methoxyisodihydroindole-1-one formate

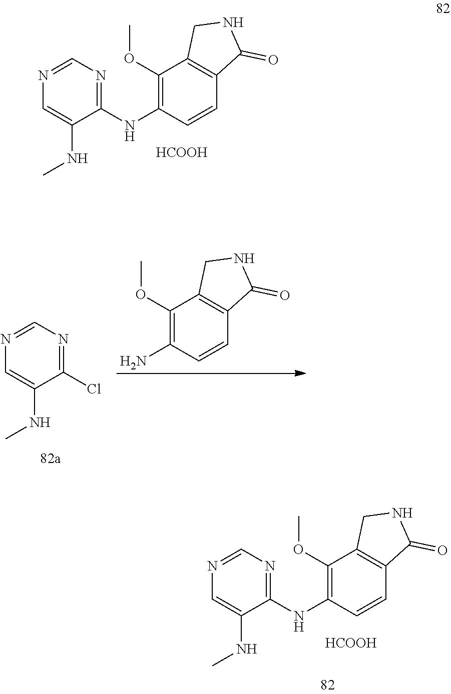

4-Chloro-N-methylpyrimidin-5-amine 82a (10 mg, 0.070 mmol), 4-methoxy-5-((5-(methylamino) pyrimidin-4-yl) amino) isodihydroindole-1-one (15 mg, 0.084 mmol), hydrochloric acid in methanol (0.5 mL, 2 M) and butan-1-ol (0.5 mL) were mixed and stirred at 130° C. for 2 hours. Triethylamine was added until pH=7˜8. Then it was concentrated under reduced pressure. Purification of the residue using Prep-HPLC (water (0.2% formic acid), 20%˜60% acetonitrile, 15 minutes) gave 5-((5-methylaminopyrimidin-4-yl) amino)-4-methoxyisodihydroindole-1-one formate 82 (2.0 mg, 0.007 mmol, white solid).

Yield: 10%.

MS m/z (ESI): 286 [M+1],

¹H NMR (400 MHz, CD₃OD) δ 8.26 (d, J=8.4 Hz, 1H), 8.17 (s, 1H), 7.73 (s, 1H), 7.51 (d, J=8.4 Hz, 1H), 4.68 (s, 2H), 4.01 (s, 3H), 2.91 (s, 3H).

### Example 83

### 5-((6-((3-Chloropyridin-2-yl)amino)pyrimidin-4-yl)amino)-4-methoxyisoindolin-1-one

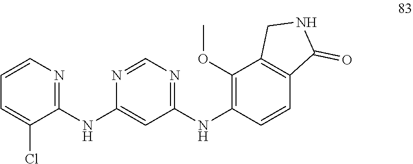 [SMILES: COc1c(Nc2cc(Nc3ncccc3Cl)ncn2)ccc2c1CNC2=O]

The synthetic procedure is similar to that of example 78. Using 6-chloro-N-(3-chloropyridin-2-yl)pyrimidine-4-amine instead of 6-chloro-N-(pyridin-2-yl)pyrimidine-4-amine, the desired compound 5-((6-((3-Chloropyridin-2-yl)amino)pyrimidin-4-yl)amino)-4-methoxyisoindolin-1-one was obtained. Yield: 17%.

MS m/z (ESI): 383 & 385 [M+1],

¹H NMR (400 MHz, DMSO-d₆) δ 9.01 (brs, 1H), 8.51 (brs, 1H), 8.32 (s, 1H), 8.31 (d, J=8.0 Hz, 1H), 8.14 (d, J=8.0 Hz, 1H), 7.96 (d, J=8.0 Hz, 1H), 7.59 (s, 1H), 7.34 (d, J=8.0 Hz, 1H), 7.12-7.09 (m, 1H), 6.05 (brs, 1H), 4.56 (s, 2H), 3.91 (s, 3H).

### Example 84

### 5-((6-(Pyrazin-2-ylamino) pyrimidin-4-yl) amino)-4-methoxyisodihydroindol-1-one formate

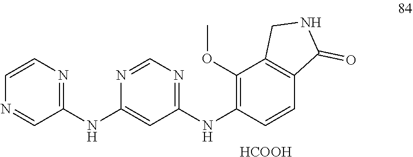 [SMILES: COc1c(Nc2cc(Nc3cnccn3)ncn2)ccc2c1CNC2=O ; "HCOOH"]

The synthetic procedure is similar to that of example 69. Using 2-chloropyrazine instead of 2-chloropyrimidine, the desired compound: 5-((6-(pyrazin-2-ylamino) pyrimidin-4-yl) amino)-4-methoxyisodihydroindol-1-one formate was obtained.

Yield: 4%.

MS m/z (ESI): 350 [M+1],

¹H NMR (400 MHz, DMSO-d6) δ 10.21 (s, 1H), 9.03 (s, 1H), 8.85 (s, 1H), 8.55 (s, 1H), 8.35 (s, 1H), 8.29 (s, 1H), 8.23 (s, 1H), 8.15 (s, 1H), 8.11 (d, J=8.2 Hz, 1H), 7.42 (s, 1H), 7.35 (d, J=8.2 Hz, 1H), 4.57 (s, 2H), 3.92 (s, 3H).

### Example 85

### 5-((6-((5-Chloropyridin-2-yl)amino)pyrimidin-4-yl)amino)-4-methoxyisoindolin-1-one

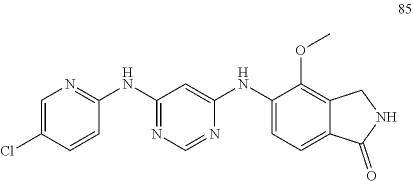 [SMILES: COc1c(Nc2cc(Nc3ccc(Cl)cn3)ncn2)ccc2c1CNC2=O]

The synthetic procedure is similar to that of example 69. Using 2, 5-dichloropyridine instead of 2-chloropyrimidine, the desired compound: 5-((6-((5-Chloropyridin-2-yl)amino)pyrimidin-4-yl)amino)-4-methoxyisoindolin-1-one was obtained. Yield: 9%.

MS m/z (ESI): 383 & 385 [M+1],

¹H NMR (400 MHz, DMSO-d6) δ 10.02 (s, 1H), 8.92 (s, 1H), 8.51 (s, 1H), 8.33 (s, 1H), 8.28 (s, 1H), 8.08 (d, J=8.0 Hz, 1H), 7.80 (d, J=8.8 Hz, 1H), 7.62 (d, J=8.8 Hz, 1H), 7.34 (d, J=8.0 Hz, 1H), 7.32 (s, 1H), 4.56 (s, 2H), 3.91 (s, 3H).

### Example 86

### 5-((6-((6-Chloropyridin-2-yl)amino)pyrimidin-4-yl)amino)-4-methoxyisoindolin-1-one

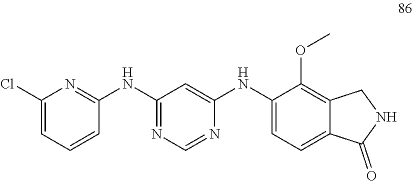 [SMILES: COc1c(Nc2cc(Nc3cccc(Cl)n3)ncn2)ccc2c1CNC2=O]

The synthetic procedure is similar to that of example 69. Using 2, 6-chloropyridine instead of 2-chloropyrimidine, the desired compound: 5-((6-((6-Chloropyridin-2-yl)amino)pyrimidin-4-yl)amino)-4-methoxyisoindolin-1-one was obtained. Yield: 9%.

MS m/z (ESI): 383 & 385 [M+1],

¹H NMR (400 MHz, DMSO-d6) δ 10.16 (s, 1H), 8.96 (s, 1H), 8.54 (s, 1H), 8.34 (s, 1H), 7.90 (d, J=7.2 Hz, 1H), 7.74 (t, J=8.0 Hz, 1H), 7.66 (d, J=8.0 Hz, 1H), 7.35 (d, J=8.0 Hz, 1H), 7.11 (s, 1H), 7.00 (d, J=7.2 Hz, 1H), 4.57 (s, 2H), 3.91 (s, 3H).

### Example 87

### 5-((6-((4-Chloropyridin-2-yl)amino)pyrimidin-4-yl)amino)-4-methoxyisoindolin-1-one

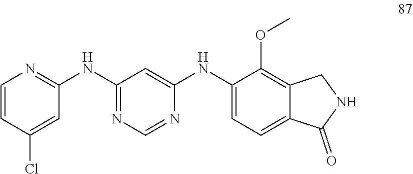 [SMILES: COc1c(Nc2cc(Nc3cc(Cl)ccn3)ncn2)ccc2c1CNC2=O]

The synthetic procedure is similar to that of example 69. Using 2-fluorine-4-chloropyridine instead of 2-chloropyrimidine, the desired compound: 5-((6-((4-Chloropyridin-2-yl)amino)pyrimidin-4-yl)amino)-4-methoxyisoindolin-1-one was obtained. Yield: 10%.

MS m/z (ESI): 383 & 385 [M+1],

¹H NMR (400 MHz, DMSO-d6) δ 9.99 (s, 1H), 8.89 (s, 1H), 8.45 (s, 1H), 8.29 (s, 1H), 8.17 (d, J=5.6 Hz, 1H), 8.00 (d, J=8.0 Hz, 1H), 7.69 (s, 1H), 7.27 (d, J=8.0 Hz, 1H), 7.26 (s, 1H), 6.98 (d, J=5.6 Hz, 1H), 4.49 (s, 2H), 3.84 (s, 3H).

MNK1 Inhibitory Activity Assay:

Evaluation of the effects of compounds of the invention on mitogen-activated protein kinase kinase 1 (MNK1) activity using in vitro kinase assays.

The experimental methods are summarized as follows: The in vitro activity of MNK1 was determined by measuring the level of ADP produced in the kinase reaction using the ADP-Glo Kinase Assay Kit. The reaction buffer consisted of the following components: 50 mM HEPES, pH 7.5, 10 mM MgCl₂, 1 mM EGTA, 0.01% Brij35. Human recombinant full-length MNK1 protein (Thermo, Cat. No. PR9138A) was diluted with a reaction buffer to a 3.13 ng/uL kinase solution. The substrate reaction solution consisted of a substrate diluted to 0.75 mg/ml in reaction buffer (GRSRSRSRSR, available from Scilight Domestic Company), 2250 uM ATP, the ADP-Glo reagent and kinase assay solution from Promega kit (Promega, V9102).

Compounds were diluted in 100% DMSO to 100 uM, then serially diluted in 4 fold each time with DMSO to a minimum concentration of 0.0061 uM, and each concentration point was diluted 20-fold with the reaction buffer. If the compound IC50 value is very low, the initial concentration of the compound can be lowered. To a 384-well assay plate (Thermo, Cat. No. 264706) were added 1 uL of compound solution and 2 uL of MNK1 kinase solution. The mixture was mixed well and incubated for 30 minutes at room temperature; then 2 uL of substrate reaction solution was added, the reaction mixture was incubated for 120 minutes at room temperature. The reaction was terminated after an equal volume of 5uLADP-Glo solution was added, and the remaining ATP was completely consumed. After mixing for 60 minutes at room temperature, 10 ul of the kinase assay solution was added, and the mixture was uniformly mixed, and allowed to stand at room temperature for 40 minutes in the dark. The detection solution converts ADP into new ATP through coupled luciferase/luciferin, and ATP is converted into optical signal by Ultr-Glo™ luciferase, which can be detected by Envision. The intensity of the light signal is proportional correlated with the amount of ADP produced in the kinase reaction, therefore, the activity of MNK1 kinase can be determined. In this experiment, the group without protein was used as a negative control (100% inhibition). The group with protein but without compounds was used as a positive control (0% inhibition).

The percent inhibition of MNK1 activity by a compound can be calculated by the following formula:

Inhibition percentage=100−100*(signal_{compound}−signal_{negative control})/(signal_{positive control}−signal_{negative control}

Compound IC₅₀ value was calculated by the following formula using 10 concentration points and XLfit (ID Business Solutions Ltd., UK) software

Y=Bottom+(Top−Bottom)/(1+10\hat{ }((Log IC₅₀−X)*slope factor))

where Y is inhibition percentage. Bottom is the bottom plateau of the curve, Top is the top plateau of the curve, and X is the logarithm of the concentration of the test compound.

The results of the enzyme experiment test are shown in Table 1 below.

TABLE 1

| Compound | MNK1 IC50 |
|---|---|
| Number | (nM) |
| 1 | 15.5 |
| 2 | 59.5 |
| 3 | 11.3 |
| 4 | 1.45 |
| 5 | 585.3 |
| 6 | 29.8 |
| 7 | 44.9 |
| 8 | 64.1 |
| 9 | 294.1 |
| 10 | 23.8 |
| 11 | 531.76 |
| 12 | 729.5 |
| 13 | 224.7 |
| 14 | 1105.9 |
| 15 | 21.0 |
| 16 | n/a |
| 17 | 27.9 |
| 18 | 10.5 |
| 19 | 45.0 |
| 20 | 29.2 |
| 21 | 78.2 |
| 22 | 23.3 |
| 23 | 31.3 |
| 24 | 42.2 |
| 25 | 40.0 |
| 26 | 36.6 |
| 27 | 43.8 |
| 28 | 539.2 |
| 29 | 57.9 |
| 30 | 140.7 |
| 31 | 126.3 |
| 32 | 165.6 |
| 33 | 94.7 |
| 34 | 450.3 |
| 35 | 32.5 |
| 36 | 7.8 |
| 37 | 23.8 |
| 38 | 11.9 |
| 39 | 13.0 |
| 40 | 1137.1 |
| 41 | 1048.4 |
| 42 | 600.1 |
| 43 | 264.5 |
| 44 | 1004.4 |
| 45 | 35.3 |
| 46 | 49.4 |
| 47 | 79.1 |
| 48 | 187.0 |
| 49 | 1052.0 |
| 50 | 253.9 |
| 51 | 197.5 |
| 52 | 14.4 |
| 53 | 22.8 |
| 54 | 34.3 |
| 55 | 187.4 |
| 56 | 19.8 |
| 57 | 323.1 |
| 58 | 6.1 |
| 59 | 432.2 |
| 60 | 792.3 |
| 61 | 593.8 |
| 62 | 75.3 |
| 63 | 97.1 |
| 64 | 437.2 |
| 65 | 693.8 |
| 66 | 38.5 |
| 67 | 2.7 |
| 68 | 8.2 |
| 69 | 27.3 |
| 70 | 17.0 |
| 71 | 128.1 |
| 72 | 30.5 |
| 73 | 188.3 |
| 74 | 38.4 |
| 75 | 34.4 |
| 76 | 571.9 |
| 77 | 899.7 |
| 78 | 19.7 |
| 79 | 20.9 |
| 80 | 11.7 |
| 81 | 102.7 |
| 82 | 154.2 |
| 83 | 96.6 |
| 84 | 5.0 |
| 85 | 13.9 |
| 86 | 18.5 |
| 87 | 19.1 |

### MV-4-11 Cell Proliferation Inhibition Assay:

The effect of the compounds of the present invention on the proliferation of MV-4-11 cells was evaluated using a luminescent cell viability assay.

The experimental methods are summarized as follows:

The cell proliferation status of MV-4-11 was detected using the CellTilter-Glo (CTG) assay kit: the luminescence signal generated in the assay is proportional to the number of viable cells in the culture medium by using a unique, stable luciferase to detect viable cell metabolism indicator ATP.

CellTilter-Glo reagent (Promega, G7572) consists of CellTilter-Glo lyophilized powder and ellTilter-Glo buffer, which can be used to dissolve the lyophilized powder into the buffer.

MV-4-11 cells (ATCC #CRL-9591, purchased from Nanjing Kezhen, item number CBP60522) were cultured in IMDM complete medium (Thermofisher, 12440053) containing 10% FBS (GBICO, 10099-141) and 100 units/ml streptomycin mixture (Thermofisher, 15140122). when the cells coverage rearched 80-90% in the culture container, dilute and plant with 0.25% trypsin (including EDTA) (Thermofisher, 25200056) in a white 384-well plate (Thermofisher, 164610), 400 cells per well (36 μl DMEM complete medium), then 384-well plates were incubated overnight (18-20 hours) in a 37° C., % CO2 incubator.

Compounds were solubilized to 10 mM in 100% DMSO, then sequentially diluted in 4-fold with DMSO to a minimum concentration of 0.61 mM, and each concentration was diluted 50-fold with FBS-free IMDM medium. If the compound IC50 value is very low, the initial concentration of the compound can be lowered. 4 μl of the diluted IMDM compound was added to each well and mixed gently by centrifugation. The GI50 (50% Growth Inhibition) of the compounds was determined in this experiment, including the T0 group before the cell proliferation (including the T0 positive and T0 negative control groups), and the T5 group after 120 hours of cell proliferation (containing the T5 positive and T5 negative control groups).). T0 represents the number of cells before cell proliferation before compound addition, including positive control: the group with the addition of cells and 0.2% DMSO was used as the positive control. The group with medium alone was used as the negative control, T0 group before the addition of compound will be used for CTG test. In addition, the same positive and negative groups were prepared and used as the control groups for cell proliferation after 120 hours. The 384-well plate was placed in a 37° C., 5% CO2 incubator for further incubation. After 120 hours, it was taken out and allowed to stand at room temperature for 30 minutes. The CTG reagent was also taken out to room temperature. 20 μl of CTG reagent was added to each well, and placed on a shaker. Gently shake for 5 minutes to ensure sufficient cell lysis, leave for 10 minutes to stabilize the luminescence signal, and then read the luminescence signal with EnVision (Perkin Elmer).

The inhibition of MV-4-11 cell proliferation of the compounds was calculated by the following formula:

Inhibition percentage=100−100*[(signal T5compound−signal T5negative)−(signal T0positive−signal T0negative)]/[(signal T5positive control−signal T5negative control)−(signal T0positive−signal T0negative)]

The IC50 value of the compound was calculated from the 8 concentration points by XLfit (ID Business Solutions Ltd., UK) software using the following formula:

Y=Bottom+(Top−Bottom)/(1+10\hat{ }((Log GI50−X)*slope factor))

where Y is the percent inhibition and Bottom is the bottom plateau of the curve, Top is the top plateau of the curve, and X is the logarithm of the concentration of the test compound.

Cellular assays showed that the compounds of the examples of the present invention have a significant inhibitory effect on the proliferation of MV-4-11 tumor cells (IC₅₀<500 nM), as shown in Table 2 below

TABLE 2

| Compound number | MV-4-11 IC50 (nM) |
|---|---|
| 4 | 301 |
| 67 | 483 |
| 68 | 295 |
| 84 | 278. |

1. A compound as shown in Formula (I), or an isomer, a prodrug, a solvate, a stable isotopic derivative and a pharmaceutically acceptable salt thereof: 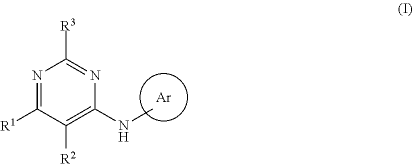
Where
R¹, R², R³ are each independently selected from the group consisting of hydrogen, halogen, cyano, C1-C8 alkyl, C3-C8 cyclyl, 3-8 membered heterocyclyl, aryl, heteroaryl, —C(O)R⁴, alkenyl, alkynyl, —OR⁴, —NR⁵ R⁶, —OC(O)NR⁵R⁶, —C(O)OR⁴, —C(O)NR⁵R⁶, —NR⁵C(O)R⁴, —NR⁴C(O)NR⁵R⁶, —S(O)ₘR⁴, —NR⁵S(O)ₘR⁴, —SR⁴, —S(O)ₘNR⁵R⁶, —NR⁴S(O)ₘNR⁵R⁶, where said alkyl, alkenyl, alkynyl, cyclyl, hetercyclyl, aryl, heteroaryl are optionally substituted by one or more substituents selected from halogen, cyano, C1-C8 alkyl, C3-C8 cyclyl, 3-8-membered heterocyclyl, —OR⁷, —OC(O)NR⁸R⁹, —C(O)OR⁷, —C(O)NR⁸R⁹, —C(O)R⁷, —NR⁸R⁹, —NRC(O)R⁷, —NR⁷C(O)NR⁸R⁹, —S(O)mR⁷, —NR⁸S(O)mR⁷, —SR⁷, —S(O)mNR⁸R⁹, —NR'S(O)mNR⁸R⁹;
Where ring Ar are each independently selected from substituted or unsubstituted aryl or heteroaryl, when Ar is substituted, it could be substituted by one or more substituents at any position, where said substituents are each independently selected from the group consisting of hydrogen, halogen, cyano, C1-C8 alkyl, C3-C8 cyclyl, 3-8 membered heterocyclyl, aryl, heteroaryl, —C(O)R⁴, —C(O)OR⁴, alkenyl, alkynyl, OR⁴, —NR⁵R⁴, —NR⁵C(O)R⁴, —NR⁴C(O)NR⁵R⁶, —S(O)mR⁴, —NR⁵S(O)mR⁴, —SR⁴, —S(O)mNR⁵R⁶, —NR⁴S(O)mNR⁵R⁶, where said alkyl, alkenyl, alkynyl, cyclyl, hetercyclyl, aryl, heteroaryl are optionally substituted by one or more substituents selected from the group consisting of halogen, cyano, C1-C8 alkyl, C3-C8 cyclyl, 3-8 membered heterocyclyl, —OR⁷, —OC(O)NR⁸R⁹, —C(O)OR⁷, —C(O)NR⁸R⁹, —C(O)R⁷, —NR⁸R⁹, —NR⁸C(O)R⁷, —NR⁷C(O)NR⁸R⁹, —S(O)mR¹, —NR⁸S(O)mR⁷, —SR⁷, —S(O)mNR⁸R⁹, —NR⁷S(O)mNR⁸R⁹.
R¹ and R² may form a 5-8 membered heterocyclyl together with the carbon atom to which they are attached;
R⁴, R⁵, R⁶, R⁷, R¹, R⁹ are each independently selected from the group consisting of hydrogen, C1-C8 alkyl, heteroalkyl, C3-C8 cyclyl, 3-8 membered monocyclic heterocyclyl, monocyclic heteroaryl or monocyclic aryl, alkenyl, alkynyl, where said R⁵ and R⁶, R⁸ and R⁹ may form a 3-7 membered heterocyclyl together with the carbon atom to which they are attached; and m is 1 or 2.

2. The compound according to claim 1, an isomer, a prodrug, a solvate, a stable isotopic derivative thereof or a pharmaceutically acceptable salt thereof, characterized in that the compound is of the following formula (II) a-g: 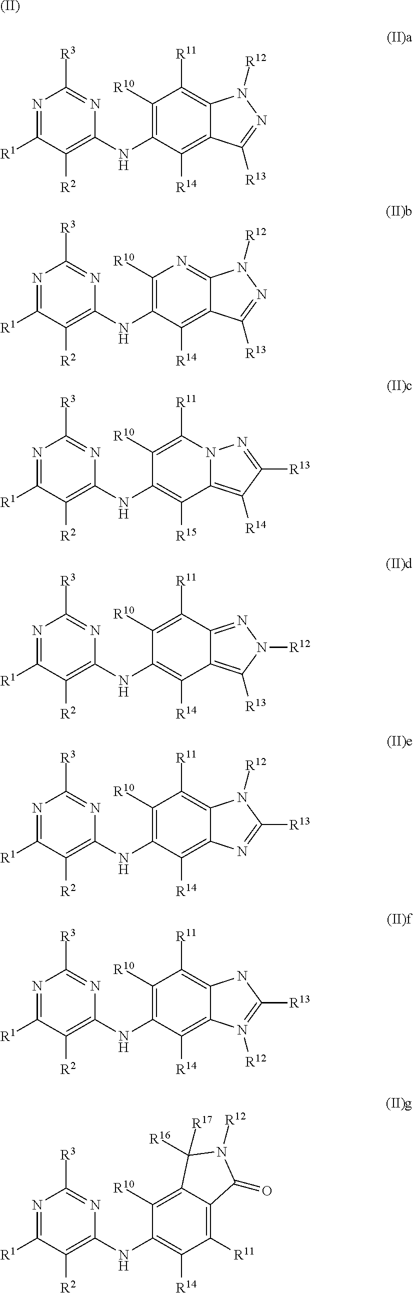
where:
R¹, R², R³, R¹⁰, R¹¹, R¹³, R¹⁴, R¹⁵ are each independently selected from the group consisting of hydrogen, halogen, cyano, C1-C8 alkyl, C3-C8 cyclyl, 3-8 membered heterocyclyl, aryl, heteroaryl, aldehyde group, —C(O)R⁴, carboxyl, alkenyl, alkynyl, —OR⁴, —NR⁵R⁶, —NR⁵C(O)R⁴, —NR⁴C(O)NR⁵R⁶, —S(O)mR⁴, —NR⁵S(O)mR⁴, —SR⁴, —S(O)mNR⁵R⁶, —NR⁴S(O)mNR⁵R⁶, where said alkyl, cyclyl, heterocyclyl, aryl, or heteroaryl are optionally substituted by one or more substituents selected from the group consisting of halogen, cyano, C1-C8 alkyl, C3-C8 cyclyl, 3-8 membered heterocyclyl, —OR⁷, —OC(O)NR⁸R⁹, —C(O)OR⁷, —C(O)NR⁸R⁹, —C(O)R⁷, —NR⁸R⁹, —NR⁷C(O)R⁷, —NR⁷C(O)NR⁸R⁹, —S(O)mR⁷, —NR'S(O)mR⁷, —SR⁷, —S(O)mNR⁸R⁹, —NR⁷S(O)mNR⁸R⁹;
R² was selected from the group consisting of hydrogen, C1-C8 alkyl, C3-C8 cyclyl, 3-8 membered monocyclic heterocyclyl, monocyclic heteroaryl or monocyclic aryl, where said alkyl, cyclyl, heterocyclyl, aryl or heteroaryl are optionally substituted by one or more substituents selected from the group consisting of halogen, cyano, C1-C8 alkyl, C3-C8 cyclyl, 3-8 membered heterocyclyl, —OR⁷, —OC(O)NR⁸R⁹, —C(O)OR⁷, —C(O)NR⁸R⁹, —C(O)R⁷, —NR⁸R⁹, —NR⁷C(O)R¹, —NR⁷C(O)NR⁸R⁹, —S(O)mR⁷, —NR'S(O)mR⁷, —SR⁷, —S(O)mNR⁸R⁹, —NR'S(O)mNR⁸R⁹;
R¹⁶, R⁷ are each independently selected from the group consisting of hydrogen, C1-C8 alkyl, C3-C8 cyclyl, 3-8 membered monocyclic heterocyclyl, monocyclic heteroaryl or monocyclic aryl, where said alkyl, cyclyl, heterocyclyl, aryl or heteroaryl are optionally substituted by one or more substituents selected from the group of halogen, cyano, C1-C8 alkyl, C3-C8 cyclyl, 3-8 membered heterocyclyl, —OR⁷, —OC(O)NR⁸R⁹, —C(O)OR⁷, —C(O)NR⁸R⁹, —C(O)R⁷, —NR⁷R⁹, —NR⁸C(O)R⁷, —NR⁷C(O)NR⁸R⁹, —S(O)mR⁷, NR⁸S(O)mR⁷, —SR⁷, —S(O)mNR⁸R⁹, —NR'S(O)mNR⁸R⁹.
R¹ and R² may form a 5-8 membered hetero-cyclyl with the atom they are attached;
R¹⁰ and R¹¹ may form a 5-8 membered heterocyclyl with the atom they are attached;
R¹⁶ and R¹⁷ may form a 3-8 membered heterocyclyl with the atom they are attached;
the definition of R⁴⁻⁹ are described as above; and
m is 1 or 2.

3. The compound according to claim 1, an isomer, a prodrug, a solvate, a stable isotopic derivative thereof or a pharmaceutically acceptable salt thereof, characterized in that the compound is of the following Formula (III) a-e 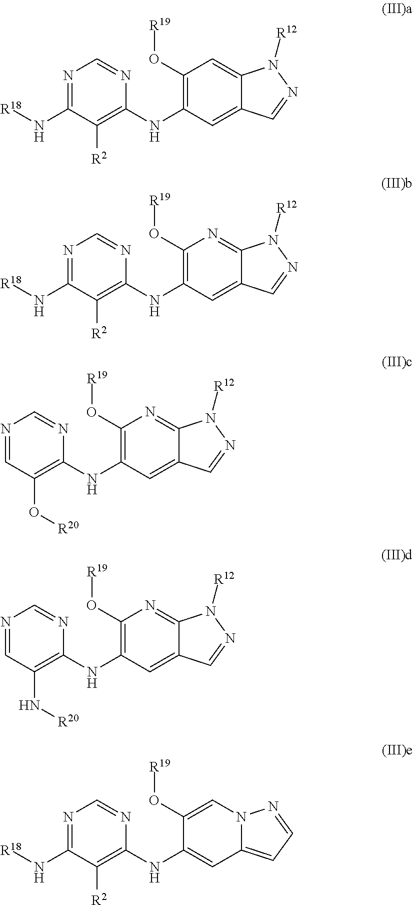
where:
R² is selected from the group consisting of hydrogen, fluoro, cyano, C1-C3 alkyl, C5-C6 cyclyl, 5-6 membered heterocyclyl, aryl, heteroaryl, —C(O)OR⁴, —C(O)NR⁵R⁶, carboxyl, —OR⁴, —NR⁵R⁶, where said cyclyl, heterocyclyl are optionally substituted by one or more substituents selected from the group consisting of —C(O)OR⁷, —C(O)NR⁸R⁹;
R¹² is selected from hydrogen, alkoxycarbonyl, alkylcarbonyl, cycloalkylcarbonyl; R¹⁸ is selected from hydrogen, C1-C5 alkyl, C3-C6 cycloalkyl, aryl, 5-6 membered heteroaryl, alkylcarbonyl, cycloalkylcarbonyl, arylcarbonyl, heteroarylcarbonyl, alkylsulfonyl, cycloalkylsulfonyl;
R¹⁹ is selected from C1-C4 alkyl;
R²⁰ is selected from C1-C5 alkyl, C1-C5 oxaalkyl, —CH₂CH₂NR⁵R⁶, phenyl;
R² and R¹⁸ may form a 5-8 membered ring with the carbon and nitrogen atoms they are attached;
R⁴, R⁵, R⁶, R⁷, R¹, R⁹ are each independently selected from the group consisting of hydrogen, C1-C5 alkyl, C3-C8 cyclyl, 3-8 membered monocyclic heterocyclyl, monocyclic heteroaryl or monocyclic aryl, alkenyl, alkynyl, where said R⁵ and R⁶, R⁸ and R⁹ may form a 3-7 membered heterocyclyl with the nitrogen they are attached.

4. The compound according to claim 1, an isomer, a prodrug, a solvate, a stable isotopic derivative thereof or a pharmaceutically acceptable salt thereof, characterized in that the compound is of Formula (III) f-g: 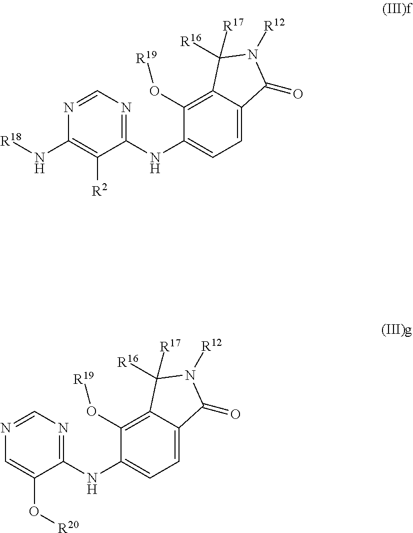
where,
R² is selected from the group consisting of hydrogen, fluoro, cyano, C1-C3 alkyl, C5-C6 cyclyl, 5-6 membered heterocyclyl, aryl, heteroaryl, —C(O)OR⁴, —C(O)NR⁵R⁶, carboxyl, —OR⁴, —NR⁵R⁶, where said cyclyl, heterocyclyl are optionally substituted by substituents selected from —C(O)OR⁷, —C(O)NR⁸R⁹;
R¹² is selected from C1-C6 alkyl, C5-C6 cyclyl;
R¹⁶, R¹⁷ are each independently selected from C1-C5 alkyl;
R¹⁸ is selected from hydrogen, C1-C5 alkyl, C3-C6 cycloalkyl, aryl, 5-6 membered heteroaryl, alkylcarbonyl, cycloalylcarbonyl, arylcarbonyl, heteroarylcarbonyl;
R¹⁹ is selected from C1-C4 alkyl;
R²⁰ is selected from C1-C5 alkyl, C1-C5 oxaalkyl, —CH₂CH₂NR⁵R⁶;
R² and R¹⁸ may form a 5-8 membered heterocyclyl with the atom they are attached;
R¹⁶ and R¹⁷ may form a 4-6 membered ring with the atom they are attached;
R⁴, R⁵, R⁶, R⁷, R¹, R⁹ are each independently selected from the group consisting of hydrogen, C1-C5 alkyl, C3-C8 cyclyl, 3-8 membered monocyclic heterocyclyl, monocyclic heteroaryl or monocyclic aryl, alkenyl, alkynyl, where said R⁵ and R⁶, R⁸ and R⁹ may form a 3-7 membered heterocyclyl with the nitrogen atoms they are attached.

5. The compound according to claim 1, an isomer, a prodrug, a solvate, a stable isotopic derivative thereof or a pharmaceutically acceptable salt thereof, which is selected from:

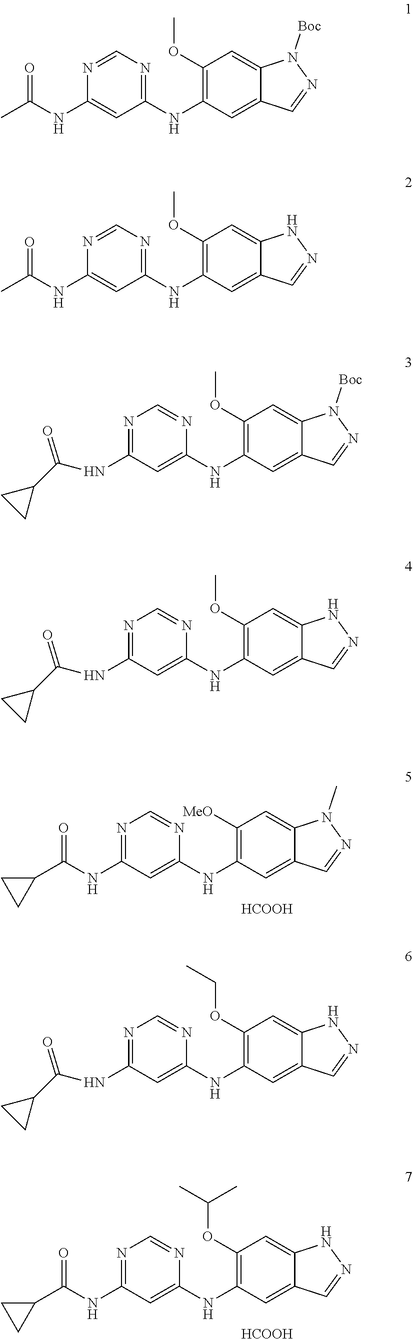

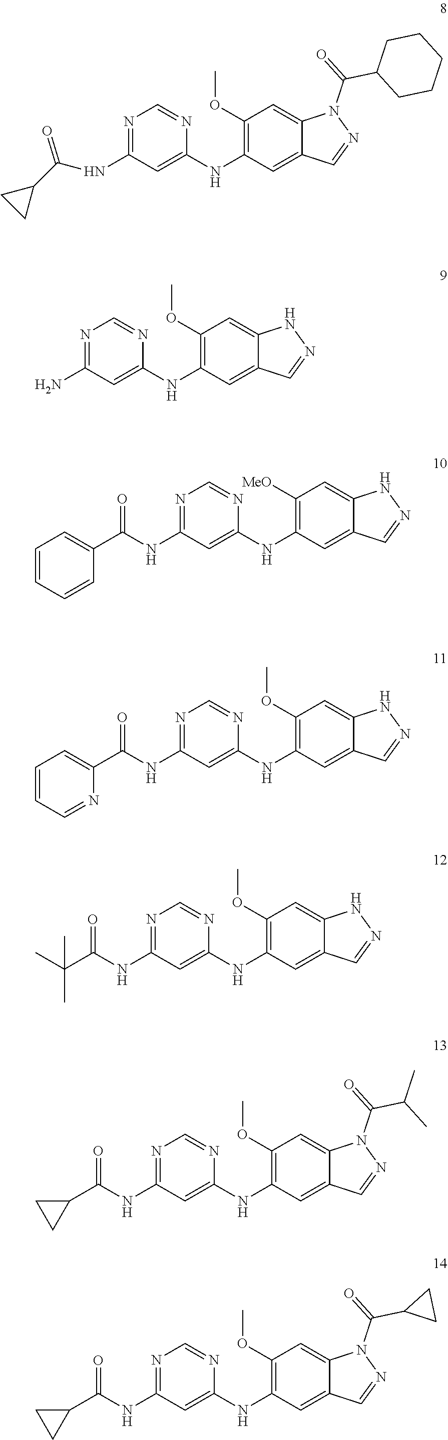

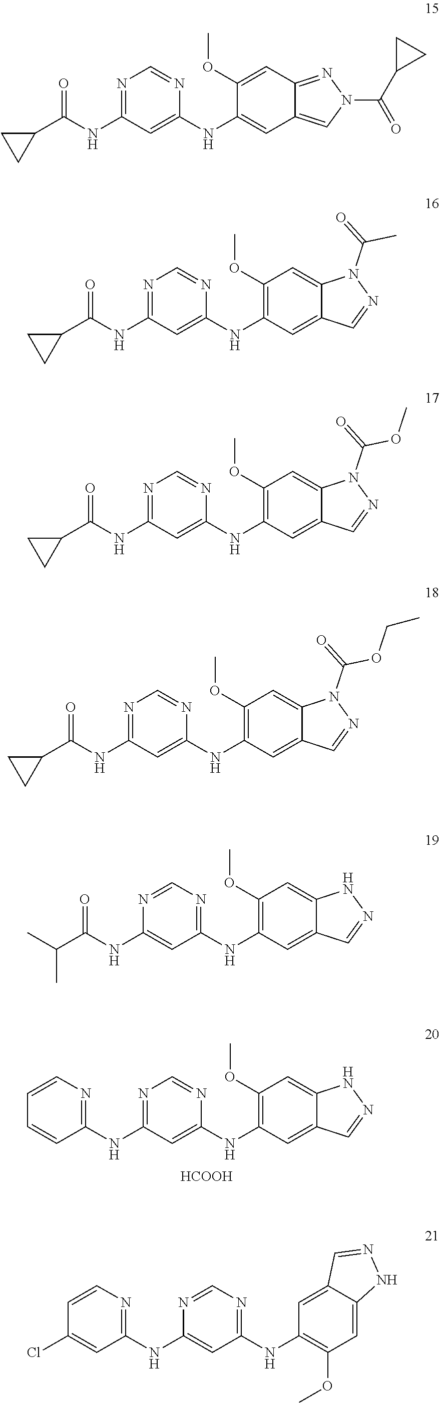

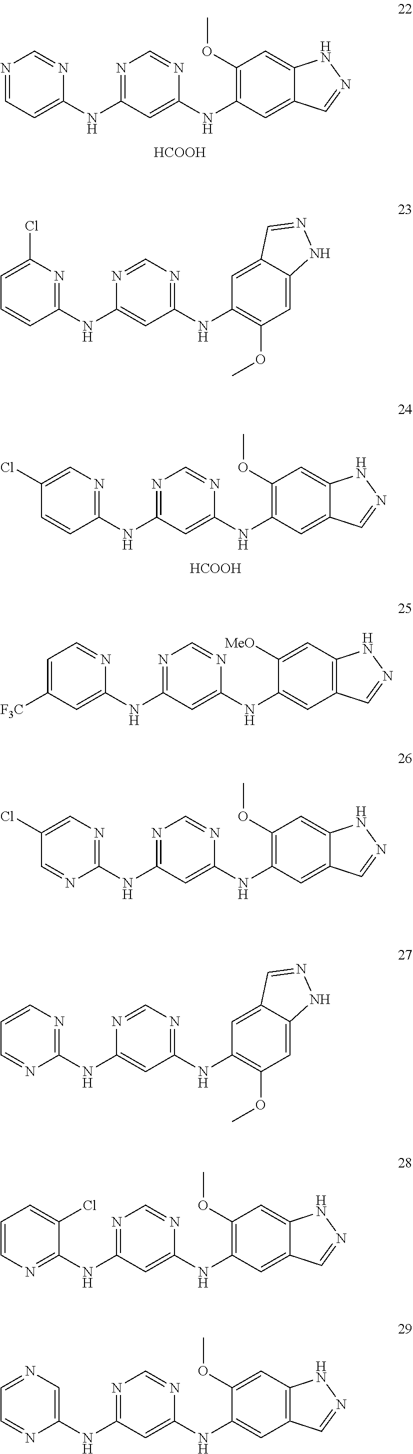

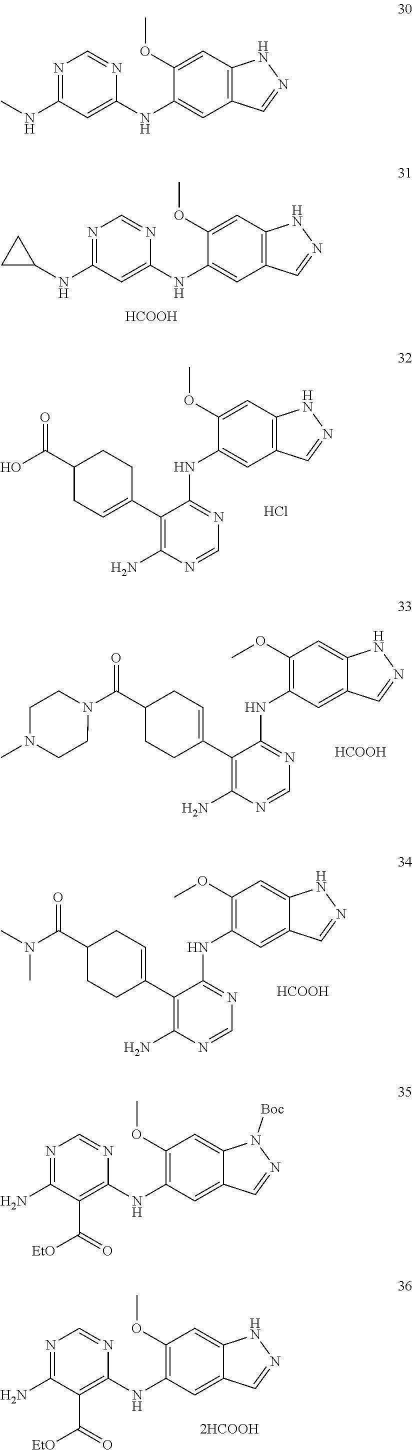

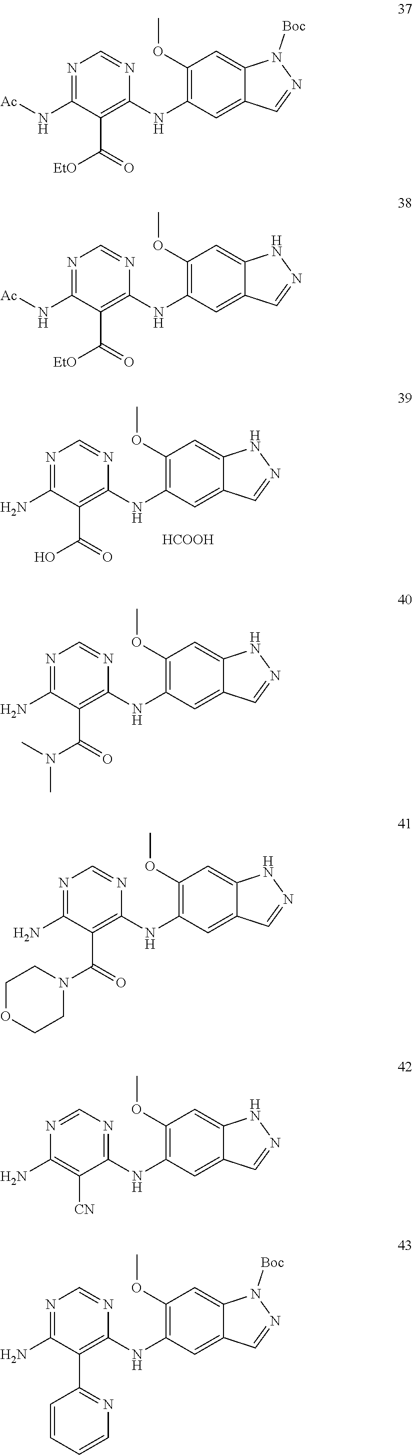

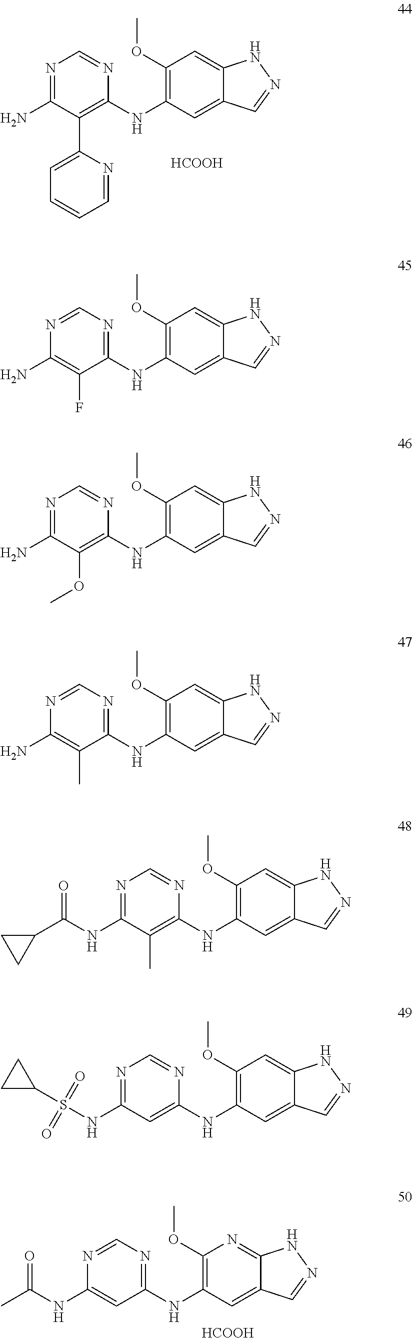

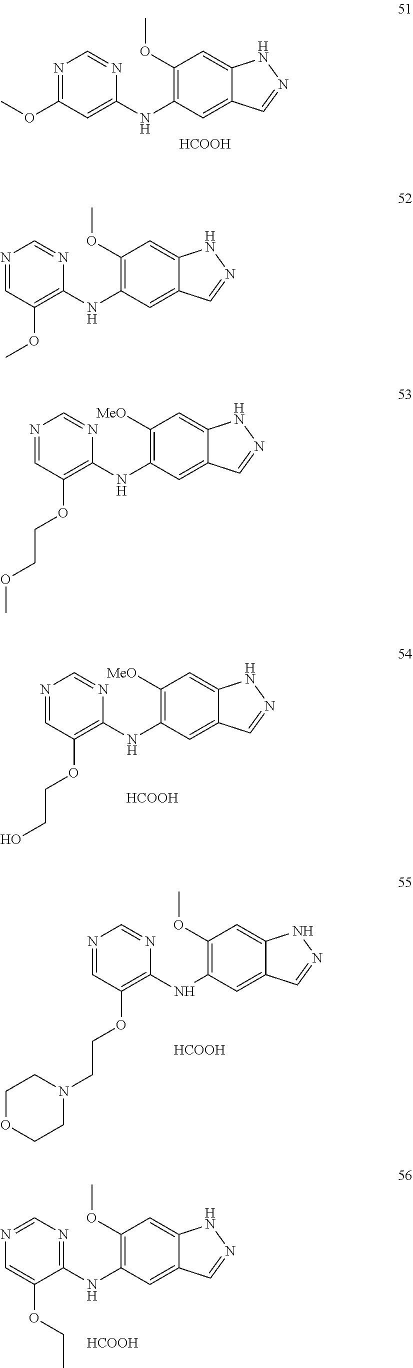

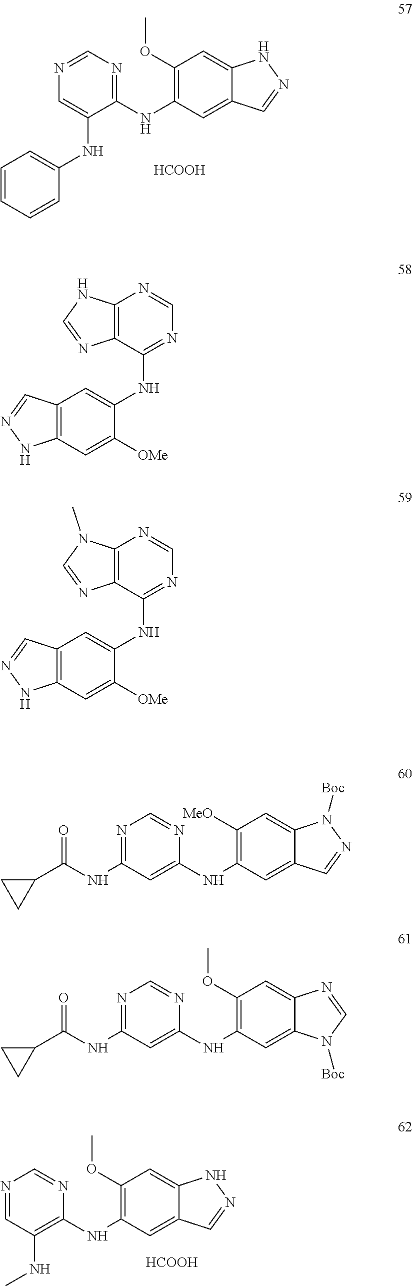

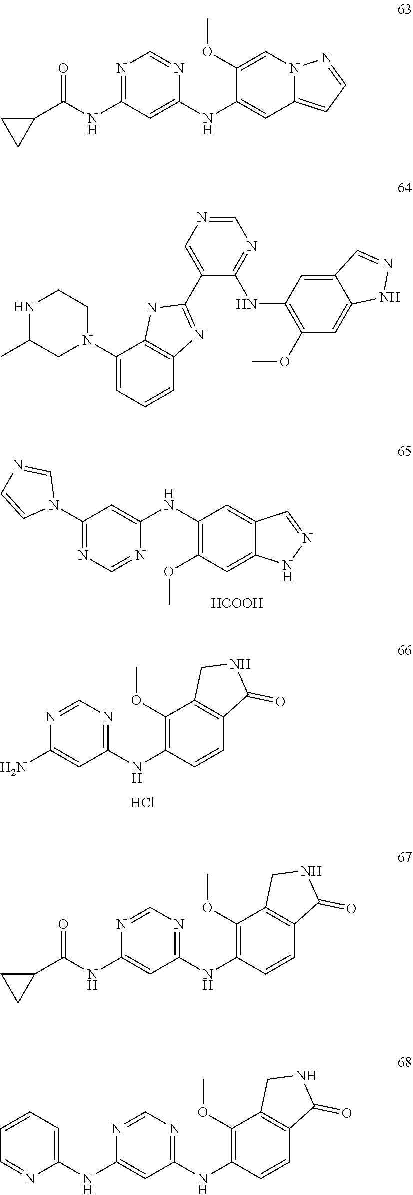

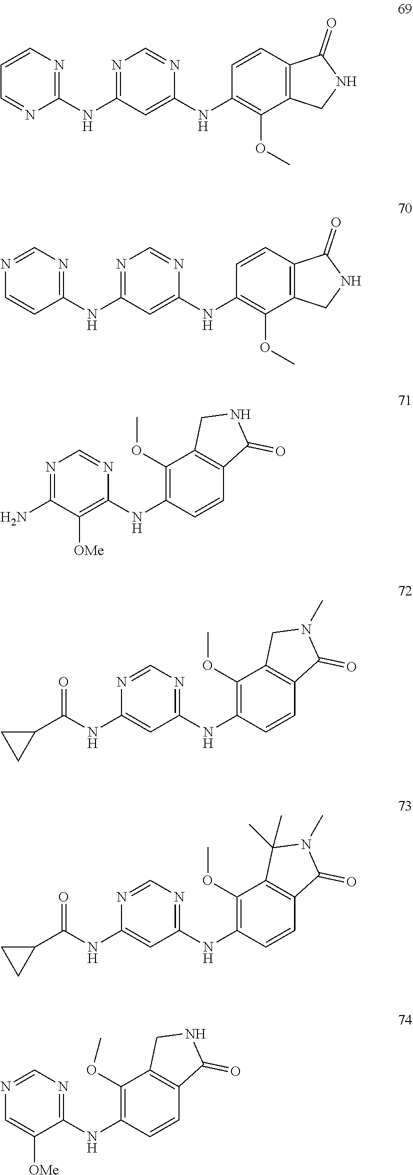 [SMILES: COc1c(Nc2cc(Nc3ncccn3)ncn2)ccc2c1CNC2=O ; COc1c(Nc2cc(Nc3ccncn3)ncn2)ccc2c1CNC2=O ; COc1c(N)ncnc1Nc1ccc2c(c1OC)CNC2=O ; COc1c(Nc2cc(NC(=O)C3CC3)ncn2)ccc2c1CN(C)C2=O ; COc1c(Nc2cc(NC(=O)C3CC3)ncn2)ccc2c1C(C)(C)N(C)C2=O ; COc1cncnc1Nc1ccc2c(c1OC)CNC2=O]

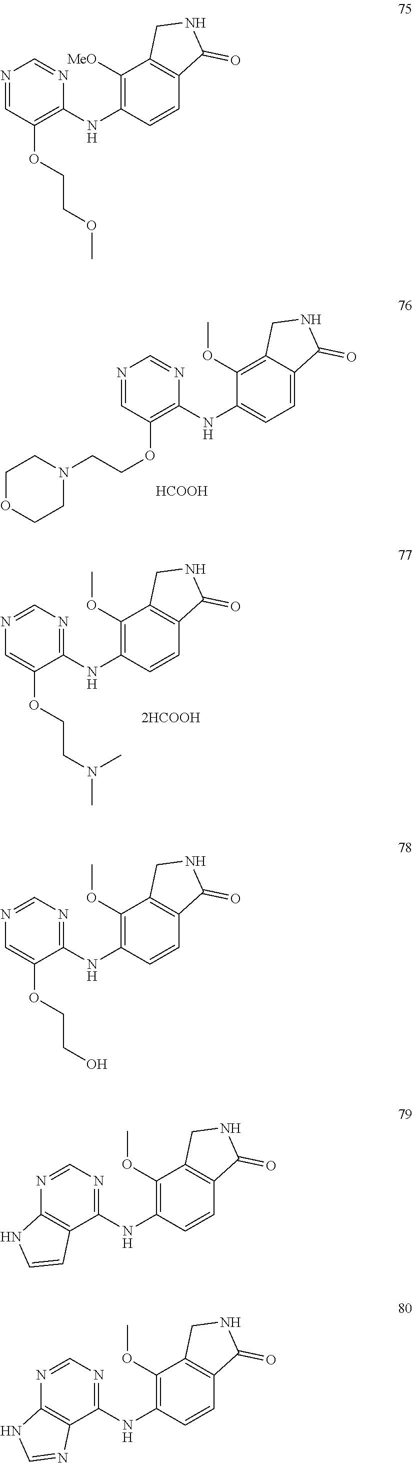

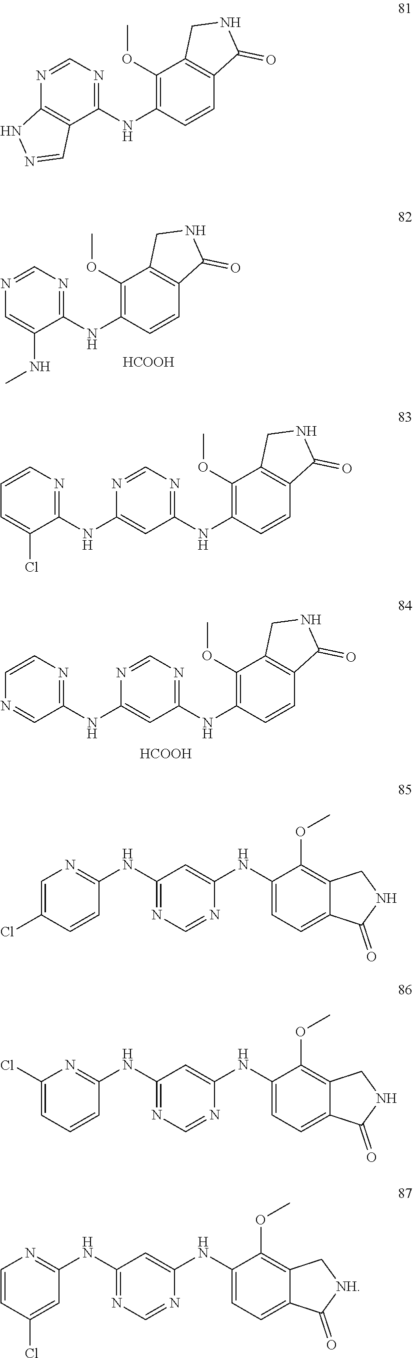

6. A pharmaceutical composition, comprising a compound according to claim 1 or an isomer, a prodrug, a stable isotopic derivative or a pharmaceutically acceptable salt thereof, and a pharmaceutically acceptable carrier, diluent and excipient.

7. (canceled)

8. A method for treating or preventing MNK-mediated diseases, comprising administering to a patient in need thereof a therapeutically effective amount of a compound according to claim 1 or an isomer thereof, a prodrug, a solvate, a stable isotope derivative or a pharmaceutically acceptable salt thereof.

9. (canceled)

10. The method of claim 8, wherein the MNK-mediated diseases are selected from malignant hematological diseases, lung cancer, breast cancer, ovarian cancer, prostate cancer, pancreatic cancer, and glioma.
